SEQUENCE LISTING (1 sequence)

SEQ ID NO: 1 - protein, 14 aa, Artificial sequence
  Synthetic construct
  MISC_FEATURE (1)..(1): The N-terminal lysine (K) forms an amide with 4-(dimethylaminoazo)benzene-4-carboxylic acid (Dabcyl)
  MISC_FEATURE (14)..(14): The C-terminal of Glutamic acid (E) forms an amide with (5-((2-Aminoethyl)amino)naphthalene-1-sulfonic acid) (Edans)
KTSAVLQSGFRKME

(19) United States
(12) Patent Application Publication
(10) Pub. No.: US 2022/0324800 A1
(43) Pub. Date: Oct. 13, 2022

(54) SARS-3CL PROTEASE INHIBITORS

(71) Applicant: Primmune Therapeutics, Inc., Carlsbad, CA (US)

(72) Inventor: Stephen E. Webber, San Diego, CA (US)

(21) Appl. No.: 17/657,454

(22) Filed: Mar. 31, 2022

Related U.S. Application Data
(60) Provisional application No. 63/169,613, filed on Apr. 1, 2021.

(51) Int. Cl. C07D 207/267; C07D 401/12; C07D 409/12; C07D 403/12; C07D 405/12; C07D 413/12; C07D 403/14; A61P 31/14

(52) U.S. Cl. CPC C07D 207/267; C07D 401/12; C07D 409/12; C07D 403/12; C07D 405/12; C07D 413/12; C07D 403/14; A61P 31/14

(57) ABSTRACT

The present invention relates to antiviral agents according to Formula I and their use in the treatment of viral infection.

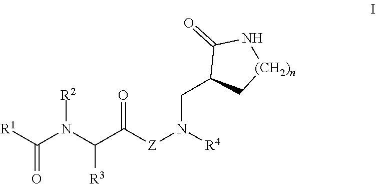

### CROSS-REFERENCE TO RELATED APPLICATION

This application claims the benefit of priority to U.S. Provisional Application No. 63/169,613, filed Apr. 1, 2021, the contents of which are incorporated herein by reference in their entirety.

### REFERENCE TO SEQUENCE LISTING

This application incorporates by reference a Computer Readable Form (CRF) of a Sequence Listing in ASCII text format submitted with this application, entitled 055014-503001WO_Sequence_Listing_ST25.TXT, was created on Mar. 28, 2022, and is 858 bytes in size.

### FIELD OF THE INVENTION

The present invention provides SARS-3CL protease inhibitors and their use in therapeutic applications.

### BACKGROUND OF THE INVENTION

Coronaviruses (CoVs) were first identified in 1960 and are classified as members of the family Coronaviridae. CoVs are enveloped, single-stranded RNA viruses that infect vertebrate animals, causing acute to chronic diseases in the respiratory, cardiac, enteric, and central nervous systems. In animals, the most common CoVs are infectious bronchitis virus (IBV), feline CoV (FeCoV), and mouse hepatitis virus (MHV), which infect chickens, felines, and rodents, respectively. To date, there are seven known CoVs that cause disease in humans: HCoV-229E, HCoV-NL63, HCoV-OC43, HCoV-HKU1, severe acute respiratory syndrome coronavirus (SARS-CoV), Middle East respiratory syndrome coronavirus (MERS-CoV) and, most recently, SARS-CoV-2, commonly known as coronavirus disease 2019 (COVID-19). The CoVs HCoV-229E, HCoV-NL63, HCoV-OC43, and HCoV-HKU1 cause mild symptoms, similar to a common cold. However, SARS-CoV, MERS-CoV, and SARS-CoV-2 can cause mild to severe symptoms related to upper respiratory infection such as fever, cough, dyspnea, pneumonia, acute respiratory distress syndrome (ARDS), and death.

The 2019 SARS-CoV-2 pandemic has seen a number of approved drugs and therapies repurposed in an attempt to find a treatment. While several antivirals and therapeutic methods have had mediocre success, none have proven overtly successful. As such, new more active and advantageous antiviral therapies, particularly those active against coronaviruses, are sought.

In the search for new antivirals the 3-chymotrypsin-like cysteine protease (3CLpro) of SARS-CoV-2 has arisen as an ideal target for direct antiviral agents for a number of reasons. First, 3CLpro is a key enzyme for SARS-CoV-2 polyprotein cleavage and viral replication. Thus, it is highly conserved within the group of CoVs, and in a related form, by members of the Enterovirus genus (picornavirus). And most importantly, there is no human homologue of 3CLpro, setting the stage for high selectivity and minor off target effects. Currently, there are only two 3CLpro inhibitors in preclinical development for the treatment of COVID-19: GC-376 and PF-07304814 (a prodrug of PF-00835231). The presently disclosed embodiments address the need for additional and improved 3CLpro targeting antivirals.

### SUMMARY OF THE INVENTION

The present invention generally relates to compounds useful as antiviral agents, compositions comprising these compounds thereof, methods for their manufacture, and methods for their use.

In one embodiment the present invention is directed to antiviral agents according to Formula I:

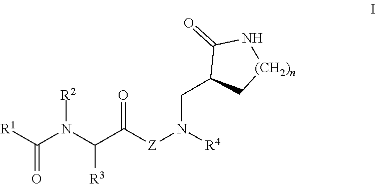

wherein
R¹ is —H, alkyl, —O-alkyl, aryl, alkylene-aryl, —O-aryl, cycloalkyl, alkylene-cycloalkyl, —O— cycloalkyl, heterocyclyl, alkylene-heterocyclyl, —O-heterocyclyl, or —NR⁵R⁶;
R² is —H or alkyl;
or R¹ and R², together with the atoms to which they are bonded, can form a 5- or 6-membered heterocyclic ring;
R³ is —H, or alkyl, —CH₂-alkenyl, or —CH₂-alkynyl;
or R² and R³, together with the atoms to which they are bonded, can form a 5- or 6-membered heterocyclic ring;
R⁴ is —CN, —C(O)R⁷, —C(S)R⁷, —SO₂R⁸, —C(O)CH₂R⁹, —SO₂CH₂R⁸ or —CH₂R¹⁰;
each of R⁵ and R⁶ are independently —H, alkyl, alkenyl, alkynyl, alkylene-X, —C(O)alkyl, —O— alkyl, or —S(O)ₘalkyl;
or R⁵ and R⁶, when bonded to the same atom can, together with the atom to which they are bonded, form a heterocyclic ring;
R⁷ is —H, alkyl, alkenyl, alkynyl, cycloalkyl, heterocyclyl, alkylene-X, alkenyl-C(O)Oalkyl, alkenyl-S(O)ₘalkyl, —C(O)alkyl, —O-alkyl, —S(O)ₘalkyl, or —S(O)ₘalkylene-aryl;
R⁸ is alkenyl, alkynyl or alkylene-X;
R⁹ is halogen, —CN or —OC(O)alkyl;
R¹⁰ is halogen or —CN;
X is halogen or —OC(O)aryl;
Z is —CH₂—, —NCH₃—, or —NH—;
m is 0, 1, or 2; and
n is 1 or 2;
wherein when Z is —NH— and R³ is alkyl, R⁴ is then —C(O)R⁷, —C(S)R⁷, —SO₂R⁸, —C(O)CH₂R⁹, —SO₂CH₂R⁸ or —CH₂R¹⁰; and,

wherein each alkyl, alkenyl, alkynyl, alkylene, aryl, cycloalkyl, and heterocyclyl is independently optionally substituted with 1, 2 or 3 groups selected from —OH, —CN, —(C₁-C₄) alkylNHC(O)(C₁-C₄)haloalkyl, alkylene-aryl-NHC(O)heteroaryl, —SH, —S(O)NH₂, halogen, —NH₂, —NH(C₁-C₄)alkyl, —N[(C₁-C₄)alkyl]2, —C(O)NH₂, —COOH, —COOMe, acetyl, —(C₁-C₅)alkyl, —O(C₁-C₈)alkyl, (C₂-C₅)alkenyl, (C₂-C₅)alkynyl, thioalkyl, cyanomethylene, —NH-heterocyclyl, —NH—C(O)(C₁-C₄)alkyl, —NH—C(O)— heterocycloalkyl, —NH-heterocycloalkyl, —NH—C(O)-alkylene, —NH—C(O)—O-alkylene, —CH₂-C(O)-alkyl, —C(O)-alkyl, cycloalkyl, —C(O)-cycloalkyl, —CH₂-C(O)-aryl, —CH₂-aryl, —C(O)-aryl, —C(O)-heterocycloalkyl, —CH₂-C(O)-heterocyclyl, —C(O)— heterocyclyl, or heterocyclyl;

or a stereoisomer, a tautomer or a pharmaceutically acceptable salt thereof.

In other embodiments, the antiviral agent(s) of the invention may be used alone, or in association with other therapeutic agents and/or therapeutic procedures, for treating or preventing viral infection in a subject in need of such treatment or prevention.

In other embodiments, the present invention provides a pharmaceutical composition comprising (i) a therapeutically effective amount of at least one compound according to Formula I or a stereoisomer, a tautomer or a pharmaceutically acceptable salt thereof; (ii) in combination with a pharmaceutically acceptable carrier, diluent or excipient. Pharmaceutical compositions comprising a pharmaceutically acceptable carrier, diluent, or excipient, in association with further therapeutic agents are also part of the present invention.

The above embodiments and other aspects of the invention are readily apparent in the detailed description that follows. To this end, various references are set forth herein which describe in more detail certain background information, procedures, compounds and/or compositions, and are each hereby incorporated by reference in their entireties.

### DETAILED DESCRIPTION

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

In the following description certain specific details are set forth in order to provide a thorough understanding of various embodiments of the invention. However, one skilled in the art will understand that the invention may be practiced without these details. Unless the context requires otherwise, throughout the present specification and claims, the word “comprise” and variations thereof, such as, “comprises” and “comprising” are to be construed in an open, inclusive sense (i.e., as “including, but not limited to”).

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, including the appended claims, the singular forms of words such as “a,” “an,” and “the,” include their corresponding plural references unless the context clearly dictates otherwise.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell.

“Treat” or “treating” means to administer a therapeutic agent, such as a composition containing any of the antibodies or antigen-binding fragments of the present invention, internally or externally to a subject or patient having one or more disease symptoms, or being suspected of having a disease, for which the agent has therapeutic activity. Typically, the therapeutic agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom.

“Hydroxy” or “hydroxyl” refers to the —OH substituent.

“Alkyl” refers to a saturated, straight or branched hydrocarbon chain radical consisting solely of carbon and hydrogen atoms, having from one to twelve carbon atoms (C₁—C₁₂alkyl), from one to eight carbon atoms (C₁-C₈ alkyl) or from one to six carbon atoms (C₁-C₆ alkyl), and which is attached to the rest of the molecule by a single bond. Exemplary alkyl groups include methyl, ethyl, n-propyl, 1-methylethyl (iso-propyl), n-butyl, n-pentyl, 1,1-dimethylethyl (t-butyl), 3-methylhexyl, 2-methylhexyl, and the like. Moieties with which the alkyl group can be substituted with are selected from but not necessarily limited to the group consisting of hydroxyl, amino, alkylamino, arylamino, alkoxy, thioalkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., “Protective Groups in Organic Synthesis”, John Wiley and Sons, Second Edition, 1991.

“Alkenyl” refers to an unsaturated alkyl group having at least one double bond and from two to twelve carbon atoms (C₂-C₁₂ alkenyl), from two to eight carbon atoms (C₂-C₈ alkenyl) or from two to six carbon atoms (C₂-C₆ alkenyl), and which is attached to the rest of the molecule by a single bond, e.g., ethenyl, propenyl, butenyl, pentenyl, hexenyl, and the like.

“Alkylene” or “alkylene chain” refers to a straight or branched divalent hydrocarbon (alkyl) chain linking the rest of the molecule to a radical group, consisting solely of carbon and hydrogen, respectively. Alkylenes can have from one to twelve carbon atoms, e.g., methylene, ethylene, propylene, n-butylene, and the like. The alkylene chain is attached to the rest of the molecule through a single or double bond. The points of attachment of the alkylene chain to the rest of the molecule can be through one carbon or any two carbons within the chain. “Optionally substituted alkylene” refers to alkylene or substituted alkylene.

“Alkynyl” refers to an unsaturated alkyl group having at least one triple bond and from two to twelve carbon atoms (C₂-C₁₂ alkynyl), from two to ten carbon atoms (C₂-C₁₀ alkynyl) from two to eight carbon atoms (C₂-C₈ alkynyl) or from two to six carbon atoms (C₂-C₆ alkynyl), and which is attached to the rest of the molecule by a single bond, e.g., ethynyl, propynyl, butynyl, pentynyl, hexynyl, and the like.

“Aryl” refers to a hydrocarbon ring system radical comprising hydrogen, 6 to 18 carbon atoms and at least one aromatic ring. Exemplary aryls are hydrocarbon ring system radical comprising hydrogen and 6 to 9 carbon atoms and at least one aromatic ring; hydrocarbon ring system radical comprising hydrogen and 9 to 12 carbon atoms and at least one aromatic ring; hydrocarbon ring system radical comprising hydrogen and 12 to 15 carbon atoms and at least one aromatic ring; or hydrocarbon ring system radical comprising hydrogen and 15 to 18 carbon atoms and at least one aromatic ring. For purposes of this invention, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. “Optionally substituted aryl” refers to an aryl group or a substituted aryl group. The aryl group can be substituted with, but not necessarily limited to, one or more moieties selected from the group consisting of hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., “Protective Groups in Organic Synthesis”, John Wiley and Sons, Second Edition, 1991.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, three to nine carbon atoms, three to eight carbon atoms, three to seven carbon atoms, three to six carbon atoms, three to five carbon atoms, a ring with four carbon atoms, or a ring with three carbon atoms. The cycloalkyl ring may be saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo (bromine), chloro (chlorine), fluoro (fluorine), or iodo (iodine).

“Heterocyclyl”, “heterocycle”, or “heterocyclic ring” refers to a stable 3- to 18-membered saturated or unsaturated radical which consists of two to twelve carbon atoms and from one to six heteroatoms, for example, one to five heteroatoms, one to four heteroatoms, one to three heteroatoms, or one to two heteroatoms selected from the group consisting of nitrogen, oxygen and sulfur. Exemplary heterocycles include without limitation stable 3-15 membered saturated or unsaturated radicals, stable 3-12 membered saturated or unsaturated radicals, stable 3-9 membered saturated or unsaturated radicals, stable 8-membered saturated or unsaturated radicals, stable 7-membered saturated or unsaturated radicals, stable 6-membered saturated or unsaturated radicals, or stable 5-membered saturated or unsaturated radicals.

Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of non-aromatic heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, thietanyl, trithianyl, tetrahydropyranyl, thiomorpholinyl, thiamorpholinyl, 1-oxo-thiomorpholinyl, and 1,1-dioxo-thiomorpholinyl. Heterocyclyls include heteroaryls as defined herein, and examples of aromatic heterocyclyls are listed in the definition of heteroaryls below.

“Heterocycloalkyl” refers to a radical of the formula —Rₑ-alkyl where Rₑ is a heterocyclyl radical as defined above and alkyl is as defined herein

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical may be a stable 5-12 membered ring, a stable 5-10 membered ring, a stable 5-9 membered ring, a stable 5-8 membered ring, a stable 5-7 membered ring, or a stable 6 membered ring that comprises at least 1 heteroatom, at least 2 heteroatoms, at least 3 heteroatoms, at least 4 heteroatoms, at least 5 heteroatoms or at least 6 heteroatoms. Heteroaryls may be a monocyclic, bicyclic, tricyclic or tetracyclic ring system, which may include fused or bridged ring systems; and the nitrogen, 2 carbon or sulfur atoms in the heteroaryl radical may be optionally oxidized; the nitrogen atom may be optionally quaternized. The heteroatom may be a member of an aromatic or non-aromatic ring, provided at least one ring in the heteroaryl is aromatic. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl).

The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran.

The compounds of the invention may exhibit the phenomenon of tautomerism. While Formula I cannot expressly depict all possible tautomeric forms, it is to be understood that Formula I is intended to represent any tautomeric form of the depicted compound and not to be limited merely to a specific compound form depicted by the formula drawing.

The term “in association with” indicates that the components administered in a method of the present invention can be formulated into a single composition for simultaneous delivery or formulated separately into two or more compositions (e.g., a kit). Each component can be administered to a subject at a different time than when the other component is administered; for example, each administration may be given non-simultaneously (e.g., separately or sequentially) at several intervals over a given period of time. Moreover, the separate components may be administered to a subject by the same or by a different route.

As used herein, the term “effective amount” refer to an amount of an antiviral agent of the invention that, when administered alone or in combination with an additional therapeutic agent to a cell, tissue, or subject, is effective to cause a measurable improvement in one or more symptoms of disease, for example viral load or the progression of a viral infection. An effective dose further refers to that amount of a compound or pharmaceutical composition thereof sufficient to result in at least partial amelioration of symptoms, e.g., decrease in viral load, decrease in symptom severity, decrease in degree of needed interventions (i.e. supplied O₂), increased survival time, etc. When applied to an individual active ingredient administered alone, an effective dose refers to that ingredient alone. When applied to a combination, an effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity.

A “subject” may be a mammal such as a human, dog, cat, horse, cow, mouse, rat, monkey (e.g., cynomolgous monkey, e.g., Macacafascicularis) or rabbit. In preferred embodiments of the invention, the subject is a human subject.

### Antiviral Agents

The present invention is directed to antiviral agents according to Formula I:

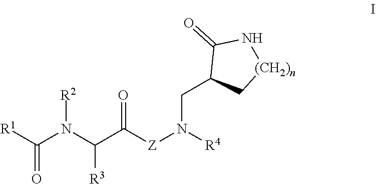

In one embodiment, R¹ is —H, alkyl, O-alkyl, aryl, alkylene-aryl, O-aryl, cycloalkyl, alkylene-cycloalkyl, O-cycloalkyl, heterocyclyl, alkylene-heterocyclyl, O-heterocyclyl, or NR⁵R⁶.

In an embodiment R¹ is —H. In an embodiment R¹ is alkyl. In an embodiment R¹ is aryl. In an embodiment R¹ is cycloalkyl. In an embodiment R¹ is heterocyclyl. In an embodiment R¹ is NR⁵R⁶.

In an embodiment R¹ is a bicyclic heterocylyl. In an embodiment R¹ is indole. In an embodiment R¹ is benzothipohene. In an embodiment R¹ is thiophene.

In an embodiment R¹ is —O(C₁-C₄)alkyl, —O-alkylene-aryl, —CH[(C₁—C₄)alkyl]NHC(O)(C₁-C₄)haloalkyl, —CH(alkylene-aryl)NHC(O)heteroaryl,

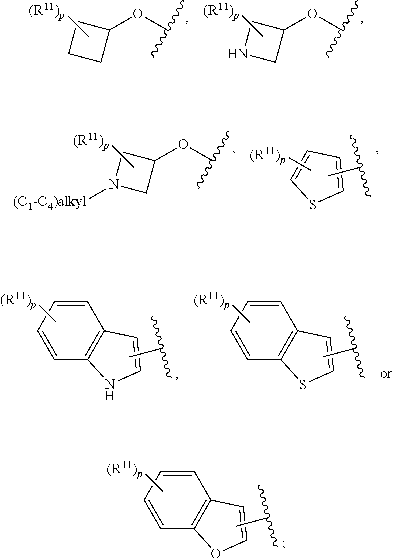

R¹¹ is —H, alkyl, alkenyl, alkynyl, halo, haloalkyl, —O(C₁-C₄)alkyl, —NH₂, —NH(C₁-C₄)alkyl or —N[(C₁-C₄)alkyl]2; and, p is 0, 1 or 2;

In an embodiment R¹ is:

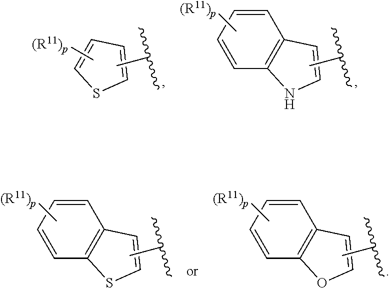

In an embodiment R¹ is:

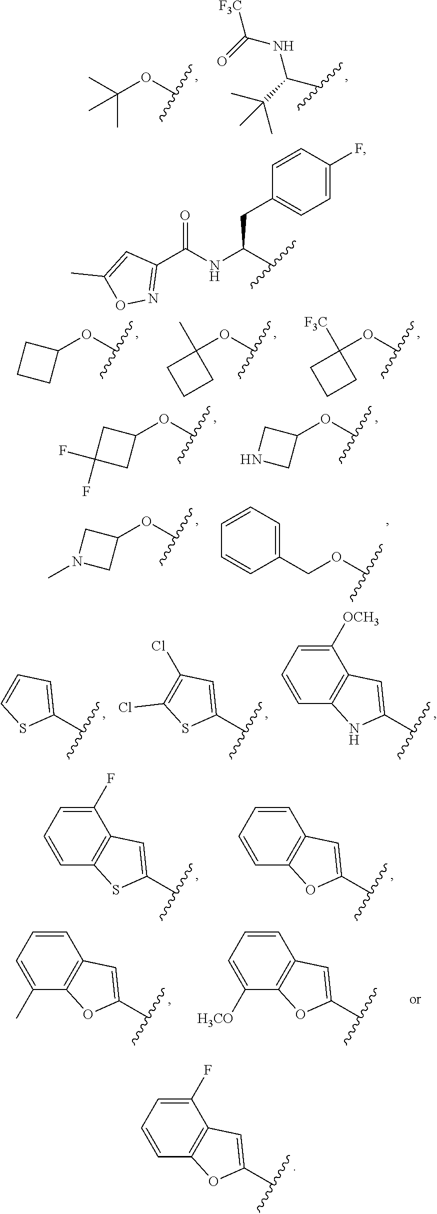

In an embodiment R² is —H or alkyl. In an embodiment R² is —H. In an embodiment R² is alkyl. In an embodiment R² is methyl, ethyl, n-propyl, t-butyl, or pentyl.

In an embodiment R¹ and R², together with the atoms to which they are bonded, form a 5- or 6-membered heterocyclic ring. In an embodiment R¹ and R², together with the atoms to which they are bonded, form a 5-membered heterocyclic ring. In an embodiment R¹ and R² together with the atoms to which they are bonded, form a 6-membered heterocyclic ring.

In an embodiment together with the atoms to which they are bonded, form an optionally substituted 5- or 6-membered heterocyclic ring which is:

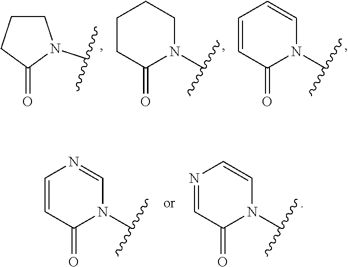

In an embodiment R³ is H, alkyl, —CH₂-alkenyl or —CH₂-alkynyl. In an embodiment R³ is —H. In an embodiment R³ is alkyl. In an embodiment R³ is methyl, ethyl, n-propyl, t-butyl, isobutyl, n-butyl, or pentyl. In an embodiment R³ is isobutyl, propyne-3-yl, n-propyl, 1-methylpropane, or 1-methylcylohexane. In an embodiment R³ is isobutyl. In an embodiment R³ is —CH₂-cyclopropyl. In an embodiment R³ is —CH₂-cyclohexyl.

In an embodiment R² and R³, together with the atoms to which they are bonded, can form a 5- or 6-membered heterocyclic ring. In an embodiment R² and R³ form a heterocyclic ring which is:

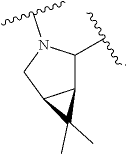 [SMILES: *C1[C@@H]2[C@H](CN1*)C2(C)C]

In an embodiment R⁴ is —CN, —C(O)R⁷, —C(S)R⁷, —SO₂R⁸, —C(O)CH₂R⁹, —SO₂CH₂R⁸ or —CH₂R¹⁰. In an embodiment R⁴ is —C(O)R⁷. In an embodiment R⁴ is —SO₂R⁸.

In an embodiment R⁴ is:

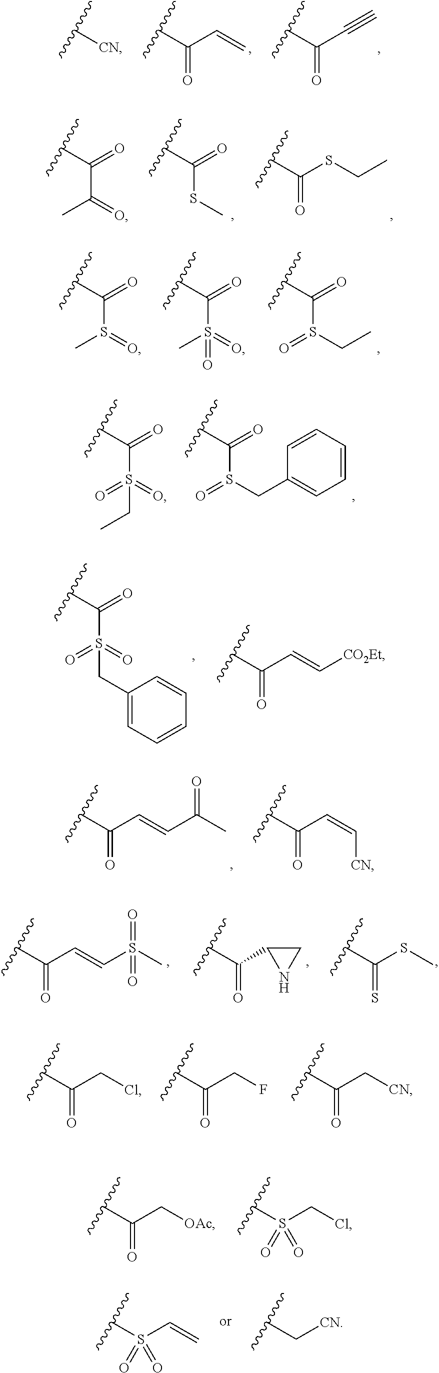

In an embodiment R⁷ is —H, alkyl, alkenyl, cycloalkyl, heterocyclyl, alkylene-X, alkenyl-C(O)Oalkyl, alkenyl-S(O)ₘalkyl, —C(O)alkyl, —O-alkyl, —S(O)ₘalkyl, or —S(O)ₘalkylene-aryl.

In an embodiment R⁸ is alkenyl, alkynyl, or alkylene-X.

In an embodiment R⁹ is halogen, —CN or —OC(O)alkyl.

In an embodiment R¹⁰ is halogen or —CN.

In an embodiment R⁵ and R⁶, together with the atom to which they are bonded, form a heterocyclic ring. In an embodiment R⁵ and R⁶, together with the atom to which they are bonded, form a heteroaryl ring.

In an embodiment X is a halogen, —OH, or —OC(O)aryl. In an embodiment X is a halogen. In an embodiment X is —OC(O)aryl. In an embodiment the halogen is selected from the group consisting of Cl, Br, and I.

In an embodiment Z is —CH₂—, —NCH₃—, or —NH—. In an embodiment Z is —CH₂-. In an embodiment Z is —NH—.

In an embodiment m is 0, 1 or 2.

In an embodiment n is 1 or 2.

In an embodiment when Z is —NH— and R³ is alkyl, R⁴ is then —C(O)R⁷, —C(S)R⁷, —SO₂R⁸, —C(O)CH₂R⁹, —SO₂CH₂R⁸ or —CH₂R¹⁰.

In embodiments of any of the preceding, each alkyl, alkenyl, alkynyl, alkylene, aryl, cycloalkyl, and heterocyclyl is independently optionally substituted with 1, 2 or 3 groups selected from —OH, —CN, —(C₁-C₄) alkylNHC(O)(C₁-C₄)haloalkyl, alkylene-aryl-NHC(O)heteroaryl, —SH, —S(O)NH₂, halogen, —NH₂, —NH(C₁-C₄)alkyl, —N[(C₁-C₄)alkyl]2, —C(O)NH₂, —COOH, —COOMe, acetyl, —(C₁-C₅)alkyl, —O(C₁-C₅)alkyl, (C₂-C₅)alkenyl, (C₂—C₈)alkynyl, thioalkyl, cyanomethylene, —NH-heterocyclyl, —NH—C(O)(C₁-C₄)alkyl, —NH—C(O)— heterocycloalkyl, —NH-heterocycloalkyl, —NH—C(O)-alkylene, —NH—C(O)—O-alkylene, —CH₂—C(O)-alkyl, —C(O)-alkyl, cycloalkyl, —C(O)-cycloalkyl, —CH₂-C(O)-aryl, —CH₂-aryl, —C(O)-aryl, —C(O)-heterocycloalkyl, —CH₂-C(O)-heterocyclyl, —C(O)-heterocyclyl, or heterocyclyl.

In an embodiment R¹ is heterocyclyl, R² is H, R³ is alkyl, and n is 1. In an embodiment R¹ is indole, R² is H, R³ is alkyl, and n is 1. In an embodiment R¹ is heterocyclyl, R² is H, R³ is isobutyl, and n is 1. In an embodiment R¹ is heterocyclyl, R² is H, R³ is alkyl, R⁴ is —C(O)R⁷, and n is 1. In an embodiment R¹ is indole, R² is H, R³ is alkyl, R⁴ is —C(O)R⁵, and n is 1. In an embodiment R¹ is heterocyclyl, R² is H, R³ is isobutyl, R⁴ is —C(O)R⁷, and n is 1.

In an embodiment R¹ and R² together with the atoms to which they are bonded form a 5- or 6-membered heterocyclic ring, R³ is alkyl, and n is 1. In an embodiment R¹ and R² together with the atoms to which they are bonded form a 5- or 6-membered heterocyclic ring, R³ is isobutyl and n is 1. In an embodiment R¹ and R² together with the atoms to which they are bonded form a 5- or 6-membered heterocyclic ring, R³ is —CH₂-cyclopropyl, and n is 1.In an embodiment R¹ and R² together with the atoms to which they are bonded form a 5-membered heterocyclic ring, R³ is alkyl and n is 1. In an embodiment R¹ and R² together with the atoms to which they are bonded form a 6-membered heterocyclic ring, R³ is alkyl and n is 1.

The inventive compounds according to Formula I may be isotopically-labeled by having one or more atoms replaced by an atom having a different atomic mass or mass number. Examples of isotopes that can be incorporated into compounds of according to Formula I include isotopes of hydrogen, carbon, nitrogen, oxygen, phosphorous, fluorine, chlorine, or iodine. Illustrative of such isotopes are ²H, ³H, ¹¹C, ¹³C, ¹⁴C, ¹³N, ¹⁵N, 150, 170, 180, ³¹P, ³²p, ³⁵S, ¹⁸F, ³⁶Cl, ¹²³I, and ¹²⁵I, respectively. These radiolabeled compounds can be used to measure the biodistribution, tissue concentration and the kinetics of transport and excretion from biological tissues including a subject to which such a labeled compound is administered. Labeled compounds are also used to determine therapeutic effectiveness, the site or mode of action, and the binding affinity of a candidate therapeutic to a pharmacologically important target. Certain radioactive-labeled compounds according to Formula I, therefore, are useful in drug and/or tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, affords certain therapeutic advantages resulting from the greater metabolic stability, for example, increased in vivo half-life of compounds containing deuterium. Substitution of hydrogen with deuterium may reduce dose required for therapeutic effect, and hence may be preferred in a discovery or clinical setting.

Substitution with positron emitting isotopes, such as ¹¹C, ¹⁸F, ¹⁵0 and ¹³N, provides labeled analogs of the inventive compounds that are useful in Positron Emission Tomography (PET) studies, e.g., for examining substrate receptor occupancy. Isotopically-labeled compounds according to Formula I, can generally be prepared by conventional techniques known to those skilled in the art or by processes analogous to those described in the Preparations and Examples section as set out below using an appropriate isotopic-labeling reagent.

Examples of compounds of Formula I include, but are not limited to:

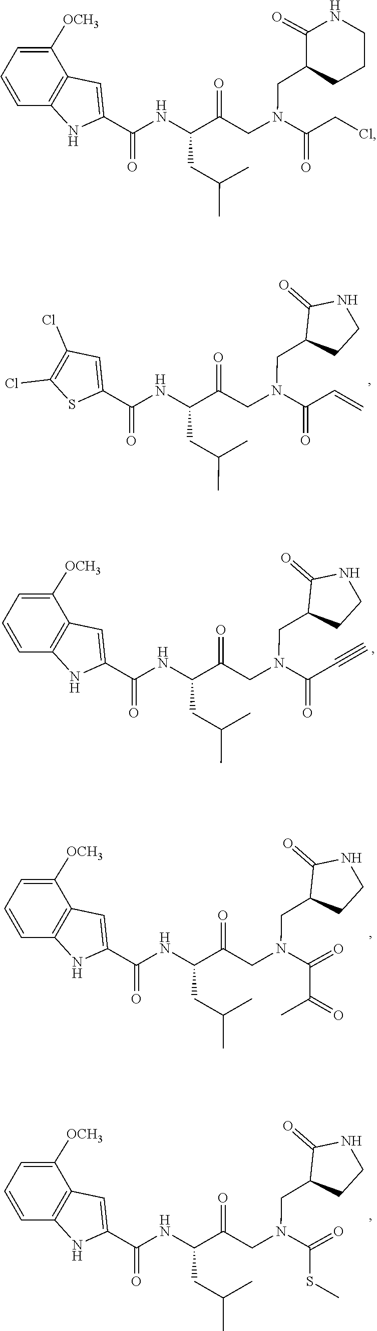 [SMILES: COc1cccc2[nH]c(C(=O)N[C@@H](CC(C)C)C(=O)CN(C[C@@H]3CCCNC3=O)C(=O)CCl)cc12 ; C=CC(=O)N(CC(=O)[C@H](CC(C)C)NC(=O)c1cc(Cl)c(Cl)s1)C[C@@H]1CCNC1=O ; C#CC(=O)N(CC(=O)[C@H](CC(C)C)NC(=O)c1cc2c(OC)cccc2[nH]1)C[C@@H]1CCNC1=O ; COc1cccc2[nH]c(C(=O)N[C@@H](CC(C)C)C(=O)CN(C[C@@H]3CCNC3=O)C(=O)C(C)=O)cc12 ; COc1cccc2[nH]c(C(=O)N[C@@H](CC(C)C)C(=O)CN(C[C@@H]3CCNC3=O)C(=O)SC)cc12]

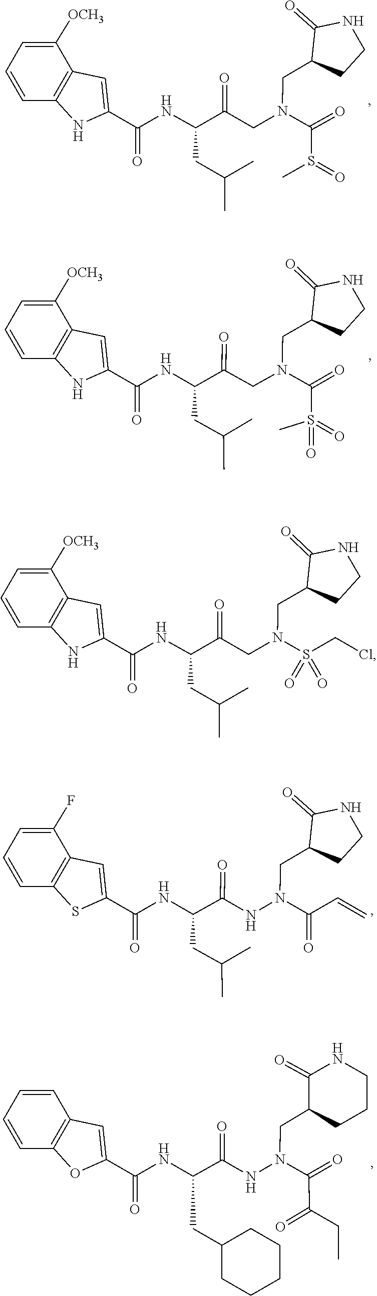 [SMILES: COc1cccc2[nH]c(C(=O)N[C@@H](CC(C)C)C(=O)CN(C[C@@H]3CCNC3=O)C(=O)S(C)=O)cc12 ; COc1cccc2[nH]c(C(=O)N[C@@H](CC(C)C)C(=O)CN(C[C@@H]3CCNC3=O)C(=O)S(C)(=O)=O)cc12 ; COc1cccc2[nH]c(C(=O)N[C@@H](CC(C)C)C(=O)CN(C[C@@H]3CCNC3=O)S(=O)(=O)CCl)cc12 ; C=CC(=O)N(C[C@@H]1CCNC1=O)NC(=O)[C@H](CC(C)C)NC(=O)c1cc2c(F)cccc2s1 ; CCC(=O)C(=O)N(C[C@@H]1CCCNC1=O)NC(=O)[C@H](CC1CCCCC1)NC(=O)c1cc2ccccc2o1]

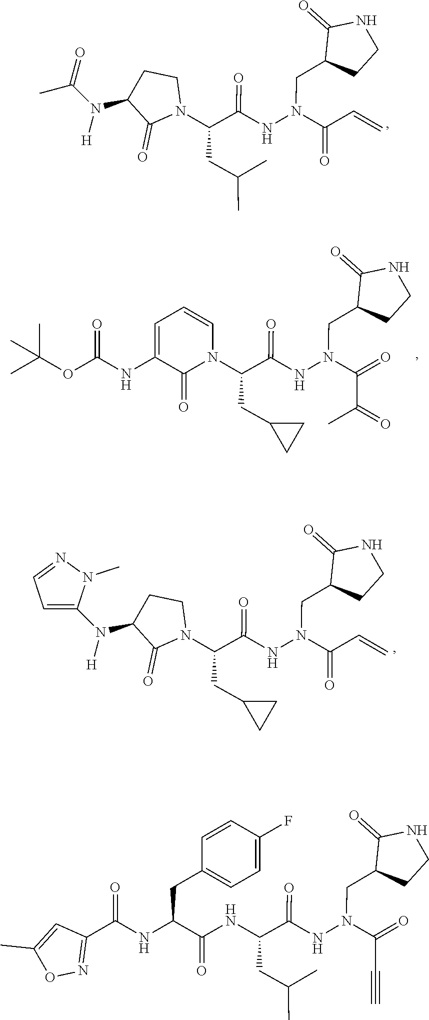

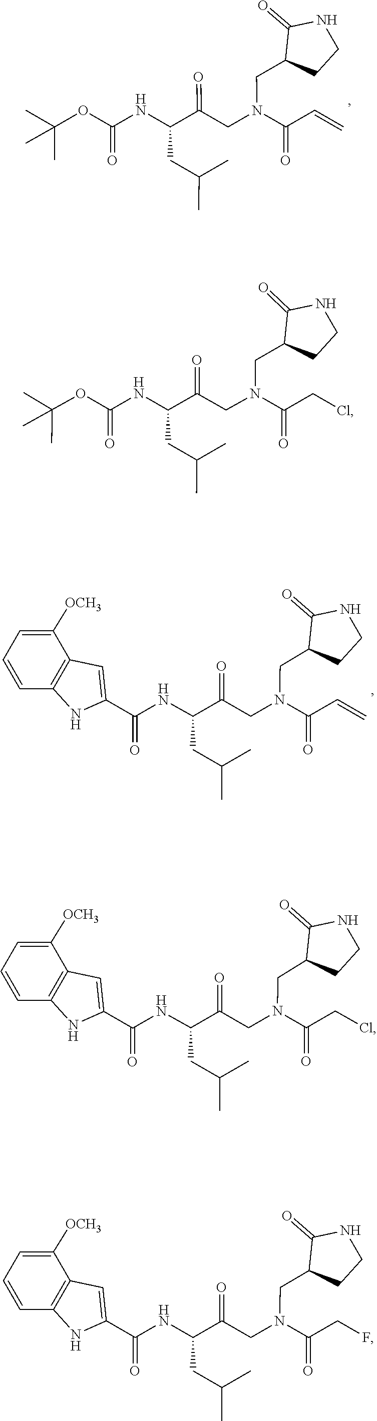

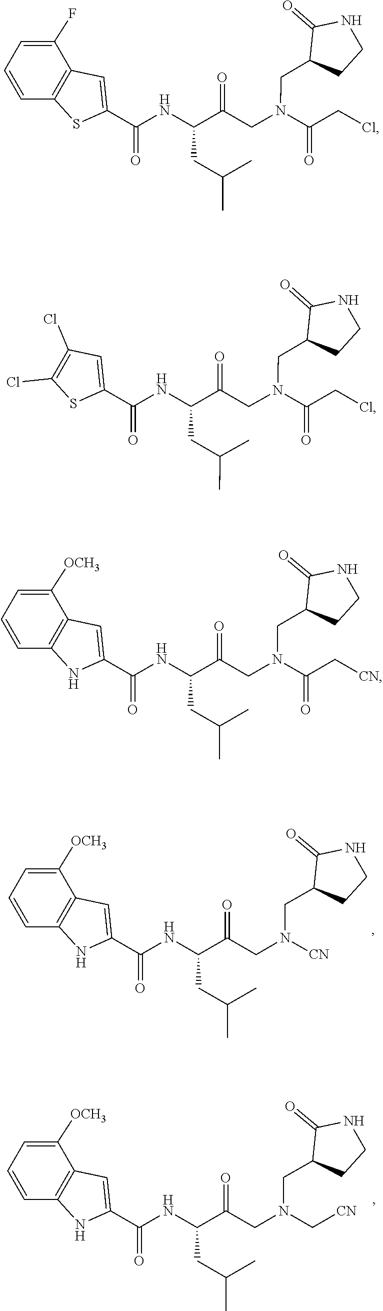 [SMILES: CC(C)C[C@H](NC(=O)c1cc2c(F)cccc2s1)C(=O)CN(C[C@@H]1CCNC1=O)C(=O)CCl ; CC(C)C[C@H](NC(=O)c1cc(Cl)c(Cl)s1)C(=O)CN(C[C@@H]1CCNC1=O)C(=O)CCl ; COc1cccc2[nH]c(C(=O)N[C@@H](CC(C)C)C(=O)CN(C[C@@H]3CCNC3=O)C(=O)CC#N)cc12 ; COc1cccc2[nH]c(C(=O)N[C@@H](CC(C)C)C(=O)CN(C#N)C[C@@H]3CCNC3=O)cc12 ; COc1cccc2[nH]c(C(=O)N[C@@H](CC(C)C)C(=O)CN(CC#N)C[C@@H]3CCNC3=O)cc12]

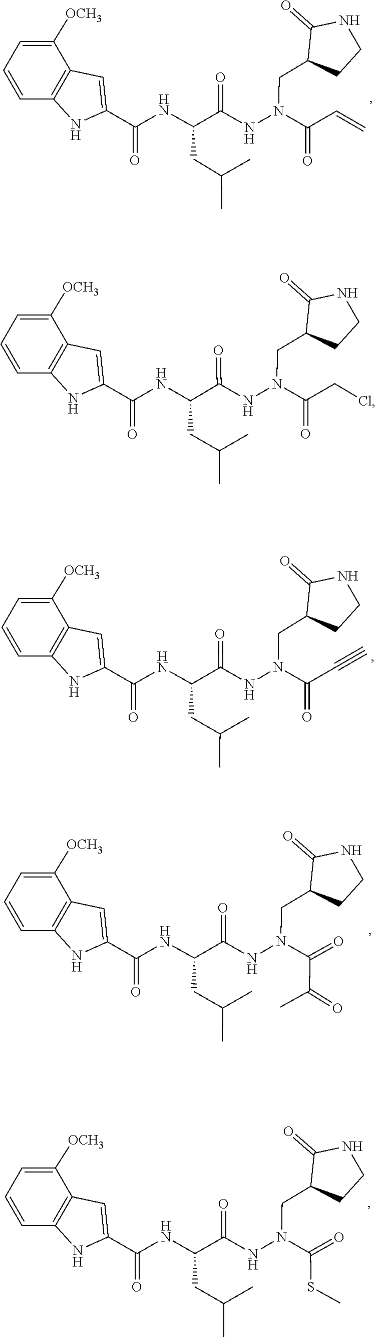 [SMILES: C=CC(=O)N(C[C@@H]1CCNC1=O)NC(=O)[C@H](CC(C)C)NC(=O)c1cc2c(OC)cccc2[nH]1 ; COc1cccc2[nH]c(C(=O)N[C@@H](CC(C)C)C(=O)NN(C[C@@H]3CCNC3=O)C(=O)CCl)cc12 ; C#CC(=O)N(C[C@@H]1CCNC1=O)NC(=O)[C@H](CC(C)C)NC(=O)c1cc2c(OC)cccc2[nH]1 ; COc1cccc2[nH]c(C(=O)N[C@@H](CC(C)C)C(=O)NN(C[C@@H]3CCNC3=O)C(=O)C(C)=O)cc12 ; COc1cccc2[nH]c(C(=O)N[C@@H](CC(C)C)C(=O)NN(C[C@@H]3CCNC3=O)C(=O)SC)cc12]

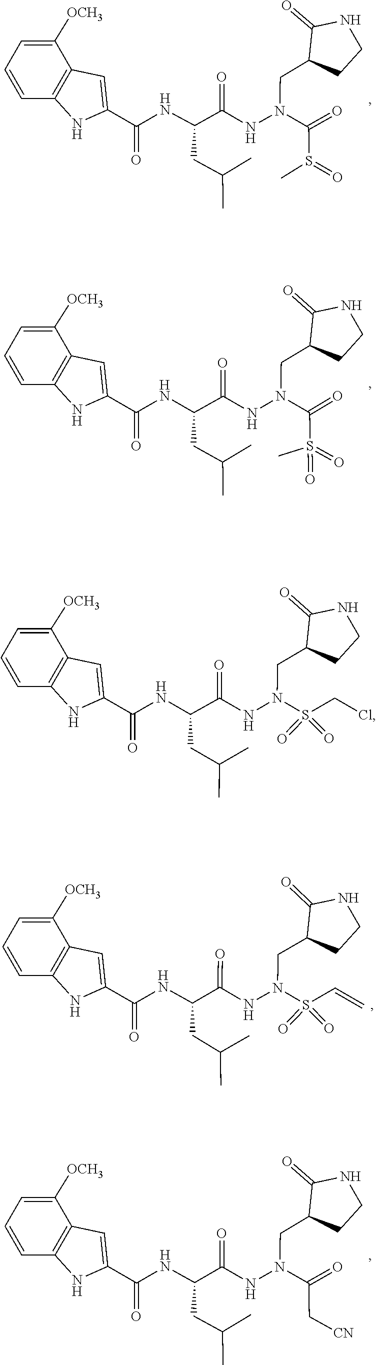 [SMILES: COc1cccc2[nH]c(C(=O)N[C@@H](CC(C)C)C(=O)NN(C[C@@H]3CCNC3=O)C(=O)S(C)=O)cc12 ; COc1cccc2[nH]c(C(=O)N[C@@H](CC(C)C)C(=O)NN(C[C@@H]3CCNC3=O)C(=O)S(C)(=O)=O)cc12 ; COc1cccc2[nH]c(C(=O)N[C@@H](CC(C)C)C(=O)NN(C[C@@H]3CCNC3=O)S(=O)(=O)CCl)cc12 ; C=CS(=O)(=O)N(C[C@@H]1CCNC1=O)NC(=O)[C@H](CC(C)C)NC(=O)c1cc2c(OC)cccc2[nH]1 ; COc1cccc2[nH]c(C(=O)N[C@@H](CC(C)C)C(=O)NN(C[C@@H]3CCNC3=O)C(=O)CC#N)cc12]

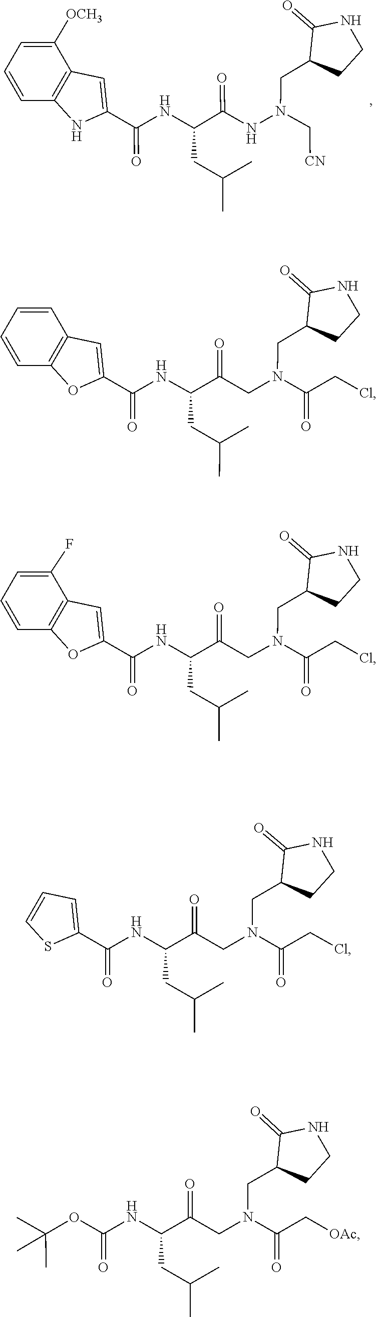 [SMILES: COc1cccc2[nH]c(C(=O)N[C@@H](CC(C)C)C(=O)NN(CC#N)C[C@@H]3CCNC3=O)cc12 ; CC(C)C[C@H](NC(=O)c1cc2ccccc2o1)C(=O)CN(C[C@@H]1CCNC1=O)C(=O)CCl ; CC(C)C[C@H](NC(=O)c1cc2c(F)cccc2o1)C(=O)CN(C[C@@H]1CCNC1=O)C(=O)CCl ; CC(C)C[C@H](NC(=O)c1cccs1)C(=O)CN(C[C@@H]1CCNC1=O)C(=O)CCl ; CC(=O)OCC(=O)N(CC(=O)[C@H](CC(C)C)NC(=O)OC(C)(C)C)C[C@@H]1CCNC1=O]

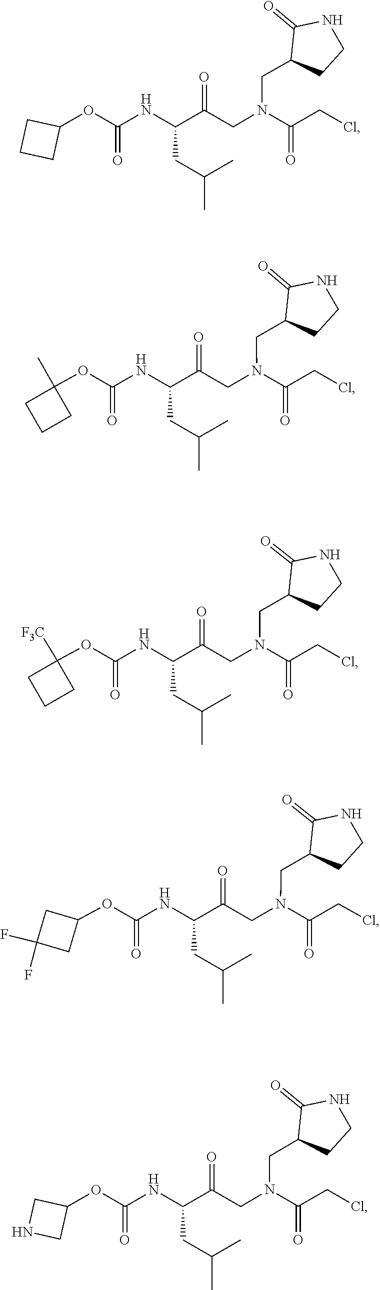 [SMILES: CC(C)C[C@H](NC(=O)OC1CCC1)C(=O)CN(C[C@@H]1CCNC1=O)C(=O)CCl ; CC(C)C[C@H](NC(=O)OC1(C)CCC1)C(=O)CN(C[C@@H]1CCNC1=O)C(=O)CCl ; CC(C)C[C@H](NC(=O)OC1(C(F)(F)F)CCC1)C(=O)CN(C[C@@H]1CCNC1=O)C(=O)CCl ; CC(C)C[C@H](NC(=O)OC1CC(F)(F)C1)C(=O)CN(C[C@@H]1CCNC1=O)C(=O)CCl ; CC(C)C[C@H](NC(=O)OC1CNC1)C(=O)CN(C[C@@H]1CCNC1=O)C(=O)CCl]

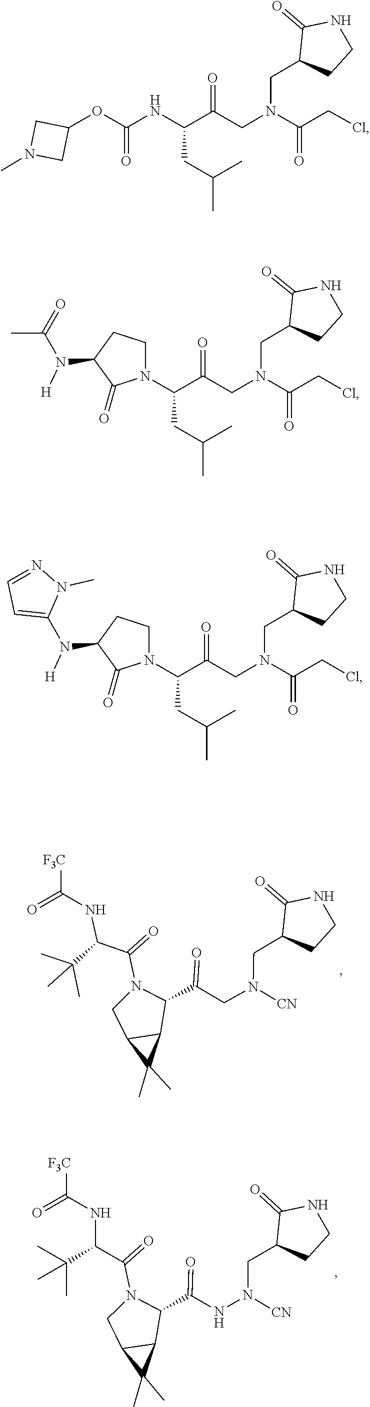 [SMILES: CC(C)C[C@H](NC(=O)OC1CN(C)C1)C(=O)CN(C[C@@H]1CCNC1=O)C(=O)CCl ; CC(=O)N[C@H]1CCN([C@@H](CC(C)C)C(=O)CN(C[C@@H]2CCNC2=O)C(=O)CCl)C1=O ; CC(C)C[C@@H](C(=O)CN(C[C@@H]1CCNC1=O)C(=O)CCl)N1CC[C@H](Nc2ccnn2C)C1=O ; CC(C)(C)[C@H](NC(=O)C(F)(F)F)C(=O)N1C[C@H]2[C@@H]([C@H]1C(=O)CN(C#N)C[C@@H]1CCNC1=O)C2(C)C ; CC(C)(C)[C@H](NC(=O)C(F)(F)F)C(=O)N1C[C@H]2[C@@H]([C@H]1C(=O)NN(C#N)C[C@@H]1CCNC1=O)C2(C)C]

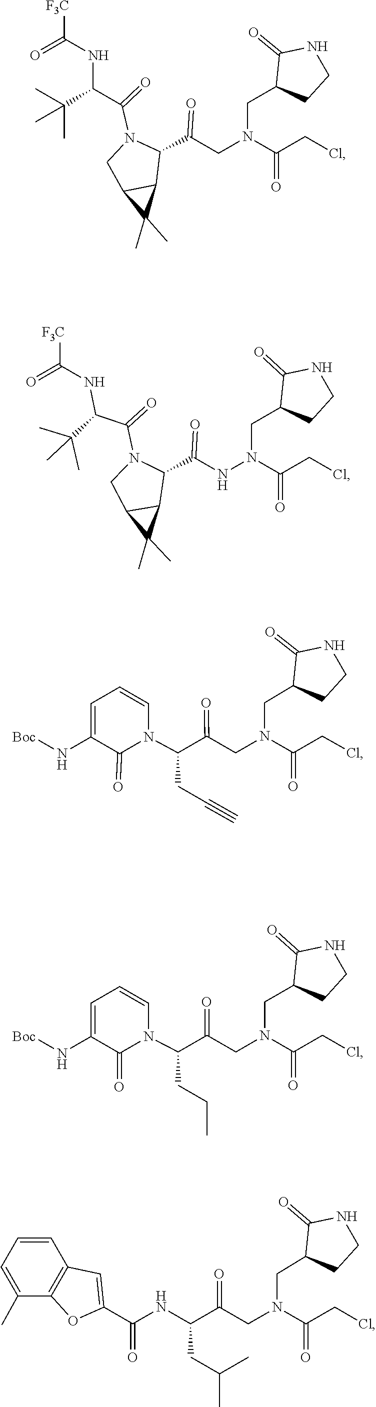 [SMILES: CC(C)(C)[C@H](NC(=O)C(F)(F)F)C(=O)N1C[C@H]2[C@@H]([C@H]1C(=O)CN(C[C@@H]1CCNC1=O)C(=O)CCl)C2(C)C ; CC(C)(C)[C@H](NC(=O)C(F)(F)F)C(=O)N1C[C@H]2[C@@H]([C@H]1C(=O)NN(C[C@@H]1CCNC1=O)C(=O)CCl)C2(C)C ; C#CC[C@@H](C(=O)CN(C[C@@H]1CCNC1=O)C(=O)CCl)n1cccc(NC(=O)OC(C)(C)C)c1=O ; CCC[C@@H](C(=O)CN(C[C@@H]1CCNC1=O)C(=O)CCl)n1cccc(NC(=O)OC(C)(C)C)c1=O ; Cc1cccc2cc(C(=O)N[C@@H](CC(C)C)C(=O)CN(C[C@@H]3CCNC3=O)C(=O)CCl)oc12]

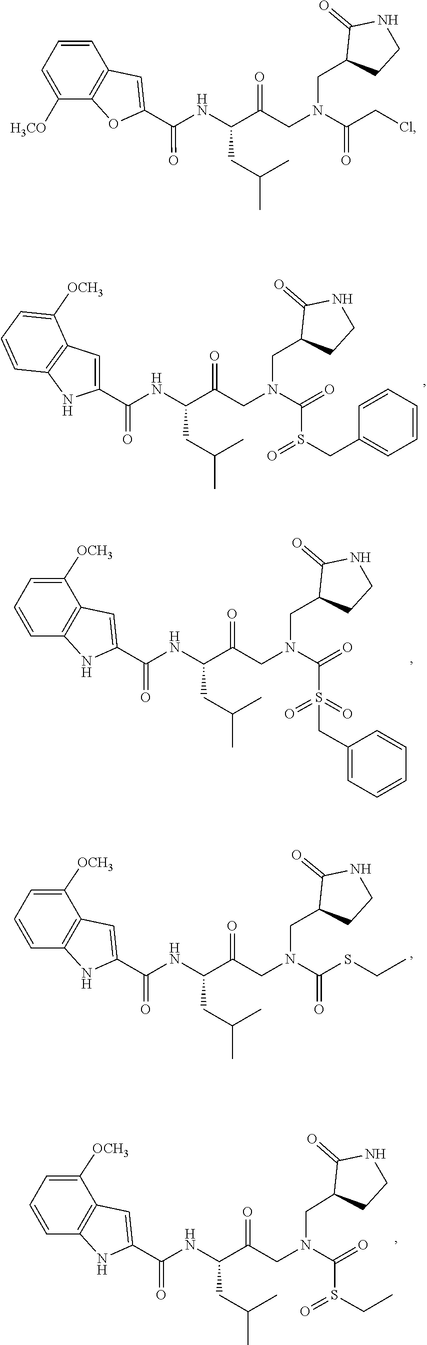 [SMILES: COc1cccc2cc(C(=O)N[C@@H](CC(C)C)C(=O)CN(C[C@@H]3CCNC3=O)C(=O)CCl)oc12 ; COc1cccc2[nH]c(C(=O)N[C@@H](CC(C)C)C(=O)CN(C[C@@H]3CCNC3=O)C(=O)S(=O)Cc3ccccc3)cc12 ; COc1cccc2[nH]c(C(=O)N[C@@H](CC(C)C)C(=O)CN(C[C@@H]3CCNC3=O)C(=O)S(=O)(=O)Cc3ccccc3)cc12 ; CCSC(=O)N(CC(=O)[C@H](CC(C)C)NC(=O)c1cc2c(OC)cccc2[nH]1)C[C@@H]1CCNC1=O ; CCS(=O)C(=O)N(CC(=O)[C@H](CC(C)C)NC(=O)c1cc2c(OC)cccc2[nH]1)C[C@@H]1CCNC1=O]

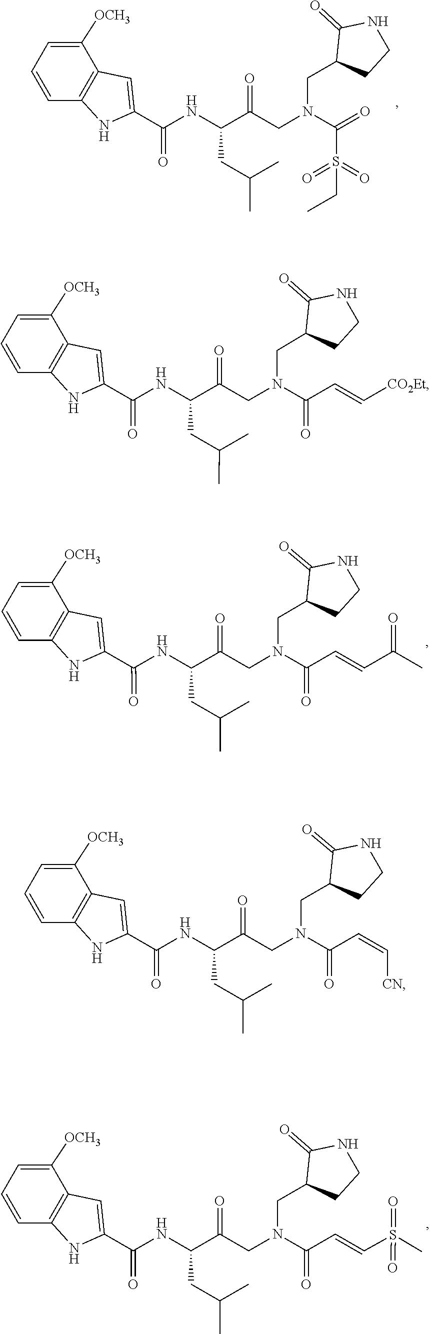 [SMILES: CCS(=O)(=O)C(=O)N(CC(=O)[C@H](CC(C)C)NC(=O)c1cc2c(OC)cccc2[nH]1)C[C@@H]1CCNC1=O ; CCOC(=O)/C=C/C(=O)N(CC(=O)[C@H](CC(C)C)NC(=O)c1cc2c(OC)cccc2[nH]1)C[C@@H]1CCNC1=O ; COc1cccc2[nH]c(C(=O)N[C@@H](CC(C)C)C(=O)CN(C[C@@H]3CCNC3=O)C(=O)/C=C/C(C)=O)cc12 ; COc1cccc2[nH]c(C(=O)N[C@@H](CC(C)C)C(=O)CN(C[C@@H]3CCNC3=O)C(=O)/C=C\C#N)cc12 ; COc1cccc2[nH]c(C(=O)N[C@@H](CC(C)C)C(=O)CN(C[C@@H]3CCNC3=O)C(=O)/C=C/S(C)(=O)=O)cc12]

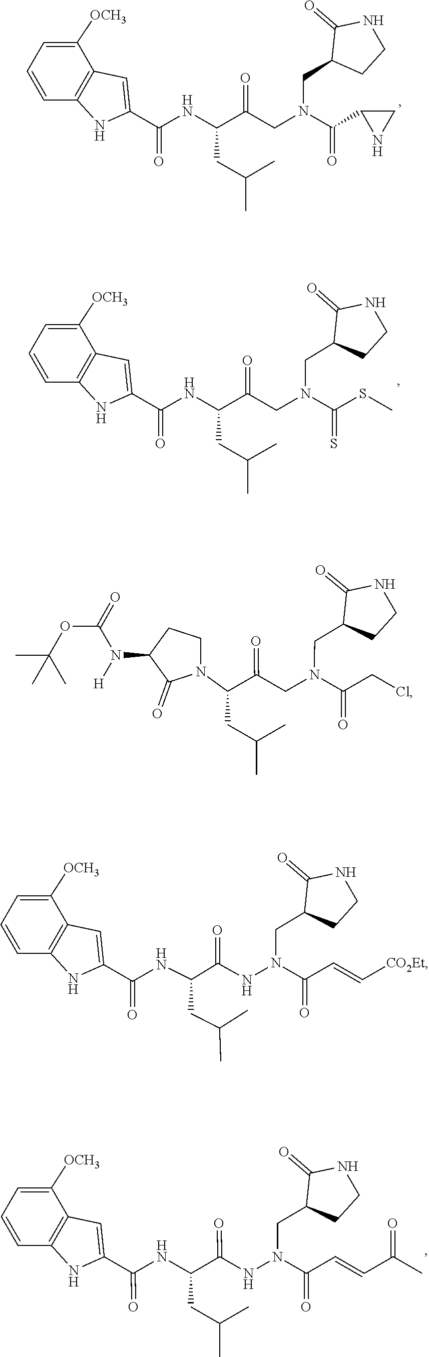

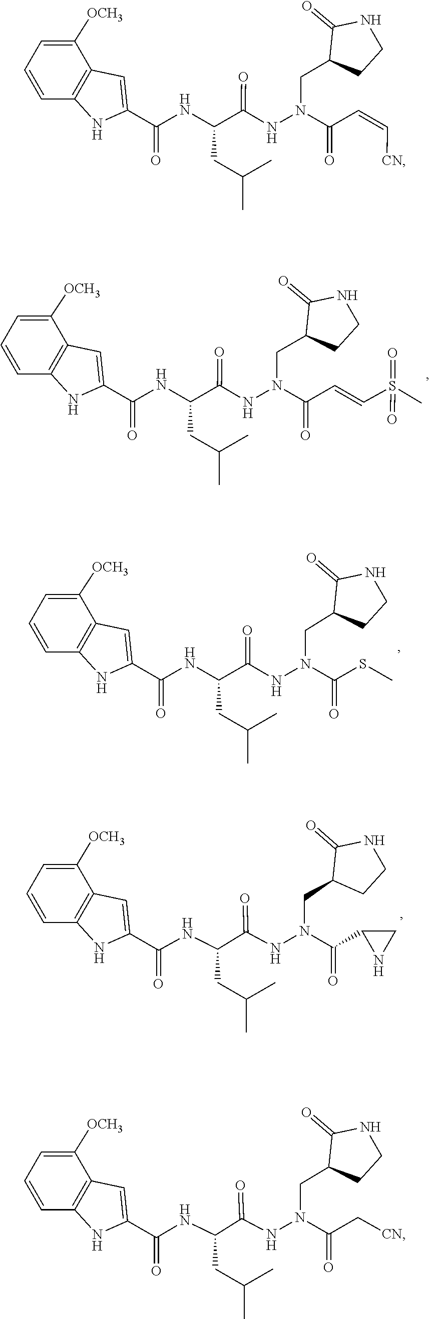

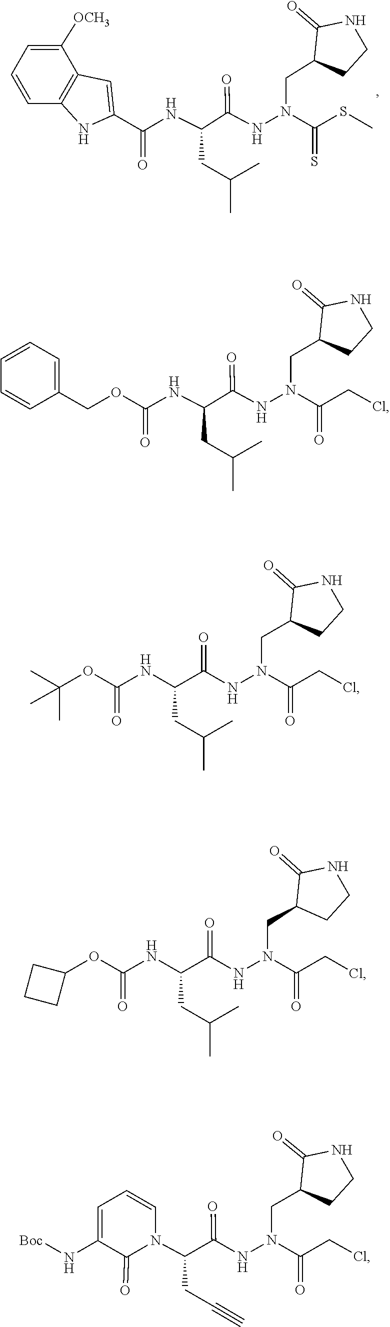

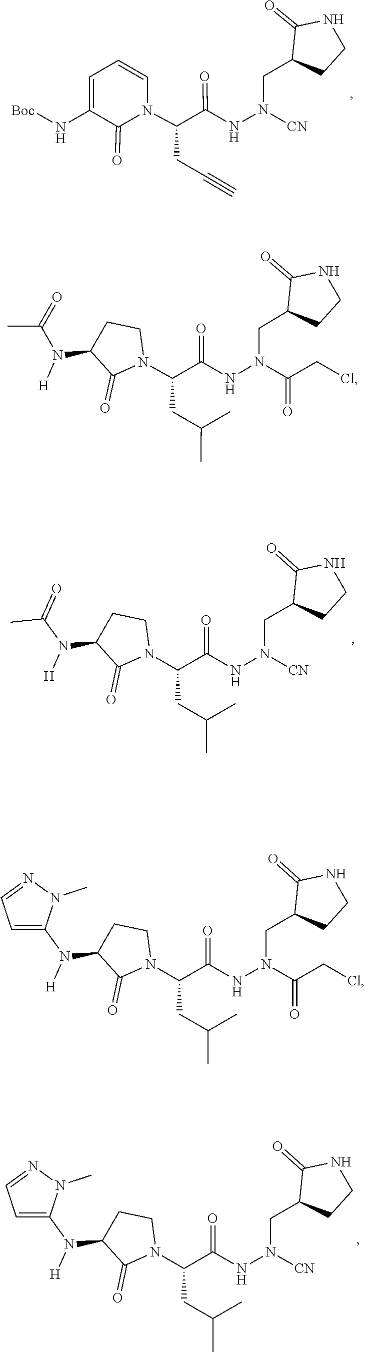 [SMILES: C#CC[C@@H](C(=O)NN(C#N)C[C@@H]1CCNC1=O)n1cccc(NC(=O)OC(C)(C)C)c1=O ; CC(=O)N[C@H]1CCN([C@@H](CC(C)C)C(=O)NN(C[C@@H]2CCNC2=O)C(=O)CCl)C1=O ; CC(=O)N[C@H]1CCN([C@@H](CC(C)C)C(=O)NN(C#N)C[C@@H]2CCNC2=O)C1=O ; CC(C)C[C@@H](C(=O)NN(C[C@@H]1CCNC1=O)C(=O)CCl)N1CC[C@H](Nc2ccnn2C)C1=O ; CC(C)C[C@@H](C(=O)NN(C#N)C[C@@H]1CCNC1=O)N1CC[C@H](Nc2ccnn2C)C1=O]

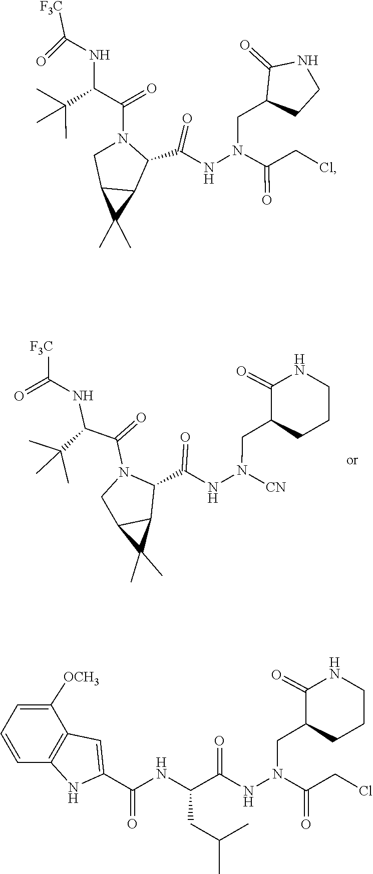 [SMILES: CC(C)(C)[C@H](NC(=O)C(F)(F)F)C(=O)N1C[C@H]2[C@@H]([C@H]1C(=O)NN(C[C@@H]1CCNC1=O)C(=O)CCl)C2(C)C ; CC(C)(C)[C@H](NC(=O)C(F)(F)F)C(=O)N1C[C@H]2[C@@H]([C@H]1C(=O)NN(C#N)C[C@@H]1CCCNC1=O)C2(C)C ; COc1cccc2[nH]c(C(=O)N[C@@H](CC(C)C)C(=O)NN(C[C@@H]3CCCNC3=O)C(=O)CCl)cc12]

### Therapeutic Uses of Antiviral Agents

In certain embodiments, the present disclosure provides a method for preventing, ameliorating, or treating a viral infection in a subject in need thereof, the method comprising administering to the subject a therapeutically effective amount of a compound of Formula I.

In some embodiments, the viral infection is associated with a virus selected from the group consisting of rhinovirus, adenovirus, influenza virus, respiratory syncytial virus, enterovirus D68, enterovirus A71, Coxsackievirus A16, the etiological agents of hand, foot, and mouth disease, (HIFMMD), Coxsackievirus B3, hepatitis C virus (HCV), West Nile virus, Sindbis virus (SIINV), dengue virus, Ebola virus, Marburg virus, Crimean-Congo hemorrhagic fever (CCHF) orthonairovirus (CCHFV), yellow fever virus, Rift Valley fever virus (RVFV), Omsk hemorrhagic fever virus (OHFV), Kyasanur Forest disease virus (KFDV), Junin virus, Machupo virus, Sabia virus, Guanarito virus, Garissa virus, Ilesha virus, Lassa fever virus, severe acute respiratory syndrome coronavirus (SARS-CoV), Middle East respiratory syndrome coronavirus (MERS-CoV), and severe acute respiratory syndrome coronavirus 2 (SARS-CoV-2).

In specific embodiments, the virus is a coronavirus. In specific embodiments, the virus is an enterovirus. In an embodiment the virus is enterovirus D68. In an embodiment the virus is enterovirus A71. In an embodiment the virus is coxsackievirus A16. In an embodiment the virus is coxsackievirus B3.

In specific embodiments, the virus is MERS-CoV. In specific embodiments, the virus is SARS-CoV. In specific embodiments, the virus is SARS-CoV-2.

In one embodiment, a compound(s) of Formula I may be used alone, or in association with other, further therapeutic agents and therapeutic procedures, for treating or preventing a viral infection in a subject in need of such treatment or prevention.

In an embodiment, a compound(s) of Formula I may be used alone, or in association with at least one other antiviral therapy. Examples of antiviral therapies include: corticosteroids (e.g., dexamethasone); TL-6 inhibitors (e.g., tocilizumab); viral entry inhibitors (e.g., chloroquine, hydroxychloroquine, convalescent plasma, umifenovir); protease inhibitors (e.g., lopinavir, ritonavir; PF-07304814; PF-00835231; GC376); RNA-dependent RNA polymerase (RdRp) inhibitors (e.g., remdesivir), and interferons (e.g., IFN-β).

In an embodiment, a compound(s) of Formula I may be used in combination with an RdRp inhibitor.

In an embodiment, a compound(s) of Formula I may be used in combination with remdesivir.

In an embodiment, a compound(s) of Formula I may be used alone, or in association with corticosteroids.

In an embodiment, a compound(s) of Formula I may be used alone, or in association with dexamethasone.

Various indicators for determining effectiveness of treatment and or/prevention are known to those skilled in the art. Examples of suitable indicators include, but are not limited to, a reduction in viral load, a reduction in viral replication, a reduction in time to seroconversion (virus undetectable in patient serum), a reduction of morbidity or mortality in clinical outcomes, and/or other indicator(s) of disease response. Further indicators include one or more overall quality of life health indicators, such as reduced illness duration, reduced illness severity, reduced time to return to normal health and normal activity, and reduced time to alleviation of one or more symptoms. In an embodiment, a compound(s) of Formula I, or a pharmaceutically acceptable salt thereof, can result in the reduction, alleviation or positive indication of one or more of the aforementioned indicators compared to a subject who is receiving the standard of care or an untreated subject.

In another embodiment, a compound(s) of Formula I decreases the viral load in a subject in need thereof. The decrease in viral load can be detected using any method known in the art.

Other side effects of viral infection include overexpression and/or dysregulation of pro-inflammatory cytokines. Accordingly, in an embodiment of the invention, the compound(s) of Formula I are used in association with an agent which treats or prevents such an overexpression and/or dysregulation. Examples of these therapies include: antibodies (e.g., toclizumab, sarilumab, clazakizumab, olokizumab, siltuximab; corticosteroids (e.g., dexamethasone); synthetic proteins (e.g., anakinra); and Janus kinase inhibitors (e.g., ruxo1itinib, tofacitinib, oclacitinib, baricitinib, fedratinib, upadacitinib, filgotinib, cerdulatinib, gandotinib, lestaurtinib, momelotinib, pacritinib, abrocitinib, BMS-986165).

Accordingly, in an embodiment of the invention, the compound(s) of Formula I is used in association with an antibody. In an embodiment of the invention, the compound(s) of Formula I are used in association with synthetic protein(s). In an embodiment of the invention, the compound(s) of Formula I are used in association with Janus kinase inhibitor(s).

In another embodiment, the present invention relates to compositions comprising one or more antiviral agents of the present invention and a pharmaceutically acceptable carrier or diluent. Such compositions can further comprise one or more other therapeutically active ingredients as discussed above.

### Pharmaceutical Compositions and Administration

The present invention also provides a pharmaceutical composition comprising (i) a therapeutically effective amount of at least one compound according to Formula I or a stereoisomer, a tautomer or a pharmaceutically acceptable salt thereof, (ii) in combination with a pharmaceutically acceptable carrier, diluent or excipient. Pharmaceutical compositions comprising a pharmaceutically acceptable carrier, diluent, or excipient, in association with further therapeutic agents are also part of the present invention.

The term “pharmaceutical” as used herein refers to a chemical substance intended for use in the cure, treatment, or prevention of disease and which is subject to an approval process by the U.S. Food and Drug Administration (or a non-U.S. equivalent thereof) as a prescription or over-the-counter drug product. Details on techniques for formulation and administration of such compositions may be found in Remington, The Science and Practice of Pharmacy 21^{5t} Edition (Mack Publishing Co., Easton, Pa.) and Nielloud and Marti-Mestres, Pharmaceutical Emulsions and Suspensions: 2^{nd} Edition (Marcel Dekker, Inc, New York). To prepare pharmaceutical or sterile compositions of the antiviral agent(s) of the invention, the compound(s) is(are) admixed with a pharmaceutically acceptable carrier or excipient. See, e.g., Remington's Pharmaceutical Sciences and U.S. Pharmacopeia: National Formulary, Mack Publishing Company, Easton, Pa. (1984).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The PharmacologicalBasis of Therapeutics, McGraw-Hill, New York, N.Y.; Gennaro (2000) Remington: The Science and Practice ofPharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Toxicity and therapeutic efficacy of the compounds or compositions of the invention, administered alone or in combination with another therapeutic agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

In a further embodiment, a further therapeutic agent that is administered to a subject in association with an antiviral agent of the invention in accordance with the Physicians' Desk Reference 2003 (Thomson Healthcare; 57th edition (Nov. 1, 2002)).

The mode of administration can vary. For the purposes of this disclosure, the pharmaceutical compositions may be administered by a variety of means including non-parenterally, parenterally, by inhalation spray, topically, or rectally in formulations containing pharmaceutically acceptable carriers, adjuvants and vehicles. “Non-parenteral administration” encompasses oral, buccal, sublingual, topical, transdermal, ophthalmic, otic, nasal, rectal, cervical, pulmonary, mucosal, and vaginal routes. The term parenteral as used here includes but is not limited to subcutaneous, intravenous, intramuscular, intraarterial, intradermal, intrathecal and epidural injections with a variety of infusion techniques. Intraarterial and intravenous injection as used herein includes administration through catheters. The term oral as used herein includes, but is not limited to oral ingestion, or delivery by a sublingual or buccal route.

Pharmaceutical compositions may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing a drug compound in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents; such as magnesium stearate, stearic acid or talc. Tablets may be uncoated, or may be coated by known techniques including enteric coating, colonic coating, or microencapsulation to delay disintegration and adsorption in the gastrointestinal tract and/or provide a sustained action over a longer period. For example, a time delay material such as glyceryl monostearate or glyceryl distearate alone or with a wax may be employed.

When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e., the capacity to occur in different crystalline forms). These different crystalline forms are typically known as “polymorphs.” It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs may have different physical properties such as density, shape, hardness, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjust the conditions used during the crystallization or recrystallization of the compound.

For solvates of compounds of this invention, or salts thereof, that are in crystalline form, the skilled artisan will appreciate that pharmaceutically acceptable solvates may be formed wherein solvent molecules are incorporated into the crystalline lattice during crystallization. Solvates may involve nonaqueous solvents such as ethanol, isopropanol, dimethyl sulfoxide, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

Because of their potential use in medicine, the salts of the compound(s) of the invention are preferably pharmaceutically acceptable. Suitable pharmaceutically acceptable salts include those described by P. Heinrich Stahl and Camille G. Wermuth in Handbook of Pharmaceutical Salts: Properties, Selection, and Use, 2^{nd} ed. (Wiley-VCH: 2011) and also Remington's Pharmaceutical Sciences, 18th ed. (Mack Publishing, Easton Pa.: 1990) and also Remington: The Science and Practice of Pharmacy, 19th ed. (Mack Publishing, Easton Pa.: 1995).

Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, trifluoroacetate, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

Salts of a compound of the present invention may be prepared by any suitable method known in the art, including treatment of the free bases with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, formic acid, alginic acid, pyruvic acid, oxalic acid, glycolic acid, salicylic acid, pyranosildyl acid, such as glucuronic acid or galacturonic acid, alphahydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like.

A pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, zinc, as well as salts made from physiologically acceptable organic bases such as diethylamine, isopropylamine, olamine, benzathine, benethamine, tromethamine (2-amino-2-(hydroxymethyl)propane-1,3-diol), morpholine, epolamine, piperidine, piperazine, picoline, dicyclohexylamine, N,N′-dibenzylethylenediamine, 2-hydroxyethylamine, tri-(2-hydroxyethyl)amine, chloroprocaine, choline, deanol, imidazole, diethanolamine, ethylenediamine, meglumine (N-methylglucamine), procaine, dibenzylpiperidine, dehydroabietylamine, glucamine, collidine, quinine, quinolone, erbumine and basic amino acids such as lysine and arginine.

If a compound containing a basic amine or other basic functional group is isolated as a salt, the corresponding free base form of that compound may be prepared by any suitable method known to the art, including treatment of the salt with an inorganic or organic base, suitably an inorganic or organic base having a higher pKₐ than the free base form of the compound. Similarly, if a compound containing a phosphate diester, phosphorothioate diester or other acidic functional group is isolated as a salt, the corresponding free acid form of that compound may be prepared by any suitable method known to the art, including treatment of the salt with an inorganic or organic acid, suitably an inorganic or organic acid having a lower pKₐ than the free acid form of the compound.

An effective amount of a compound or a pharmaceutically acceptable salt, pharmaceutically acceptable solvate or pharmaceutically acceptable hydrate thereof, as described herein, for a particular patient may vary depending on factors such as the condition being treated, the overall health of the patient, the route and dose of administration and the severity of side effects.

Methods for co-administration with an additional therapeutic agent are well known in the art (Hardman, et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeuticsfor Advanced Practice:A Practical Approach, Lippincott, Williams & Wilkins, Phila., PA). Generally, co-administration or administration together indicates treating a subject with two or more agents, where the agents can be administered simultaneously or at different times. For example, such agents may be delivered to a single subject as separate administrations, which may be at essentially the same time or different times, and which may be by the same route or different routes of administration. Such agents may be delivered to a single subject in the same administration (e.g., same formulation) such that they are administered at the same time by the same route of administration.

Generally, each administration of a compound of the invention comprises between about 10 mg to about 2000 mg in an individual, e.g., from about 10 mg to about 50 mg, from about 50 mg to about 100 mg, from about 100 mg to about 250 mg, from about 250 mg to about 500 mg, from about 500 mg to about 1000 mg, from about 1000 mg to about 2000 mg. In an embodiment the compound(s) of Formula I are administered in doses of about 10 mg to about 2000 mg. In an embodiment the compound(s) of Formula I are administered in doses of about 10 mg to about 2000 mg. In an embodiment the compound(s) of Formula I are administered in doses of about 10 mg to about 200 mg. In an embodiment the compound(s) of Formula I are administered in doses of about 100 mg to about 1000 mg.

### General Synthetic Methods

All synthetic chemistry was performed in standard laboratory glassware unless indicated otherwise in the examples. Commercial reagents were used as received. Microwave reactions were performed in a Biotage Initiator using the instrument software to control heating time and pressure. Analytical LC/MS was performed on an Agilent 1290 infinity, Mass:6150 SQD(ESI/APCI) or an Agilent 1200 SERIES, Mass:6130SQD(ESI/APCI); variable wavelength detector and Agilent 6130 single quadrupole mass spectrometer, alternating positive and negative ion scans using Chemistation software. Retention times were determined from the extracted 220 nm UV chromatogram. HPLC was performed on a Waters 2695 system with a variable wavelength detector using Empower software. Retention times were determined from the extracted 210 nm and 300 nm UV chromatograms. ¹H NMR was performed on a Bruker Avance 400 at 400 MHz or a Bruker Avance DRX-500 at 500 MHz using Topspin software. For complicated splitting patterns, the apparent splitting is tabulated. Analytical thin layer chromatography was performed on silica (Macherey-Nagel ALUGRAM Xtra SIL G, 0.2 mm, UV₂₅₄ indicator) and was visualized under UV light. Silica gel chromatography was performed manually, or with Grace automated chromatography for gradient elution. Melting points were collected using a Buchi B-540 melting point apparatus.

In one method (Scheme 1) the synthetic preparation may originate from the acylation of (R)-4-benzyloxazolidin-2-one [102029-44-7] II with III, where Lv is defined as a leaving group such as a halogen atom or a mixed anhydride such as —OC(O)^{t}Bu. In other incidences Lv can also be defined as a sulfonate such as —OSO₂CH₃ (mesylate), —OSO₂CF₃ (triflate), or —OSO₂Ar where Ar is 4-methylphenyl (tosylate). The N-acyl-oxazolinone IV formed can be subjected to an asymmetric alkylation reaction with benzyloxymethyl chloride under conditions originally described by Evans, D.A.; et al., JACS, 1990, pp. 8215, to form the (R,S) diastereomer of V. The olefin of V may then be subjected to ozone or periodate oxidative cleavage to form aldehyde VI. Under reductive amination conditions with a benzylic amine such as 4-methoxy-benzyl amine, VI can be transformed to cyclic lactam VII. The benzylic ether of VII can be remove via catalytic hydrogenation to form alcohol VIII. Intermediate VIII can then be transformed to IX where Lv is defined above. Displacement of the Lv group of IX with sodium azide can yield X. Exposure of the azide of X to appropriate reduction conditions can be used to form primary amine XI. α—Chloroketones XII can be produced in a 3-step process via its' corresponding α-diazoketone from amino acids where P¹ is a carbamyl protecting group utilizing methods known in the literature. N-alkylation of XI with XII under basic conditions may be used to form intermediate XIII. The newly formed secondary amine of XIII can be protected with an appropriate reagent where P² is a carbamyl protecting group different than P¹ giving intermediate XIV. The selective deprotection of P¹ from XIV can give primary amine XV or XVa if the benzylic group is simultaneously removed from the lactam. Intermediate XVI or XVIa may be generated from XV or XVa respectively with R¹-CO₂H or R¹-C(O)Lv utilizing an amide forming reaction where R¹ and Lv are defined above. Secondary amines XVII or XVIIa may be prepared by deprotection of XVI or XVIa respectively. Further, the benzylic group of lactam XVII may be removed with 2,3-dichloro-5,6-dicyano-1,4-benzoquinone (DDQ) or under acidic condition with trifluoroacetic acid (TFA). Under basic conditions, exposing XVII or XVIIa to R⁴-Lv, where R⁴ and Lv as defined above, can form compounds of XVIII or Formula I respectively, where Z is CH₂. Removal of the benzylic group from the lactam nitrogen of XVIII with DDQ or TFA may afford compounds of Formula I, where Z is CH₂.

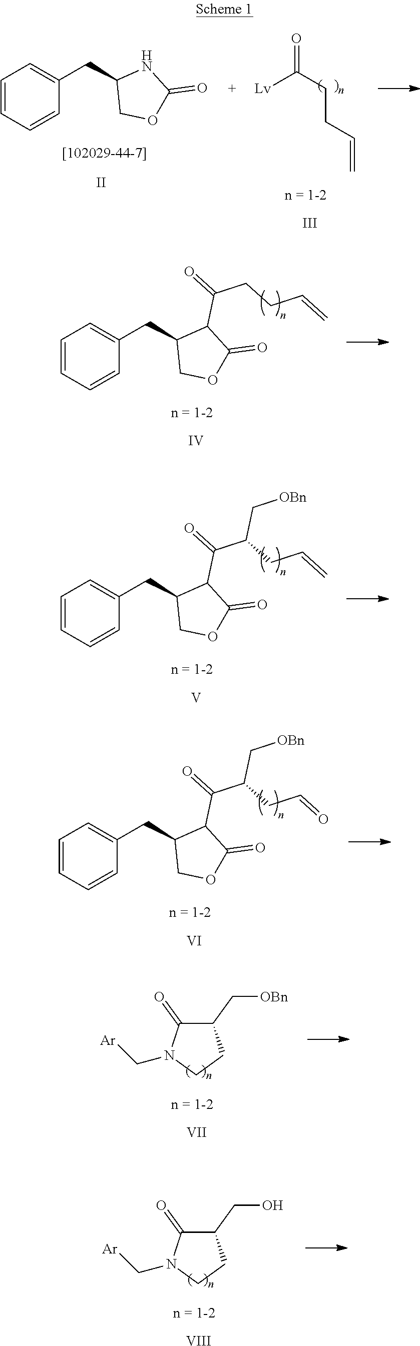

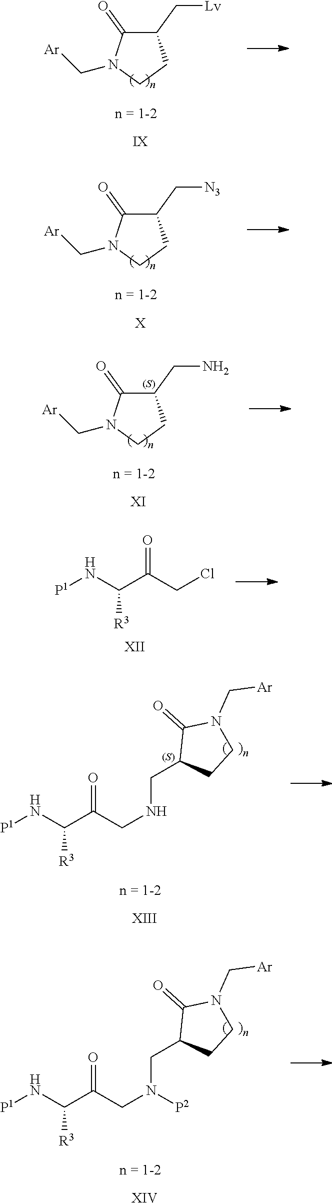

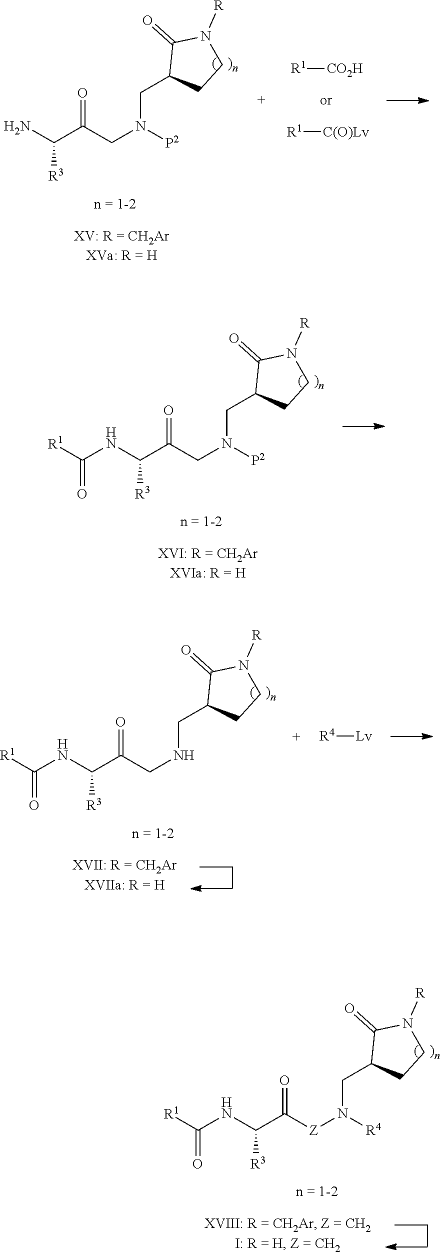

In another method (Scheme 2) intermediate XVa can be synthesized by a slightly modified route. The nitrogen atom of II can be acylated with XIX or XIXa, where Lv is defined as a leaving group such as a halogen atom or a mixed anhydride such as OC(O)^{t}Bu. When R is Br, XXa can be converted to azide XX with sodium azide. Asymmetric introduction of a hydroxymethyl or benzyloxymethyl group to XX can be performed to give either XXI or XXIa respectively. Reduction of the azide of XXI, or both azide and benzyl ether simultaneously of XXIa followed by cyclization can produce lactam XXII. The hydroxyl group of XXII can then be transformed to a leaving group in XXIII where Lv is define above. Displacement of the leaving group of XXIII can yield azide XXIV, which can be reduce to primary amine XXV. Alkylation of the amine with α-chloroketone XII may form intermediate XXVI. The secondary amine of XXVI can be protected with an appropriate reagent where P² is a carbamyl protecting group different than P¹ giving intermediate XXVII. The selective deprotection of P¹ from XXVII can give primary amine XVa.

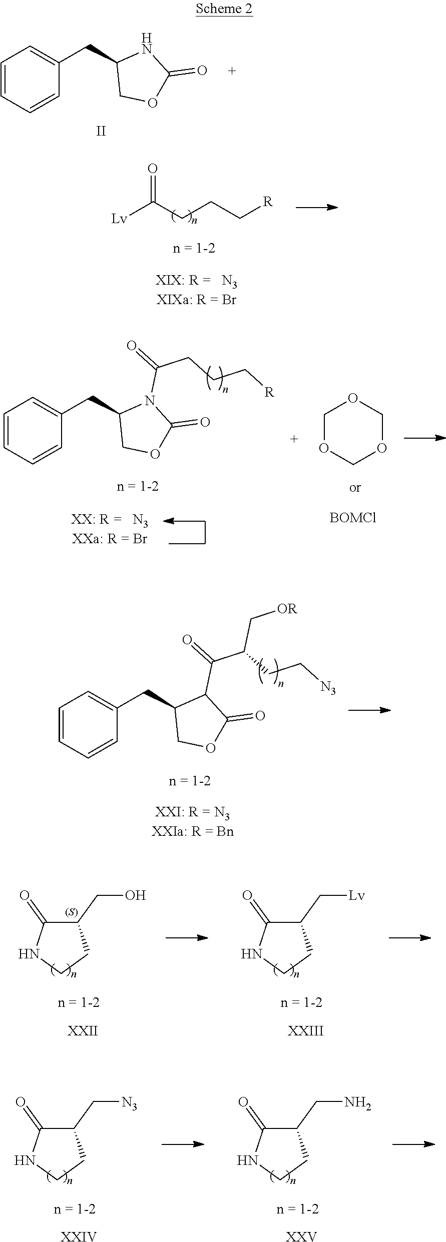

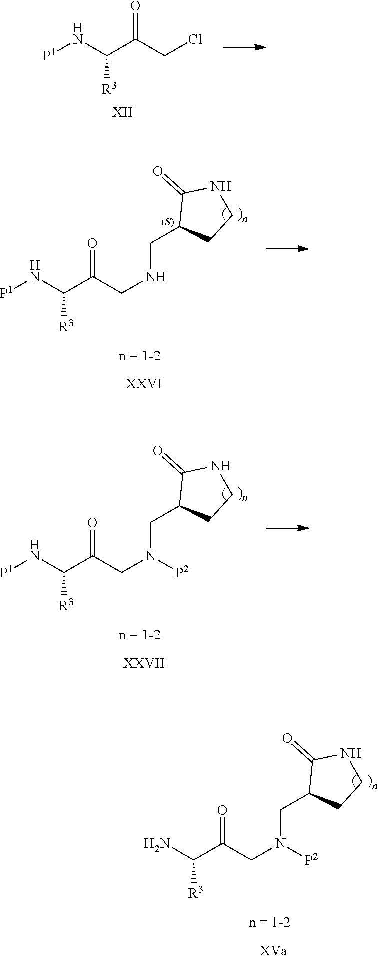

In a third method (Scheme 3) the synthesis starts with lactam intermediate IX. Alkylation of tert-butyl 2-(propan-2-ylidene)hydrazine-1-carboxylate [16689-34-2] XXVIII can be conducted under phase transfer conditions to afford hydrazide XXIX. Exposing XXIX to HCl can form hydrazine intermediate XXX. Under various amide forming conditions, XXX and an N-protected amino acid XXXI, where P¹ is defined above, the hydrazide XXXII may be generated. The hydrazide XXXII can be protected with an appropriate reagent where P² is a carbamyl protecting group different than P¹ giving intermediate XXXIII. The selective deprotection of P¹ from XXXIII can give intermediates XXXIV or XXXIVa if the benzylic group is simultaneously removed from the lactam. Intermediate XXXV or XXXVa may be generated from XXXIV or XXXIVa respectively with R¹-CO₂H or R¹-C(O)Lv utilizing an amide forming reaction and where R¹ and Lv are defined above. Hydrazides XXXVI or XXXVIa may be prepared by deprotection of XXXVI or XXXVa respectively. Further, the benzylic group of lactam XXXVI may be removed with DDQ or under acidic condition with TFA. Under basic conditions, exposing XXXVI or XXXVIa to R⁴-Lv, where R⁴ and Lv are defined above, can form compounds of XXXVII or Formula I respectively, where Z is NH. Removal of the benzylic group from the lactam nitrogen of XXXVII with DDQ or TFA may afford compounds of Formula I, where Z is NH.

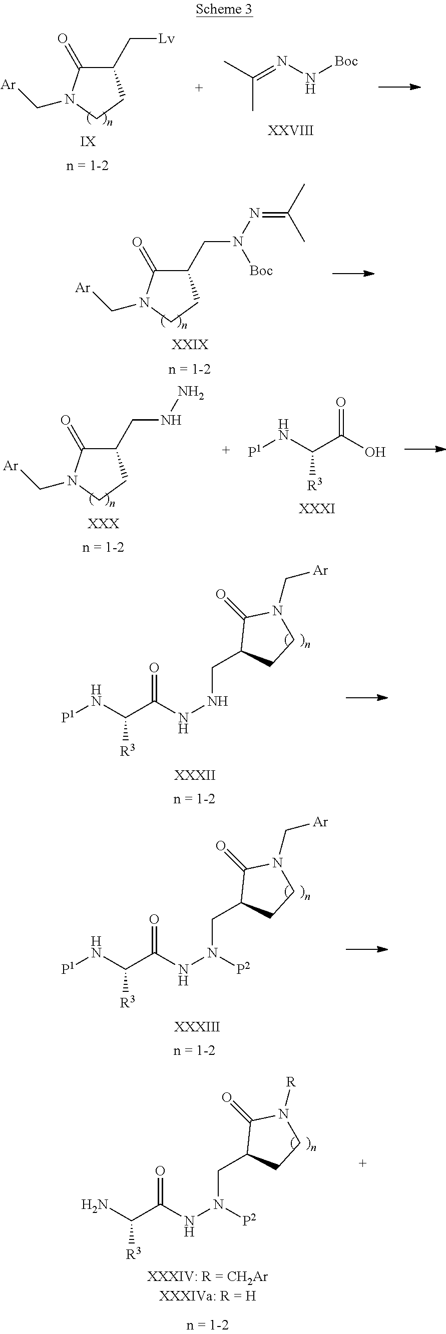

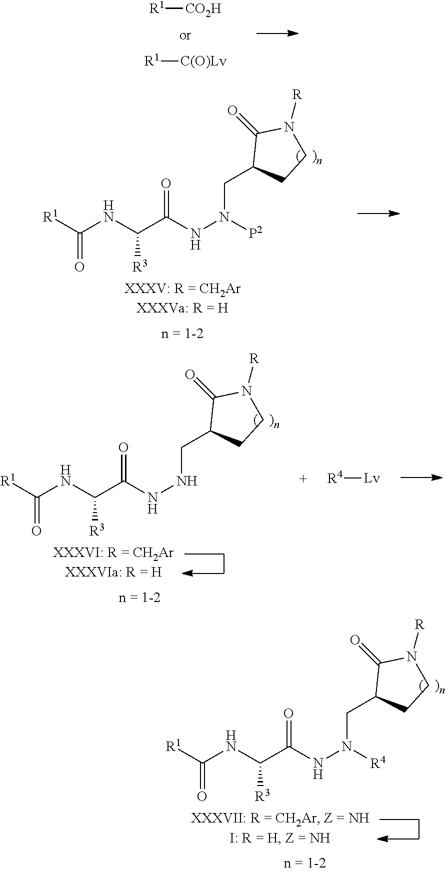

### EXAMPLES

### Example 1: Tert-butyl ((S)-1-(2-chloro-N—(((S)-2-oxopyrrolidin-3-yl)methyl)acetamido)-5-methyl-2-oxohexan-3-yl)carbamate, Compound 1

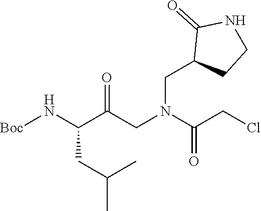 [SMILES: CC(C)C[C@H](NC(=O)OC(C)(C)C)C(=O)CN(C[C@@H]1CCNC1=O)C(=O)CCl]

Compound 1 was prepared according to the following multistep procedures.

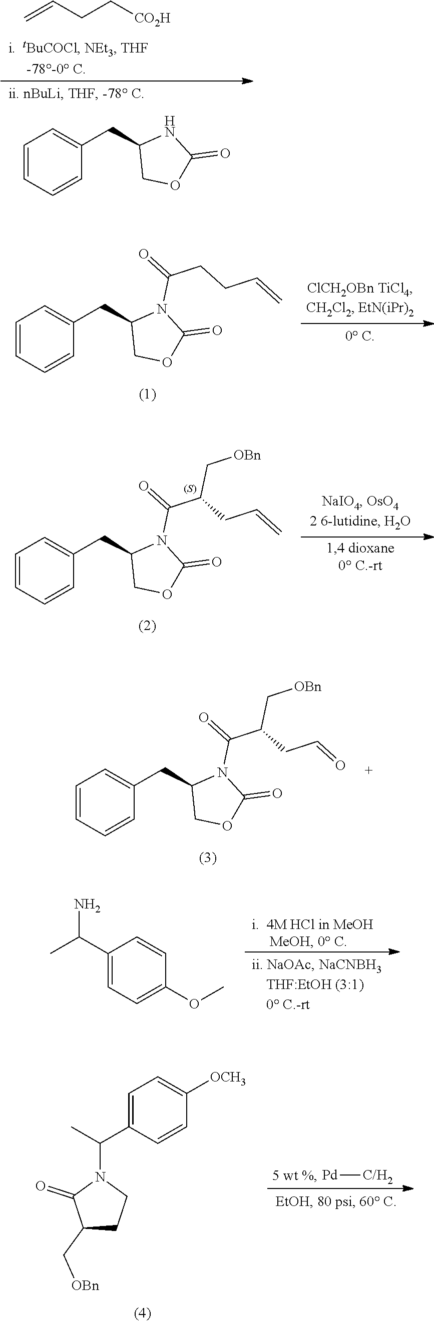

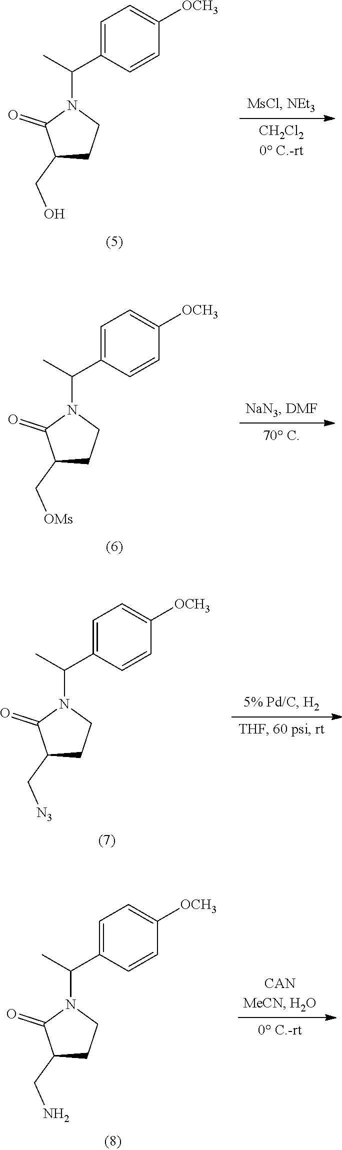 [SMILES: COc1ccc(C(C)N2CC[C@@H](CO)C2=O)cc1 ; COc1ccc(C(C)N2CC[C@@H](COS(C)(=O)=O)C2=O)cc1 ; COc1ccc(C(C)N2CC[C@@H](CN=[N+]=[N-])C2=O)cc1 ; COc1ccc(C(C)N2CC[C@@H](CN)C2=O)cc1]

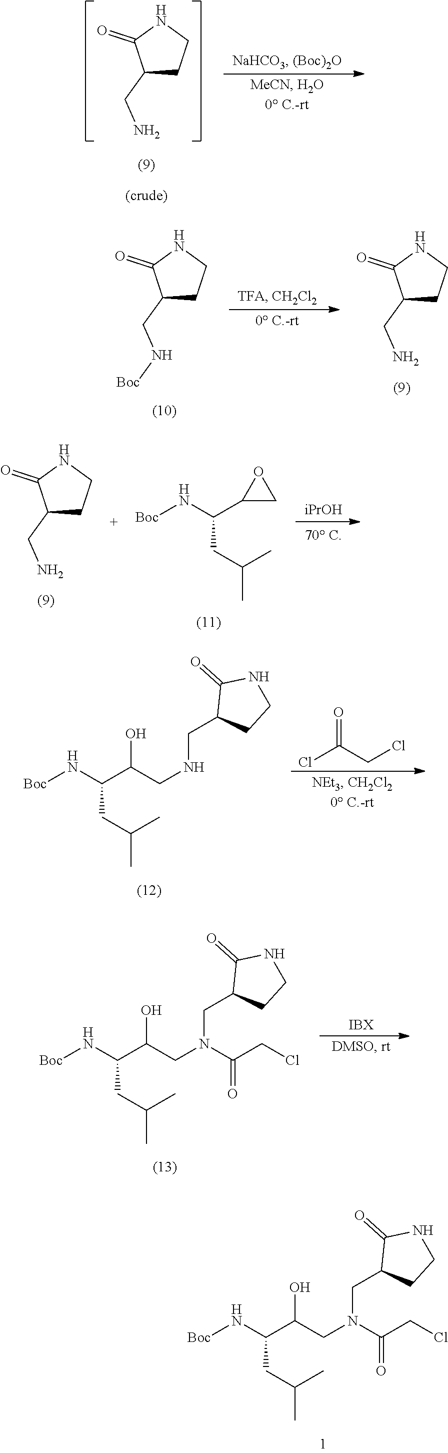

Step-1: (R)-4-Benzyl-3-(pent-4-enoyl)oxazolidin-2-one (1)

To a stirred solution of 4-pentenoic acid (75 mL, 0.734 mol, 1.3 equiv.) and Et₃N (140 mL, 1.005 mol, 1.78 equiv.) in anhydrous THE (1.5 L) was added pivaloyl chloride (97 mL, 0.79 mol, 1.4 equiv.) at −78° C. The reaction mixture was gradually warmed to 0° C. over 1 h and stirred for additional 1 h at the same temperature. In a separate flask, a solution of (R)-4-benzyloxazolidine-2-one (100 g, 0.564 mol, 1 equiv.) in anhydrous THE (1 L) was cooled to −78° C. whereupon nBuLi (2.5 M in hexane, 237 mL, 592 mmol, 1.05 equiv.) was added slowly. The solution was stirred for 1 h at −78° C. The flask containing the mixed anhydride was cooled to −78° C., and the lithium anion of oxazolidinone was transferred via cannula into the mixed anhydride. After being stirred for 1 h at −78° C. the reaction mixture was slowly warmed to rt and stirred for 6 h. The reaction was quenched with saturated NH₄Cl (500 mL) at 0° C., the layers were separated, and the aqueous layer was extracted with EtOAc (3×200 mL). The combined organic layers were washed with brine (500 mL), dried over Na₂SO₄ and concentrated under reduced pressure. The resulted crude compound was purified by column chromatography using 20-30% EtOAc/Pet ether (v/v) as the eluent to afford (R)-4-benzyl-3-(pent-4-enoyl) oxazolidin-2-one (1) (125 g, 85.6%) as a colorless oil. TLC: R_{f} 0.2 (20% EtOAc/Pet ether); C₁₅H₁₇NO₃; ¹H—NMR (400 MHz, CDCl₃): δ 7.35-7.26 (m, 3H), 7.21-7.19 (m, 2H), 5.93-5.83 (m, 1H), 5.13-5.02 (m, 2H), 4.69-4.65 (m, 1H), 4.22-4.15 (m, 2H), 3.30 (dd, J=3.2, 13.6 Hz, 1H), 3.12-2.99 (m, 2H), 2.76 (dd, J=9.6, 13.2 Hz, 1H), 2.48-2.43 (m, 2H); LC/MS: Rt=2.03 min (96.7%); m/z 260.4 [M+H]*; HPLC: 98% (6.61 min) X-Bridge C18 (50 mm×4.6 mm, 3.5 m).

Step 2: (R)-4-Benzyl-3-((S)-2-((benzyloxy)methyl)pent-4-enoyl)oxazolidin-2-one (2)

To a stirred solution of (R)-4-benzyl-3-(pent-4-enoyl) oxazolidin-2-one (1) (125 g, 482 mmol, 1 equiv.) in CH₂Cl₂ (1.25 L) at 0° C. was drop wise added TiCl₄ (56 mL, 507 mmol, 1.05 equiv.) via syringe. The mixture was stirred at 0° C. for 30 min and then DIPEA (92.5 mL, 531 mmol, 1.1 equiv.) was added. The mixture was stirred at 0° C. for 30 min and then benzyl chloromethyl ether (134 mL, 965 mmol, 2 equiv.) was added, and the resulting mixture was stirred for another 3 h at 0° C. After 3 h, the reaction mixture was quenched with 1 L of saturated NaHCO₃ solution at 0° C. The aqueous layer was extracted with CH₂Cl₂ (3×300 mL). The combined organic layers were washed with saturated NaHCO₃ (2×500 mL) and brine (500 mL). The organic layer was dried over Na₂SO₄ and concentrated in vacuo. The residue was purified by column chromatography on 100-200 silica gel using 20-30% EtOAc/Pet ether (v/v) as the eluent to provide (R)-4-benzyl-3-((S)-2-((benzyloxy)methyl)pent-4-enoyl)oxazolidin-2-one (2) (132 g, 72.5%) as colorless oil.

TLC: R_{f} 0.2 (20% EtOAc/Pet ether); C₂₃H₂₅NO₄; ¹H NMR (400 MHz, CDCl₃): δ 7.32-7.30 (m, 4H), 7.29-7.24 (m, 4H), 7.22-7.17 (m, 2H), 5.78 (m, 1H), 5.07 (d, J=17.0, 1.8 Hz, 1H), 5.02 (dt, J=10.2, 0.8 Hz, 1H), 4.70 (m, 1H), 4.55 (s, 2H), 4.31 (m, 1H), 4.17-4.10 (m, 2H), 3.81 (dd, J=7.6, 9.2 Hz, 1H), 3.66 (dd, J=5.2, 9.2 Hz, 1H), 3.22 (dd, J=3.2, 13.6 Hz, 1H), 2.70 (dd, J=9.2, 13.6 Hz, 1H), 2.5-2.4 (m, 1H), 2.39-2.29 (m, 1H); LC/MS: Rt=2.21 min (95.5%); m/z 380.5 [M+H]*; HPLC: 98% (7.12 min.) Acquity UPLC BEH C18 (100 mm×2.1 mm) 1.7 m).

Step-3: (S)-4-((R)-4-Benzyl-2-oxooxazolidin-3-yl)-3-((benzyloxy)methyl)-4-oxobutanal (3)

To a stirred solution of (R)-4-benzyl-3-((S)-2-((benzyloxy)methyl)pent-4-enoyl)oxazolidin-2-one (2) (130 g, 343 mmol, 1 equiv.) in 1,4-dioxane and water (3:1, 2.6 L) at 0° C. was added 2,6 lutidine (81 mL, 686 mmol, 2 equiv.), OsO₄ (4% in H₂O, 21.8 mL, 0.01 equiv.) and NaIO₄ (293 g, 1.372 mol, 4 equiv.). The mixture was stirred at 0° C. to rt for 6 h. The progress of the reaction was monitored by TLC and LC/MS. After complete consumption of compound (2), the residual solvent was evaporated and the aqueous layer extracted with CH₂Cl₂ (3×500 mL). The combined organic layers were washed with saturated NaHCO₃ (2×500 mL), brine (2×300 mL), dried over sodium sulfate and concentrated under reduced pressure. The crude residue was purified by column chromatography on 100-200 silica gel using 15-25% EtOAc/Pet ether (v/v) as the eluent to get (S)-4-((R)-4-benzyl-2-oxooxazolidin-3-yl)-3-((benzyloxy)methyl)-4-oxobutanal (3) (92 g, 70%) as colorless oil. TLC: R_{f} 0.3 (50% EtOAc/Pet ether); C₂₂H₂₃NO₅; ¹H NMR (400 MHz, CDCl₃): δ 9.7 (s, 1H), 7.35-7.23 (m, 8H), 7.17-7.16 (m, 2H), 4.71-4.69 (m, 1H), 4.54 (s, 2H), 4.49-4.46 (m, 1H), 4.27 (t, J=8.0 Hz, 1H), 4.17-4.11 (m, 1H), 3.72 (d, J=5.6 Hz, 2H), 3.23-3.16 (m, 2H), 2.79-2.66 (m, 2H); LC/MS: Rt=1.97 min (59%); m/z 382.3 [M+H]*.

Step 4: (3S)-3-((Benzyloxy)methyl)-1-(1-(4-methoxyphenyl)-ethyl)pyrrolidin-2-one (4)

To a stirred solution cooled to 0° C. of racemic 1-(4-methoxyphenyl)ethan-1-amine (43 g, 283 mmol, 1.2 equiv.) in MeOH (450 mL) was added 4M HCl in MeOH (88 mL, 354 mmol, 1.5 equiv.). After 3 h, the residual solvent was evaporated under reduced pressure to get racemic 1-(4-methoxyphenyl)ethan-1-amine hydrochloride salt as a white solid. In a separate flask, a solution of (S)-4-((R)-4-benzyl-2-oxooxazolidin-3-yl)-3-((benzyloxy)methyl)-4-oxobutanal (3) (90 g, 236 mmol, 1.0 equiv.) in THF and EtOH (4:1, 1.8 L) cooled to O ° C. was added racemic the 1-(4-methoxyphenyl)ethan-1-amine hydrochloride salt, NaOAc (77.4 g, 944 mmol, 4.0 equiv.) and NaCNBH₃ (29.6 g, 472 mmol, 2.0 equiv.). After being stirred for 30 min at 0° C., the reaction mixture was warmed to rt and stirred for 12 h. The reaction was monitored by TLC. After complete consumption of compound (3), the reaction mixture was cooled to 0° C. and quenched with water (500 mL). The residual solvent was evaporated, and aqueous layer was extracted with EtOAc (3×500 mL). The combined organic layers were washed with brine (500 mL), dried over Na₂SO₄ and concentrated under reduced pressure. The crude compound was purified by column chromatography on 100-200 silica gel using 50-75% EtOAc/Pet-ether (v/v) as the eluent to afford (3S)-3-((benzyloxy)methyl)-1-(1-(4-methoxyphenyl)ethyl)pyrrolidin-2-one (4) (55 g, 68%) as colorless oil. TLC: R_{f} 0.1 (50% EtOAc/Pet ether); C₂₁H₂₅NO₃; ¹H NMR (400 MHz, CDCl₃): δ 7.36-7.25 (m, 5H), 7.23-7.16 (m, 2H), 6.87-6.83 (m, 1H), 6.74-6.72 (m, 1H), 5.47-5.42 (m, 1H), 4.57-4.47 (m, 2H), 3.80-3.70 (m, 5H), 3.32-3.21 (m, 1H), 2.99-2.84 (m, 1H), 2.75-2.68 (m, 1H), 2.14-2.04 (m, 1H), 1.99-1.93 (m, 1H), 1.48 (t, J=8.0 Hz, 3H); LC/MS: Rt=2.10 min (36.5%), 2.14 min (52.8%); m/z 340.2 [M+H]*.

Step-5: (3S)-3-(Hydroxymethyl)-1-(1-(4-methoxyphenyl)ethyl)pyrrolidin-2-one (5) [00115] (3S)-3-((benzyloxy)methyl)-1-(1-(4-methoxyphenyl)ethyl)pyrrolidin-2-one (4) (55 g, 162 mmol, 1 equiv.) was taken up in 1 L parr shaker flask in EtOH (220 mL). Pd/C catalyst (2.75 g, 5% (w/w), 50% wet) was added at rt and stirred at 60° C. under a hydrogen atmosphere at 80 psi for 24 h. Progress of the reaction was monitored by TLC and LC/MS. The reaction mixture was filtered through a pad of celite and washed with 10% MeOH/CH₂Cl₂ (2×100 mL). The combined organic layers were concentrated under reduced pressure and the residue was purified by column chromatography on 100-200 silica gel using 5% MeOH/CH₂Cl₂ (v/v) as the eluent to afford (3S)-3-(hydroxymethyl)-1-(1-(4-methoxyphenyl)ethyl)pyrrolidin-2-one (5) (28 g, 70%) as a white solid. TLC: R_{f} 0.1 (50% EtOAc/Pet ether); C₁₄H₁₉NO₃; ¹H NMR (400 MHz, CDCl₃): δ 7.22-7.17 (m, 2H), 6.88-6.85 (m, 2H), 5.46-5.39 (m, 1H), 3.89-3.84 (m, 1H), 3.80 (s, 3H), 3.75-3.67 (m, 1H), 3.31-3.25 (m, 1H), 3.01-2.89 (m, 1H), 2.78-2.60 (m, 1H), 2.07-2.02 (m, 1H), 1.76-1.61 (m, 2H), 1.52-1.49 (m, 3H); LC/MS: Rt=1.41 min (97.6%); m/z 250.1 [M+H]*.

Step-6: ((3S)-1-(1-(4-Methoxyphenyl)ethyl)-2-oxopyrrolidin-3-yl)methyl methanesulfonate (6)

To a stirred solution of (3S)-3-(hydroxymethyl)-1-(1-(4-methoxyphenyl)ethyl)pyrrolidin-2-one (5) (28 g, 112 mmol., 1.0 equiv.) and Et₃N (47 mL, 336 mmol, 3.0 equiv.) in anhydrous dichloromethane (140 mL) cooled at 0° C. was slowly added methanesulfonyl chloride (13 mL, 168 mmol, 1.5 equiv.). The mixture was stirred at 0° C. and gradually warm to rt over 3 h. The reaction progress was monitored by TLC. After consumption of (5) water (100 mL) was added and the reaction mixture was extracted with dichloromethane (2×80 mL). The combined organic extracts were washed with brine (100 mL), dried over anhydrous Na₂SO₄, concentrated under reduced pressure to result a crude ((3S)-1-(1-(4-methoxyphenyl)ethyl)-2-oxopyrrolidin-3-yl)methyl methanesulfonate (6) (40 g, quantitative) as a yellow liquid which was used in the next step without further purification. TLC: R_{f} 0.3 (50% EtOAc/Pet ether); C₁₅H₂₁NO₅S; ¹H NMR (400 MHz, CDCl₃): δ 7.22-7.18 (m, 2H), 6.88-6.85 (m, 2H), 5.43-5.40 (m, 1H), 4.53-4.49 (m, 1H), 4.45-4.41 (m, 1H), 3.8 (s, 3H), 3.32-3.28 (m, 1H), 3.04 (s, 3H), 2.93-2.89 (m, 1H), 2.80-2.78 (m, 1H), 2.20-1.76 (m, 2H), 1.51 (d, J=7.2 Hz, 3H); LC-MS: Rt=1.61 min (33.8%), 1.64 min (58%); m/z 328.17 [M+H]*.

Step-7: (3S)-3-(Azidomethyl)-1-(1-(4-methoxyphenyl)ethyl)pyrrolidin-2-one (7)

To a stirred solution of ((3S)-1-(1-(4-methoxyphenyl)ethyl)-2-oxopyrrolidin-3-yl)methyl methanesulfonate (6) (40 g, 122 mmol, 1.0 equiv.) in DMF (200 mL) was added NaN₃ (12 g, 183 mmol, 1.5 equiv.) at 0° C. and the resultant reaction mixture was stirred at 70° C. for 4 h under a nitrogen atmosphere. The reaction progress was monitored by TLC and LC/MS. After complete consumption of (7), the reaction mixture was cooled to rt, and ice-water (200 mL) was added. The aqueous layer was extracted with EtOAc (3×200 mL), the combined organic extracts were washed with brine (2×100 mL), dried over anhydrous Na₂SO₄, filtered and concentrated under reduced pressure. The crude residue was purified by column chromatography using 20-30% EtOAc/Pet-ether (v/v) as the eluent to get (3S)-3-(azidomethyl)-1-(1-(4-methoxyphenyl)ethyl)pyrrolidin-2-one (7) (27 g, 88% over 2 steps) as a colorless liquid. TLC: R_{f}0.5 (50% EtOAc/Pet ether); C₁₄H₁₈N₄O₂; ¹H NMR (400 MHz, CDCl₃): δ 7.22-7.20 (m, 2H), 6.88-6.85 (m, 2H), 5.48-5.43 (m, 1H), 5.46-5.43 (m, 1H), 3.80-3.60 (m, 4H), 3.32-3.23 (m, 1H), 2.97-2.88 (m, 1H), 2.72-2.64 (m, 1H), 2.15-2.09 (m, 1H), 1.90-1.81 (m, 1H), 1.50 (t, J=6.8 Hz, 3H); LC-MS: Rt=1.81 min (37.1%), 1.83 min (53%); m/z 275.2 [M+H]*.

Step-8: (3S)-3-(Aminomethyl)-1-(1-(4-methoxyphenyl)ethyl)pyrrolidin-2-one (8)

To (3S)-3-(azidomethyl)-1-(1-(4-methoxyphenyl) ethyl) pyrrolidin-2-one (7) (27 g, 98.5 mmol, 1.0 equiv.) in 500 mL in parr shaker flask was added THE (135 mL) and Pd/C catalyst (1.35 g, 5% (w/w), 50% wet). The heterogenous reaction mixture was stirred at rt for 12 h under a hydrogen atmosphere at 60 psi. The reaction progress was monitored by TLC and LC/MS. After completion of reaction, the reaction mixture was filtered through a pad of celite and washed with 10% MeOH in CH₂Cl₂ (2×100 mL). The combined organic layers were concentrated under reduced pressure and the residue was purified by column chromatography on 100-200 silica gel, using 5-10% MeOH in CH₂Cl₂ (v/v) as the eluent to afford the (3S)-3-(amino methyl)-1-(1-(4-methoxyphenyl)ethyl) pyrrolidin-2-one (8) (23 g, 94%) as an off-white semi solid. TLC: R_{f} 0.10 (10% MeOH/CH₂Cl₂); C₁₄H₂₀N₂O₂; ¹H NMR (500 MHz, DMSO-d₆) δ 8.02 (bs, 2H), 7.21-7.19 (m, 2H), 6.91-6.89 (m, 2H), 5.20-5.16 (m, 1H), 3.74 (s, 3H), 3.39-3.26 (m, 1H), 3.07-3.06 (m, 1H), 2.96-2.71 (m, 3H), 2.25-2.10 (m, 1H), 1.76-1.64 (m, 1H), 1.45-1.42 (m, 3H); LC/MS: Rt=1.05 min (97.1%); m/z 249 [M+H]*.

Step-9: (S)-3-(Aminomethyl)pyrrolidin-2-one (9) crude

To a stirred solution of (3S)-3-(amino methyl)-1-(1-(4-methoxyphenyl) ethyl) pyrrolidin-2-one (8) (23 g, 92.7 mmol, 1.0 equiv.) in acetonitrile (3:1, 345 mL) was added ceric ammonium nitrite (CAN) (152 g, 278 mmol, 3.0 equiv.) in water (115 mL) at 0° C. The above mixture was stirred at 0° C. to rt for 6 h. The reaction progress was monitored by TLC and LC/MS. After complete consumption of compound (8) the solvent was evaporated and aqueous layer was extracted with Et₂O (2×100 mL). To the aqueous layer was added saturated NaHCO₃ (200 mL) slowly at 0° C., the solids were filtered, and filtrate was concentrated under reduced pressure to get the crude (S)-3-(amino methyl) pyrrolidin-2-one (9) (10.5 g, quantitative). The crude compound was used for next step without further purification. TLC: R_{f}=0.1 (20% MeOH/CH₂Cl₂); Rt=0.30 min (99.3%); m/z 115 [M+H]*.

Step-10: Tert-butyl (S)-((2-oxopyrrolidin-3-yl)methyl)carbamate (10)

To a stirred solution of crude (S)-3-(amino methyl) pyrrolidin-2-one (9) (10.5 g, 92.1 mmol, 1.0 equiv.) in acetonitrile (105 mL) and H₂O (315 mL) were added solid NaHCO₃ (23 g, 276 mmol, 3.0 equiv.) and (Boc)₂0 (31 mL, 138 mmol, 1.5 equiv.) at 0° C. The reaction mixture was stirred at 0° C. to rt for 10 h. and monitored by TLC and LC-MS. After consumption of (9), the residual solvent was evaporated, and the aqueous phase extracted with 10% MeOH in CH₂Cl₂(3×100 mL). The combined organic extracts were washed with brine (100 mL), dried over anhydrous Na₂SO₄, filtered and concentrated under reduced pressure. The residue was purified by column chromatography using 3% MeOH in CH₂Cl₂ (v/v) as the eluent to afford tert-butyl (S)-((2-oxopyrrolidin-3-yl) methyl) carbamate (10) (19 g, 95% over 2 steps) as colorless gummy liquid. TLC: R_{f}=0.25 (10% MeOH/CH₂Cl₂); C₁₀H₁₈N₂O₃; ¹H NMR (400 MHz, CDCl₃) δ 5.98 (s, 1H), 5.36 (s, 1H), 3.50-3.48 (m, 1H), 3.36-3.25 (m, 3H), 2.55-2.49 (m, 1H), 2.29-2.24 (m, 1H), 1.98-1.89 (m, 1H), 1.45 (s, 9H), LC/MS: Rt=1.38 min (99.5%); m/z 215.1 [M+H]*.

Step-11: (S)-3-(Aminomethyl)pyrrolidin-2-one (9) pure

To a stirred solution of tert-butyl (S)-((2-oxopyrrolidin-3-yl) methyl) carbamate (10) (19 g, 88.7 mmol, 1.0 equiv.) in CH₂Cl₂ (190 mL) was added TFA (40 mL, 532 mmol, 6.0 equiv.) at 0° C. and the resultant reaction mixture was stirred at rt for 12 h. until TLC indicated consumption of starting material. The reaction mixture was concentrated under reduced pressure. The residue was taken up 3-4 times in CH₂Cl₂ (100 mL) and reconcentrated to remove excess TFA. The crude TFA salt was dissolved in 10% MeOH in CH₂Cl₂ (150 mL) and made basic with Amberlyst A21 at 0° C. The resin was filtered and washed with 20% MeOH in CH₂Cl₂ (3×50 mL). The filtrate was concentrated under reduced pressure to get (S)-3-(aminomethyl) pyrrolidin-2-one (9) (9 g, 90%) as a brown color gummy liquid, which was used for next step without further purification. TLC: R_{f} 0.1 (20% MeOH/CH₂Cl₂); ¹H NMR (400 MHz, DMSO-d₆) δ 7.83 (s, 1H), 3.20-3.16 (m, 2H), 2.96 (dd, J=6.4, 12.8 Hz, 1H), 2.81 (dd, J=7.6, 12.4 Hz, 1H), 2.50-2.44 (m, 1H), 2.22-2.14 (m, 1H), 1.83-1.73 (m, 1H,); LC-MS: Rt=0.28 min (95.5%); m/z 115 [M+H]+.

Step-12: Tert-butyl ((3S)-2-hydroxy-5-methyl-]-((((S)-2-oxopyrrolidin-3-yl)methyl)amino)hexan-3-yl)carbamate (12)

A solution of (S)-3-(aminomethyl)pyrrolidin-2-one (9) (1.0 g, 8.77 mmol, 1.0 equiv.) and tert-butyl ((1S)-3-methyl-1-(oxiran-2-yl)butyl)carbamate (11) (Konno et al. Synthesis 2003, 14, p. 2161-2164) (2.0 g, 8.77 mmol, 1.0 equiv.) in IPA (60 mL) was stirred at 70° C. for 18 h. The reaction mixture was concentrated under reduced pressure to give the crude product. This was purified by GRACE flash chromatography using 10% MeOH in CH₂Cl₂ as the eluent to provide tert-butyl ((3S)-2-hydroxy-5-methyl-1-((((S)-2-oxopyrrolidin-3-yl)methyl)amino)hexan-3-yl)carbamate (12) (1.2 g, 40%) as an off-white solid as a diastereomeric mixture. C₁₇H₃₃N₃O₄: ¹H NMR (400 MHz, DMSO-d₆) δ 8.82-8.63 (bs, 1H), 8.04-7.97 (m, 1H), 6.70-6.51 (m, 1H), 5.71-4.92 (m, 1H), 3.59-3.33 (m, 2H), 3.19-3.16 (m, 4H), 3.0-2.94 (m, 2H), 2.84-2.68 (m, 2H), 2.31-2.22 (m, 1H), 1.84-1.77 (m, 1H), 1.56-1.54 (m, 1H), 1.38-1.32 (m, 1OH), 0.88-0.82 (m, 6H); LC/MS (ELSD): Rt=1.46 min (99.1%); m/z 344.3 [M+H]+.

Step-13: Tert-butyl ((3S)-1-(2-chloro-N—(((S)-2-oxopyrrolidin-3-yl)methyl)acetamido)-2-hydroxy-5-methylhexan-3-yl)carbamate (13)

To a stirred solution of tert-butyl ((3S)-2-hydroxy-5-methyl-1-((((S)-2-oxopyrrolidin-3-yl)methyl)amino)hexan-3-yl)carbamate (12) (350 mg, 1.02 mmol, 1.0 equiv.) in CH₂Cl₂ (10 mL) at 0° C. was added Et₃N (0.28 mL, 2.04 mmol, 2.0 equiv.), chloroacetyl chloride (90 μL, 1.12 mmol, 1.1 equiv.). The reaction mixture was stirred for 4 h at rt. After complete consumption of (12) by TLC, the reaction mixture was quenched with water (10 mL) and extracted with CH₂Cl₂(2×20 mL). The combined organic layers were washed with brine (2×30 mL), dried over sodium sulfate and concentrated. The crude product was purified by GRACE flash chromatography using 10% MeOH in CH₂Cl₂ as eluent to get tert-butyl ((3S)-1-(2-chloro-N—(((S)-2-oxopyrrolidin-3-yl)methyl) acetamido)-2-hydroxy-5-methylhexan-3-yl) carbamate (13) (0.25 g, 58%) an off-white solid as a diastereomers mixture. C₁₉H₃₄ClN₃O₅: ¹H NMR (400 MHz, DMSO-d₆) δ 7.73-7.67 (m, 1H), 6.68-6.27 (m, 1H), 5.34-5.21 (m, 1H), 4.52-4.23 (m, 2H), 3.76-3.62 (m, 2H), 3.52-3.41 (m, 2H), 3.31-3.08 (m 4H), 2.67-2.50 (m, 1H), 2.30-2.03 (m, 1H), 1.73-1.66 (m, 1H), 1.56-1.54 (m, 1H), 1.38 (m, 10H), 1.26-1.21 (m, 1H), 0.88-0.83 (m 6H). LC/MS: Rt=3.91 min (26.2%) & 3.99 min (68.8%); m/z 420.3 [M+H],; HPLC: 20.9% (9.27 min.) & 77.49 (9.39 min.) X-Bridge C18 (150 mm×4.6 mm, 3.5 m).

Step-14: Tert-butyl ((S)-1-(2-chloro-N—(((S)-2-oxopyrrolidin-3-yl)methyl)acetamido)-5-methyl-2-oxohexan-3-yl)carbamate, Compound 1

To a stirred solution of tert-butyl ((3S)-1-(2-chloro-N—(((S)-2-oxopyrrolidin-3-yl)methyl)acetamido)-2-hydroxy-5-methylhexan-3-yl)carbamate (13) (250 mg, 0.597 mmol, 1.0 equiv.) in DMSO (10 mL) at 0° C. was added 2-iodoxybenzoic acid (IBX) (835 mg, 2.98 mmol, 5.0 equiv.). The stirred reaction mixture was warmed to rt and monitored by LC/MS. After 16 h the reaction mixture was quenched with a saturated NaHCO₃ solution, extracted with CH₂Cl₂(2×50 mL) and washed with NaHCO₃ (3×50 mL), and then by brine (2×50 mL). The combined organic layers were dried over sodium sulfate and evaporated to give a residue that was purified by GRACE flash chromatography as eluent 10% MeOH in DCM giving tert-butyl ((S)-1-(2-chloro-N—(((S)-2-oxopyrrolidin-3-yl)methyl)acetamido)-5-methyl-2-oxohexan-3-yl)carbamate, Compound 1 (170 mg, 68% yield) C₁₉H₃₂ClN₃O₅: ¹H NMR (400 MHz, DMSO-d₆) (rotamers observed as confirmed by VT NMR) δ7.74-7.64 (m, 1H), 7.48-7.28 (m, 1H), 4.57-4.43 (m, 2H), 4.30-3.97 (m, 3H), 3.61-3.31 (m, 3H), 3.17-3.07 (m, 2H), 2.54-2.46 (m, 1H), 2.20-2.00 (m, 1H), 1.77-1.70 (m, 1H), 1.65-1.58 (m, 1H), 1.47-1.39 (m, 1OH), 0.89-0.84 (m, 6H); LC/MS: Rt=2.08 min (99.4%); m/z 418.3 [M+H],; HPLC: 98.35% (9.61 min.) X-Bridge C18 (150 mm×4.6 mm, 3.5 m).

### Example 2: N—((S)-1-(2-Chloro-N—(((S)-2-oxopyrrolidin-3-yl)methyl)acetamido)-5-methyl-2-oxohexan-3-yl)-4-methoxy-1H-indole-2-carboxamide, Compound 2

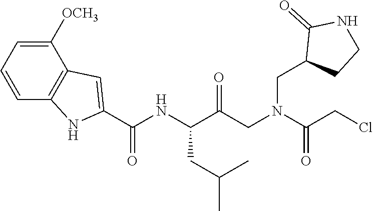 [SMILES: COc1cccc2[nH]c(C(=O)N[C@@H](CC(C)C)C(=O)CN(C[C@@H]3CCNC3=O)C(=O)CCl)cc12]

Step-1: N—((S)-3-Amino-5-methyl-2-oxohexyl)-2-chloro-N—(((S)-2-oxopyrrolidin-3-yl)methyl)acetamide trifluoroacetic acid salt (14)

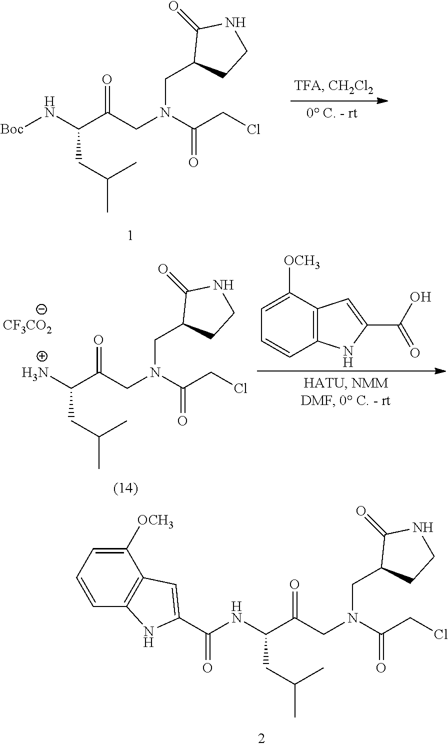

To a stirred solution of tert-butyl ((S)-1-(2-chloro-N—(((S)-2-oxopyrrolidin-3-yl)methyl)acetamido)-5-methyl-2-oxohexan-3-yl)carbamate, Compound 1 (170 mg, 0.407 mmol, 1.0 equiv.) in CH₂Cl₂ (30 mL) at 0° C. was added trifluoro acetic acid (0.16 mL, 2.038 mmol, 5.0 equiv.). The reaction mixture was stirred for 5 h at rt until the disappearance of Compound 1 by TLC. The reaction mixture was concentrated to give crude N—((S)-3-amino-5-methyl-2-oxohexyl)-2-chloro-N—(((S)-2-oxopyrrolidin-3-yl)methyl)acetamide trifluoroacetic acid salt (14) (175 mg). which was used in the next step without further purification. LC/MS (ELSD): Rt=1.04 min (74.7%); m/z 318.2 [M+H]*.

Step-2: N—((S)-1-(2-Chloro-N—(((S)-2-oxopyrrolidin-3-yl)methyl)acetamido)-5-methyl-2-oxohexan-3-yl)-4-methoxy-]H-indole-2-carboxamide, Compound 2

To a stirred solution N—((S)-3-amino-5-methyl-2-oxohexyl)-2-chloro-N—(((S)-2-oxopyrrolidin-3-yl)methyl)acetamide trifluoroacetic acid salt (14) (175 mg, 0.407 mmol, 1.0 equiv.) in DMF (5 mL) at 0° C. was added HATU (232.1 mL, 0.61 mmol, 1.5 equiv.), NMM (0.134 mL, 1.22 mmol, 3.0 equiv.) followed by 4-methoxy-1H-indole-2-carboxylic acid (92 mg, 0.488 mmol, 1.2 equiv.). The reaction mixture was stirred at rt for 6 h., quenched with a saturated NaHCO₃ solution (20 mL) extracted with 10% MeOH in CH₂Cl₂(3×20 mL) and washed with cold brine solution (2×30 mL). The combined organic layers were dried over sodium sulfate and evaporated to a residue that was subjected to GRACE flash chromatography using 10% MeOH in DCM as eluent to get N—((S)-1-(2-chloro-N—(((S)-2-oxopyrrolidin-3-yl)methyl)acetamido)-5-methyl-2-oxohexan-3-yl)-4-methoxy-1H-indole-2-carboxamide (20 mg, 10% over 2 steps) C₂₄H₃₁ClN₄O_{5;} ¹H NMR (400 MHz, DMSO-d₆) δ 11.63 (s, 1H), 8.78-8.65 (m, 1H), 7.73-7.64 (m, 1H), 7.35 (s, 1H), 7.13-6.98 (m, 2H), 6.51 (d, J=8.0 Hz, 1H), 4.68-4.08 (m, 5H), 3.89 (s, 3H), 3.65-3.31 (m, 2H), 3.16-3.07 (m, 2H), 2.20-2.0 (m, 1H), 1.77-1.50 (m, 4H), 1.23 (s, 2H), 0.89-0.83 (m, 6H); LC/MS: Rt=2.08 min (99.9%); m/z 258.1 [491.2]*; HPLC: 98.71% (9.83 min.) X-Bridge C18 (150 mm×4.6 mm, 3.5 m).

### Example 3: Tert-butyl ((S)-5-methyl-2-oxo-1-(N—(((S)-2-oxopyrrolidin-3-yl)methyl)acrylamido)hexan-3-yl)carbamate, Compound 3

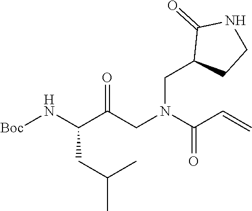 [SMILES: C=CC(=O)N(CC(=O)[C@H](CC(C)C)NC(=O)OC(C)(C)C)C[C@@H]1CCNC1=O]

Compound 3 was prepared according to the following two step procedure.

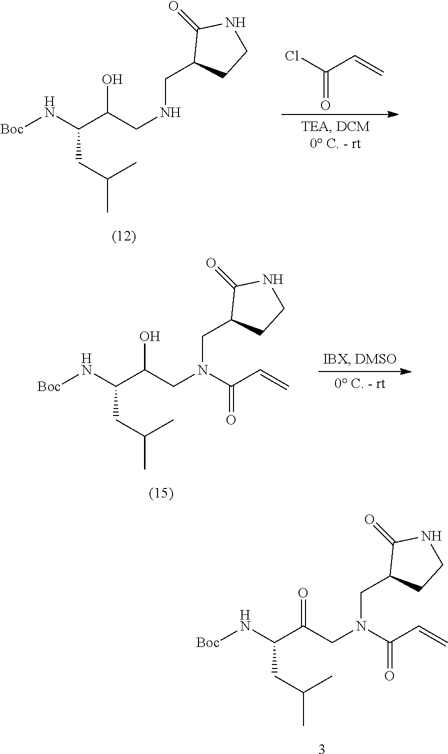

Step-1: Tert-butyl ((3S)-2-hydroxy-5-methyl-]-(N—(((S)-2-oxopyrrolidin-3-yl)methyl)acrylamido)hexan-3-yl)carbamate (15)

To a stirred solution of tert-butyl ((3S)-2-hydroxy-5-methyl-1-((((S)-2-oxopyrrolidin-3-yl)methyl)amino)hexan-3-yl)carbamate (12) (400 mg, 1.16 mmol, 1.0 equiv.) in CH₂Cl₂ (40 mL) at 0° C. was added acryloyl chloride (0.1 mL, 1.28 mmol, 1.1 equiv.). The reaction mixture was warmed to rt and stirred for 3 h. Upon consumption of (12) as indicated by TLC the reaction mixture was quenched with saturated NaHCO₃, extracted with CH₂Cl₂ (2×50 mL) and washed with brine (2×30 mL). The combined organic layers were dried over sodium sulfate and evaporated to give a residue that was purified by GRACE flash chromatography using 10% MeOH in CH₂Cl₂ as the eluent to yield tert-butyl ((3S)-2-hydroxy-5-methyl-1-(N—(((S)-2-oxopyrrolidin-3-yl)methyl)acrylamido)hexan-3-yl)carbamate (15) (380 mg, 82%) as a mixture of diastereomers. C₂₀H₃₅N₃O₅: ¹H NMR (400 MHz, DMSO-d₆) δ 7.74-7.57 (m, 1H), 6.87-6.25 (m, 2H), 6.13-6.06 (m, 1H), 5.66-5.58 (m, 1H), 5.16-4.71 (m, 1H), 3.77-3.31 (m, 6H), 3.16-3.09 (m, 2H), 2.67-2.49 (m, 1H), 2.08-2.03 (m, 1H), 1.74-1.69 (m, 1H), 1.55-1.54 (m, 1H), 1.38-1.23 (m, 11H), 0.92-0.82 (m, 6H); ELSD: Rt=3.32 min (53.8%) m/z 398.3 [M+H]+& 3.38 min (41.1%) m/z 398.3 [M+H]*; HPLC: 54.09% (4.51 min.) & 38.15% (4.59 min); Acquity UPLC BEH C18 (100 mm×2.1 mm, 1.7 m).

Step-2: Tert-butyl ((S)-5-methyl-2-oxo-1-(N—(((S)-2-oxopyrrolidin-3-yl)methyl)acrylamido)hexan-3-yl)carbamate, Compound 3

To a stirred solution of tert-butyl ((3S)-2-hydroxy-5-methyl-1-(N—(((S)-2-oxopyrrolidin-3-yl)methyl)acrylamido)hexan-3-yl)carbamate (15) (380 g, 0.957 mmol, 1.0 equiv.) in CH₂Cl₂ (60 mL) at 0° C. was added IBX (1.6 g, 5.74 mmol, 6.0 eq), stirred for 24 h at ambient temperature. To the reaction mixture was added excess sat. NaHCO₃, extracted with CH₂Cl₂(2×50 mL) and washed with brine (2×50 mL). The combined organic layers were dried over anh. Na₂SO₄, filtered and evaporated to get crude solid which was purified by GRACE flash chromatography (10% MeOH in CH₂Cl₂ eluent) to yield tert-butyl ((S)-5-methyl-2-oxo-1-(N—(((S)-2-oxopyrrolidin-3-yl)methyl)acrylamido)hexan-3-yl)carbamate, Compound 3 (0.18 mg, 48%) C₂₀H₃₃N₃O₅: ¹H NMR (400 MHz, DMSO-d₆) (rotamers) δ7.72 (m, 0.5H), 7.64 (bd, J=8.4 Hz, 0.5H), 7.43 (dd, J=7.2, 4.0 Hz, 0.5H), 7.29 (d, J=7.6 Hz, 0.5H), 6.85 (m, 0.5H), 6.36 (dd, J=16.6, 10.2 Hz, 0.5H), 6.11 (m, 1H), 5.71 (bd, J=10.0 Hz, 0.5H), 5.56 (dd, J=10.4, 2.4 Hz, 0.5H), 4.62-4.40 (m, 1H), 4.31-4.27 (m, 1H), 4.02-4.01 (m, 1H), 3.60-3.31 (m, 2H), 3.17-3.08 (m, 2H), 2.49-2.44 (m, 1H), 2.20-2.01 (m, 1H), 1.77-1.58 (m, 2H), 1.45-1.39 (m, 11H), 0.89-0.84 (m, 6H); LC/MS: Rt=2.04 min (98.2%); m/z 396.3 [M+H]*; HPLC: 98.2% (9.41 min.) X-Bridge C18 (150 mm×4.6 mm, 3.5 m).

### Example 4: 4-Methoxy-N—((S)-5-methyl-2-oxo-1-(N—(((S)-2-oxopyrrolidin-3-yl)methyl)acrylamido)hexan-3-yl)-1H-indole-2-carboxamide, Compound 4

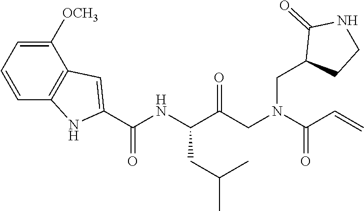 [SMILES: C=CC(=O)N(CC(=O)[C@H](CC(C)C)NC(=O)c1cc2c(OC)cccc2[nH]1)C[C@@H]1CCNC1=O]

Compound 4 was prepared according to the following two step procedure.

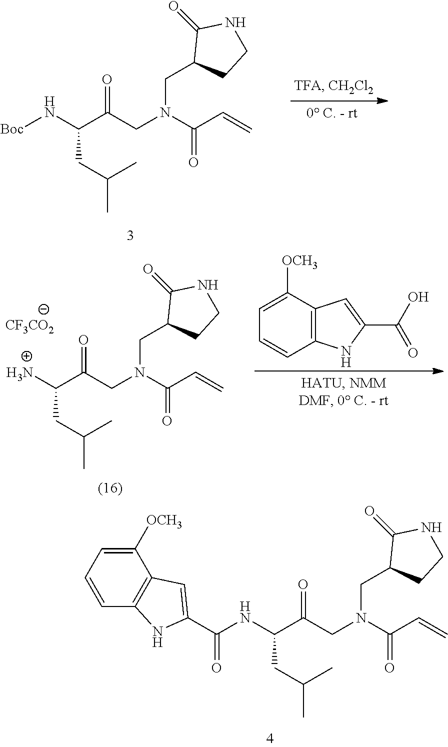

Step-1: N—((S)-3-Amino-5-methyl-2-oxohexyl)—N—(((S)-2-oxopyrrolidin-3-yl)methyl)acrylamide trifluoroacetic acid salt (16)

To a stirred solution of tert-butyl ((S)-5-methyl-2-oxo-1-(N—(((S)-2-oxopyrrolidin-3-yl)methyl)acrylamido)hexan-3-yl)carbamate, Compound 3 (160 mg, 0.4 mmol, 1.0 equiv.) in CH₂Cl₂ (25 mL) at 0° C. was added trifluoroacetic acid (0.16 mL, 2.0 mmol, 5.0 equiv.). The reaction mixture was warm to rt and stirred for 12 h. until TLC showed no Compound 3. The reaction mixture was concentrated to give a crude N—((S)-3-amino-5-methyl-2-oxohexyl)—N—(((S)-2-oxopyrrolidin-3-yl)methyl)acrylamide trifluoroacetic acid salt (16) (190 mg; C₁₅H₂₅N₃O₃) that was used in the next reaction without further purification.

Step-2: 4-Methoxy-N—((S)-5-methyl-2-oxo-1-(N—(((S)-2-oxopyrrolidin-3-yl)methyl)acrylamido)hexan-3-yl)-]H-indole-2-carboxamide, Compound 4

To a stirred solution of N—((S)-3-amino-5-methyl-2-oxohexyl)—N—(((S)-2-oxopyrrolidin-3-yl)methyl)acrylamide trifluoroacetic acid salt (16) (0.19 g, 0.644 mmol, 1.0 equiv.) in DMF (30 mL) at 0° C. was added N-methyl morpholine (0.21 mL, 1.93 mmol, 3 equiv.), HATU (0.36 mg, 0.966 mmol, 1.5 equiv.) followed by 4-methoxy-1H-indole-2-carboxylic acid (0.147 mg, 0.77 mmol, 1.2 equiv.) stirred for 12 h at rt. The reaction mixture was quenched with ice cold H₂O (30 mL), extracted with CH₂Cl₂(2×30 mL) and washed with brine (2×30 mL). The combined organic layers were dried over anh. Na₂SO₄, filtered, and evaporated to give a residue that was purified by GRACE flash chromatography using 10% MeOH in CH₂Cl₂ to yield 80 mg (85%) of a solid. This was subjected to further purification by Prep-HPLC (Column: KROMOSIL-C18 (150*25MM), 7p; Mobile phase: 0.1% TFA in H₂O:CH₃CN Gradient: (T % B): 0/25,8/60,9/60,9.1/98,12/98,12.1/25,14/25. Flow Rate: 22 mL/min) to yield 4-methoxy-N—((S)-5-methyl-2-oxo-1-(N—(((S)-2-oxopyrrolidin-3-yl)methyl)acrylamido)hexan-3-yl)-1H-indole-2-carboxamide, Compound 4. (50 mg, 12% over two steps) C₂₅H₃₂N₄O₅: ¹H NMR (500 MHz, DMSO-d₆) (rotamers) δ11.7 (bs, 1H), 8.78 (bs, 0.5H,) 8.70 (m, 0.5H), 7.74 (d, J=5.5 Hz, 0.5H), 7.66 (d, J=15.5 Hz, 0.5H), 7.35 (s, 1H), 7.11 (t, J=7.8 Hz, 1H), 7.01 (dd, J=8.0, 2.0 Hz, 1H), 6.51 (d, J=8.0 Hz, 1H)), 6.85 (dd, J=16.5, 10.5 Hz, 0.5H), 6.52 (d, J=8.0 Hz, 1H), 6.42 (dd, J=16.5, 10.5 Hz, 0.5H), 6.12 (m, 1H), 5.71 (dd, J=10.3, 2.3 Hz, 0.5H), 5.60 (dd, J=10.3, 2.3 Hz, 0.5H), 4.80-4.50 (m, 2H), 4.45-4.31 (m, 1H), 3.89 (s, 3H), 3.68-3.40 (m, 2H), 3.17-3.08 (m, 2H), 2.28-1.99 (m, 1H), 1.75-1.60 (m, 4H), 0.95-0.89 (m, 6H). LC/MS: Rt=2.04 min (96.16%); m/z 469.3 [M+H]*. HPLC: 95.2% (5.41 min.) X-Bridge C18 (50 mm×4.6 mm, 3.5 m).

### Example 5: Tert-butyl ((S)-5-methyl-2-oxo-1-(N—(((S)-2-oxopyrrolidin-3-yl)methyl)propiolamido)hexan-3-yl)carbamate, Compound 5

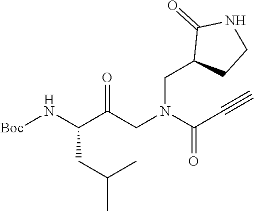 [SMILES: C#CC(=O)N(CC(=O)[C@H](CC(C)C)NC(=O)OC(C)(C)C)C[C@@H]1CCNC1=O]

Compound 5 was prepared according to the following two step procedure.

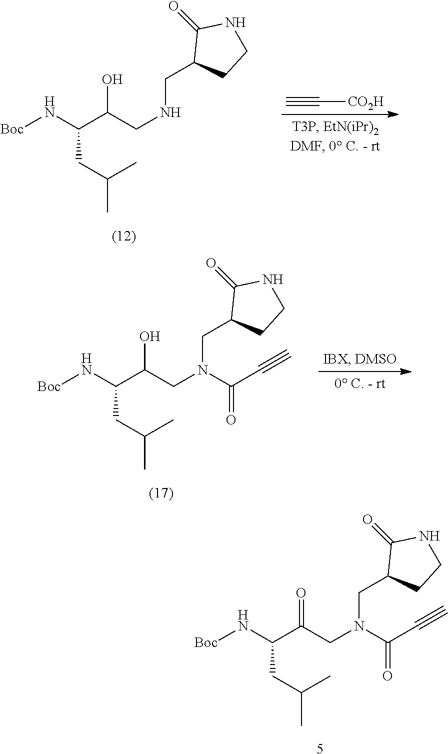

Step-1: Tert-butyl ((3S)-2-hydroxy-5-methyl-]-(N—(((S)-2-oxopyrrolidin-3-yl)methyl)propiolamido)hexan-3-yl)carbamate (17)

To a stirred solution of tert-butyl ((3S)-2-hydroxy-5-methyl-1-((((S)-2-oxopyrrolidin-3-yl)methyl)amino)hexan-3-yl)carbamate (12) (500 mg, 1.45 mmol, 1.0 equiv.) in DMF (10 mL) cooled to 0° C. was added propanephosphonic acid anhydride (T3P) (1.8 mL, 2.915 mmol, 2.0 equiv.), DIPEA (0.76 mL, 4.35 mmol, 3.0 equiv.) and propiolic acid (0.13 mL, 2.175 mmol, 1.5 equiv.). The stirred reaction mixture was gradually warmed to rt and stirring continued for 12 h. After TLC indicated consumption of amine 12, a saturated solution NaHCO₃ solution (30 mL) was added and extracted with 10% MeOH in CH₂Cl₂(2×50 mL). The organic phase was washed with cold brine (2×50 mL), combined, and dried over anh. sodium sulfate, filtered and concentrated under vacuum to give a crude residue. The residue was purified by GRACE flash chromatography using 10% MeOH in CH₂Cl₂ to get tert-butyl ((3S)-2-hydroxy-5-methyl-1-(N—(((S)-2-oxopyrrolidin-3-yl)methyl)propiolamido)hexan-3-yl) carbamate (17) (170 mg, 29%) as a yellow color gummy liquid. (Diastereomeric mixture) C₂₀H₃₃N₃O₅: ELSD: Rt=1.79 min (68.6%); m/z 396.32 [M+H]*.

Step-2: Tert-butyl ((S)-5-methyl-2-oxo-1-(N—(((S)-2-oxopyrrolidin-3-yl)methyl)propiolamido)hexan-3-yl)carbamate, Compound 5

To a stirred solution of tert-butyl ((3S)-2-hydroxy-5-methyl-1-(N—(((S)-2-oxopyrrolidin-3-yl)methyl)propiolamido)hexan-3-yl) carbamate (17) (170 mg, 0.43 mmol, 1.0 equiv.) in DMSO (5 mL) at 0° C. was added IBX (602 mg, 2.15 mmol, 5 eq). The reaction mixture was warmed to rt and monitored by LC/MS. After stirring for 18 h. a saturated NaHCO₃ solution (20 mL) was added to the reaction mixture, extracted with CH₂Cl₂(2×50 mL). The organic phase was washed with NaHCO₃ (2×50 mL), brine (2×50 mL), combined and dried over anh. Na₂SO₄, filtered and evaporated to give a residue that was purified by GRACE flash chromatography using 10% MeOH in CH₂Cl₂ as eluent to yield tert-butyl ((S)-5-methyl-2-oxo-1-(N—(((S)-2-oxopyrrolidin-3-yl)methyl)propiolamido)hexan-3-yl)carbamate, Compound 5 (100 mg, 59%) C₂₀H₃₁N₃O₅: ¹H NMR (400 MHz, CDCl₃): δ 5.80-5.50 (m, 1H), 4.98-4.92 (m, 1H), 4.75-4.74 (m, 1H), 4.56-4.25 (m, 2H), 3.94-3.92 (m, 1H), 3.80-3.50 (m, 1H), 3.40-3.28 (m, 2H), 2.67-2.61 (m, 2H), 2.45-2.25 (m, 1H), 2.16-2.06 (m, 1H), 1.72-1.64 (m, 1H), 1.44 (s, 1OH), 1.28-1.25 (m, 1H), 0.96-0.88 (m, 6H); LC/MS: Rt=1.86 min (92.0%); m/z 394.34 [M+H]+.

### Example 6: 4-Methoxy-N—((S)-5-methyl-2-oxo-1-(N—(((S)-2-oxopyrrolidin-3-yl)methyl)propiolamido)hexan-3-yl)-1H-indole-2-carboxamide, Compound 6

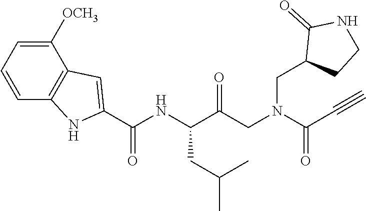 [SMILES: C#CC(=O)N(CC(=O)[C@H](CC(C)C)NC(=O)c1cc2c(OC)cccc2[nH]1)C[C@@H]1CCNC1=O]

Compound 6 was prepared according to the following two step procedure.

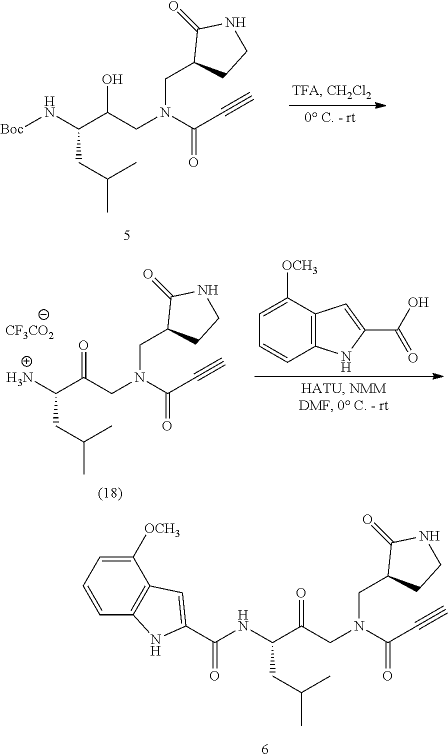

Step-1: N—((S)-3-Amino-5-methyl-2-oxohexyl)—N—(((S)-2-oxopyrrolidin-3-yl)methyl)propiolamide trifluoroacetic acid salt (18)

To a stirred solution of tert-butyl ((S)-5-methyl-2-oxo-1-(N—(((S)-2-oxopyrrolidin-3-yl)methyl)propiolamido)hexan-3-yl)carbamate, Compound 5 (98 mg, 0.25 mmol, 1.0 equiv.) in CH₂Cl₂ (20 mL) at 0° C. was added trifluoro acetic acid (94 μL, 1.246 mmol, 5.0 equiv.). The reaction mixture was warmed to rt and stirred for 6 h. The reaction mixture was concentrated under vacuum to give N—((S)-3-amino-5-methyl-2-oxohexyl)—N—(((S)-2-oxopyrrolidin-3-yl)methyl)propiolamide trifluoroacetic acid salt (18) (120 mg) that was used as is in the next step. LC/MS: Rt=0.90 min (88.4%); m/z 294.3 [M+H]*.

Step-2: 4-Methoxy-N—((S)-5-methyl-2-oxo-1-(N—(((S)-2-oxopyrrolidin-3-yl)methyl)propiolamido)hexan-3-yl)-]H-indole-2-carboxamide, Compound 6

Using similar conditions as described for Compound 4, DIPEA (0.15 mL, 0.885 mmol, 3.0 equiv.), T3P (0.28 mL, 0.442 mmol, 1.5 equiv.) followed by 4-methoxy-1H-indole-2-carboxylic acid (84.4 mg, 0.442 mmol, 1.5 equiv.) was added to a stirred solution of N—((S)-3-amino-5-methyl-2-oxohexyl)—N—(((S)-2-oxopyrrolidin-3-yl)methyl) propiolamide trifluoroacetic acid salt (18) (120 mg, 0.295 mmol, 1.0 equiv.) in DMF (10 mL) at 0° C. The reaction mixture was gradually warmed to rt and stirred for 12 h. Upon disappearance of (18) by TLC and LC/MS saturated NaHCO₃ (10 mL) was added to the mixture and then extracted with 10% MeOH in CH₂Cl₂(2×50 mL). The organic layers were combined, washed with cold brine (2×50 mL), dried over anh. sodium sulfate, filter and concentrated under vacuum to give a residue that was purified by GRACE flash chromatography with 10% MeOH in CH₂Cl₂ as the eluent to give 4-methoxy-N—((S)-5-methyl-2-oxo-1-(N—(((S)-2-oxopyrrolidin-3-yl)methyl)propiolamido)hexan-3-yl)-1H-indole-2-carboxamide, Compound 6 as an off-white solid (35 mg, 30% over two steps) C₂₅H₃₀N₄O₅: ¹H NMR (400 MHz, DMSO-d₆) δ 11.64 (s, 1H), 8.74-8.68 (dd, J=8.0 Hz, 16.0 Hz, 1H), 7.75-7.67 (m, 1H), 7.36-7.34 (dd, 1H, J=1.2 Hz, 8.0 Hz, 1H), 7.13-7.09 (m, 1H), 7.01 (d, J=8.4 Hz, 1H), 6.51 (d, J=8.0 Hz, 1H), 4.74-4.40 (m, 4H), 3.88 (s, 3H), 3.77-3.44 (m, 1H), 3.42-3.07 (m, 2H), 2.56-2.49 (m, 1H), 2.15-2.13 (m, 1H), 1.84-1.57 (m, 4H), 0.95-0.88 (m, 6H). LC/MS: Rt=3.71 min (98.6%); m/z 467.27 [M+H]*. HPLC: Rt=9.66 min (98.3%); X-Bridge C18 (150 mm×4.6 mm, 3.5 m).

### Example 7: Tert-butyl ((S)-1-(2-fluoro-N—(((S)-2-oxopyrrolidin-3-yl)methyl)acetamido)-5-methyl-2-oxohexan-3-yl)carbamate, Compound 7

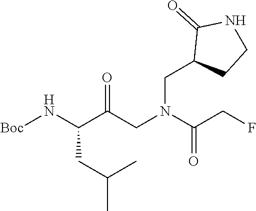 [SMILES: CC(C)C[C@H](NC(=O)OC(C)(C)C)C(=O)CN(C[C@@H]1CCNC1=O)C(=O)CF]

Compound 7 was prepared according to the following two step procedure.

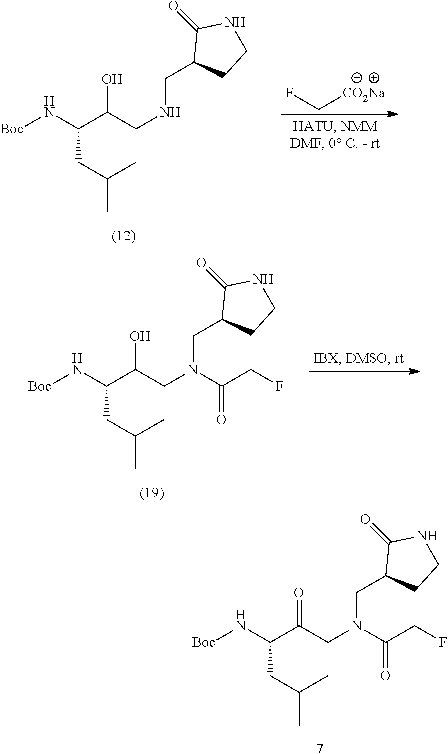

Step-1: Tert-butyl ((3S)-1-(2-fluoro-N—(((S)-2-oxopyrrolidin-3-yl)methyl)acetamido)-2-hydroxy-5-methylhexan-3-yl)carbamate (19)

To a stirred solution of tert-butyl ((3S)-2-hydroxy-5-methyl-1-((((S)-2-oxopyrrolidin-3-yl)methyl)amino)hexan-3-yl)carbamate (12) (750 mg, 2.186 mmol, 1.0 eq.) in DMF (20 mL) at 0° C. was added NMM (0.96 mL, 8.744 mmol, 4 eq.), HATU (1.66 g, 4.372 mmol, 2.0 eq.) followed by sodium 2-fluoroacetate (generated from the saponification of ethyl 2-fluoroacetate) (546.6 mg, 5.466 mmol, 2.5 eq.). The reaction was warmed to rt and stirred for 16 h. (The reaction was monitored by TLC) To the reaction mixture was added ice cold H₂O (50 mL), and extracted with 10% MeOH in CH₂Cl₂(2×70 mL). The combined organic layers were washed with brine (2×40 mL), dried over anh. sodium sulfate, filtered and evaporated. The crude product was purified by GRACE flash chromatography using 10% MeOH in CH₂Cl₂ as eluent to give tert-butyl ((3S)-1-(2-fluoro-N—(((S)-2-oxopyrrolidin-3-yl)methyl)acetamido)-2-hydroxy-5-methylhexan-3-yl)carbamate (19) as an off-white solid (480 mg, LC/MS-98%, 55%).

Step-2: Tert-butyl ((S)-1-(2-fluoro-N—(((S)-2-oxopyrrolidin-3-yl)methyl)acetamido)-5-methyl-2-oxohexan-3-yl)carbamate, Compound 7

To a stirred solution of tert-butyl ((3S)-1-(2-fluoro-N—(((S)-2-oxopyrrolidin-3-yl)methyl)acetamido)-2-hydroxy-5-methylhexan-3-yl)carbamate (19) (260 mg, 0.645 mmol, 1.0 equiv.) in DMSO (20 mL) at rt was added IBX (1.08 g, 3.87 mmol, 6.0 equiv.). After 24 h, a saturated NaHCO₃ solution was added and extracted with 10% MeOH in CH₂Cl₂(2×50 mL). The combined organic layers were washed with brine (2×50 mL, dried over anh. sodium sulfate, filtered and concentrated under vacuum. The remaining residue was purified by GRACE flash chromatography with 10% MeOH in CH₂Cl₂ to get tert-butyl ((S)-1-(2-fluoro-N—(((S)-2-oxopyrrolidin-3-yl)methyl)acetamido)-5-methyl-2-oxohexan-3-yl)carbamate, Compound 7 (250 mg, 96%) as an off-white solid. C₁₉H₃₂FN₃O₅: LC/MS: Rt=1.71 min (99.4%); m/z 402.2 [M+H]+.

### Example 8: N—((S)-1-(2-Fluoro-N—(((S)-2-oxopyrrolidin-3-yl)methyl)acetamido)-5-methyl-2-oxohexan-3-yl)-4-methoxy-1H-indole-2-carboxamide, Compound 8

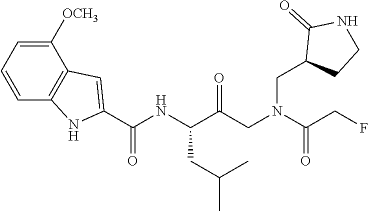 [SMILES: COc1cccc2[nH]c(C(=O)N[C@@H](CC(C)C)C(=O)CN(C[C@@H]3CCNC3=O)C(=O)CF)cc12]

Compound 8 was prepared according to the following two step procedure.

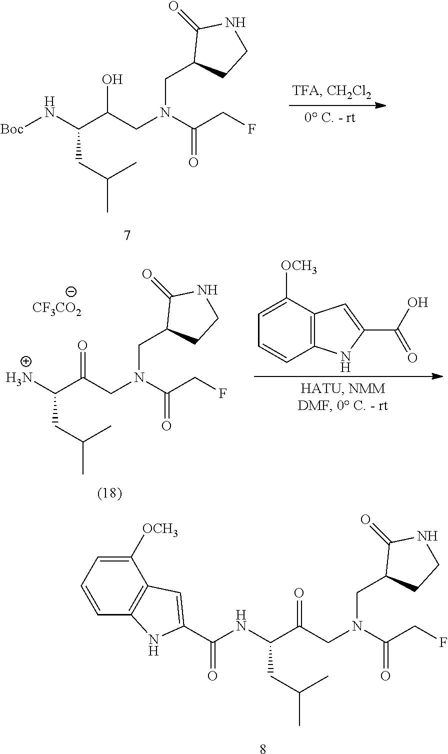

Step-1: N—((S)-3-Amino-5-methyl-2-oxohexyl)-2-fluoro-N—(((S)-2-oxopyrrolidin-3-yl)methyl)acetamide trifluoroacetic acid salt (20)

To a stirred solution of tert-butyl ((S)-1-(2-fluoro-N—(((S)-2-oxopyrrolidin-3-yl)methyl)acetamido)-5-methyl-2-oxohexan-3-yl)carbamate, Compound 7 (250 mg, 0.623 mmol, 1.0 eq.) in CH₂Cl₂ (25 mL) at 0° C. was added trifluoroacetic acid (0.24 mL, 3.11 mmol, 5.0 eq.). The reaction mixture was warm to rt and stirred for 6 h. The reaction mixture was concentrated under vacuum to give a crude N—((S)-3-amino-5-methyl-2-oxohexyl)-2-fluoro-N—(((S)-2-oxopyrrolidin-3-yl)methyl)acetamide trifluoroacetic acid salt (20) (250 mg) that was used in the next step without purification.

Step-2: N—((S)-1-(2-Fluoro-N—(((S)-2-oxopyrrolidin-3-yl)methyl)acetamido)-5-methyl-2-oxohexan-3-yl)-4-methoxy-]H-indole-2-carboxamide, Compound 8

To a stirred solution of N—((S)-3-amino-5-methyl-2-oxohexyl)-2-fluoro-N—(((S)-2-oxopyrrolidin-3-yl)methyl)acetamide (20) (250 mg, 0.602 mmol, 1.0 eq.) in MeCN (60 mL) at 0° C. was added T3P (0.57 mL, 0.903 mmol, 1.5 eq.), DIPEA (0.3 mL, 1.80 mmol, 3.0 eq.) followed by 4-methoxy-1H-indole-2-carboxylic acid (126 mg, 0.662 mmol, 1.1 eq.). The stirred reaction mixture was brought to rt. and monitored by TLC. After 6 h the reaction mixture was quenched with a saturated Na₂HCO₃ solution, extracted with 10% MeOH in CH₂Cl₂(2×50 mL) and washed with brine (2×50 mL). The combined organic layers were dried over sodium sulfate, filtered and evaporated to get crude solid that was subjected to GRACE flash chromatography using 10% MeOH in CH₂Cl₂ as eluent to get N—((S)-1-(2-fluoro-N—(((S)-2-oxopyrrolidin-3-yl)methyl)acetamido)-5-methyl-2-oxohexan-3-yl)-4-methoxy-1H-indole-2-carboxamide, Compound 8 (20 mg, 6.8% over two steps) as a white solid. C₂₄H₃₁FN₄O_{5:} ¹H NMR (500 MHz, DMSO-d₆) (rotamers) δ11.64 (s, 1H), 8.75 (d, J=7.0 Hz, 0.5H), 8.68 (d, J=7.5 Hz, 0.5H), 7.73-7.65 (m, 1H), 7.35 (bs, 1H), 7.10 (t, J=8.5 Hz, 1H), 7.01 (d, J=8.5 Hz, 1H), 6.52 (d, J=8.0 Hz, 1H), 5.31-5.15 (m, 1H), 4.93-4.83 (m, 1H), 4.60-4.36 (m, 3H), 3.88 (s, 3H), 3.46-3.39 (m, 1H), 3.15-3.09 (m, 3H), 2.50-2.49 (m, 1H), 2.19-2.0 (m, 1H), 1.74-1.59 (m, 4H), 0.95-0.89 (m, 6H). LC/MS: Rt=2.11 min (98.8%); m/z 475.3 [M+H]*; HPLC: Rt=9.50 min (97.6%) X-Bridge C18 (150 mm×4.6 mm, 3.5 m).

### Example 9: Tert-butyl ((S)-1-((1-chloro-N—(((S)-2-oxopyrrolidin-3-yl)methyl)methyl)sulfonamido)-5-methyl-2-oxohexan-3-yl)carbamate, Compound 9

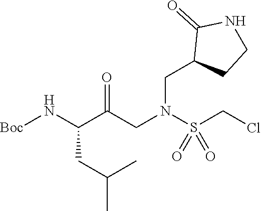 [SMILES: CC(C)C[C@H](NC(=O)OC(C)(C)C)C(=O)CN(C[C@@H]1CCNC1=O)S(=O)(=O)CCl]

Compound 9 was prepared using the two-step procedure outlined for Compound 1 in Example 1 from tert-butyl ((3S)-2-hydroxy-5-methyl-1-((((S)-2-oxopyrrolidin-3-yl)methyl)amino)hexan-3-yl)carbamate (12), except chloroacetyl chloride was replaced with chloromethanesulfonyl chloride. Off-white solid (220 mg, 91%) C₁₈H₃₂C1N₃06S: ¹H NMR (500 MHz, DMSO-d₆) δ 7.71 (bs, 1H), 7.36-7.35 (m, 1H), 5.05-4.96 (m, 2H), 4.50-4.31 (m, 2H), 4.09-4.00 (m, 2H), 3.52-3.49 (m, 1H), 3.26-3.10 (m, 4H), 2.16 (bs, 1H), 1.84-1.80 (m, 1H), 1.60 (bs, 1H), 1.38 (s, 10H), 0.88-0.84 (m, 6H); LC/MS: Rt=2.14 min (99.4%); m/z 454.2 [M+H]+.

### Example 10: N—((S)-1-((1-Chloro-N—(((S)-2-oxopyrrolidin-3-yl)methyl)methyl)sulfonamido)-5-methyl-2-oxohexan-3-yl)-4-methoxy-1H-indole-2-carboxamide, Compound 10

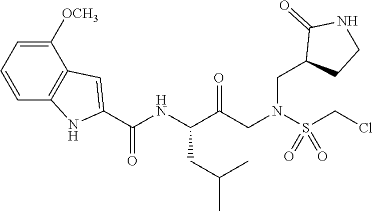 [SMILES: COc1cccc2[nH]c(C(=O)N[C@@H](CC(C)C)C(=O)CN(C[C@@H]3CCNC3=O)S(=O)(=O)CCl)cc12]

Compound 10 was prepared using the two-step procedure outlined for Compound 1 in Example 1 from tert-butyl ((S)-1-((1-chloro-N—(((S)-2-oxopyrrolidin-3-yl)methyl)methyl)sulfonamido)-5-methyl-2-oxohexan-3-yl)carbamate, Compound 9 to yield N—((S)-1-((1-chloro-N—(((S)-2-oxopyrrolidin-3-yl)methyl)methyl)sulfonamido)-5-methyl-2-oxohexan-3-yl)-4-methoxy-1H-indole-2-carboxamide, Compound 10 in 25% yield over 2 steps as an off-white solid. C₂₃H₃₁ClN₄O₆S: ¹H NMR (400 MHz, DMSO-d₆, VT=90° C.) δ11.32 (s, 1H), 8.43 (d, J=7.6 Hz, 1H), 7.37 (bs, 1H), 7.30 (d, J=2.0 Hz, 1H), 7.10 (t, J=8.0 Hz, 1H), 7.03 (d, J=8.4 Hz, 1H), 6.52 (d, J 7.2 Hz, 1H), 4.90 (s, 2H), 4.58 (m, 1H), 4.50 (s, 2H), 3.90 (s, 3H), 3.57 (dd, J=14.8, 4.8 Hz, 1H), 3.33 (dd, J=14.8, 8.4 Hz, 1H), 3.16-3.10 (m, 2H), 2.54-2.51 (m, 1H), 2.17-2.11 (m, 1H), 1.90-1.80 (m, 1H), 1.72 (m, 1H), 1.55 (m, 2H), 0.95-0.88 (m, 6H); LC/MS: Rt=4.09 min (96.0%); m/z 527.1 [M+H]+; HPLC: Rt=10.32 min (96.5%).

### Example 11: 4,5-Dichloro-N—((S)-1-(2-chloro-N—(((S)-2-oxopyrrolidin-3-yl)methyl)acetamido)-5-methyl-2-oxohexan-3-yl)thiophene-2-carboxamide, Compound 11

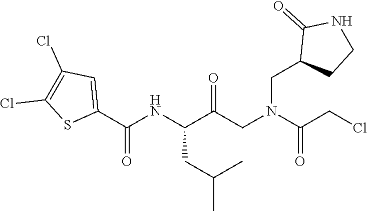 [SMILES: CC(C)C[C@H](NC(=O)c1cc(Cl)c(Cl)s1)C(=O)CN(C[C@@H]1CCNC1=O)C(=O)CCl]

Compound 11 was prepared was prepared using the procedure outlined for Compound 5 in Example 5 from N—((S)-3-amino-5-methyl-2-oxohexyl)-2-chloro-N—(((S)-2-oxopyrrolidin-3-yl)methyl)acetamide trifluoroacetic acid salt (14) and 4,5-dichlorothiophene-2-carboxylic acid in 16% yield overall as an off-white solid. C₁₉H₂₄Cl₃N₃O₄S: ¹H NMR (500 MHz, DMSO-d₆) (rotamers) δ9.03 (d, J=7.0 Hz, 0.5H), 8.92(d, J=8.0 Hz, 0.5H), 7.97 (s, 0.5H), 7.95 (s, 0.5H), 7.75 (bs, 0.5H), 7.66 (bs, 0.5H), 4.59 (d, J=3.5 Hz, 1H), 4.55-4.43 (m, 2H), 4.35-4.30 (m, 1H), 4.12 (s, 1H), 3.59 (m, 1H), 3.40 (m, 1H), 3.17-3.08 (m, 2H), 2.54-2.50 (m, 1H), 2.26 (m, 0.5H), 2.02 (m, 0.5H), 1.73-1.61 (m, 4H), 0.93-0.87 (m, 6H). LC/MS: Rt=2.28 min (98.2%); m/z 496.1 [M+H]*; HPLC: Rt=10.48 min (97.6%).

### Example 12: N—((S)-1-(2-Chloro-N—(((S)-2-oxopyrrolidin-3-yl)methyl)acetamido)-5-methyl-2-oxohexan-3-yl)-4-fluorobenzo[b]thiophene-2-carboxamide, Compound 12

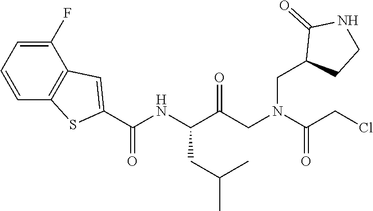 [SMILES: CC(C)C[C@H](NC(=O)c1cc2c(F)cccc2s1)C(=O)CN(C[C@@H]1CCNC1=O)C(=O)CCl]

Compound 12 was prepared was prepared using the procedure outlined for Compound 5 in Example 5 from N—((S)-3-amino-5-methyl-2-oxohexyl)-2-chloro-N—(((S)-2-oxopyrrolidin-3-yl)methyl)acetamide trifluoroacetic acid salt (14) and 4-fluorobenzo[b]thiophene-2-carboxylic acid in 21% yield overall as an off-white solid. C₂₃H₂₇ClFN₃O₄S: ¹H NMR (500 MHz, DMSO-d₆) (rotamers) δ9.22 (m, 0.5H), 9.12 (d, J=7.5 Hz, 0.5H), 8.38 (s, 0.5H), 8.35 (s, 0.5H), 7.90 (d, J=8.0 Hz, 1H), 7.76 (bs, 0.5H), 7.70 (bs, 0.5H), 7.30-7.26 (m, 1H), 4.64-4.15 (m, 6H), 3.62 (m, 0.5H), 3.45-3.35 (m, 1.5H), 3.20-3.09 (m, 2H), 2.60 (m, 1H), 2.15 (m, 0.5H), 2.01 (m, 0.5H), 1.74-1.65 (m, 4H), 0.96-0.87 (m, 6H). LC/MS: Rt=2.23 min (96.4%); m/z 496.2 [M+H]; HPLC: Rt=10.18 min (96.03%); X-Bridge C₁₈ (150 mm×4.6 mm, 3.5 m).

### Example 13: N—((S)-1-(2-Chloro-N—(((S)-2-oxopyrrolidin-3-yl)methyl)acetamido)-5-methyl-2-oxohexan-3-yl)thiophene-2-carboxamide, Compound 13

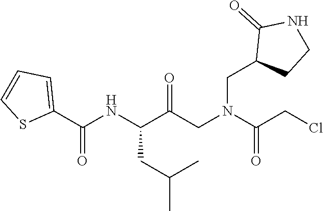 [SMILES: CC(C)C[C@H](NC(=O)c1cccs1)C(=O)CN(C[C@@H]1CCNC1=O)C(=O)CCl]

Compound 13 was prepared in a one-step procedure from (14).

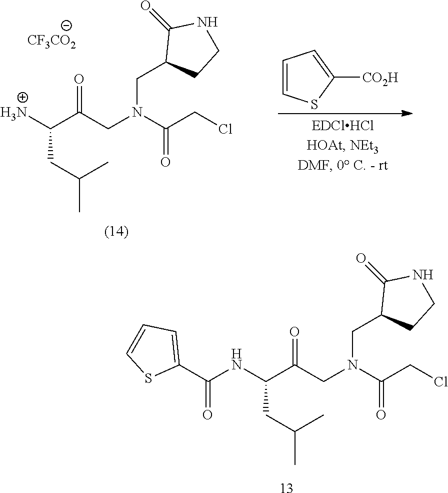

Step-1 N—((S)-1-(2-Cholro—N—(((S)-2-oxopyrrolidin-3-yl)methyl)acetamido)-5 methyl-2-oxohexan-3-yl)thiophene-2-carboxamide, Compound 13

To a stirred solution of N—((S)-3-amino-5-methyl-2-oxohexyl)-2-chloro-N—(((S)-2-oxopyrrolidin-3-yl)methyl)acetamide trifluoroacetic acid salt (14) (260 mg, 0.820 mmol, 1.0 eq.) in DMF (20 mL) at 0° C. was added EDCI HCl (314 mg, 1.64 mmol, 2.0 eq.), HOAt (0.167 mg, 1.23 mmol, 1.5 equiv.) and NEt₃ (0.4 mL, 3.28 mmol, 4.0 equiv.) followed by thiophene-2-carboxylic acid (189 mg, 1.47 mmol, 1.8 eq.). The stirred reaction mixture was gradually warmed to rt. The reaction progress was monitored by TLC, and after being stirred for 6 h ice cold H₂O (30 mL) was added followed by a saturated NaHCO₃ solution. The mixture was extracted with 10% MeOH in CH₂Cl₂(2×50 mL) and washed with brine (2×20 mL). The combined organic layers were dried over anh. sodium sulfate, filtered and evaporated to get crude residue that was subjected to GRACE flash chromatography using an eluent of 10% MeOH in CH₂Cl₂ to yield N—((S)-1-(2-chloro-N—(((S)-2-oxopyrrolidin-3-yl)methyl)acetamido)-5-methyl-2-oxohexan-3-yl)thiophene-2-carboxamide, Compound 13 as a white solid (40 mg, 16%) C₁₉H₂₆ClN₃04S: ¹H NMR (400 MHz, DMSO-d₆) (rotamers) δ8.87 (d, J=7.6 Hz, 0.5H), 8.75 (d, J=7.6 Hz, 0.5H), 7.87 (m, 1H), 7.80 (t, J=5.2 Hz, 1H), 7.75-7.65 (m, 1H), 7.18 (m, 1H), 4.59-4.10 (m, 5H), 3.61 (m, 0.5H), 3.41-3.37 (m, 1.5H), 3.17-3.07 (m, 2H), 2.54-2.50 (m, 1H), 2.25 (m, 0.5H), 2.00 (m, 0.5H), 1.75-1.59 (m, 4H), 0.93-0.87 (m, 6H); LC/MS: Rt=3.4 min (95.8%); m/z 428.1 [M+H]*; HPLC: 95.8% (8.927 min.) X-Bridge C₁₈ (150 mm×4.6 mm, 3.5 m).

### Example 14: N—((S)-1-(2-Chloro-N—(((S)-2-oxopyrrolidin-3-yl)methyl)acetamido)-5-methyl-2-oxohexan-3-yl)benzofuran-2-carboxamide, Compound 14

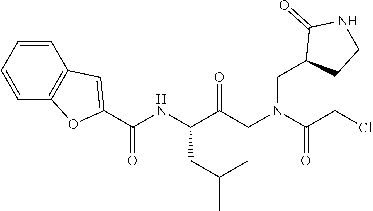 [SMILES: CC(C)C[C@H](NC(=O)c1cc2ccccc2o1)C(=O)CN(C[C@@H]1CCNC1=O)C(=O)CCl]

Compound 14 was prepared was prepared using the procedure outlined for Compound 5 in Example 5 from N—((S)-3-amino-5-methyl-2-oxohexyl)-2-chloro-N—(((S)-2-oxopyrrolidin-3-yl)methyl)acetamide trifluoroacetic acid salt (14) and benzofuran-2-carboxylic acid to give N—((S)-1-(2-chloro-N—(((S)-2-oxopyrrolidin-3-yl)methyl)acetamido)-5-methyl-2-oxohexan-3-yl)benzofuran-2-carboxamide, Compound 14 as a pale brown solid (18 mg, 11%): C₂₃H₂₈ClN₃O_{5:} ¹H NMR (400 MHz, DMSO-d₆) (rotamers) δ9.15 (m, 0.5H), 9.02 (d, J=8.0 Hz, 0.5H), 7.80 (d, J=8.0 Hz, 1H), 7.75-7.68 (m, 2H), 7.65 (s, 1H), 7.49 (m, 1H), 7.36 (t, J=7.4 Hz, 1H), 4.69-4.35 (m, 4H), 4.19-4.12 (m, 1H), 3.65-3.37 (m, 2H), 3.16-3.06 (m, 2H), 2.20-1.98 (m, 1H), 1.77-1.61 (m, 4H), 0.93-0.89 (m, 6H). LC/MS: Rt=2.14 min (97.5%); m/z 462.2 [M+H]*; HPLC: Rt=9.63 min (97.8%); X-Bridge C₁₈ (150 mm×4.6 mm, 3.5 m).

### Example 15: N—((S)-1-(2-Chloro-N—(((S)-2-oxopyrrolidin-3-yl)methyl)acetamido)-5-methyl-2-oxohexan-3-yl)-4-fluorobenzofuran-2-carboxamide, Compound 15

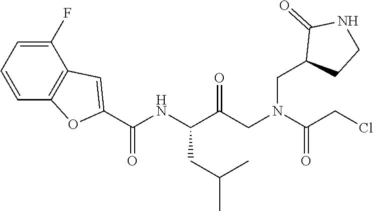 [SMILES: CC(C)C[C@H](NC(=O)c1cc2c(F)cccc2o1)C(=O)CN(C[C@@H]1CCNC1=O)C(=O)CCl]

Compound 15 was prepared was prepared using the procedure outlined for Compound 5 in Example 5 from N—((S)-3-amino-5-methyl-2-oxohexyl)-2-chloro-N—(((S)-2-oxopyrrolidin-3-yl)methyl)acetamide trifluoroacetic acid salt (14) and 4-fluorobenzofuran-2-carboxylic acid to give N—((S)-1-(2-chloro-N—(((S)-2-oxopyrrolidin-3-yl)methyl)acetamido)-5-methyl-2-oxohexan-3-yl)-4-fluorobenzofuran-2-carboxamide, Compound 15 as pale yellow solid (20 mg, 10%). C₂₃H₂₇ClFN₃O_{5:} ¹H NMR (500 MHz, DMSO-d₆) (rotamers) δ9.18 (M, 0.5H), 9.08 (d, J=8.0 Hz, 0.5H), 7.75-7.64 (m, 2H), 7.59-7.57 (m, 1H), 7.54-7.49 (m, 1H), 7.21 (m, 1H), 4.68-4.35 (m, 4H), 4.19-4.12 (m, 1H), 3.45-3.35 (m, 2H), 3.20-3.05 (m, 2H), 2.54-2.49 (m, 1H), 2.20-2.15 (m, 0.5 Hz), 2.04 (m, 0.5H), 1.74-1.62 (m, 4 Hz), 0.94-0.84 (m, 6H); LC/MS: Rt=3.94 min (98.2%); m/z 480.2 [M+H]*; HPLC: Rt=9.83 min (98.1%).

### Example 16: Tert-butyl ((S)-1-(((benzylthio)carbonyl)(((S)-2-oxopyrrolidin-3-yl)methyl)amino)-5-methyl-2-oxohexan-3-yl)carbamate, Compound 16

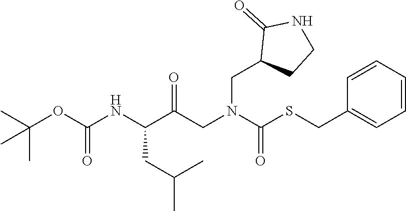 [SMILES: CC(C)C[C@H](NC(=O)OC(C)(C)C)C(=O)CN(C[C@@H]1CCNC1=O)C(=O)SCc1ccccc1]

Compound 16 was prepared according to the following two step procedure.

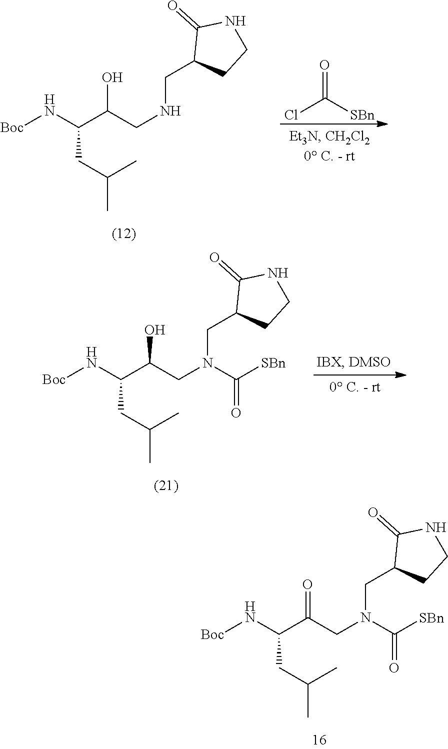

Step-1: Tert-butyl ((2S,3S)-1-(((benzylthio)carbonyl)(((S)-2-oxopyrrolidin-3-yl)methyl)amino)-2-hydroxy-5-methylhexan-3-yl)carbamate (21)

To a stirred solution of tert-butyl ((3S)-2-hydroxy-5-methyl-1-((((S)-2-oxopyrrolidin-3-yl)methyl)amino)hexan-3-yl)carbamate (12) (100 mg, 0.291 mmol, 1.0 equiv.) in CH₂Cl₂ (15 mL) at 0° C. was added triethylamine (0.121 mL, 0.873 mmol, 3.0 eq.) and benzyl chlorothioformate (81 mg, 0.437 mmol, 1.5 equiv.). The mixture was gradually warmed to rt and progress of the reaction was monitored by TLC. After 5 h, ice cold H₂O (30 mL) was added and extracted with 10% MeOH in CH₂Cl₂(2×30 mL). The organic layers were combined, washed with aq. sat NaHCO₃ (2×20 mL), brine (2×30 mL) and dried over anh. Na₂SO₄. The organic phase was filtered and evaporated under vacuum to give a residue that was purified by GRACE flash chromatography using 10% MeOH/CH₂Cl₂ as eluent yielding tert-butyl ((2S,3S)-1-(((benzylthio)carbonyl)(((S)-2-oxopyrrolidin-3-yl)methyl)amino)-2-hydroxy-5-methylhexan-3-yl)carbamate (21) (80 mg, 57%). C₂₅H₃₉N₃O₅S: ¹H NMR (500 MHz, CDCl₃) δ 7.33-7.20 (m, 5H), 5.58 (bs, 1H), 4.80-4.50 (m, 1H), 4.19-4.10 (m, 2H), 3.86-3.72 (m, 2H), 3.63-3.40 (m, 6H), 2.77 (bs, 1H), 2.33 (bs, 1H), 1.9 (bs, 1H), 1.67-1.61 (m, 1H), 1.57-1.50 (m, 1H), 1.42-1.29 (m, 10 Hz), 0.92 (d, J=6.5 Hz, 6H). LC/MS: Rt=2.15 min (98.2%); m/z 494.2 [M+H]*.

Step-2: Tert-butyl ((S)-1-(((benzylthio)carbonyl)(((S)-2-oxopyrrolidin-3-yl)methyl)amino)-5-methyl-2-oxohexan-3-yl)carbamate, Compound 16

To a stirred solution of tert-butyl ((2S,3S)-1-(((benzylthio)carbonyl)(((S)-2-oxopyrrolidin-3-yl)methyl)amino)-2-hydroxy-5-methylhexan-3-yl)carbamate (21) (80 mg, 0.162 mmol, 1.0 eq.) in DMSO (10 mL) at 0° C. was added IBX (272 mg, 0.972 mmol, 6.0 eq). The reaction mixture was brought to rt and stirred for 2 h at which time a saturated NaHCO₃ solution was and then extracted with CH₂Cl₂(2×20 mL). The combined organic layers were further washed with NaHCO₃ (2×40 mL) and brine (2×20 mL), dried over anh. sodium sulfate and evaporated to give a residue that was purified by GRACE flash chromatography using 10% MeOH/CH₂Cl₂ to yield tert-butyl ((S)-1-(((benzylthio)carbonyl)(((S)-2-oxopyrrolidin-3-yl)methyl)amino)-5-methyl-2-oxohexan-3-yl)carbamate (50 mg, 63%) as a white solid. C₂₅H₃₇N₃O₅S: ¹H NMR (500 MHz, DMSO-d₆) δ 7.71-7.66 (m, 1H), 7.52-7.12 (m, 5H), 4.46-4.38 (m, 2H), 4.21-3.85 (m, 3H), 3.58-3.30 (m, 2H), 3.16-3.07 (m, 2H), 2.50-2.49 (m, 1H), 2.11-2.10 (m, 1H), 1.79-1.74 (m, 1H), 1.61-1.60 (m, 1H), 1.45-1.20 (m, 12H), 0.89-0.81 (m, 6H); LC-MS: Rt=2.35 min (98.6%); m/z 492.3 [M+H]*; HPLC: Rt=11.02 min (98.7%); X-Bridge C₁₈ (4.6×150 mm) 3.5.

### Example 17: 2-(((S)-3-((Tert-butoxycarbonyl)amino)-5-methyl-2-oxohexyl)(((S)-2-oxopyrrolidin-3-yl)methyl)amino)-2-oxoethyl acetate, Compound 17

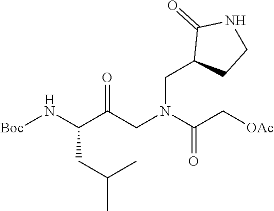 [SMILES: CC(=O)OCC(=O)N(CC(=O)[C@H](CC(C)C)NC(=O)OC(C)(C)C)C[C@@H]1CCNC1=O]

Compound 17 was prepared using the two-step procedure outlined for Compound 7 in Example 7 from tert-butyl ((3S)-2-hydroxy-5-methyl-1-((((S)-2-oxopyrrolidin-3-yl)methyl)amino)hexan-3-yl)carbamate (12), except sodium 2-fluoroacetate was replaced with 2-acetoxyacetic acid as a white solid (24% overall). C₂₁H₃₅N₃O₇: ¹H NMR (500 MHz, DMSO-d₆) (rotamers) δ7.74-7.64 (m, 1H), 7.43-7.28 (m, 1H), 4.84 (m, 1H), 4.60-4.40 (m, 2H), 4.30-4.15 (m, 1H), 4.00 (m, 1H), 3.50-3.40 (m, 1H), 3.34-3.29 (m, 1H), 3.18-3.08 (m, 2H), 2.49-2.43 (m, 1H), 2.18 (m, 0.5H), 2.06 (s, 1.5H), 2.05 (s, 1.5H), 2.01 (m, 0.5H), 1.82-1.60 (m, 2H), 1.47 (m, 2H), 1.44-1.36 (m, 9H), 0.89-0.84 (m, 6H). LC/MS: Rt=2.06 min (98.1%); m/z 442.3 [M+H]*; HPLC: Rt=9.13 min (98.9%).

### Example 18: S-Benzyl ((S)-3-(4-methoxy-1H-indole-2-carboxamido)-5-methyl-2-oxohexyl)(((S)-2-oxopyrrolidin-3-yl)methyl)carbamothioate, Compound 18

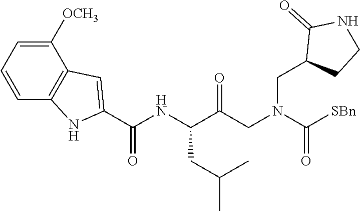

Compound 18 was prepared in a two-step procedure from Compound 16.

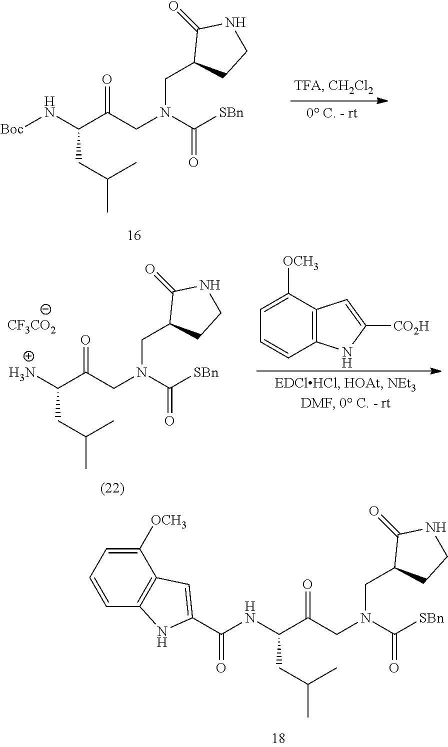

Step-1: S-Benzyl ((S)-3-amino-5-methyl-2-oxohexyl)(((S)-2-oxopyrrolidin-3-yl)methyl)carbamothioate trifluoroacetic acid salt (22)

To a stirred solution of tert-butyl ((S)-1-(((benzylthio)carbonyl)(((S)-2-oxopyrrolidin-3-yl)methyl)amino)-5-methyl-2-oxohexan-3-yl)carbamate, Compound 16 (180 mg, 0.366 mmol, 1.0 equiv.) in CH₂Cl₂ (6 mL) was added trifluoroacetic acid (0.14 mL, 1.832 mmol, 5.0 equiv.) at 0° C. The mixture was stirred for 5 h at ambient temperature. The reaction progress was monitored by TLC. After complete consumption of 16, the reaction mixture was concentrated to give 190 mg of S-benzyl ((S)-3-amino-5-methyl-2-oxohexyl)(((S)-2-oxopyrrolidin-3-yl)methyl)carbamothioate trifluoroacetic acid salt (22). The crude compound was used without further purification. LC/MS (ELSD): Rt=1.44 min (87.9%); m/z 392.2 [M+H]*.

Step-2: S-Benzyl ((S)-3-(4-methoxy-]H-indole-2-carboxamido)-5-methyl-2-oxohexyl)(((S)-2-oxopyrrolidin-3-yl)methyl)carbamothioate, Compound 18

To a stirred solution of S-benzyl ((S)-3-amino-5-methyl-2-oxohexyl)(((S)-2-oxopyrrolidin-3-yl)methyl)carbamothioate trifluoroacetic acid salt (22) (190 mg, 0.48 mmol, 1.0 equiv.) in DMF (5 mL) at 0° C. was added Et₃N (0.23 mL, 1.94 mmol, 4.0 equiv.), 4-methoxy-1H-indole-2-carboxylic acid (2) (139 mg, 0.73 mmol, 1.5 equiv.), HOAt (99 mg, 0.73 mmol, 1.5 equiv.) followed by EDCI HCl (186 mg, 0.97 mmol, 2.0 equiv.). The reaction progress was monitored by TLC, being stirred at room temperature for 6 h. At this time saturated NaHCO₃ solution (20 mL) was added, and the mixture was extracted with 10% MeOH in CH₂Cl₂ (3×20 mL) and washed with brine solution (2×30 mL). The combined organic layers were dried over anh. sodium sulfate, filtered and evaporated to give a crude residue that was subjected to purification by GRACE flash chromatography using 5% MeOH in CH₂Cl₂ as eluent to get S-benzyl ((S)-3-(4-methoxy-1H-indole-2-carboxamido)-5-methyl-2-oxohexyl)(((S)-2-oxopyrrolidin-3-yl)methyl)carbamothioate, Compound 18 (70 mg, 34% over 2 steps) C₃₀H₃₆N₄O₅S; ¹H NMR (500 MHz, DMSO-d₆) δ 11.64 (s, 1H), 8.69-8.68 (m, 1H), 7.71-7.67 (m, 1H), 7.35-7.21 (m, 6H), 7.10 (t, J=8.0 Hz, 1H), 7.00 (d, J=8.0 Hz, 1H), 6.51 (d, J=7.5 Hz, 1H), 4.57-4.40 (m, 3H), 4.10-4.07 (m, 2H), 3.88 (s, 3H), 3.62-3.56 (m, 1H), 3.49-3.47 (m, 1H), 3.13-3.08 (m, 2H), 2.50-2.49 (m, 1H), 2.19-2.02 (m, 1H), 1.80-1.58 (m, 4H) 0.94-0.83 (m, 6H); LC/MS: Rt=4.42 min (95.6%); m/z 565.2 [M+H]*; HPLC: 95.3% (11.05 min.), X-Bridge C₁₈ (4.6×150 mm) 3.5.

### Example 19: N—((S)-1-(2-Chloro-N—(((S)-2-oxopyrrolidin-3-yl)methyl)acetamido)-5-methyl-2-oxohexan-3-yl)-2,2,2-trifluoroacetamide, Compound 19

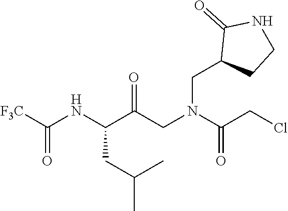 [SMILES: CC(C)C[C@H](NC(=O)C(F)(F)F)C(=O)CN(C[C@@H]1CCNC1=O)C(=O)CCl]

Compound 19 was prepared in one step from (14).

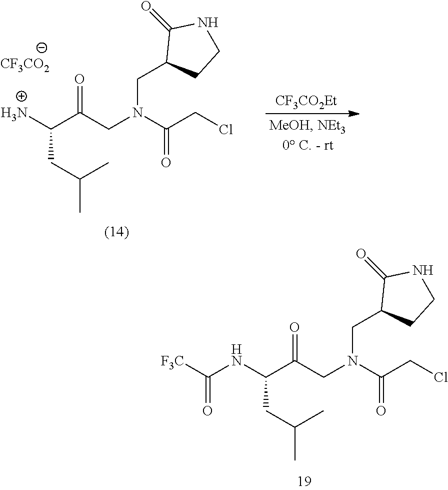

Step-1: N—((S)-1-(2-Chloro-N—(((S)-2-oxopyrrolidin-3-yl)methyl)acetamido)-5-methyl-2-oxohexan-3-yl)-2,2,2-trifluoroacetamide, Compound 19

To a stirred solution of N—((S)-3-amino-5-methyl-2-oxohexyl)-2-chloro-N—(((S)-2-oxopyrrolidin-3-yl)methyl)acetamide trifluoroacetic acid salt (14) (82 mg, 0.19 mmol, 1.0 equiv.) in MeOH (3 mL) was added triethylamine (0.11 mL, 0.946 mmol, 5.0 eq.) and ethyl 2,2,2-trifluoroacetate (0.13 mL, 0.946 mmol, 5.0 equiv.) at 0° C. The reaction was monitored by TLC. Stirring was continued at rt for 12 h. To the reaction mixture was added water (5 mL) followed by extraction with 10% MeOH in CH₂Cl₂(2×10 mL). The combined organic layers were washed with brine (2×10 mL), dried over anh. sodium sulfate, filtered and concentrated to give the crude This was purified by GRACE flash chromatography using 5% MeOH in CH₂Cl₂ as eluent to give N—((S)-1-(2-chloro-N—(((S)-2-oxopyrrolidin-3-yl)methyl)acetamido)-5-methyl-2-oxohexan-3-yl)-2,2,2-trifluoroacetamide (35 mg) that was further purified by SFC to give pure Compound 19 (18 mg, 23%). C₁₆H₂₃ClF₃N₃O₄: ¹H NMR (400 MHz, DMSO-d₆) (rotamers observed by ¹H NMR and confirmed by VT NMR): δ 9.79-9.73 (m, 1H), 7.75-7.67 (m, 1H), 4.65-4.25 (m, 4H), 4.18-4.07 (m, 1H), 3.59-3.31 (m, 2H), 3.18-3.08 (m, 2H), 2.55-2.49 (m, 1H), 2.16-2.03 (m, 1H), 1.77-1.54 (m, 4H), 0.92-0.86 (m, 6H); LC-MS: Rt=2.02 min (97.7%); m/z 414.2 [M+H]*; HPLC: 97% (9.20 min.) X-Bridge C₁₈ (4.6×150 mm) 3.5 μ.

### Example 20: Benzyl ((S)-1-(2-chloro-N—(((S)-2-oxopyrrolidin-3-yl)methyl)acetamido)-5-methyl-2-oxohexan-3-yl)carbamate, Compound 20

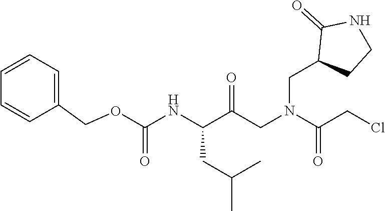 [SMILES: CC(C)C[C@H](NC(=O)OCc1ccccc1)C(=O)CN(C[C@@H]1CCNC1=O)C(=O)CCl]

Compound 20 was prepared according to the following three step procedure.

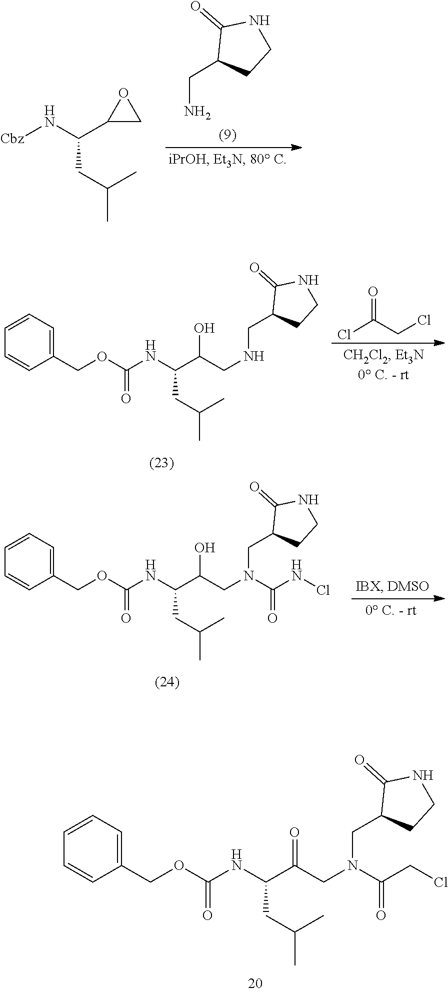 [SMILES: CC(C)C[C@H](NC(=O)OCc1ccccc1)C1CO1 ; NC[C@@H]1CCNC1=O ; CC(C)C[C@H](NC(=O)OCc1ccccc1)C(O)CNC[C@@H]1CCNC1=O ; O=C(Cl)CCl ; CC(C)C[C@H](NC(=O)OCc1ccccc1)C(O)CN(C[C@@H]1CCNC1=O)C(=O)NCl ; CC(C)C[C@H](NC(=O)OCc1ccccc1)C(=O)CN(C[C@@H]1CCNC1=O)C(=O)CCl]

Step-1: Benzyl ((3S)-2-hydroxy-5-methyl-]-((((S)-2-oxopyrrolidin-3-yl)methyl)amino)hexan-3-yl)carbamate (23)

To a stirred solution of (S)-3-(amino methyl) pyrrolidin-2-one (9) (300 mg, 2.63 mmol, 1.0 equiv.) in iPrOH (18 mL) at 80° C. was added benzyl ((1S)-3-methyl-1-(oxiran-2-yl)butyl)carbamate (Albeck et al., Tetrahedron 1994, 50, p. 6333) (761 mg, 2.89 mmol, 1.1 eq.). Benzyl ((1S)-3-methyl-1-(oxiran-2-yl)butyl)carbamate can also be prepared using the method described by Konno, et al., Synthesis 2003, 14, p.2161-2164. After 16 h, TLC indicated disappearance of the epoxide. The reaction mixture was concentrated to get a crude residue that was subjected to GRACE flash chromatography purification using 10% MeOH in CH₂Cl₂ as the eluent to provide benzyl ((3S)-2-hydroxy-5-methyl-1-((((S)-2-oxopyrrolidin-3-yl)methyl)amino)hexan-3-yl)carbamate (23) (280 mg, 28%) as an off-white solid (diastereomeric mixture). C₂₀H₃₁N₃O₄: ¹H NMR (400 MHz, DMSO-d₆) δ 7.74-7.26 (m, 5H), 5.30-5.04 (m, 2H), 4.06 (bs, 1H), 3.73-3.65 (m, 1H), 3.50 (s, 3H), 3.36-3.30 (m, 2H), 3.17-2.99 (m, 2H), 2.40-2.20 (m, 2H), 1.75-1.25 (m, 4H), 0.94-0.80 (m, 6H) LC/MS (ELSD): Rt=1.38 min (92.1%); m/z 378.2 [M+H]+.

Step-2: Benzyl ((2S,3S)-1-(2-chloro-N—(((S)-2-oxopyrrolidin-3-yl)methyl)acetamido)-2-hydroxy-5-methylhexan-3-yl)carbamate (24)

To a stirred solution of benzyl ((3S)-2-hydroxy-5-methyl-1-((((S)-2-oxopyrrolidin-3-yl)methyl)amino)hexan-3-yl)carbamate (23) (280 mg, 0.742 mmol, 1.0 equiv.) in CH₂Cl₂ (10 mL) cooled to 0° C. was added Et₃N (0.26 mL, 1.85 mmol, 2.5 equiv.) followed by chloroacetyl chloride (65 μL, 0.817 mmol, 1.1 equiv.). After stirring for 5 h at rt, TLC indicated disappearance of (23). At this time the reaction mixture was quenched with water (10 mL) and extracted with CH₂Cl₂(2×20 mL). The combined organic layers were washed with brine (2×30 mL), dried over sodium sulfate, filtered and concentrated to get crude product, which was purified by GRACE flash chromatography using 10% MeOH in CH₂Cl₂ as eluent to yielding benzyl ((2S,3S)-1-(2-chloro-N—(((S)-2-oxopyrrolidin-3-yl)methyl)acetamido)-2-hydroxy-5-methylhexan-3-yl)carbamate (24) as the major diastereomer (150 mg, 44%) as an off-white solid. C₂₂H₃₂ClN₃O_{5:} ¹H NMR (500 MHz, DMSO-d₆) (rotamers observed confirmed by VT NMR) δ7.77-7.68, 7.05-6.86 (m, 1H), 5.38-5.30 (m, 1H), 5.07-5.0 (m, 2H), 4.49-4.25 (m, 2H), 3.80-3.50 (m, 3H), 3.29-3.12 (m, 4H), 2.58-2.49 (m, 1H), 2.10-2.0 (m, 1H), 1.78-1.65 (m, 1H), 1.60-1.51 (m, 1H), 1.48-1.32 (m, 1H), 1.30-1.20 (m, 1H), 0.88-0.83 (m, 6H); LC/MS: Rt=2.11 min (97.2%); m/z 454.2 [M+H]*; HPLC: 20.9% (9.27 min.) & 77.49 (9.39 min.), X-Bridge C₁₈ (4.6×150 mm) 3.5.

Step-3: Benzyl ((S)-1-(2-chloro-N—(((S)-2-oxopyrrolidin-3-yl)methyl)acetamido)-5-methyl-2-oxohexan-3-yl)carbamate, Compound 20

To a stirred solution of benzyl ((2S,3S)-1-(2-chloro-N—(((S)-2-oxopyrrolidin-3-yl)methyl)acetamido)-2-hydroxy-5-methylhexan-3-yl)carbamate (110 mg, 0.24 mmol, 1.0 equiv.) in DMSO (5 mL) at 0° C. was added IBX (407 mg, 1.45 mmol, 6.0 equiv.). The reaction progress was monitored by LC/MS. After stirring at rt for 15 h the reaction mixture a saturated NaHCO₃ solution (20 mL) was added and extracted with CH₂Cl₂(2×50 mL). The combined organic phase was washed with NaHCO₃ (3×50 mL) followed by brine (2×50 mL), dried over anh. sodium sulfate, filtered, and evaporated to give a residue that was purified by GRACE flash chromatography using 10% MeOH in CH₂Cl₂ as the eluent yielding benzyl ((S)-1-(2-chloro-N—(((S)-2-oxopyrrolidin-3-yl)methyl)acetamido)-5-methyl-2-oxohexan-3-yl)carbamate, Compound 20, (50 mg, 46%). C₂₂H₃₀ClN₃O_{5:} ¹H NMR (500 MHz, DMSO-d₆) (VT NMR indicates rotamers) δ7.86-7.66 (m, 2H), 7.38-7.31 (m, 5H), 5.06-5.05 (m, 2H), 4.54-4.44 (m, 2H), 4.29-4.23 (m, 1H), 4.14-4.05 (m, 2H), 3.38-3.33 (m, 2H), 2.51-2.46 (m, 1H), 2.18-2.0 (m, 1H), 1.78-1.60 (m, 2H), 1.49-1.47 (m, 2H), 0.89-0.85 (m, 6H); LC-MS: Rt=2.13 min (98.8%); m/z 452.2.3 [M+H]+; HPLC: 98.94% (9.91 min.), X-Bridge C₁₈ (4.6×150 mm) 3.5 μ.

### Example 21: Benzyl ((R)-1-(2-chloro-N—(((S)-2-oxopyrrolidin-3-yl)methyl)acetamido)-5-methyl-2-oxohexan-3-yl)carbamate, Compound 21

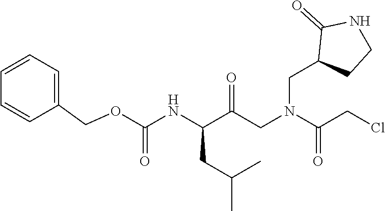 [SMILES: CC(C)C[C@@H](NC(=O)OCc1ccccc1)C(=O)CN(C[C@@H]1CCNC1=O)C(=O)CCl]

Compound 21 was prepared according to the following multi-step procedure.

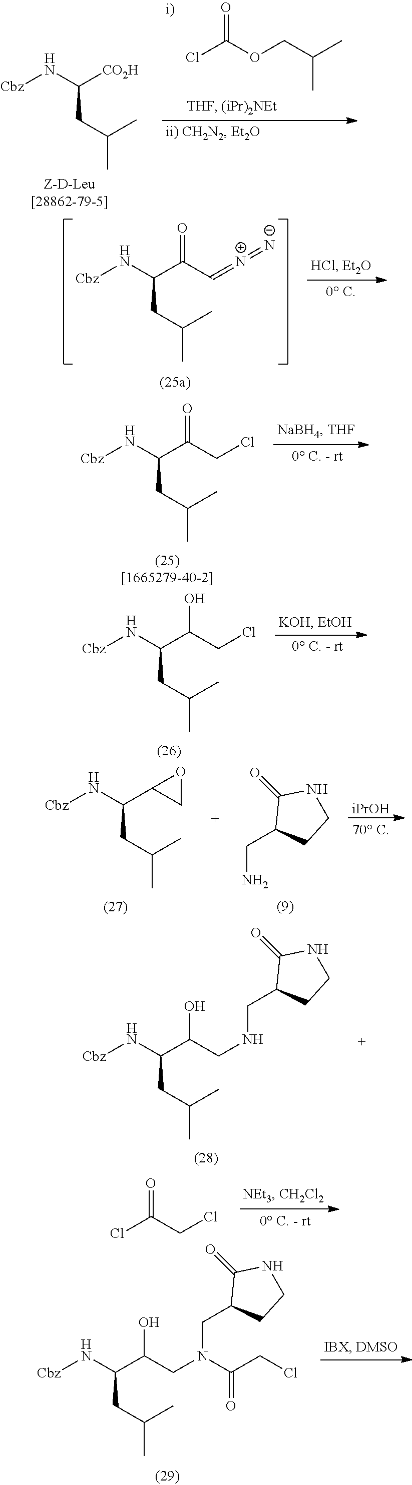

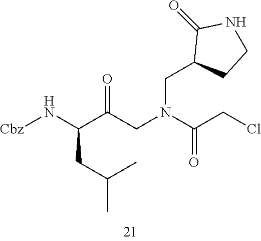 [SMILES: CC(C)C[C@@H](NC(=O)OCc1ccccc1)C(=O)CN(C[C@@H]1CCNC1=O)C(=O)CCl]

Step-1: Benzyl (R)-(I-chloro-5-methyl-2-oxohexan-3-yl)carbamate (25)

At 0° C., to a stirred solution of Cbz-D-Leucine (10 g, 37.70 mmol, 1.0 eq.) in THF (20 mL) was added DIPEA (7.3 mL, 41.619 mmol, 1.1 eq.) followed by the addition of isobutyl chloroformate (5.4 mL, 41.619 mmol, 1.1 eq.). The reaction mixture was stirred for 1 h, quickly filtered and added to a precooled (−15° C.) 0.2M solution of diazomethane (90 mL, 45.390 mmol) in dry diethyl ether. The stirred reaction mixture was allowed to warm to rt and upon disappearance of starting material by TLC, re-cooled to 0° C. To the cooled mixture was added 2M HCl in diethyl ether slowly till the yellow color disappeared. After approximately 1 h the reaction mixture was concentrated to give an oil that was subjected to GRACE flash chromatography purification using 10% ethyl acetate in petroleum ether as the eluent yielding benzyl (R)-(1-chloro-5-methyl-2-oxohexan-3-yl)carbamate (25) (6 g, 33%) as a colorless oil. C₁₅H₂OClNO₃: ¹H NMR (500 MHz, DMSO-d₆) δ 7.77-7.72 (m, 1H), 7.39-7.30 (m, 5H), 5.05-5.03 (m, 2H), 4.62 (s, 2H), 4.26-4.23 (m, 1H), 1.66-1.60 (m, 1H), 1.49-1.42 (m, 2H), 0.88-0.84 (m, 6H).

Step-2: Benzyl ((3R)-1-chloro-2-hydroxy-5-methylhexan-3-yl)carbamate (26)

To a stirred solution of benzyl (R)-(1-chloro-5-methyl-2-oxohexan-3-yl)carbamate (25) (8 g, 26.86 mmol, 1.0 eq.) in THE (80 mL) was added sodium borohydride (1.22 g, 32.32 mmol, 1.2 eq.) at 0° C. The reaction mixture was warmed to rt and stirred for 2 h. The mixture was quenched with cold water (50 mL) and extracted with ethyl acetate (2×50 mL). The combined organic layers were washed with brine (2×40 mL), dried over anh. sodium sulfate, filtered and concentrated to give 6.0 gms crude benzyl ((3R)-1-chloro-2-hydroxy-5-methylhexan-3-yl)carbamate (26) as a colorless oil that was used in the next reaction without further purification.

Step-3: Benzyl ((]R)-3-methyl-]-(oxiran-2-yl)butyl)carbamate (27)

To a stirred solution of benzyl ((3R)-1-chloro-2-hydroxy-5-methylhexan-3-yl)carbamate (26) (6 g, 20.06 mmol, 1.0 eq.) in ethanol (20 mL) was added KOH (1.35 g, 24.06 mmol, 1.2 eq.) at 0° C. The reaction mixture was stirred for 4 h and extracted with CH₂Cl₂(2×20 mL). The combined organic layers were washed with brine (2×30 mL), dried over anh. sodium sulfate, filtered and concentrated to give a residue that was purified by GRACE FLASH chromatography using 20% ethyl acetate in pet-ether as eluent to yield 1.4 g of benzyl ((1R)-3-methyl-1-(oxiran-2-yl)butyl)carbamate (27) (26%) as a colorless oil. C₁₅H₂₁NO₃; ¹H NMR (500 MHz, DMSO-d₆) (diastereomers) δ7.38-7.22 (m, 6H), 5.04-5.01 (m, 2H), 3.50-3.30 (2m, 1H), 2.92-2.81 (2m, 1H), 2.69-2.63 (2m, 1H), 2.59-2.49 (2m, 1H), 1.70-1.56 (m, 1H), 1.43-1.40 (m, 1H), 1.35-1.20 (m, 1H), 0.88-0.82 (m, 6H); LC/MS: Rt=2.03 min (99.9%); m/z 264.2 [M+H]*.

Step-4: Benzyl ((3R)-2-hydroxy-5-methyl-]-((((S)-2-oxopyrrolidin-3-yl)methyl)amino)hexan-3-yl)carbamate (28)

A stirred solution of (S)-3-(amino methyl) pyrrolidin-2-one (9) (606 mg, 5.32 mmol, 1.0 eq.) and benzyl ((1R)-3-methyl-1-(oxiran-2-yl)butyl)carbamate (27) (1.4 g, 5.32 mmol, 1.0 equiv.) in iPrOH (20 mL) was heated at 70° C. for 12 h until LC/MS indicated no starting materials remained. The reaction mixture was concentrated to get a residue that was subjected to by GRACE flash chromatography purification using 10% MeOH in CH₂Cl₂ as the eluent to provide 650 mg (33%) of benzyl ((3R)-2-hydroxy-5-methyl-1-((((S)-2-oxopyrrolidin-3-yl)methyl)amino)hexan-3-yl)carbamate (28) as an off-white solid that was used as is. C₂₀H₃₁N₃O₄; LC/MS: Rt=1.34 min (88.2%); m/z 378.9 [M+H]*.

Step-5: Benzyl ((3R)-1-(2-chloro-N—(((S)-2-oxopyrrolidin-3-yl)methyl)acetamido)-2-hydroxy-5-methylhexan-3-yl)carbamate (29)

To a stirred solution of benzyl ((3R)-2-hydroxy-5-methyl-1-((((S)-2-oxopyrrolidin-3-yl)methyl)amino)hexan-3-yl)carbamate (28) (650 mg, 1.72 mmol, 1.0 eq.) in CH₂Cl₂ (10 mL) at 0° C. was added Et₃N (0.30 mL, 2.58 mmol, 1.5 equiv.) followed by 2-chloroacetyl chloride (139 μL, 1.72 mmol, 1.0 eq.). The reaction mixture was stirred at rt for 4 h. Upon disappearance of (28) by TLC the reaction mixture was quenched with water (10 mL) and extracted with CH₂Cl₂ (2×20 mL). The combined organic layers were washed with brine (2×30 mL), dried over anh. sodium sulfate, filtered and concentrated to get crude product that was purified by GRACE flash chromatography using 10% MeOH in CH₂Cl₂ as eluent yielding 380 mg (48%) of benzyl ((3R)-1-(2-chloro-N—(((S)-2-oxopyrrolidin-3-yl)methyl)acetamido)-2-hydroxy-5-methylhexan-3-yl)carbamate (29) as an off-white solid. C₂₂H₃₂ClN₃O₅: LC/MS (diastereomers): Rt=2.09 (99.5%) and 2.11 min (99.7%); m/z 454.3 [M+H]*.

Step-6: Benzyl ((R)-1-(2-chloro-N—(((S)-2-oxopyrrolidin-3-yl)methyl)acetamido)-5-methyl-2-oxohexan-3-yl)carbamate, Compound 21

To a stirred solution of benzyl ((3R)-1-(2-chloro-N—(((S)-2-oxopyrrolidin-3-yl)methyl)acetamido)-2-hydroxy-5-methylhexan-3-yl)carbamate (29), 90 mg, 0.198 mmol, 1.0 eq.) in DMSO (3 mL) at 0° C. was added IBX (330 mg, 1.19 mmol, 6.0 eq.). The reaction mixture was warmed to rt and stirred for 16 h. A saturated NaHCO₃ solution was added and extracted with CH₂Cl₂(2×50 mL) and washed with NaHCO₃ (3×50 mL) and brine (2×50 mL). The combined organic layers were dried over anh. sodium sulfate, filtered and concentrated to give crude residue. Purification of the product was performed using GRACE flash chromatography with 10% MeOH in CH₂Cl₂ to yield benzyl ((R)-1-(2-chloro-N—(((S)-2-oxopyrrolidin-3-yl)methyl)acetamido)-5-methyl-2-oxohexan-3-yl)carbamate, Compound 21 (80 mg, 90% yield) as an off-white solid. C₂₂H₃₀ClN₃O_{5:} ¹H NMR (400 MHz, DMSO-d₆) (rotamers observed confirmed by VT NMR) δ7.84-7.68 (m, 2H), 7.37-7.31 (m, 5H), 5.09-5.02 (m, 2H), 4.64-4.02 (m, 4H), 3.61-3.31 (m, 2H), 3.16-3.08 (m, 2H), 2.49-2.44 (m, 1H), 2.13-2.10 (m, 1H), 1.75-1.60 (m, 2H), 1.50-1.44 (m, 1H), 0.90-0.85 (m, 6H); LC/MS: Rt=2.13 min (99.1%); m/z 452.2 [M+H]*; HPLC: 99.2% (9.61 min.), X-Bridge C₁₈ (4.6×150 mm) 3.5.

### Example 22: Cyclobutyl ((S)-1-(2-chloro-N—(((S)-2-oxopyrrolidin-3-yl)methyl)acetamido)-5-methyl-2-oxohexan-3-yl)carbamate, Compound 22

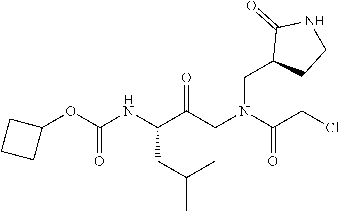 [SMILES: CC(C)C[C@H](NC(=O)OC1CCC1)C(=O)CN(C[C@@H]1CCNC1=O)C(=O)CCl]

Compound 22 was prepared according to the following multi-step procedure.

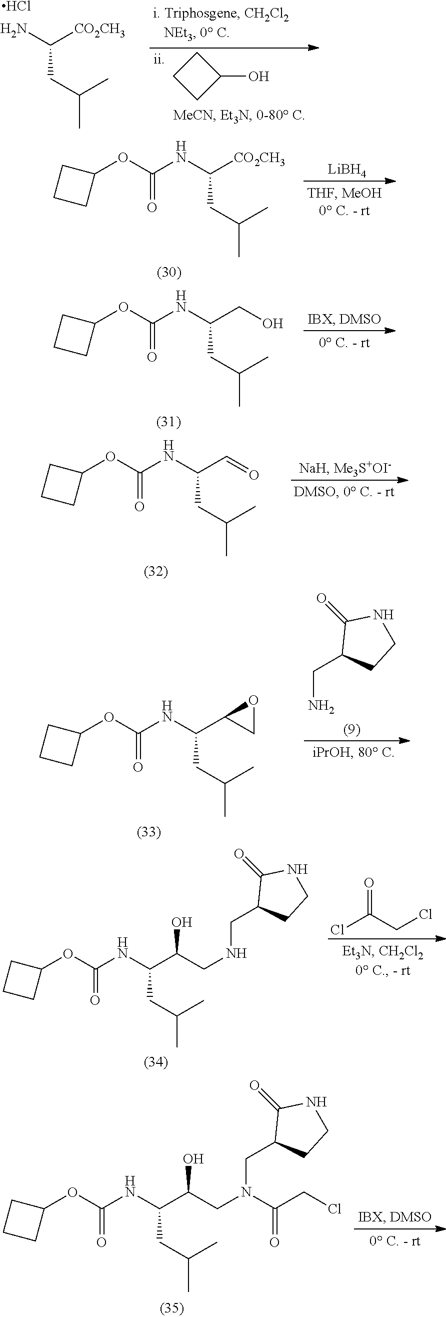

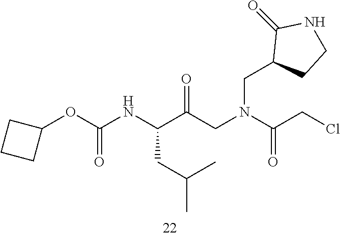 [SMILES: CC(C)C[C@H](NC(=O)OC1CCC1)C(=O)CN(C[C@@H]1CCNC1=O)C(=O)CCl]

Step-1: Methyl (cyclobutoxycarbonyl)-L-leucinate (30)

To a stirred solution of methyl L-leucinate hydrochloride (5.0 g, 27.47 mmol, 1.0 eq.) in CH₂Cl₂ (100 mL) was added triethylamine (11.12 g, 109.75 mmol, 4.0 eq.) at 0° C. followed by the dropwise addition of triphosgene (4.0 g, 13.74 mmol, 0.5 eq.). After the reaction mixture was stirred at 0° C. for 2 h. TLC indicated the consumption of the starting amino acid ester. At this time the reaction mixture was concentrated to get a crude residue that was dissolved in acetonitrile (75.0 mL) and cooled to 0° C. Triethylamine (8.34 g, 82.42 mmol, 3.0 eq.) was added followed by cyclobutanol (2.4 g, 32.97 mmol, 1.2 eq.) and the stirred the reaction mixture was gradually heated to 80° C. and maintained for 16 h. To the reaction mixture was added cold water (100 mL) and then extracted with EtOAc (3×100 mL). The combined organic layers were washed with brine (2×80 mL), dried over anh. sodium sulfate, filtered and concentrated to obtain a crude residue that was purified by column chromatography using 15% EtOAc in pet-ether as the eluent to yield methyl (cyclobutoxycarbonyl)-L-leucinate (30) (1.5 g, 22.5%) as a colorless liquid. C₁₂H₂₁NO₄: ¹H NMR (500 MHz, DMSO-d₆) δ 7.54 (d, J=8.0 Hz, 1H), 4.80 (m, 1H), 4.01-3.97 (m, 1H), 3.64 (s, 3H), 2.23-2.21 (m, 2H), 1.97-1.93 (m, 2H), 1.71-1.40 (m, 5H), 0.87 (d, J=6.5 Hz, 3H), 0.84 (d, J=6.5 Hz, 3H); LC/MS (ELSD): Rt=2.25 min (77.23%); m/z 244.2 [M+H]+.

Step-2: Cyclobutyl (S)-(I-hydroxy-4-methylpentan-2-yl)carbamate (31)

To a stirred solution of methyl (cyclobutoxycarbonyl)-L-leucinate (30), 1.5 g, 6.17 mmol, 1.0 eq.) in a mixture of THF (7.5 mL) and MeOH (7.5 mL) cooled to O ° C. was added LiBH₄ (0.679 g, 30 86 mmol, 5.0 eq.). The stirred reaction mixture was stirred at rt for 16 h. The progress of the reaction was followed by TLC and LC/MS. After 16 h ester (30) was consumed. The reaction mixture was quenched with cold water (20 mL) and extracted with CH₂Cl₂ (3×30 mL). The organic layers were combined, washed with brine (2×50 mL), dried over anh. sodium sulfate, filtered and concentrated to give a residue that was purified by column chromatography using 40% EtOAc in petroleum ether as the eluent. Cyclobutyl (S)-(1-hydroxy-4-methylpentan-2-yl)carbamate (31) (900 mg, 68%) was obtained as a pale yellow oil. C₁₁H₂₁NO3: ¹H NMR (400 MHz, DMSO-d₆) δ 6.74 (d, J=8.8 Hz, 1H), 4.85-4.74 (m, 1H), 4.58 (t, J=6.0 Hz, 1H), 3.48-3.39 (m, 1H), 3.31-3.24 (m, 1H), 3.21-3.12 (m, 1H), 2.22-2.19 (m, 2H), 1.95-1.90 (m, 2H), 1.75-1.42 (m, 3H), 1.56-1.19 (m, 2H), 0.90-0.81 (m, 6H).; LC-MS (ELSD): Rt=1.68 min (99.14%); m/z 216.16 [M+H]+.

Step-3: Cyclobutyl (S)-(4-methyl-]-oxopentan-2-yl)carbamate (32)

To a stirred solution of cyclobutyl (S)-(1-hydroxy-4-methylpentan-2-yl)carbamate (31), 900 mg, 4.186 mmol, 1.0 eq.) in DMSO (9.0 mL) was added IBX (4.6 g, 16.774 mmol, 4.0 equiv.) at 0° C. The reaction mixture at rt for 16 h. TLC indicated no starting alcohol remained. To the reaction mixture was added a saturated NaHCO₃ solution (30 mL) which was extracted with diethyl ether (3×50 mL) and washed with NaHCO₃ (3×50 mL) and brine (2×50 mL). The combined organic layers were dried over anh. sodium sulfate, filtered and concentrated to give crude residue that was subjected to column chromatography using 10% EtOAc in petroleum ether yielding cyclobutyl (S)-(4-methyl-1-oxopentan-2-yl)carbamate (32) (350 mg, 39%) as a pale yellow oil. C₁₁H₁₉NO₃: ¹H NMR (400 MHz, CDCl₃) δ 9.59 (s, 1H), 5.09-4.92 (m, 2H), 4.35-4.25 (m, 1H), 2.36-2.29 (m, 3H), 2.11-2.03 (m, 1H), 1.80-1.54 (m, 5H), 1.43-1.39 (m, 1H), 1.0-0.85 (m, 6H); LC/MS: Rt=1.55 min (41.36%); m/z 214.4 [M+H]*.

Step-4: Cyclobutyl ((S)-3-methyl-]-((R)-oxiran-2-yl)butyl)carbamate (33)

To a stirred solution of trimethylsulfoxonium iodide (0.723 g, 3.286 mmol, 2.0 eq.) in DMSO (3.5 mL) was added NaH (0.080 g, 3.286 mmol, 2.0 eq.) portion-wise at room temperature. After 0.5 h, cyclobutyl (S)-(4-methyl-1-oxopentan-2-yl)carbamate (32) (0.350 g, 1.643 mmol, 1.0 eq.) in DMSO (3.5 mL) was added to the reaction mixture and stirring was continued at rt for 2 h. After consumption of aldehyde (32) as indicated by TLC and LC/MS, the reaction mixture was quenched with cold water (10 mL) and extracted with diethyl ether (3×20 mL). The combined organic layers were washed with brine (2×30 mL), dried over anh. sodium sulfate, filtered and concentrated leaving a crude residue that was purified by column chromatography using 10% EtOAc in petroleum ether as eluent to give cyclobutyl ((S)-3-methyl-1-((R)-oxiran-2-yl)butyl)carbamate (33) (170 mg, 45.5%) as a colorless liquid. C₁₂H₂1NO₃: ¹H NMR (500 MHz, DMSO-d₆) δ 7.10-7.0 (m, 1H), 4.90-4.74 (m, 1H), 3.45-3.34 (m, 1H), 2.88-2.87 (m, 1H), 2.68-2.63 (m, 1H), 2.30-2.15 (m, 2H), 1.95-1.91 (m, 2H), 1.69-1.52 (m, 3H), 1.45-1.38 (m, 1H), 1.25-1.20 (m, 2H), 087-0.81 (m, 6H); LC/MS (ELSD): Rt=1.89 min (99.69%) m/z 228.1 [M+H]*.

Step-5: Cyclobutyl ((2S,3S)-2-hydroxy-5-methyl-]-((((S)-2-oxopyrrolidin-3-yl)methyl)amino)hexan-3-yl)carbamate (34)

To a stirred solution of (S)-3-(aminomethyl)pyrrolidin-2-one (9) (85 mg, 0.749 mmol, 1.0 eq.) in isopropanol (10 mL) at rt was added cyclobutyl ((S)-3-methyl-1-((R)-oxiran-2-yl)butyl)carbamate (33) (170 mg, 0.749 mmol, 1.0 eq.). The reaction mixture was heated to 85° C. and stirred for 18 h. TLC indicated disappearance of starting materials. The reaction mixture was concentrated to get crude product that was purified by column chromatography using 5% MeOH in CH₂Cl₂ as eluent to afford cyclobutyl ((2S,3S)-2-hydroxy-5-methyl-1-((((S)-2-oxopyrrolidin-3-yl)methyl)amino)hexan-3-yl)carbamate (34) (220 mg, 86.2%) as a pale yellow oil. C₁₇H₃₁N₃O₄: ¹H NMR (400 MHz, DMSO-d₆) δ 8.0-7.98 (m, 1H), 6.85-6.82 (m, 1H), 5.50-5.45 (m, 1H), 4.82-4.80 (m, 1H), 3.80-3.70 (m, 1H), 3.58-3.54 (m, 1H), 3.25-3.16 (m, 2H), 3.10-2.85 (m, 2H), 2.80-2.61 (m, 2H), 2.32-2.21 (m, 3H), 1.98-1.93 (m, 2H), 1.81-1.65 (m, 2H), 1.57-1.53 (m, 2H), 1.40-1.30 (m, 1H), 1.23-1.14 (m, 3H), 0.88-0.82 (m, 6H); LC/MS (ELSD): Rt=1.26 min (94.35%); m/z 342.2 [M+H]*.

Step-6: Cyclobutyl ((2S,3S)-1-(2-chloro-N—(((S)-2-oxopyrrolidin-3-yl)methyl)acetamido)-2-hydroxy-5-methylhexan-3-yl)carbamate (35)

To a stirred solution of cyclobutyl ((2S,3S)-2-hydroxy-5-methyl-1-((((S)-2-oxopyrrolidin-3-yl)methyl)amino)hexan-3-yl)carbamate (34) (220 mg, 0.645 mmol, 1.0 eq.) in CH₂Cl₂ (4 mL) cooled to O ° C. was added Et₃N (0.18 mL, 1.29 mmol, 2.0 eq.) followed by chloroacetyl chloride (50 μL, 0.645 mmol, 1.0 eq.). Stirring was continued at 0° C. for 1 h, at which time TLC indicated consumption of secondary amine (34). Cold water (10 mL) was added and the mixture was extracted with CH₂Cl₂(3×20 mL). The combined organic layers were washed with brine (2×30 mL), dried over sodium sulfate, filtered and concentrated to give a crude product that was purified by prep-HPLC (Method: Column: SUNFIRE-C18 (150*19), 5p Mobile phase: 10 mM (NH₄)HCO₃ in H₂O:CH₃CN; Gradient: (T/% of B): 0/20, 8/50, 10/50, 10.1/98, 13/98, 13.1/20, 15/20; Flow Rate: 17 mL/min, Diluent: CH₃CN+H₂O). The collected fractions were lyophilized to obtain cyclobutyl ((2S,3S)-1-(2-chloro-N—(((S)-2-oxopyrrolidin-3-yl)methyl)acetamido)-2-hydroxy-5-methylhexan-3-yl)carbamate (35) (90 mg, 33.46%) as a white solid. C₁₉H₃₂ClN₃O_{5:} ¹H NMR (400 MHz, DMSO-d₆) (rotamers observed in ¹H NMR & confirmed by VT NMR) δ7.78-7.65 (m, 1H), 6.84-6.60 (m, 1H), 5.35-5.23 (m, 1H), 4.80-4.70 (m, 1H), 4.49-4.24 (m, 2H), 3.77-3.40 (m, 4H), 3.32-3.10 (m, 4H), 2.59-2.50 (m, 1H), 2.23-2.20 (m, 2H), 2.19-1.92 (m, 3H), 1.73-1.51 (m, 4H), 1.40-1.20 (m, 2H), 0.87-0.82 (m, 6H); LC/MS: Rt=2.06 min (99.2%), m/z 418.2 [M+H]*; HPLC: 99.0% (9.27 min.), X-Bridge C18 (4.6×150 mm) 3.5; Mobile phase A; 0.05% TFA in(aq), B: 100% Acetonitrile: Gradient (T/% B): 0/5, 1.5/5, 3/15, 7/55, 10/95, 14/95, 17/5, 20/5. Flow Rate: 1.0 mL/min, Diluent: 70% CH₃CN, 30% H₂O).

Step-7: Cyclobutyl ((S)-1-(2-chloro-N—(((S)-2-oxopyrrolidin-3-yl)methyl)acetamido)-5-methyl-2-oxohexan-3-yl)carbamate, Compound 22

To a stirred solution of cyclobutyl ((2S,3S)-1-(2-chloro-N—(((S)-2-oxopyrrolidin-3-yl)methyl)acetamido)-2-hydroxy-5-methylhexan-3-yl)carbamate (35) (60 mg, 0.144 mmol, 1.0 eq.) in DMSO (2 mL) at rt was added IBX (201 mg, 0.719 mmol, 5.0 eq.). The reaction mixture was stirred for 16 h. A saturated NaHCO₃ solution was added, extracted with diethyl ether (3×20 mL) and washed with NaHCO₃ (2×50 mL) and brine (2×50 mL). The combined organic layers were dried over sodium sulfate, filtered and concentrated to give a residue that was subjected to prep-HPLC purification (Method: Column: SUNFIRE-C18 (150*19), 5p Mobile phase: 10 mM (NH₄)HCO₃ in H₂O: CH₃CN, Gradient: (T/% of B): 0/10, 8/60, 10/60, 10.1/98, 13/98, 13.1/10, 15/10. Flow Rate: 17 mL/min. Diluent: CH₃CN+H₂O). The fractions collected were lyophilized to afford cyclobutyl ((S)-1-(2-chloro-N—(((S)-2-oxopyrrolidin-3-yl)methyl)acetamido)-5-methyl-2-oxohexan-3-yl)carbamate, Compound 22 (25 mg, 41.6%) as a white solid. C₁₉H₃₀ClN₃O_{5:} ¹H NMR (500 MHz, DMSO-d₆) (rotamers observed in ¹H NMR and confirmed by VT NMR) δ7.75-7.54 (m, 2H), 4.82-4.79 (m, 1H), 4.61-4.45 (m, 2H), 4.40-4.20 (m, 1H), 4.15-4.0 (m, 2H), 3.60-3.35 (m, 2H), 3.20-3.11 (m, 2H), 2.29-1.95 (m, 5H), 1.80-1.65 (m, 2H), 1.61-1.53 (m, 2H), 1.47-1.44 (m, 2H), 1.30-1.24 (m, 1H), 0.89-0.84 (m, 6H); LC/MS: Rt=2.07 min (98.1%); m/z 416.3 [M+H]*; HPLC: 97.4% (9.27 min.) X-Bridge C₁₈ (4.6×150 mm) 3.5. Mobile phase A; 10 mmol (NH₄)OAc (aq), B: 100% Acetonitrile: Gradient (T/% B): 0/5, 1.5/5, 3/15, 7/55, 10/95, 14/95, 17/5, 20/5. Flow Rate: 1.0 mL/min, Diluent: 70% CH₃CN, 30% H₂O).

### Example 23: N—((S)-1-((Cyanomethyl)(((S)-2-oxopyrrolidin-3-yl)methyl)amino)-5-methyl-2-oxohexan-3-yl)-4-methoxy-1H-indole-2-carboxamide, Compound 23

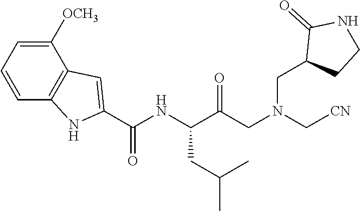 [SMILES: COc1cccc2[nH]c(C(=O)N[C@@H](CC(C)C)C(=O)CN(CC#N)C[C@@H]3CCNC3=O)cc12]

Compound 23 was prepared in four steps from tert-butyl ((3S)-2-hydroxy-5-methyl-1-((((S)-2-oxopyrrolidin-3-yl)methyl)amino)hexan-3-yl)carbamate (12).

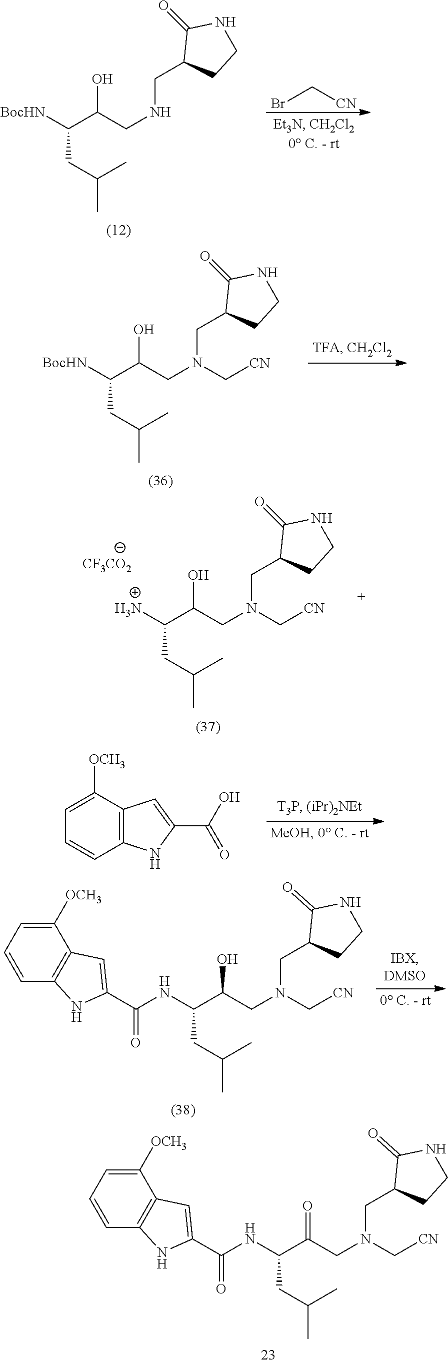

Step-1: Tert-butyl ((3S)-1-((cyanomethyl)(((S)-2-oxopyrrolidin-3-yl)methyl)amino)-2-hydroxy-5-methylhexan-3-yl)carbamate (36)

To a stirred solution of tert-butyl ((3S)-2-hydroxy-5-methyl-1-((((S)-2-oxopyrrolidin-3-yl)methyl)amino)hexan-3-yl)carbamate (12) (600 mg, 1.74 mmol, 1.0 eq.) in CH₂Cl₂ (10 mL) cooled to 0° C. was added Et₃N (0.3 mL, 2.623 mmol, 1.5 eq.) followed by 2-bromoacetonitrile (0.482 mg, 4.02 mmol, 2.3 eq.). The reaction mixture was warmed to rt and stirred for 16 h. At this time ice cold H₂O was added to the reaction mixture and extracted with 10% MeOH in CH₂Cl₂(2×80 mL). The combined organic layers were washed with brine (2×50 mL), dried over anh. sodium sulfate, filtered and evaporated to get crude residue that was purified by GRACE flash chromatography using 5% MeOH in CH₂Cl₂ as eluent to give tert-butyl ((3S)-1-((cyanomethyl)(((S)-2-oxopyrrolidin-3-yl)methyl)amino)-2-hydroxy-5-methylhexan-3-yl)carbamate (36) (400 mg, 60% yield) C₁₉H₃₄N₄O₄: ¹H NMR (500 MHz, DMSO-d₆) (diastereomers) δ7.64-7.62 (m, 1H), 6.17-6.15 (m, 1H), 4.71-4.57 (m, 1H), 3.82-3.73 (m, 3H), 3.60-3.50 (bs, 1H), 3.48-3.47 (m, 1H), 3.18-3.11 (m, 2H), 2.80-2.70 (m, 1H), 2.49-2.36 (m, 4H), 2.20-2.10 (m, 1H), 1.85-1.71 (m, 1H), 1.60-1.51 (m, 1H), 1.37 (s, 9H), 1.20-1.81 (m, 1H), 0.86-0.83 (m, 6H); LC/MS: Rt=1.71 min (61.5%); m/z 383.2 [M+H]⁺& Rt=1.72 min (23.8%); m/z 383.2 [M+H]⁺.

Step-2: 2-(((3S)-3-Amino-2-hydroxy-5-methylhexyl)(((S)-2-oxopyrrolidin-3-yl)methyl)amino)acetonitrile trifluoroacetic acid salt (37)

To a stirred solution of tert-butyl ((3S)-1-((cyanomethyl)(((S)-2-oxopyrrolidin-3-yl)methyl)amino)-2-hydroxy-5-methylhexan-3-yl)carbamate (36) (400 mg, 1.04 mmol, 1.0 eq.) in CH₂Cl₂ (15 mL) at 0° C. was added trifluoroacetic acid (0.41 mL, 5.23 mmol, 5.0 eq.). Stirring was continued at rt for 12 h. at which TLC indicated consumption of starting material. The reaction mixture was concentrated to give 300 mg of 2-(((3S)-3-amino-2-hydroxy-5-methylhexyl)(((S)-2-oxopyrrolidin-3-yl)methyl)amino)acetonitrile trifluoroacetic acid salt (37). The crude salt was used in the next step without purification.

Step-3: N—((2S,3S)-1-((Cyanomethyl)(((S)-2-oxopyrrolidin-3-yl)methyl)amino)-2-hydroxy-5-methylhexan-3-yl)-4-methoxy-]H-indole-2-carboxamide (38)

To a stirred solution of 2-(((3S)-3-amino-2-hydroxy-5-methylhexyl)(((S)-2-oxopyrrolidin-3-yl)methyl)amino)acetonitrile trifluoroacetic acid salt (37) (250 mg, 0.88 mmol, 1.0 eq.) in DMF (40 mL) at 0° C. was added EDCI HCl (254 mg, 1.32 mmol, 1.5 eq.), HOAt (362 mg, 2.66 mmol, 3.0 eq.), and triethylamine (0.4 mL, 1.78 mmol, 2 eq.) followed by 4-methoxy-1H-indole-2-carboxylic acid (170 mg, 0.88 mmol, 1.0 eq.). The reaction progress was monitored by TLC. After stirring at rt for 12 h. a saturated NaHCO₃ solution was added to the reaction mixture and then extracted with 10% MeOH in CH₂Cl₂(2×40 mL) and washed with brine (2×20 mL). The combined organic layers were dried over anh. sodium sulfate and evaporated to get crude residue that was subjected to GRACE flash chromatography purification using 10% MeOH in CH₂Cl₂ as the eluent to yield N—((2S,3S)-1-((cyanomethyl)(((S)-2-oxopyrrolidin-3-yl)methyl)amino)-2-hydroxy-5-methylhexan-3-yl)-4-methoxy-1H-indole-2-carboxamide (38) (80 mg, 17% over two steps) C₂₄H₃₃N₅O₄: ¹H NMR (500 MHz, DMSO-d₆) δ 11.56 (s, 1H), 7.84-7.81 (m, 1H), 7.62-7.61 (m, 1H), 7.28 (s, 1H), 7.08 (t, 1H, J=8 Hz), 6.99 (d, 1H, J=8.5 Hz), 6.50 (d, 1H, J=7.5 Hz), 4.95-4.78 (m, 1H), 4.20-4.15 (m, 1H), 3.87 (s, 3H), 3.84-3.78 (m, 2H), 3.61 (bs, 1H), 3.15-3.08 (m, 2H), 2.78-2.76 (m, 1H), 2.65-2.55 (m, 1H), 2.46-2.40 (m, 3H), 2.17-2.05 (m, 1H), 1.90-1.80 (m, 1H), 1.64-1.58 (m, 2H), 1.33-1.31 (m, 1H), 0.90-0.86 (m, 6H); LC/MS: Rt=2.06 min (99.2%); m/z 456.3 [M+H]*; HPLC: Rt=9.23 min (98.3%), X-Bridge C₁₈ (4.6×150 mm) 3.5 μ.

Step-4: N—((S)-1-((Cyanomethyl)(((S)-2-oxopyrrolidin-3-yl)methyl)amino)-5-methyl-2-oxohexan-3-yl)-4-methoxy-]H-indole-2-carboxamide, Compound 23

To a stirred solution of N—((2S,3S)-1-((cyanomethyl)(((S)-2-oxopyrrolidin-3-yl)methyl)amino)-2-hydroxy-5-methylhexan-3-yl)-4-methoxy-1H-indole-2-carboxamide (38) (100 mg, 0.219 mmol, 1.0 eq.) in DMSO (10 mL) at 0° C. was added IBX (370 mg, 1.31 mmol, 6.0 eq.). The reaction mixture was stirred at rt for 12 h. To the reaction mixture was added a saturated NaHCO₃ solution which was extracted with CH₂Cl₂ (2×50 mL), washed with NaHCO₃ (2×40 mL) and brine (2×30 mL). The combined organic layers were dried over sodium sulfate, filtered and evaporated to get crude residue that was purified by Prep-HPLC (Column: KROMOSIL-C₁₈ (150*25MM), 7u; Mobile phase: 0.1% formic acid in H₂O/MeCN. Gradient (T % B): 0/15, 9/65, 10.6/65, 10.7/98, 12/98, 12.1/15, 15/15. Flow Rate: 22 mL/min diluent: MeCN+H₂O) to give N—((S)-1-((cyanomethyl)(((S)-2-oxopyrrolidin-3-yl)methyl)amino)-5-methyl-2-oxohexan-3-yl)-4-methoxy-1H-indole-2-carboxamide, Compound 23 (20 mg, 22%) C₂₄H₃₁N₅O₄: ¹H NMR (400 MHz, DMSO-d₆) δ 11.65 (s, 1H), 8.65-8.59 (m, 1H), 7.65 (bs, 1H), 7.34 (bs, 1H), 7.10 (t, 1H, J=8 Hz), 6.92 (d, 1H, J 7.2 Hz), 6.51 (d, 1H, J=7.6 Hz), 4.70-4.54 (m, 1H), 3.95-3.77 (m, 6H), 3.71-3.50 (m, 1H), 3.40-3.32 (m, 2H), 3.19-3.09 (m, 2H), 2.81-2.60 (m, 1H), 2.20-2.10 (m, 1H), 1.96-1.85 (m, 1H), 1.80-1.53 (m, 3H), 0.94-0.85 (m, 6H); LC/MS: Rt=1.77 min (98.1%); m/z 454.2 [M+H]*; HPLC: 98.93% (9.77 min.), X-Bridge C₁₈ (4.6×150 mm) 3.5 μ.

### Example 24: Ethyl (E)-4-(((S)-3-(4-methoxy-1H-indole-2-carboxamido)-5-methyl-2-oxohexyl)(((S)-2-oxopyrrolidin-3-yl)methyl)amino)-4-oxobut-2-enoate, Compound 24

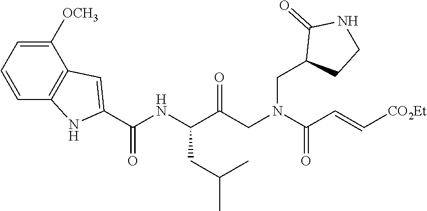 [SMILES: CCOC(=O)/C=C/C(=O)N(CC(=O)[C@H](CC(C)C)NC(=O)c1cc2c(OC)cccc2[nH]1)C[C@@H]1CCNC1=O]

Compound 24 was prepared in four steps from tert-butyl ((2R,3S)-2-hydroxy-5-methyl-1-((((S)-2-oxopyrrolidin-3-yl)methyl)amino)hexan-3-yl)carbamate (12S).

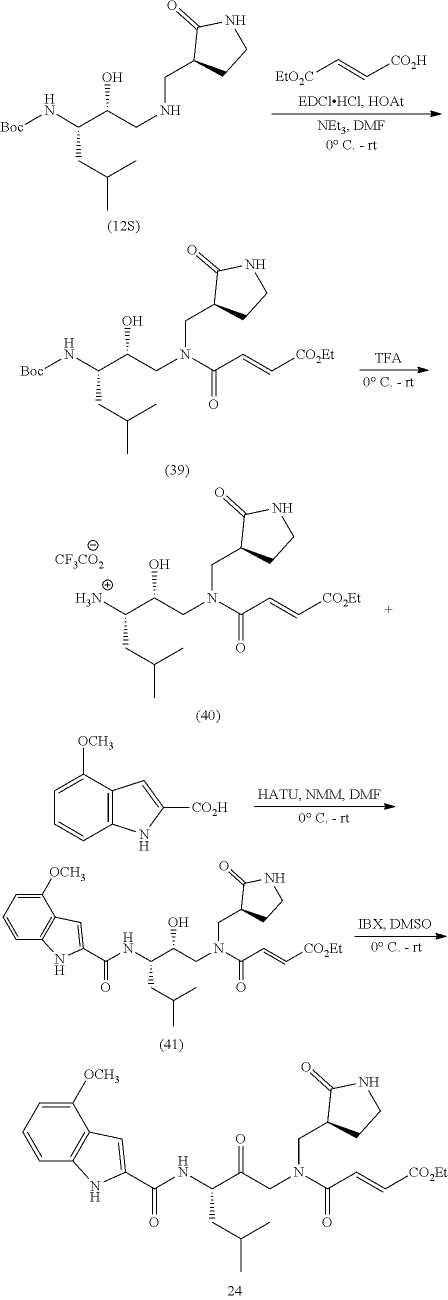

Step-1: Ethyl (E)-4-(((2R,3S)-3-((tert-butoxycarbonyl)amino)-2-hydroxy-5-methylhexyl)(((S)-2-oxopyrrolidin-3-yl)methyl)amino)-4-oxobut-2-enoate (39)

To a stirred solution of tert-butyl ((2R,3S)-2-hydroxy-5-methyl-1-((((S)-2-oxopyrrolidin-3-yl)methyl)amino)hexan-3-yl)carbamate (12S) (prepared according to the procedure described in Example 1, Step-12, but predominantly with the (S,S) diastereomer of epoxide (11)) (500 mg, 1.45 mmol, 1.0 equiv.) in DMF (5 mL) at 0° C. was added EDCI HCl (418 mg, 2.18 mmol, 1.5 eq.), HOAt (600 mg, 4.37 mmol, 3.0 eq.), Et₃N (0.40 mL, 2.91 mmol, 2.0 equiv.), (E)-4-ethoxy-4-oxobut-2-enoic acid (315 g, 2.18 mmol, 1.5 eq.). The reaction mixture was warmed to rt and stirring was continued for 4 h. Upon consumption of compound (12S) as indicated by TLC, cold water (15 mL) was added to the reaction mixture and then extracted with CH₂Cl₂(2×30 mL). The combined organic layers were washed with brine (2×40 mL), dried over sodium sulfate, filtered and concentrated to leave a crude residue which was purified by GRACE flash chromatography using 10% MeOH in CH₂Cl₂ as eluent giving 330 mg (49%) of ethyl (E)-4-(((2R,3S)-3-((tert-butoxycarbonyl)amino)-2-hydroxy-5-methylhexyl)(((S)-2-oxopyrrolidin-3-yl)methyl)amino)-4-oxobut-2-enoate (39) as an pale yellow solid. C₂₃H₃₉N₃O₇: ¹H NMR (400 MHz, DMSO-d₆) δ 7.77 (bs, 1H), 6.6-6.4 (m, 2H), 4.21-4.16 (m, 2H), 3.84-3.81 (m, 1H), 3.60-3.50 (m, 1H), 3.42-3.27 (m, 3H), 3.17-3.13 (m, 1H), 2.66-2.64 (m, 1H), 2.13-2.11 (m, 1H), 1.75-1.70 (m, 1H), 1.60-1.51 (m, 1H), 1.37-13.1 (m, 10H), 1.24 (t, 3H, J=6.8 Hz), 0.88-0.82 (m, 6H).

Step-2: Ethyl (E)-4-(((2R,3S)-3-amino-2-hydroxy-5-methylhexyl)(((S)-2-oxopyrrolidin-3-yl)methyl)amino)-4-oxobut-2-enoate trifluoroacetic acid salt (40)

To a stirred solution of ethyl (E)-4-(((2R,3S)-3-((tert-butoxycarbonyl)amino)-2-hydroxy-5-methylhexyl)(((S)-2-oxopyrrolidin-3-yl)methyl)amino)-4-oxobut-2-enoate (39) (330 mg, 0.703 mmol, 1.0 eq.) in CH₂Cl₂ (20 mL) at 0° C. was added trifluoroacetic acid (0.31 mL, 4.22 mmol, 6.0 eq.). After stirring at rt 12 h. TLC indicated consumption of (39). The reaction mixture was concentrated to give ethyl (E)-4-(((2R,3S)-3-amino-2-hydroxy-5-methylhexyl)(((S)-2-oxopyrrolidin-3-yl)methyl)amino)-4-oxobut-2-enoate trifluoroacetic acid salt (40) (260 mg). that was used in next step without purification.

Step-3: Ethyl (E)-4-(((2R,3S)-2-hydroxy-3-(4-methoxy-]H-indole-2-carboxamido)-5-methylhexyl)(((S)-2-oxopyrrolidin-3-yl)methyl)amino)-4-oxobut-2-enoate (41)

To a stirred solution of ethyl (E)-4-(((2R,3S)-3-amino-2-hydroxy-5-methylhexyl)(((S)-2-oxopyrrolidin-3-yl)methyl)amino)-4-oxobut-2-enoate trifluoroacetic acid salt (40) (260 mg, 0.704 mmol, 1.0 eq.) in DMF (15 mL) at 0° C. was added EDCI HCl (202 mg, 1.05 mmol,1.5 eq.), HOAt (286.6 mg, 2.11 mmol,1.2 eq.) and NEt₃ (0.2 mL, 1.40 mmol, 2 eq.) followed by 4-methoxy-1H-indole-2-carboxylic acid (136 mg, 0.704 mmol, 1.0 eq.). Stirring was continued for 12 h at rt. at which time the reaction mixture was neutralized with a saturated NaHCO₃ solution, and extracted with 10% MeOH in CH₂Cl₂(2×30 mL). The combined organic layers were washed with brine (2×40 mL), dried over sodium sulfate, filtered and evaporated to give a crude residue that was subjected to Prep-HPLC purification. (Column: X-BRIDGE-C₁₈ (250*10), 5u Mobile phase: 0.1% HCO₂H in H₂O/MeCN; Gradient: (T % B):-0/20, 8/55, 9.1/55, 9.2/98, 10/98, 10.1/20, 13/20. Flow Rate: 8 mL/min. Diluent: MeCN+H₂O. Concentration of the collected fractions gave ethyl (E)-4-(((2R,3S)-2-hydroxy-3-(4-methoxy-1H-indole-2-carboxamido)-5-methylhexyl)(((S)-2-oxopyrrolidin-3-yl)methyl)amino)-4-oxobut-2-enoate, (41) (150 mg, 39%, over two steps). C₂₈H₃₈N₄O₇: ¹H NMR (400 MHz, DMSO-d₆) δ 11.53 (s, 1H), 8.0-7.99 (m, 1H), 7.75-7.63 (m, 1H), 7.49-7.48 (m, 1H), 7.32-7.26 (m, 1H), 7.10-7.02 (m, 1H), 7.00-6.98 (m, 1H), 6.55-6.41 (m, 2H), 5.40-5.36 (m, 1H), 4.18-4.11 (m, 2H), 4.09-3.73 (m, 6H), 3.59-3.51 (m, 1H), 3.45-3.42 (m, 1H), 3.36-3.27 (m, 1H), 3.13-3.02 (m, 2H), 2.62-2.56 (m, 1H), 2.07-2.02 (m, 1H), 1.70-1.49 (m, 4H), 1.25-1.15 (m, 3H), 0.98-0.94 (m, 6H). LC/MS: Rt=2.157 min (99.5%); m/z 543.3 [M+H]*; HPLC: Rt=9.724 min (99.5%); X-Bridge C₁₈ (4.6×150 mm), 3.5 μ.

Step-4: Ethyl (E)-4-(((S)-3-(4-methoxy-]H-indole-2-carboxamido)-5-methyl-2-oxohexyl)(((S)-2-oxopyrrolidin-3-yl)methyl)amino)-4-oxobut-2-enoate, Compound 24

To a stirred solution of ethyl (E)-4-(((2R,3S)-2-hydroxy-3-(4-methoxy-1H-indole-2-carboxamido)-5-methylhexyl)(((S)-2-oxopyrrolidin-3-yl)methyl)amino)-4-oxobut-2-enoate (41) 150 mg, 0.276 mmol, 1.0 eq.) in DMSO (3 mL) at 0° C. was added IBX (470 mg, 1.66 mmol, 6.0 eq.). The reaction mixture was warmed to rt and stirred for 16 h. at which time LC/MS indicated none of the alcohol (41) remained. To the reaction mixture was added a saturated NaHCO₃ solution. Extraction of the mixture with CH₂Cl₂(2×40 mL) were followed by washes with NaHCO₃ (3×80 mL) and brine (2×50 mL). The combined organic layers were dried over sodium sulfate, filtered and concentrated to get crude residue that was subjected to prep-HPLC purification. (Column: YMC-TRIART-C₁₈ (150×20 mm), 5u; Mobile phase: 0.1% HCO₂H in H₂O/MeCN. Gradient (T % B): -0/20, 8/70, 10/70, 10.1/98, 13/98, 13.1/20, 15/20; Flow Rate: 22 mL/min; Diluent: MeCN+H₂O) to give ethyl (E)-4-(((S)-3-(4-methoxy-1H-indole-2-carboxamido)-5-methyl-2-oxohexyl)(((S)-2-oxopyrrolidin-3-yl)methyl)amino)-4-oxobut-2-enoate, Compound 24 (80 mg, 57% yield). C₂₈H₃₆N₄O₇: ¹H NMR (500 MHz, DMSO-d₆) δ 11.70-11.60 (m, 1H), 8.71-8.67 (m, 1H), 7.74-7.60 (m, 0.5H), 7.52-7.49 (d, 1H, J=15.0 Hz), 7.35 (s, 1H), 7.12-7.07 (m, 1.5H), 7.02-7.0 (m, 1H), 6.59-6.53 (m, 0.5H), 6.53-6.50 (m, 1.5H), 4.78 (q, 1H, J=14.0 Hz), 4.65-4.58 (m, 1H), 4.45 (q, 1H, J=7.0 Hz), 4.19-4.11 (m, 2H), 3.88 (s, 3H), 3.72-3.50 (m, 2H), 3.11-3.09 (m, 2H), 2.50-2.49 (m, 1H), 2.15-2.0 (m, 1H), 1.80-1.60 (m, 4H), 1.25-1.18 (m, 3H), 0.94-0.89 (m, 6H). LC/MS: Rt=5.19 min (97.6%); m/z 541.3 [M+H]*; HPLC: 97.5% (3.63 min.), Acquity UPLC BEH C₁₈ (100×2.1 mm, 1.7).

### Example 25: Tert-butyl ((S)-1-((S)-1-(2-chloro-N—(((S)-2-oxopyrrolidin-3-yl)methyl)acetamido)-5-methyl-2-oxohexan-3-yl)-2-oxopyrrolidin-3-yl)carbamate, Compound

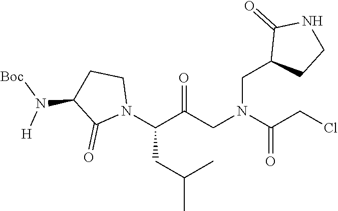 [SMILES: CC(C)C[C@@H](C(=O)CN(C[C@@H]1CCNC1=O)C(=O)CCl)N1CC[C@H](NC(=O)OC(C)(C)C)C1=O]

Compound 25 was prepared in eight steps from methyl (tert-butoxycarbonyl)-L-methionyl-L-leucinate.

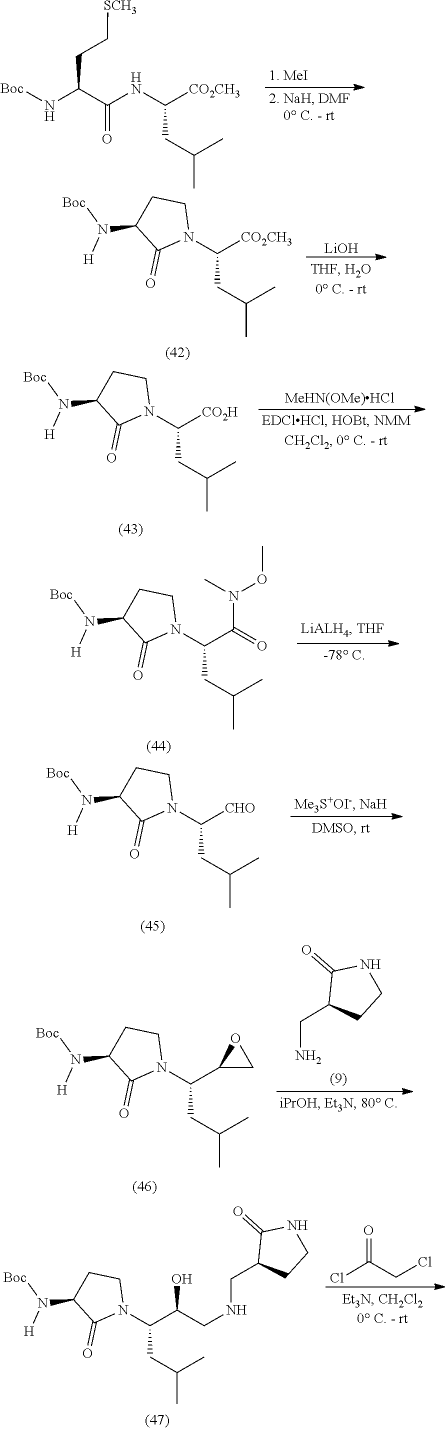

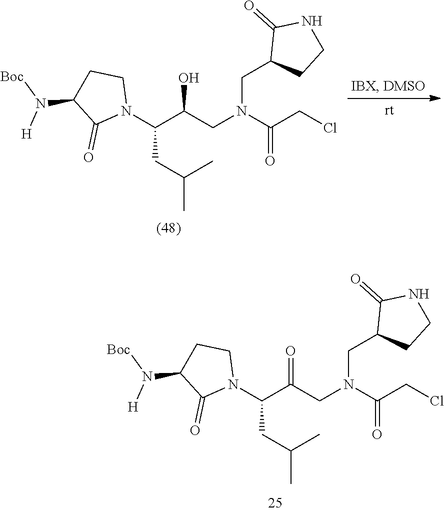 [SMILES: CC(C)C[C@@H]([C@@H](O)CN(C[C@@H]1CCNC1=O)C(=O)CCl)N1CC[C@H](NC(=O)OC(C)(C)C)C1=O ; CC(C)C[C@@H](C(=O)CN(C[C@@H]1CCNC1=O)C(=O)CCl)N1CC[C@H](NC(=O)OC(C)(C)C)C1=O]

Step-1: Methyl (S)-2-((S)-3-((tert-butoxycarbonyl)amino)-2-oxopyrrolidin-1-yl)-4-methylpentanoate (42)

Following the procedure of Freidinger, et al., JOC 1982, 47, p.104-109, methyl (tert-butoxycarbonyl)-L-methionyl-L-leucinate (36.0 g, 95.744 mmol, 1.0 eq.) was dissolved in Mel (360 mL) at 0° C. and stirred the reaction mixture for 16 h. The reaction mixture was concentrated to give a crude methyl sulfonium iodide salt. This oil was dissolved in DMF (400 mL) followed by the addition of NaH (3.46 g, 143.62 mmol, 1.5 eq.) at 0° C. The reaction mixture was allowed to warm to rt and stirred overnight. Cold water (300 mL) was cautiously added, and the mixture was extracted with ethyl acetate (3×500 mL). The combined organic layers were washed with brine (2×300 mL), dried over anh. sodium sulfate, filtered and concentrated to give a residue, which was purified by column chromatography using 30% EtOAc in pet. ether as eluent to obtain methyl (S)-2-((S)-3-((tert-butoxycarbonyl)amino)-2-oxopyrrolidin-1-yl)-4-methylpentanoate (42) (26 g, 82.8%) as an off-white solid. C₁₆H₂₈N₂O₅: ¹H NMR (400 MHz, DMSO-d₆) δ 7.13 (d, 1H, J=9.2 Hz), 4.61-4.57 (m, 1H), 4.21-4.19 (m, 1H), 3.64 (s, 3H), 3.21-3.18 (m, 1H), 2.24-2.21 (m, 1H), 1.80-1.71 (m, 2H), 1.58-1.51 (m, 1H), 1.38 (s, 11H), 0.90-0.83 (m, 6H); LC/MS (ELSD): Rt=1.92 min (89.36%); m/z 329.2 [M+H]*.

Step-2: (S)-2-((S)-3-((Tert-butoxycarbonyl)amino)-2-oxopyrrolidin-1-yl)-4-methylpentanoic acid (43)

To a stirred solution of methyl (S)-2-((S)-3-((tert-butoxycarbonyl)amino)-2-oxopyrrolidin-1-yl)-4-methylpentanoate (42) (5 g, 15.24 mmol, 1.0 eq.) in a mixture of THF:H₂O (4:1, 50 mL), was added LiOH H₂O (765 mg, 18.29 mmol, 1.2 eq.) at 0° C. and The reaction mixture was stirred at rt for 1 h. until TLC indicated complete consumption of the methyl ester. At this time the pH of reaction mixture was adjusted to 4 by the addition of 5% aq. acetic acid solution which was extracted with ethyl acetate (3×100 mL). The combined organic layers were washed with brine (2×50 mL), dried over sodium sulfate, filtered and concentrated to give 4.5 g of crude carboxylic acid, (S)-2-((S)-3-((tert-butoxycarbonyl)amino)-2-oxopyrrolidin-1-yl)-4-methylpentanoic acid (43), (94.1%) as an oil which was used without further purification. C₁₅H₂₆N₂O₅: ¹H NMR (400 MHz, DMSO-d₆) δ 12.4 (bs, 1H), 7.12 (d, 1H, J 9.2 Hz), 4.52-4.48 (m, 1H), 4.21-4.18 (m, 1H), 3.20-3.16 (m, 1H), 2.25-2.19 (m, 1H), 1.82-1.77 (m, 1H), 1.71-1.65 (m, 1H), 1.58-1.51 (m, 1H), 1.44 (s, 11H), 0.89 (d, 3H, J=6.4 Hz), 0.84 (d, 3H, J=6.4 Hz). LC/MS (ELSD): Rt=1.69 min (93.79%); m/z 315.25 [M+H]*.

Step-3: Tert-butyl ((S)-1-((S)-1-(methoxy(methyl)amino)-4-methyl-1-oxopentan-2-yl)-2-oxopyrrolidin-3-yl)carbamate (44)

To a stirred solution of (S)-2-((S)-3-((tert-butoxycarbonyl)amino)-2-oxopyrrolidin-1-yl)-4-methylpentanoic acid (43) (4.5 g, 14.30 mmol, 1.0 equiv.) in DMF (45 mL) at 0° C., was added HOBt (2.90 g, 21.50 mmol, 1.5 eq.), EDCI HCl (4.10 g, 21.50 mmol, 1.5 eq.). After 0.5 h. N-methylmorpholine (10 mL, 8.58 mmol, 6.0 eq.) and CH₃NH(OMe) HCl (2.78 g, 28.60 mmol, 2 eq.) were added. Maintaining the temperature at 0° C., the reaction mixture was stirred for 4 h. Once TLC indicated consumption of carboxylic acid (43) the mixture was quenched with H₂O (30 mL), extracted with ethyl acetate (3×200 mL) and washed with water (3×100 mL) and brine (2×100 mL). The combined organic layers were dried over anh. sodium sulfate, filtered and concentrated to give a crude residue that was subjected to silica gel column chromatography using 80% EtOAc in pet. ether to obtain 3.2 g of tert-butyl ((S)-1-((S)-1-(methoxy(methyl)amino)-4-methyl-1-oxopentan-2-yl)-2-oxopyrrolidin-3-yl)carbamate (44) (62.4%) C₁₇H₃₁N₃O₅: ¹H NMR (500 MHz, DMSO-d₆) δ 7.09 (d, 1H, J=8.5 Hz), 5.14 (bs, 1H), 4.21-4.15 (m, 1H), 3.70 (s, 3H), 3.6 (bs, 1H), 3.22-3.17 (m, 1H), 3.08 (s, 3H), 2.21-2.17 (m, 1H), 1.81-1.70 (m, 1H), 2.17 (m, 1H), 1.69-1.60 (m, 1H), 1.38 (s, 9H), 1.37-1.28 (m, 1H), 0.90-0.85 (m, 6H). LC/MS (ELSD): Rt=1.82 min (96.24%); m/z 358.29 [M+H]*.

Step-4: Tert-butyl ((S)-1-((S)-4-methyl-1-oxopentan-2-yl)-2-oxopyrrolidin-3-yl)carbamate (45)

To a stirred solution of tert-butyl ((S)-1-((S)-1-(methoxy(methyl)amino)-4-methyl-1-oxopentan-2-yl)-2-oxopyrrolidin-3-yl)carbamate (44) (2 g, 5.586 mmol, 1.0 eq.) in diethyl ether (20 mL) at −78° C. was added LAH (4.46 mL, 4.47 mmol, 0.8 eq., 1M in THF). The reaction mixture was stirred at −78° C. for 3 h. and monitored by TLC and LCMS. The reaction mixture was quenched with saturated Na₂SO₄ (10 mL) and filtered through a pad of celite and washed with ethyl acetate (3×100 mL). The filtrate was collected, dried over Na₂SO₄, filtered, and concentrated to give the crude product that was purified by column chromatography using 40% EtOAc in pet. ether as eluent to provide 1.5 g of tert-butyl ((S)-1-((S)-4-methyl-1-oxopentan-2-yl)-2-oxopyrrolidin-3-yl)carbamate (45) (90.14%) as a yellow oil. C₁₅H₂₆N₂O₄: ¹H NMR (500 MHz, DMSO-d₆) δ 9.46 (d, J=1.6 Hz, 1H), 7.19-7.13 (m, 1H), 5.78-5.73 (m, 1H), 4.46-3.70 (m, 2H), 3.31-3.09 (m, 2H), 2.33-2.18 (m, 1H), 1.87-1.9 (m, 3H), 1.38 (s, 9H), 0.92-0.80 (m, 6H). LC/MS (ELSD): Rt=1.57 min (72.48%) m/z 299.24 [M+H]*.

Step-5: Tert-butyl ((S)-1-((S)-3-methyl-]-((R)-oxiran-2-yl)butyl)-2-oxopyrrolidin-3-yl)carbamate (46)

To a stirred solution of trimethylsulfoxonium iodide (2.2 g, 10.07 mmol, 2.0 eq.) in DMSO (15 mL) was added NaH (241 mg, 10.07 mmol, 2.0 eq.) portion wise at rt. The reaction mixture was stirred 0.5 h. and tert-butyl ((S)-1-((S)-4-methyl-1-oxopentan-2-yl)-2-oxopyrrolidin-3-yl)carbamate (45) (1.5 g, 5.033 mmol, 1.0 eq.) in DMSO (15 mL) was added to the ylid and stirred at rt for 2 h. After consumption of (45), as indicated by TLC and LC/MS, the reaction mixture was quenched with water (50 mL) and extracted with diethyl ether (3×100 mL). The combined organic layers were washed with brine (2×50 mL), dried over anh. sodium sulfate, filtered, and concentrated to give a residue that was subjected to column chromatography using 20% EtOAc in pet. ether as eluent to provide 500 mg of tert-butyl ((S)-1-((S)-3-methyl-1-((R)-oxiran-2-yl)butyl)-2-oxopyrrolidin-3-yl)carbamate (46) (31.8%) as a semi-solid. C₁₆H₂₈N₂O₄: ¹H NMR (400 MHz, CDCl₃) δ 5.13 (bs, 1H), 4.19-4.14 (m, 2H)), 3.44 (t, 1H, J=9.6 Hz), 3.25-3.23 (m, 1H), 3.05-3.03 (m, 1H), 2.79-2.78 (m, 1H), 2.64-2.59 (m, 2H), 1.84-1.82 (m, 1H), 1.69-1.66 (m, 1H), 1.49 (s, 9H), 1.48-1.39 (m, 1H), 0.95-0.88 (m, 6H). LC/MS (ELSD): Rt=1.83 min (99.74%) m/z 313.29 [M+H]*.

Step-6: Tert-butyl ((S)-1-((2S,3S)-2-hydroxy-5-methyl-]-((((S)-2-oxopyrrolidin-3-yl)methyl)amino)hexan-3-yl)-2-oxopyrrolidin-3-yl)carbamate (47)

To a stirred solution of (S)-3-(aminomethyl)pyrrolidin-2-one (9) (1.8 g, 5.760 mmol, 1.0 equiv.) in iPrOH (60 mL) at rt, was added tert-butyl ((S)-1-((S)-3-methyl-1-((R)-oxiran-2-yl)butyl)-2-oxopyrrolidin-3-yl)carbamate (46) (0.657 g, 5.760 mmol, 1.0 equiv.), a cat. amount of Et₃N, and molecular sieves powder (1 g). The stirred reaction mixture was heated to 85° C. After 18 h, the molecular sieves were removed by filtration and washed with iPrOH. The solvent was evaporated and the crude product was purified by column chromatography using 3% MeOH in CH₂Cl₂ as eluent to afford 1.0 g of tert-butyl ((S)-1-((2S,3S)-2-hydroxy-5-methyl-1-((((S)-2-oxopyrrolidin-3-yl)methyl)amino)hexan-3-yl)-2-oxopyrrolidin-3-yl)carbamate (47) as a thick syrup in 41% yield. C₂₁H₃₈N₄O₅: ¹H NMR (400 MHz, DMSO-d₆) (Rotamers observed) δppm: 7.93 (s, 1H), 7.17-7.15 (m, 1H), 5.70-5.60 (bs, 1H), 4.05-4.03 (m, 1H), 3.87-3.75 (m, 2H), 3.53-3.51 (m, 1H), 3.22-3.11 (m, 5H), 2.88-2.66 (m, 3H), 2.30-2.22 (m, 2H), 1.90-1.75 (m, 3H), 1.38 (s, 12H), 0.90-0.78 (m, 8H). LC/MS (ELSD): Rt=1.24 min (86.59%); m/z 427.34 [M+H]*.

Step-7: Tert-butyl ((S)-1-((2S,3S)-1-(2-chloro-N—(((S)-2-oxopyrrolidin-3-yl)methyl)acetamido)-2-hydroxy-5-methylhexan-3-yl)-2-oxopyrrolidin-3-yl)carbamate (48)

To a stirred solution of tert-butyl ((S)-1-((2S,3S)-2-hydroxy-5-methyl-1-((((S)-2-oxopyrrolidin-3-yl)methyl)amino)hexan-3-yl)-2-oxopyrrolidin-3-yl)carbamate (47) (1.0 g, 2.30 mmol, 1.0 eq.) in CH₂Cl₂ (10 mL) at 0° C. was added Et₃N (0.48 mL, 3.40 mmol, 1.5 equiv.) followed by chloroacetyl chloride (0.18 mL, 2.30 mmol, 1.0 eq.) The temperature of the stirred reaction mixture was maintained at 0° C. for 1 h. When TLC indicated no more of diamine (47) remained, the reaction mixture was quenched with cold water (10 mL) and extracted with CH₂Cl₂(3×50 mL). The combined organic layers were washed with brine (2×20 mL), dried over anh. sodium sulfate, filtered and concentrated to give the crude product that was purified by column chromatography with 2% MeOH in CH₂Cl₂ as eluent to afford tert-butyl ((S)-1-((2S,3S)-1-(2-chloro-N—(((S)-2-oxopyrrolidin-3-yl)methyl)acetamido)-2-hydroxy-5-methylhexan-3-yl)-2-oxopyrrolidin-3-yl)carbamate (48) (550 mg, 33.46%) as an off-white solid. C₂₃H₃₉ClN₄O₆: ¹H NMR (400 MHz, DMSO-d₆) δ 7.79-7.65 (m, 1H), 7.16-7.06 (m, 1H), 5.58-5.50 (m, 1H), 4.66 (d, 1H, J 14.4 Hz), 4.45-4.36 (m, 2H), 4.06-4.03 (m, 1H), 3.88-3.59 (m, 4H), 3.25-3.10 (m, 5H), 2.21 (bs, 1H), 2.02 (bs, 1H), 1.81-1.69 (m, 3H), 1.39 (s, 1OH), 1.19-1.15 (m, 3H) 0.89-0.82 (m, 6H). LC/MS: Rt=1.70 min (79.4%), m/z 503.3 [M+H]*.

Step-8: Tert-butyl ((S)-1-((S)-1-(2-chloro-N—(((S)-2-oxopyrrolidin-3-yl)methyl)acetamido)-5-methyl-2-oxohexan-3-yl)-2-oxopyrrolidin-3-yl)carbamate, Compound 24

To a stirred solution of tert-butyl ((S)-1-((2S,3S)-1-(2-chloro-N—(((S)-2-oxopyrrolidin-3-yl)methyl)acetamido)-2-hydroxy-5-methylhexan-3-yl)-2-oxopyrrolidin-3-yl)carbamate (48) (550 mg, 1.09 mmol, 1.0 eq.) in DMSO (6 mL) was added IBX (1.53 g, 5.467 mmol, 5.0 eq.) at rt. The reaction mixture was stirred for 16 h. To the mixture was added a saturated NaHCO₃ solution. The mixture was extracted with diethyl ether (3×50 mL) and washed with NaHCO₃ (2×50 mL) and brine (2×50 mL). The combined organic layers were dried over sodium sulfate, filtered and concentrated to give a residue that was subjected to column chromatography using 90% ethyl acetate in pet. ether as eluent to afford 450 mg of tert-butyl ((S)-1-((S)-1-(2-chloro-N—(((S)-2-oxopyrrolidin-3-yl)methyl)acetamido)-5-methyl-2-oxohexan-3-yl)-2-oxopyrrolidin-3-yl)carbamate, Compound 24, as a white solid in 82% yield. C₂₃H₃₇ClN₄O₆: ¹H NMR (400 MHz, DMSO-d₆) δ 7.76-7.72 (m, 1H), 7.21-7.16 (m, 1H), 4.71-4.60 (m, 1H), 4.49-4.26 (m, 2H), 4.21-4.14 (m, 3H), 3.60-3.50 (m, 2H), 3.17-3.09 (m, 4H), 2.70-2.60 (m, 2H), 2.20-2.10 (m, 1H), 1.90-1.50 (m, 3H), 1.50-1.36 (m, 11H), 0.88-0.83 (m, 6H); 1.36 (m, 1OH), 0.89-0.83 (m, 6H); LC/MS: Rt=1.75 min (98.8%); m/z 501.26 [M+H]*.

### Example 26: N—((S)-3-((S)-3-Acetamido-2-oxopyrrolidin-1-yl)-5-methyl-2-oxohexyl)-2-chloro-N—(((S)-2-oxopyrrolidin-3-yl)methyl)acetamide, Compound 26

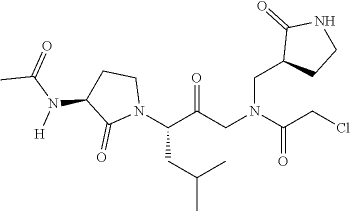 [SMILES: CC(=O)N[C@H]1CCN([C@@H](CC(C)C)C(=O)CN(C[C@@H]2CCNC2=O)C(=O)CCl)C1=O]

Compound 26 was prepared according to the following two step procedure.

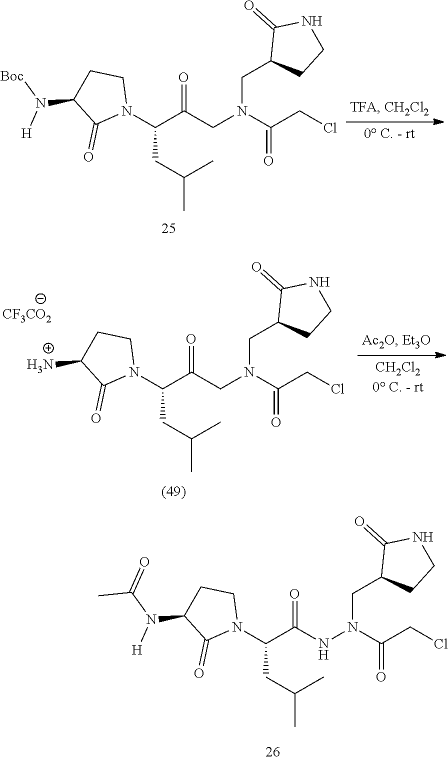 [SMILES: CC(C)C[C@@H](C(=O)CN(C[C@@H]1CCNC1=O)C(=O)CCl)N1CC[C@H](NC(=O)OC(C)(C)C)C1=O ; "CF3CO2" ; CC(C)C[C@@H](C(=O)CN(C[C@@H]1CCNC1=O)C(=O)CCl)N1CC[C@H]([NH3+])C1=O ; CC(=O)N[C@H]1CCN([C@@H](CC(C)C)C(=O)NN(C[C@@H]2CCNC2=O)C(=O)CCl)C1=O]

Step-1: N—((S)-3-((S)-3-Amino-2-oxopyrrolidin-1-yl)-5-methyl-2-oxohexyl)-2-chloro-N—(((S)-2-oxopyrrolidin-3-yl)methyl)acetamide trifluoroacetic acid salt (49)

To a stirred solution tert-butyl ((S)-1-((S)-1-(2-chloro-N—(((S)-2-oxopyrrolidin-3-yl)methyl)acetamido)-5-methyl-2-oxohexan-3-yl)-2-oxopyrrolidin-3-yl)carbamate, Compound 24, (450 mg, 1 eq.) in CH₂Cl₂ (10 mL) cooled to 0° C., was added trifluoroacetic acid (0.34 mL, 4.49 mmol, 5.0 eq.). The reaction mixture was gradually warmed to rt and stirred for 16 h. Once the carbamate 24 was completely exhausted, as indicated by TLC, the reaction mixture was concentrated to give 360 mg of N—((S)-3-((S)-3-amino-2-oxopyrrolidin-1-yl)-5-methyl-2-oxohexyl)-2-chloro-N—(((S)-2-oxopyrrolidin-3-yl)methyl)acetamide trifluoroacetic acid salt (49) that was used without purification. C₁₄H₂₄ClN₃03: LC/MS: Rt=1.12 min, m/z 401.24 [M+H]*.

Step-2: N—((S)-3-((S)-3-Acetamido-2-oxopyrrolidin-1-yl)-5-methyl-2-oxohexyl)-2-chloro-N—(((S)-2-oxopyrrolidin-3-yl)methyl)acetamide, Compound 26

To a stirred solution of N—((S)-3-((S)-3-amino-2-oxopyrrolidin-1-yl)-5-methyl-2-oxohexyl)-2-chloro-N—(((S)-2-oxopyrrolidin-3-yl)methyl)acetamide trifluoroacetic acid salt (49) (360 mg, 0.89 mmol, 1.0 eq.) in CH₂Cl₂ (5 mL) cooled to 0° C., was added triethylamine (0.37 mL, 2.69 mmol, 3.0 eq.) and acetic anhydride (0.08 mL, 0.90 mmol, 1.0 eq.). The reaction mixture was warmed to rt and stirring was continued for 12 h. The reaction mixture was quenched with cold water (15 mL) and extracted with 10% MeOH in CH₂Cl₂(2×20 mL). The combined organic layers were washed with brine (2×10 mL), dried over sodium sulfate, filtered and concentrated to give a solid that was purified by SFC (SFC-150-008; Torus-2-PIC; 100×19 mm, 5; 90% CO₂/MeOH; Flow=70 g/min; BP=100 bar; 30° C.; k=215 nM) to afford 80 mg of N—((S)-3-((S)-3-acetamido-2-oxopyrrolidin-1-yl)-5-methyl-2-oxohexyl)-2-chloro-N—(((S)-2-oxopyrrolidin-3-yl)methyl)acetamide, Compound 26 as a white solid in 21.1% yield over two steps. C₂₀H₃₁ClN₄O_{5:} ¹H NMR (500 MHz, DMSO-d₆) (rotamers) δppm 8.24 (t, J=9.0 Hz, 1H), 7.75 (d, 1H, J 5.0 Hz), 4.69-4.68 (m, 1H), 4.51-4.41 (m, 3H), 4.27-4.09 (m, 2H), 3.61-3.53 (m, 1H), 3.41-3.39 (m, 0.5 Hz), 3.23-3.11 (m, 4.5 Hz), 2.30-2.25 (m, 1H), 2.20-1.98 (m, 2H), 1.90 (m, 1H), 1.85 (s, 1.5H), 1.84 (s, 1.5H), 1.82-1.38 (m, 4H), 0.91 (t, J=5.8 Hz, 3H), 0.85 (t, J=6.0 Hz, 3H); LC/MS (ELSD): Rt=1.49 min (98.8%); m/z 443.3 [M+H]*; HPLC (ELSD): 99.8% (7.50 min.), X-Bridge C₁₈ (4.6×150 mm), 3.5.

### Example P1: N—((S)-1-(2-Chloro-N—(((S)-2-oxopyrrolidin-3-yl)methyl)acetamido)-5-methyl-2-oxohexan-3-yl)-7-methylbenzofuran-2-carboxamide, Compound P1

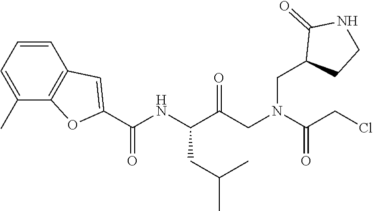 [SMILES: Cc1cccc2cc(C(=O)N[C@@H](CC(C)C)C(=O)CN(C[C@@H]3CCNC3=O)C(=O)CCl)oc12]

Compound P1 is prepared using the procedure outlined for Compound 5 in Example 5 from N—((S)-3-amino-5-methyl-2-oxohexyl)-2-chloro-N—(((S)-2-oxopyrrolidin-3-yl)methyl)acetamide trifluoroacetic acid salt (14) and 7-methylbenzofuran-2-carboxylic acid.

### Example P2: N—((S)-1-(2-chloro-N—(((S)-2-oxopyrrolidin-3-yl)methyl)acetamido)-5-methyl-2-oxohexan-3-yl)-7-methoxybenzofuran-2-carboxamide, Compound P2

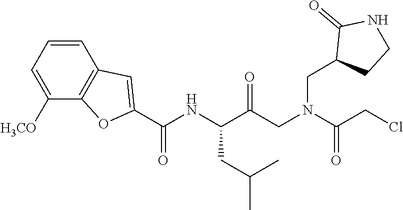 [SMILES: COc1cccc2cc(C(=O)N[C@@H](CC(C)C)C(=O)CN(C[C@@H]3CCNC3=O)C(=O)CCl)oc12]

Compound P2 is prepared using the procedure outlined for Compound 5 in Example 5 from N—((S)-3-amino-5-methyl-2-oxohexyl)-2-chloro-N—(((S)-2-oxopyrrolidin-3-yl)methyl)acetamide trifluoroacetic acid salt (14) and 7-methoxybenzofuran-2-carboxylic acid.

### Example P3: N—((3S)-1-(2-chloro-N—((2-oxopiperidin-3-yl)methyl)acetamido)-5-methyl-2-oxohexan-3-yl)-4-methoxy-1H-indole-2-carboxamide, Compound P3

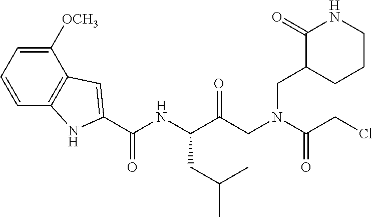 [SMILES: COc1cccc2[nH]c(C(=O)N[C@@H](CC(C)C)C(=O)CN(CC3CCCNC3=O)C(=O)CCl)cc12]

Compound P3 is prepared using the procedures described in Examples 1 and 2 for Compound 2 starting from (2-oxopiperidin-3-yl)methyl methanesulfonate.

### Example P4: N—((3S)-1-(1-(Benzylsulfinyl)—N—(((S)-2-oxopyrrolidin-3-yl)methyl)methanamido)-5-methyl-2-oxohexan-3-yl)-4-methoxy-1H-indole-2-carboxamide, Compound P4

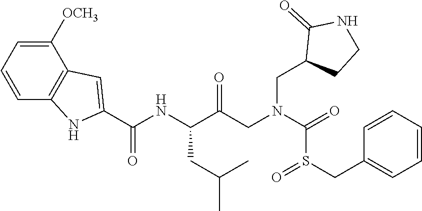 [SMILES: COc1cccc2[nH]c(C(=O)N[C@@H](CC(C)C)C(=O)CN(C[C@@H]3CCNC3=O)C(=O)S(=O)Cc3ccccc3)cc12]

Compound P4 is prepared by the oxidation of S-benzyl ((S)-3-(4-methoxy-1H-indole-2-carboxamido)-5-methyl-2-oxohexyl)(((S)-2-oxopyrrolidin-3-yl)methyl)carbamothioate, Compound 18 with oxone, hydrogen peroxide or other oxidants.

### Example P5: N—((S)-1-(1-(Benzylsulfonyl)—N—(((S)-2-oxopyrrolidin-3-yl)methyl)methanamido)-5-methyl-2-oxohexan-3-yl)-4-methoxy-1H-indole-2-carboxamide, Compound P5

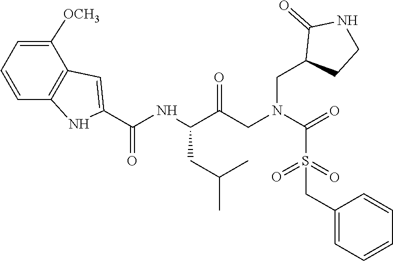 [SMILES: COc1cccc2[nH]c(C(=O)N[C@@H](CC(C)C)C(=O)CN(C[C@@H]3CCNC3=O)C(=O)S(=O)(=O)Cc3ccccc3)cc12]

Compound P5 is prepared by the oxidation of N—((3S)-1-(1-(Benzylsulfinyl)—N—(((S)-2-oxopyrrolidin-3-yl)methyl)methanamido)-5-methyl-2-oxohexan-3-yl)-4-methoxy-1H-indole-2-carboxamide, Compound P4 or S-benzyl ((S)-3-(4-methoxy-1H-indole-2-carboxamido)-5-methyl-2-oxohexyl)(((S)-2-oxopyrrolidin-3-yl)methyl)carbamothioate, Compound 18 with oxone, hydrogen peroxide or other oxidants.

### Example P6: S-Methyl ((S)-3-(4-methoxy-1H-indole-2-carboxamido)-5-methyl-2-oxohexyl)(((S)-2-oxopyrrolidin-3-yl)methyl)carbamothioate, Compound P6

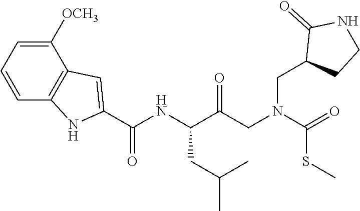 [SMILES: COc1cccc2[nH]c(C(=O)N[C@@H](CC(C)C)C(=O)CN(C[C@@H]3CCNC3=O)C(=O)SC)cc12]

Compound P6 is prepared using the procedures outlined for Compound 16 in Example 16 and Compound 18 in Example 18 starting from tert-butyl ((3S)-2-hydroxy-5-methyl-1-((((S)-2-oxopyrrolidin-3-yl)methyl)amino)hexan-3-yl)carbamate (12) and S-methyl carbonochloridothioate.

### Example P7: 4-Methoxy-N—((3S)-5-methyl-1-(1-(methylsulfinyl)—N—(((S)-2-oxopyrrolidin-3-yl)methyl)methanamido)-2-oxohexan-3-yl)-1H-indole-2-carboxamide, Compound P7

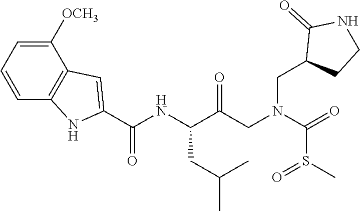 [SMILES: COc1cccc2[nH]c(C(=O)N[C@@H](CC(C)C)C(=O)CN(C[C@@H]3CCNC3=O)C(=O)S(C)=O)cc12]

Compound P7 is prepared by the oxidation of S-methyl ((S)-3-(4-methoxy-1H-indole-2-carboxamido)-5-methyl-2-oxohexyl)(((S)-2-oxopyrrolidin-3-yl)methyl)carbamothioate, Compound P6 with oxone, hydrogen peroxide or other oxidants.

### Example P8: 4-Methoxy-N—((S)-5-methyl-1-(1-(methylsulfonyl)—N—(((S)-2-oxopyrrolidin-3-yl)methyl)methanamido)-2-oxohexan-3-yl)-1H-indole-2-carboxamide, Compound P8

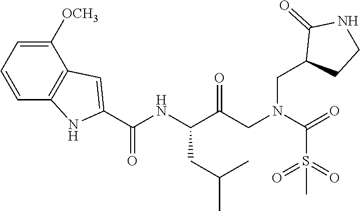 [SMILES: COc1cccc2[nH]c(C(=O)N[C@@H](CC(C)C)C(=O)CN(C[C@@H]3CCNC3=O)C(=O)S(C)(=O)=O)cc12]

Compound P8 is prepared by the oxidation of S-methyl ((S)-3-(4-methoxy-1H-indole-2-carboxamido)-5-methyl-2-oxohexyl)(((S)-2-oxopyrrolidin-3-yl)methyl)carbamothioate, Compound P6 or 4-methoxy-N—((3S)-5-methyl-1-(1-(methylsulfinyl)—N—(((S)-2-oxopyrrolidin-3-yl)methyl)methanamido)-2-oxohexan-3-yl)-1H-indole-2-carboxamide, Compound P7 with oxone, hydrogen peroxide or other oxidants.

### Example P9: S-Ethyl ((S)-3-(4-methoxy-1H-indole-2-carboxamido)-5-methyl-2-oxohexyl)(((S)-2-oxopyrrolidin-3-yl)methyl)carbamothioate, CompoundP9

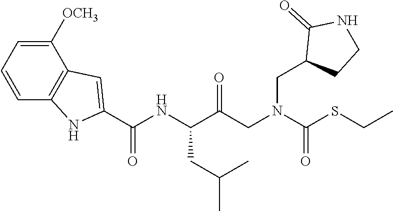 [SMILES: CCSC(=O)N(CC(=O)[C@H](CC(C)C)NC(=O)c1cc2c(OC)cccc2[nH]1)C[C@@H]1CCNC1=O]

Compound P9 is prepared using the procedures outlined for Compound 16 in Example 16 and Compound 18 in Example 18 starting from tert-butyl ((3S)-2-hydroxy-5-methyl-1-((((S)-2-oxopyrrolidin-3-yl)methyl)amino)hexan-3-yl)carbamate (12) and S-ethyl carbonochloridothioate.

### Example P10: N—((3S)-1-(1-(Ethylsulfinyl)—N—(((S)-2-oxopyrrolidin-3-yl)methyl)methanamido)-5-methyl-2-oxohexan-3-yl)-4-methoxy-1H-indole-2-carboxamide, Compound P10

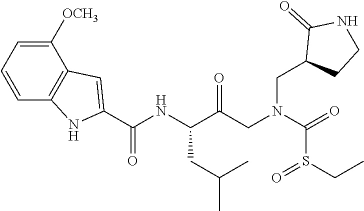 [SMILES: CCS(=O)C(=O)N(CC(=O)[C@H](CC(C)C)NC(=O)c1cc2c(OC)cccc2[nH]1)C[C@@H]1CCNC1=O]

Compound P10 is prepared by the oxidation of S-ethyl ((S)-3-(4-methoxy-1H-indole-2-carboxamido)-5-methyl-2-oxohexyl)(((S)-2-oxopyrrolidin-3-yl)methyl)carbamothioate, Compound P9 with oxone, hydrogen peroxide or other oxidants.

### Example P11: N—((S)-1-(1-(Ethylsulfonyl)—N—(((S)-2-oxopyrrolidin-3-yl)methyl)methanamido)-5-methyl-2-oxohexan-3-yl)-4-methoxy-1H-indole-2-carboxamide, Compound P11

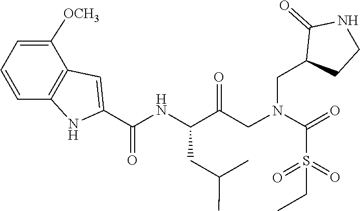 [SMILES: CCS(=O)(=O)C(=O)N(CC(=O)[C@H](CC(C)C)NC(=O)c1cc2c(OC)cccc2[nH]1)C[C@@H]1CCNC1=O]

Compound P11 is prepared by the oxidation S-ethyl ((S)-3-(4-methoxy-1H-indole-2-carboxamido)-5-methyl-2-oxohexyl)(((S)-2-oxopyrrolidin-3-yl)methyl)carbamothioate, Compound P9 or N—((3S)-1-(1-(ethylsulfinyl)—N—(((S)-2-oxopyrrolidin-3-yl)methyl)methanamido)-5-methyl-2-oxohexan-3-yl)-4-methoxy-1H-indole-2-carboxamide, Compound P10 with oxone, hydrogen peroxide or other oxidants.

### Example P13: 4-Methoxy-N—((S)-5-methyl-2-oxo-1-((E)-4-oxo-N—(((S)-2-oxopyrrolidin-3-yl)methyl)pent-2-enamido)hexan-3-yl)-1H-indole-2-carboxamide, Compound P13

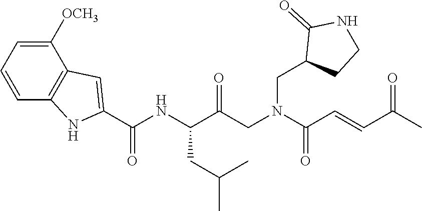 [SMILES: COc1cccc2[nH]c(C(=O)N[C@@H](CC(C)C)C(=O)CN(C[C@@H]3CCNC3=O)C(=O)/C=C/C(C)=O)cc12]

Compound P13 is prepared using the procedure outlined for Compound P12 in Example P12 substituting (E)-4-ethoxy-4-oxobut-2-enoic acid with (E)-4-oxopent-2-enoic acid.

### Example P14: N—((S)-1-((Z)-3-Cyano-N—(((S)-2-oxopyrrolidin-3-yl)methyl)acrylamido)-5-methyl-2-oxohexan-3-yl)-4-methoxy-1H-indole-2-carboxamide, Compound P14

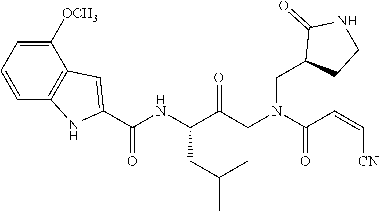 [SMILES: COc1cccc2[nH]c(C(=O)N[C@@H](CC(C)C)C(=O)CN(C[C@@H]3CCNC3=O)C(=O)/C=C\C#N)cc12]

Compound P14 is prepared using the procedure outlined for Compound P12 in Example P12 substituting (E)-4-ethoxy-4-oxobut-2-enoic acid with (Z)-3-cyanoacrylic acid.

### Example P15: 4-Methoxy-N—((S)-5-methyl-1-((E)-3-(methylsulfonyl)—N—(((S)-2-oxopyrrolidin-3-yl)methyl)acrylamido)-2-oxohexan-3-yl)-1H-indole-2-carboxamide, Compound P15

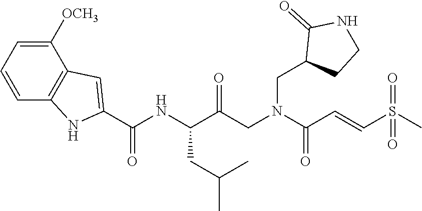 [SMILES: COc1cccc2[nH]c(C(=O)N[C@@H](CC(C)C)C(=O)CN(C[C@@H]3CCNC3=O)C(=O)/C=C/S(C)(=O)=O)cc12]

Compound P15 is prepared using the procedure outlined for Compound P12 in Example P12 substituting (E)-4-ethoxy-4-oxobut-2-enoic acid with (E)-3-(methylsulfonyl)acrylic acid.

### Example P17: 1-Methylcyclobutyl ((S)-1-(2-chloro-N—(((S)-2-oxopyrrolidin-3-yl)methyl)acetamido)-5-methyl-2-oxohexan-3-yl)carbamate, Compound P17

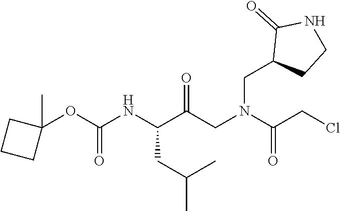 [SMILES: CC(C)C[C@H](NC(=O)OC1(C)CCC1)C(=O)CN(C[C@@H]1CCNC1=O)C(=O)CCl]

Compound P17 is prepared using the procedure outlined for Compound 22 in Example 22 replacing cyclobutanol with 1-methylcyclobutan-1-ol.

### Example P18: 4-Methoxy-N—((S)-5-methyl-2-oxo-1-((S)—N—(((S)-2-oxopyrrolidin-3-yl)methyl)aziridine-2-carboxamido)hexan-3-yl)-1H-indole-2-carboxamide, Compound P18

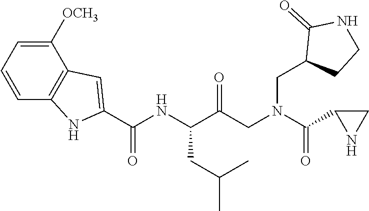

Compound P18 is prepared using the procedure outlined for Compound 20 in Example 20 from (23), replacing chloroacetyl chloride with (S)-1-trityl-aziridine-2-carboxylic acid and amide forming reaction conditions such as EDCI HCl, HOAt, Et₃N and DMF.

### Example P19: 4-Methoxy-N—((S)-5-methyl-2-oxo-1-((S)—N—(((S)-2-oxopyrrolidin-3-yl)methyl)aziridine-2-carboxamido)hexan-3-yl)-1H-indole-2-carboxamide, Compound P19

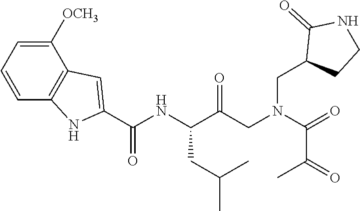 [SMILES: COc1cccc2[nH]c(C(=O)N[C@@H](CC(C)C)C(=O)CN(C[C@@H]3CCNC3=O)C(=O)C(C)=O)cc12]

Compound P19 is prepared using the procedure outlined below starting from (12).

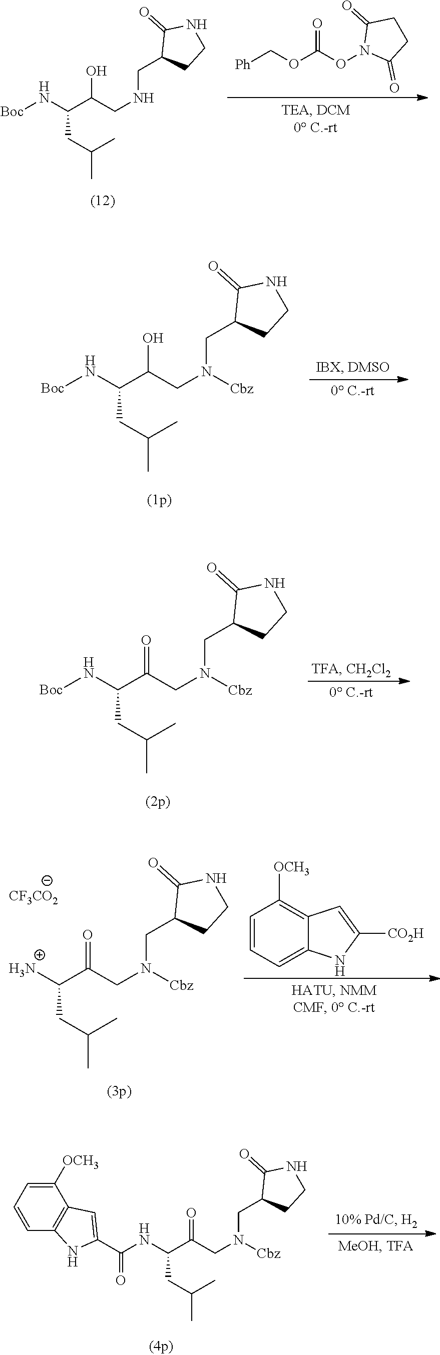

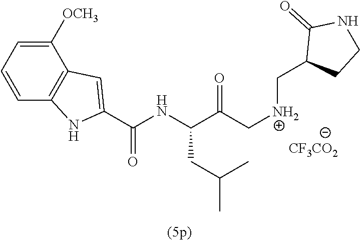

### 4-Methoxy-N—((S)-5-methyl-2-oxo-1-((((S)-2-oxopyrrolidin-3-yl)methyl)amino)hexan-3-yl)-1H-indole-2-carboxamide trifluoroacetic acid salt (5p) is converted to Compound P19 with 2-oxopropanoyl chloride under mild basic conditions

### Example P20: 4-Methoxy-N—((S)-5-methyl-2-oxo-1-(N—(((S)-2-oxopyrrolidin-3-yl)methyl)cyanamido)hexan-3-yl)-1H-indole-2-carboxamide, Compound P20

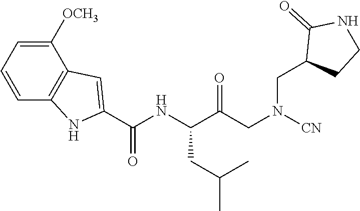 [SMILES: COc1cccc2[nH]c(C(=O)N[C@@H](CC(C)C)C(=O)CN(C#N)C[C@@H]3CCNC3=O)cc12]

Compound P20 is prepared using the procedure described for Compound P19, Example P19 from (5p) and cyanogen bromide.

### Example P21: Methyl ((S)-3-(4-methoxy-1H-indole-2-carboxamido)-5-methyl-2-oxohexyl)(((S)-2-oxopyrrolidin-3-yl)methyl)carbamodithioate, Compound P21

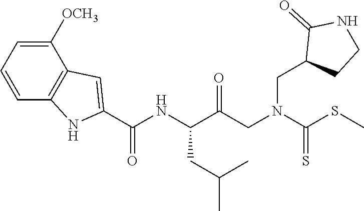 [SMILES: COc1cccc2[nH]c(C(=O)N[C@@H](CC(C)C)C(=O)CN(C[C@@H]3CCNC3=O)C(=S)SC)cc12]

Compound P21 is prepared using the procedure described for Compound P19, Example P19 from (5p), carbon disulfide and methyl iodide.

### Example P24: 2-Chloro-N—((S)-5-methyl-3-((S)-3-((1-methyl-1H-pyrazol-5-yl)amino)-2-oxopyrrolidin-1-yl)-2-oxohexyl)—N—(((S)-2-oxopyrrolidin-3-yl)methyl)acetamide, Compound P24

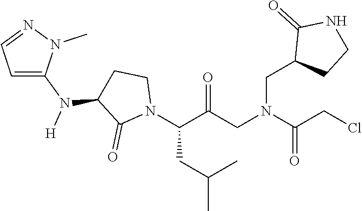 [SMILES: CC(C)C[C@@H](C(=O)CN(C[C@@H]1CCNC1=O)C(=O)CCl)N1CC[C@H](Nc2ccnn2C)C1=O]

Compound P24 is prepared using the procedures described in Example P22, by first converting methyl (S)-2-((S)-3-((tert-butoxycarbonyl)amino)-2-oxopyrrolidin-1-yl)-4-methylpentanoate in two steps to methyl (S)-4-methyl-2-((S)-3-((1-methyl-1H-pyrazol-5-yl)amino)-2-oxopyrrolidin-1-yl)pentanoate via methyl (S)-2-((R)-3-bromo-2-oxopyrrolidin-1-yl)-4-methylpentanoate and 1-methyl-1H-pyrazol-5-amine.

### Example P25: Tert-butyl (1-((S)-1-(2-chloro-N—(((S)-2-oxopyrrolidin-3-yl)methyl)acetamido)-2-oxohex-5-yn-3-yl)-2-oxo-1,2-dihydropyridin-3-yl)carbamate, Compound P25

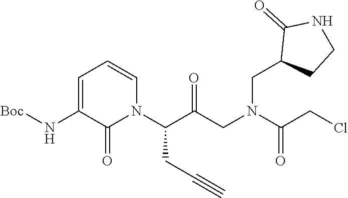 [SMILES: C#CC[C@@H](C(=O)CN(C[C@@H]1CCNC1=O)C(=O)CCl)n1cccc(NC(=O)OC(C)(C)C)c1=O]

Compound P25 is prepared using the procedures described in Example 1, replacing Boc-L-Leu with methyl (S)-2-(3-((tert-butoxycarbonyl)amino)-2-oxopyridin-1(2H)-yl)pent-4-ynoate, prepared from methyl (R)-2-(((trifluoromethyl)sulfonyl)oxy)pent-4-ynoate and tert-butyl (2-oxo-1,2-dihydropyridin-3-yl)carbamate according to Dragovich et al. JMC 2003, 47, 4572-4585.

### Example P26: Tert-butyl (1-((S)-1-(2-chloro-N—(((S)-2-oxopyrrolidin-3-yl)methyl)acetamido)-2-oxohexan-3-yl)-2-oxo-1,2-dihydropyridin-3-yl)carbamate, Compound P26

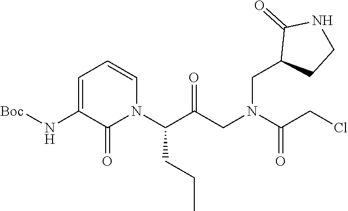 [SMILES: CCC[C@@H](C(=O)CN(C[C@@H]1CCNC1=O)C(=O)CCl)n1cccc(NC(=O)OC(C)(C)C)c1=O]

Compound P26 is prepared using the procedure described for Compound P25, Example P25 using methyl (S)-2-(3-((tert-butoxycarbonyl)amino)-2-oxopyridin-1(2H)-yl)pentanoate from the catalytic reduction of methyl (S)-2-(3-((tert-butoxycarbonyl)amino)-2-oxopyridin-1(2H)-yl)pent-4-ynoate.

### Example P27: N—((S)-1-((1R,2S,5S)-2-(N—(2-chloroacetyl)—N—(((S)-2-oxopyrrolidin-3-yl)methyl)glycyl)-6,6-dimethyl-3-azabicyclo[3.1.0]hexan-3-yl)-3,3-dimethyl-1-oxobutan-2-yl)-2,2,2-trifluoroacetamide, Compound P27

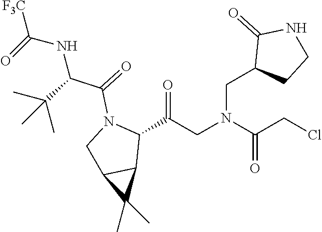 [SMILES: CC(C)(C)[C@H](NC(=O)C(F)(F)F)C(=O)N1C[C@H]2[C@@H]([C@H]1C(=O)CN(C[C@@H]1CCNC1=O)C(=O)CCl)C2(C)C]

Compound P27 is prepared using the procedures described in Examples 1 and 2, replacing Boc-L-Leu with (1R,2S,5S)-3-(tert-butoxycarbonyl)-6,6-dimethyl-3-azabicyclo[3.1.0]hexane-2-carboxylic acid [219754-02-6], and 4-methoxy-1H-indole-2-carboxylic acid with (S)-3,3-dimethyl-2-(2,2,2-trifluoroacetamido)butanoic acid.

### Example P28: N—((S)-1-((1R,2S,5S)-2-(N-cyano-N—(((S)-2-oxopyrrolidin-3-yl)methyl)glycyl)-6,6-dimethyl-3-azabicyclo[3.1.0]hexan-3-yl)-3,3-dimethyl-1-oxobutan-2-yl)-2,2,2-trifluoroacetamide, Compound P28

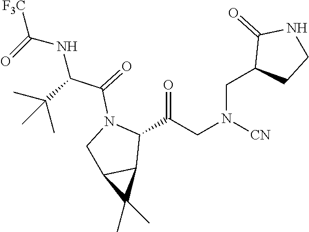 [SMILES: CC(C)(C)[C@H](NC(=O)C(F)(F)F)C(=O)N1C[C@H]2[C@@H]([C@H]1C(=O)CN(C#N)C[C@@H]1CCNC1=O)C2(C)C]

Using similar procedures described for Examples P19 and P20, Compound P28 is prepared from N—((S)-1-((1R,2S,5S)-6,6-dimethyl-2-((((S)-2-oxopyrrolidin-3-yl)methyl)glycyl)-3-azabicyclo[3.1.0]hexan-3-yl)-3,3-dimethyl-1-oxobutan-2-yl)-2,2,2-trifluoroacetamide and cyanogen bromide.

### Example P29: N—((S)-1-(2-(2-Chloroacetyl)-2-(((S)-2-oxopyrrolidin-3-yl)methyl)hydrazineyl)-4-methyl-1-oxopentan-2-yl)-4-methoxy-1H-indole-2-carboxamide, Compound P29

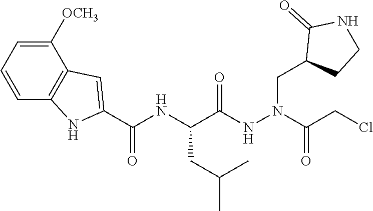 [SMILES: COc1cccc2[nH]c(C(=O)N[C@@H](CC(C)C)C(=O)NN(C[C@@H]3CCNC3=O)C(=O)CCl)cc12]

Compound P29 is prepared using the procedure outlined below starting from (9).

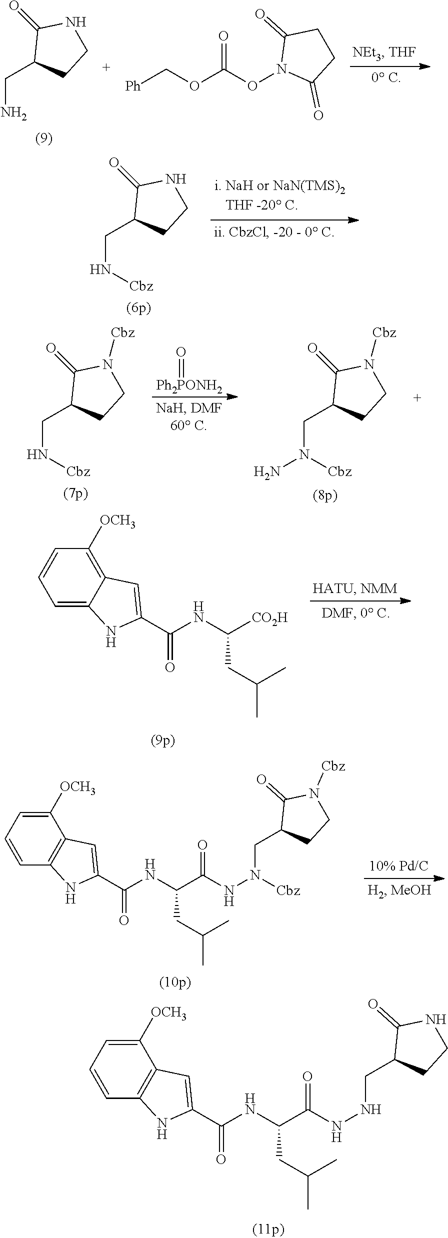

### 4-Methoxy-N—((S)-4-methyl-1-oxo-1-(2-(((S)-2-oxopyrrolidin-3-yl)methyl)hydrazineyl)pentan-2-yl)-1H-indole-2-carboxamide (lip) is converted to Compound P29 with chloroacetyl chloride using the conditions described to synthesize (13) in Example 1

### Example P30: N—((S)-1-(2-Acryloyl-2-(((S)-2-oxopyrrolidin-3-yl)methyl)hydrazineyl)-4-methyl-1-oxopentan-2-yl)-4-methoxy-1H-indole-2-carboxamide, Compound P30

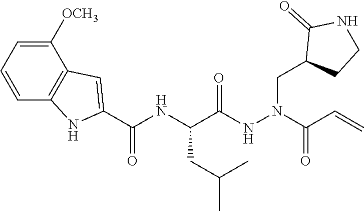 [SMILES: C=CC(=O)N(C[C@@H]1CCNC1=O)NC(=O)[C@H](CC(C)C)NC(=O)c1cc2c(OC)cccc2[nH]1]

### 4-Methoxy-N—((S)-4-methyl-1-oxo-1-(2-(((S)-2-oxopyrrolidin-3-yl)methyl)hydrazineyl)pentan-2-yl)-1H-indole-2-carboxamide (lip) is converted to Compound P30 with acryloyl chloride using the conditions described to synthesize (15) in Example 3

### Example P31: 4-Methoxy-N—((S)-4-methyl-1-oxo-1-(2-(((S)-2-oxopyrrolidin-3-yl)methyl)-2-propioloylhydrazineyl)pentan-2-yl)-1H-indole-2-carboxamide, Compound P31

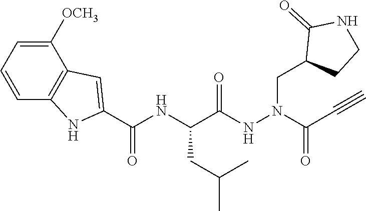 [SMILES: C#CC(=O)N(C[C@@H]1CCNC1=O)NC(=O)[C@H](CC(C)C)NC(=O)c1cc2c(OC)cccc2[nH]1]

### 4-Methoxy-N—((S)-4-methyl-1-oxo-1-(2-(((S)-2-oxopyrrolidin-3-yl)methyl)hydrazineyl)pentan-2-yl)-1H-indole-2-carboxamide (lip) is converted to Compound P31 with propiolic acid using the conditions described to synthesize (17) in Example 5

### Example P32: 4-Methoxy-N—((S)-4-methyl-1-oxo-1-(2-(2-oxopropanoyl)-2-(((S)-2-oxopyrrolidin-3-yl)methyl)hydrazineyl)pentan-2-yl)-1H-indole-2-carboxamide, Compound P32

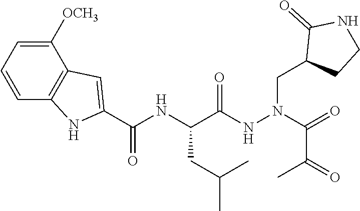 [SMILES: COc1cccc2[nH]c(C(=O)N[C@@H](CC(C)C)C(=O)NN(C[C@@H]3CCNC3=O)C(=O)C(C)=O)cc12]

### 4-Methoxy-N—((S)-4-methyl-1-oxo-1-(2-(((S)-2-oxopyrrolidin-3-yl)methyl)hydrazineyl)pentan-2-yl)-1H-indole-2-carboxamide (lip) is converted to Compound P32 with 2-oxopropanoyl chloride under mild basic conditions

### Example P33: Ethyl (E)-4-(2-((4-methoxy-1H-indole-2-carbonyl)-L-leucyl)-1-(((S)-2-oxopyrrolidin-3-yl)methyl)hydrazineyl)-4-oxobut-2-enoate, Compound P33

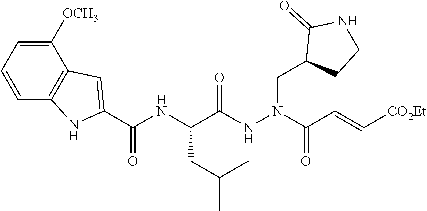 [SMILES: CCOC(=O)/C=C/C(=O)N(C[C@@H]1CCNC1=O)NC(=O)[C@H](CC(C)C)NC(=O)c1cc2c(OC)cccc2[nH]1]

4-Methoxy-N—((S)-4-methyl-1-oxo-1-(2-(((S)-2-oxopyrrolidin-3-yl)methyl)hydrazineyl)pentan-2-yl)-1H-indole-2-carboxamide (lip) is converted to Compound P33 with and (E)-4-ethoxy-4-oxobut-2-enoic acid [2459—O₅-4] using an amide forming reaction conditions with EDCI HCl, HOAt, Et₃N and DMF.

### Example P34: 4-Methoxy-N—((S)-4-methyl-1-oxo-1-(2-((E)-4-oxopent-2-enoyl)-2-(((S)-2-oxopyrrolidin-3-yl)methyl)hydrazineyl)pentan-2-yl)-1H-indole-2-carboxamide, Compound P34

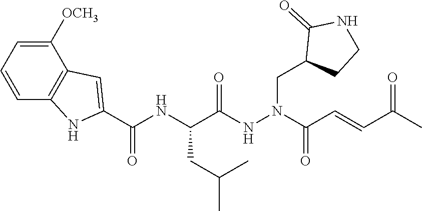 [SMILES: COc1cccc2[nH]c(C(=O)N[C@@H](CC(C)C)C(=O)NN(C[C@@H]3CCNC3=O)C(=O)/C=C/C(C)=O)cc12]

Compound P34 is prepared using the procedure outlined for Compound P33 in Example P33 substituting (E)-4-ethoxy-4-oxobut-2-enoic acid with (E)-4-oxopent-2-enoic acid.

### Example P35: N—((S)-1-(2-((Z)-3-Cyanoacryloyl)-2-(((S)-2-oxopyrrolidin-3-yl)methyl)hydrazineyl)-4-methyl-1-oxopentan-2-yl)-4-methoxy-1H-indole-2-carboxamide, Compound P35

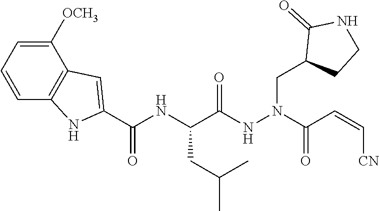 [SMILES: COc1cccc2[nH]c(C(=O)N[C@@H](CC(C)C)C(=O)NN(C[C@@H]3CCNC3=O)C(=O)/C=C\C#N)cc12]

Compound P35 is prepared using the procedure outlined for Compound P33 in Example P33 substituting (E)-4-ethoxy-4-oxobut-2-enoic acid with (Z)-3-cyanoacrylic acid.

### Example P36: 4-Methoxy-N—((S)-4-methyl-1-(2-((E)-3-(methylsulfonyl)acryloyl)-2-(((S)-2-oxopyrrolidin-3-yl)methyl)hydrazineyl)-1-oxopentan-2-yl)-1H-indole-2-carboxamide, Compound P36

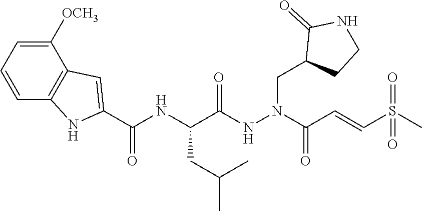 [SMILES: COc1cccc2[nH]c(C(=O)N[C@@H](CC(C)C)C(=O)NN(C[C@@H]3CCNC3=O)C(=O)/C=C/S(C)(=O)=O)cc12]

Compound P36 is prepared using the procedure outlined for Compound P33 in Example P33 substituting (E)-4-ethoxy-4-oxobut-2-enoic acid with (E)-3-(methylsulfonyl)acrylic acid.

### Example P37: S-Methyl 2-((4-methoxy-1H-indole-2-carbonyl)-L-leucyl)-1-(((S)-2-oxopyrrolidin-3-yl)methyl)hydrazine-1-carbothioate, Compound P37

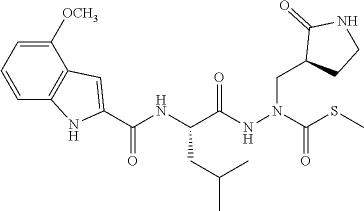 [SMILES: COc1cccc2[nH]c(C(=O)N[C@@H](CC(C)C)C(=O)NN(C[C@@H]3CCNC3=O)C(=O)SC)cc12]

### 4-Methoxy-N—((S)-4-methyl-1-oxo-1-(2-(((S)-2-oxopyrrolidin-3-yl)methyl)hydrazineyl)pentan-2-yl)-1H-indole-2-carboxamide (lip) is converted to Compound P37 with S-methyl carbonochloridothioate using similar conditions described to synthesize (21) in Example 16

### Example P38: 4-Methoxy-N—((2S)-4-methyl-1-(2-((methylsulfinyl)carbonyl)-2-(((S)-2-oxopyrrolidin-3-yl)methyl)hydrazineyl)-1-oxopentan-2-yl)-1H-indole-2-carboxamide, Compound P38

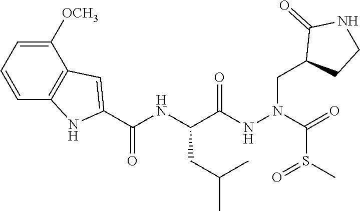 [SMILES: COc1cccc2[nH]c(C(=O)N[C@@H](CC(C)C)C(=O)NN(C[C@@H]3CCNC3=O)C(=O)S(C)=O)cc12]

Compound P38 is prepared by the oxidation of S-methyl 2-((4-methoxy-1H-indole-2-carbonyl)-L-leucyl)-1-(((S)-2-oxopyrrolidin-3-yl)methyl)hydrazine-1-carbothioate, Compound P37 with oxone, hydrogen peroxide or other oxidants.

### Example P39: 4-Methoxy-N—((S)-4-methyl-1-(2-((methylsulfonyl)carbonyl)-2-(((S)-2-oxopyrrolidin-3-yl)methyl)hydrazineyl)-1-oxopentan-2-yl)-1H-indole-2-carboxamide, Compound P39

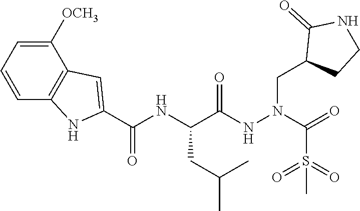 [SMILES: COc1cccc2[nH]c(C(=O)N[C@@H](CC(C)C)C(=O)NN(C[C@@H]3CCNC3=O)C(=O)S(C)(=O)=O)cc12]

Compound P39 is prepared by the oxidation of S-methyl 2-((4-methoxy-1H-indole-2-carbonyl)-L-leucyl)-1-(((S)-2-oxopyrrolidin-3-yl)methyl)hydrazine-1-carbothioate, Compound P37, or 4-methoxy-N—((2S)-4-methyl-1-(2-((methylsulfinyl)carbonyl)-2-(((S)-2-oxopyrrolidin-3-yl)methyl)hydrazineyl)-1-oxopentan-2-yl)-1H-indole-2-carboxamide, Compound P38 with oxone, hydrogen peroxide or other oxidants.

### Example P40: N—((S)-1-(2-((S)-Aziridine-2-carbonyl)-2-(((S)-2-oxopyrrolidin-3-yl)methyl)hydrazineyl)-4-methyl-1-oxopentan-2-yl)-4-methoxy-1H-indole-2-carboxamide, Compound P40

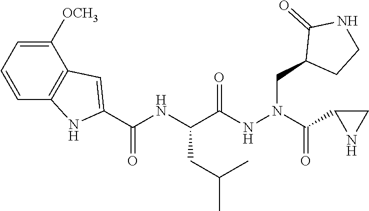

4-Methoxy-N—((S)-4-methyl-1-oxo-1-(2-(((S)-2-oxopyrrolidin-3-yl)methyl)hydrazineyl)pentan-2-yl)-1H-indole-2-carboxamide (11p) is converted to Compound P40 with (S)-1-trityl-aziridine-2-carboxylic acid under amide forming reaction conditions such as EDCI HCl, HOAt, Et₃N and DMF.

### Example P41: N—((S)-1-(2-(2-Cyanoacetyl)-2-(((S)-2-oxopyrrolidin-3-yl)methyl)hydrazineyl)-4-methyl-1-oxopentan-2-yl)-4-methoxy-1H-indole-2-carboxamide, Compound P41

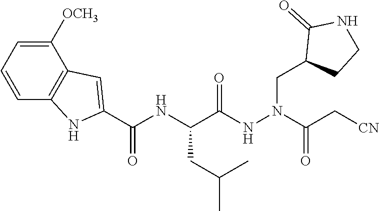 [SMILES: COc1cccc2[nH]c(C(=O)N[C@@H](CC(C)C)C(=O)NN(C[C@@H]3CCNC3=O)C(=O)CC#N)cc12]

4-Methoxy-N—((S)-4-methyl-1-oxo-1-(2-(((S)-2-oxopyrrolidin-3-yl)methyl)hydrazineyl)pentan-2-yl)-1H-indole-2-carboxamide (11p) is converted to Compound P41 with 2-cyanoacetic acid [372-09-8] under amide forming reaction conditions such as EDCI HCl, HOAt, Et₃N and DMF.

### Example P42: N—((S)-1-(2—(Cyanomethyl)-2-(((S)-2-oxopyrrolidin-3-yl)methyl)hydrazineyl)-4-methyl-1-oxopentan-2-yl)-4-methoxy-1H-indole-2-carboxamide, Compound P42

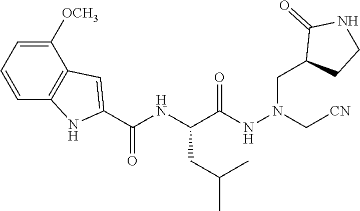 [SMILES: COc1cccc2[nH]c(C(=O)N[C@@H](CC(C)C)C(=O)NN(CC#N)C[C@@H]3CCNC3=O)cc12]

### 4-Methoxy-N—((S)-4-methyl-1-oxo-1-(2-(((S)-2-oxopyrrolidin-3-yl)methyl)hydrazineyl)pentan-2-yl)-1H-indole-2-carboxamide (lip) is converted to Compound P42 with 2-bromoacetonitrile and NEt₃

### Example P43: Methyl 2-((4-methoxy-1H-indole-2-carbonyl)-L-leucyl)-1-(((S)-2-oxopyrrolidin-3-yl)methyl)hydrazine-1-carbodithioate, Compound P43

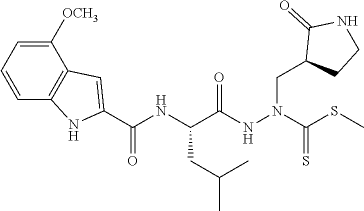 [SMILES: COc1cccc2[nH]c(C(=O)N[C@@H](CC(C)C)C(=O)NN(C[C@@H]3CCNC3=O)C(=S)SC)cc12]

### 4-Methoxy-N—((S)-4-methyl-1-oxo-1-(2-(((S)-2-oxopyrrolidin-3-yl)methyl)hydrazineyl)pentan-2-yl)-1H-indole-2-carboxamide (lip) is converted to Compound P43 with carbon disulfide and methyl iodide

### Example P44: N—((S)-1-(2-(2-Chloroacetyl)-2-(((S)-2-oxopiperidin-3-yl)methyl)hydrazineyl)-4-methyl-1-oxopentan-2-yl)-4-methoxy-1H-indole-2-carboxamide, Compound P44

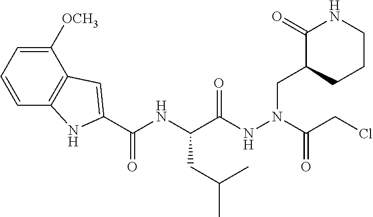 [SMILES: COc1cccc2[nH]c(C(=O)N[C@@H](CC(C)C)C(=O)NN(C[C@@H]3CCCNC3=O)C(=O)CCl)cc12]

Using a similar procedure described in Example P29 to prepare Compound P29, (S)-3-(aminomethyl)pyrrolidin-2-one (9) is replaced with (S)-3-(aminomethyl)piperidin-2-one to synthesize 4-methoxy-N—((S)-4-methyl-1-oxo-1-(2-(((S)-2-oxopiperidin-3-yl)methyl)hydrazineyl)pentan-2-yl)-1H-indole-2-carboxamide (12p), which is then further converted to Compound P44 with chloroacetyl chloride using the conditions described to synthesize (13) in Example 1.

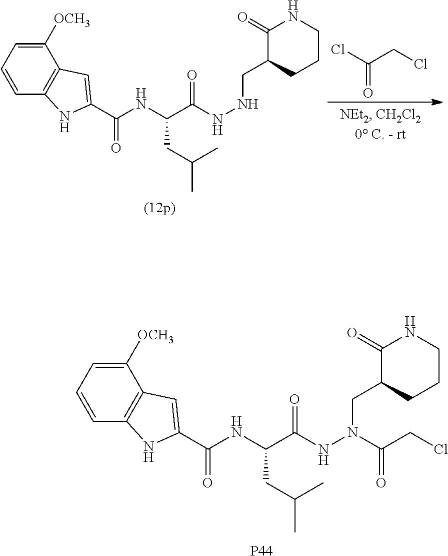

### Example P45: N—((S)-1-(2-Cyano-2-(((S)-2-oxopiperidin-3-yl)methyl)hydrazineyl)-4-methyl-1-oxopentan-2-yl)-4-methoxy-1H-indole-2-carboxamide, Compound P45

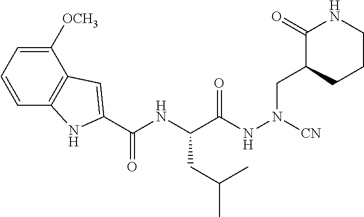 [SMILES: COc1cccc2[nH]c(C(=O)N[C@@H](CC(C)C)C(=O)NN(C#N)C[C@@H]3CCCNC3=O)cc12]

### 4-Methoxy-N—((S)-4-methyl-1-oxo-1-(2-(((S)-2-oxopiperidin-3-yl)methyl)hydrazineyl)pentan-2-yl)-1H-indole-2-carboxamide (12p) is converted to Compound P45 with cyanogen bromide

### Example P46: Benzyl ((R)-1-(2-(2-chloroacetyl)-2-(((S)-2-oxopyrrolidin-3-yl)methyl)hydrazineyl)-4-methyl-1-oxopentan-2-yl)carbamate, Compound P46

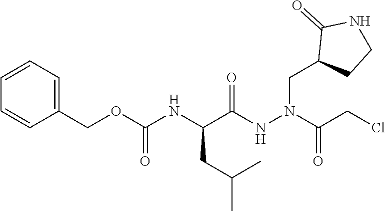 [SMILES: CC(C)C[C@@H](NC(=O)OCc1ccccc1)C(=O)NN(C[C@@H]1CCNC1=O)C(=O)CCl]

Using a similar procedure described in Example P29 to prepare Compound P29, (4-methoxy-1H-indole-2-carbonyl)-L-leucine (9p) is replaced with Cbz-D-Leu to synthesize benzyl ((R)-4-methyl-1-oxo-1-(2-(((S)-2-oxopyrrolidin-3-yl)methyl)hydrazineyl)pentan-2-yl)carbamate (13p), which is then further converted to Compound P46 with chloroacetyl chloride using the conditions described to synthesize (13) in Example 1.

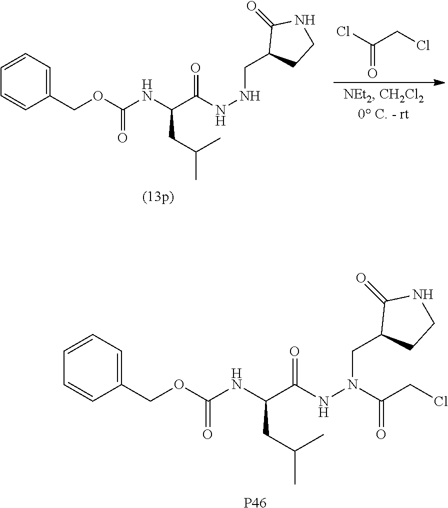

### Example P47: Tert-butyl ((S)-1-(2-(2-chloroacetyl)-2-(((S)-2-oxopyrrolidin-3-yl)methyl)hydrazineyl)-4-methyl-1-oxopentan-2-yl)carbamate, Compound P47

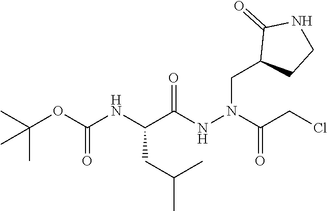 [SMILES: CC(C)C[C@H](NC(=O)OC(C)(C)C)C(=O)NN(C[C@@H]1CCNC1=O)C(=O)CCl]

Using a similar procedure described in Example P29 to prepare Compound P29, (4-methoxy-1H-indole-2-carbonyl)-L-leucine (9p) is replaced with Boc-L-Leu to synthesize tert-butyl ((S)-4-methyl-1-oxo-1-(2-(((S)-2-oxopyrrolidin-3-yl)methyl)hydrazineyl)pentan-2-yl)carbamate (14p), which is then further converted to Compound P47 with chloroacetyl chloride using the conditions described to synthesize (13) in Example 1.

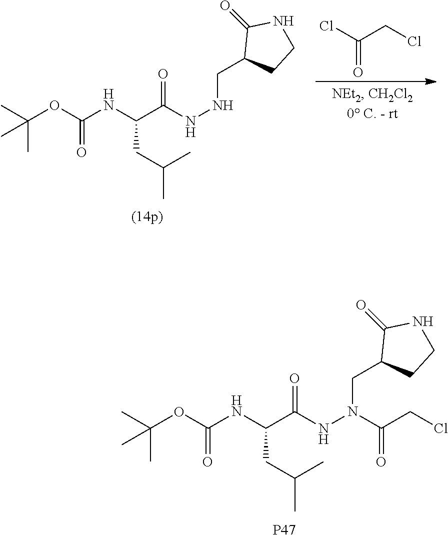

### Example P48: Cyclobutyl ((S)-1-(2-(2-chloroacetyl)-2-(((S)-2-oxopyrrolidin-3-yl)methyl)hydrazineyl)-4-methyl-1-oxopentan-2-yl)carbamate, Compound P48

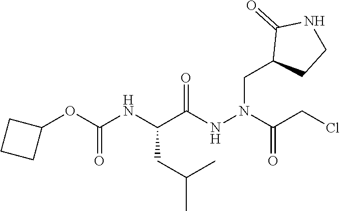 [SMILES: CC(C)C[C@H](NC(=O)OC1CCC1)C(=O)NN(C[C@@H]1CCNC1=O)C(=O)CCl]

Using a similar procedure described in Example P29 to prepare Compound P29, (4-methoxy-1H-indole-2-carbonyl)-L-leucine (9p) is replaced with methyl (cyclobutoxycarbonyl)-L-leucinate (30) to synthesize cyclobutyl ((S)-4-methyl-1-oxo-1-(2-(((S)-2-oxopyrrolidin-3-yl)methyl)hydrazineyl)pentan-2-yl)carbamate (15p), which is then further converted to Compound P48 with chloroacetyl chloride using the conditions described to synthesize (13) in Example 1.

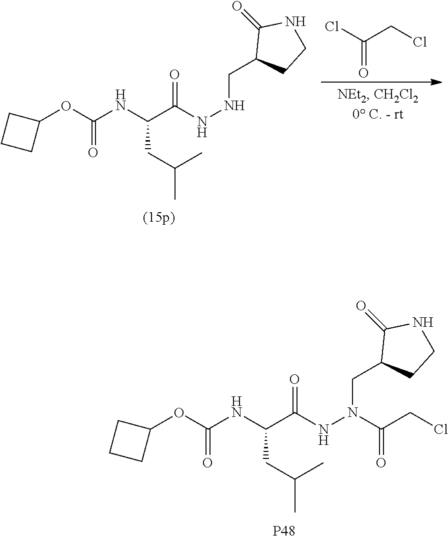

### Example P49: Tert-butyl (1-((5)-1-(2-(2-chloroacetyl)-2-(((S)-2-oxopyrrolidin-3-yl)methyl)hydrazineyl)-1-oxopent-4-yn-2-yl)-2-oxo-1,2-dihydropyridin-3-yl)carbamate, Compound P49

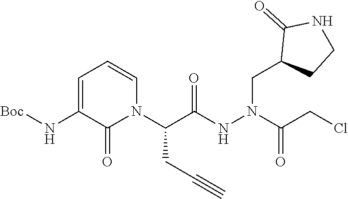 [SMILES: C#CC[C@@H](C(=O)NN(C[C@@H]1CCNC1=O)C(=O)CCl)n1cccc(NC(=O)OC(C)(C)C)c1=O]

Using a similar procedure described in Example P29 to prepare Compound P29, (4-methoxy-1H-indole-2-carbonyl)-L-leucine (9p) is replaced with (S)-2-(3-((tert-butoxycarbonyl)amino)-2-oxopyridin-1(2H)-yl)pent-4-ynoic acid (method of Dragovich et al. JMC 2003, 47, 4572-4585) to synthesize tert-butyl (2-oxo-1-((S)-1-oxo-1-(2-(((S)-2-oxopyrrolidin-3-yl)methyl)hydrazineyl)pent-4-yn-2-yl)-1,2-dihydropyridin-3-yl)carbamate (16p) which is then further converted to Compound P49 with chloroacetyl chloride using the conditions described to synthesize (13) in Example 1.

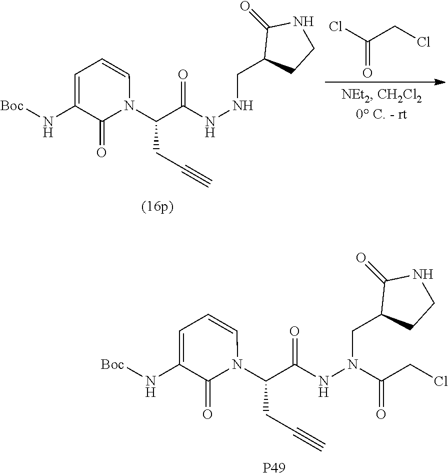

### Example P50: Tert-butyl (1-((S)-1-(2-cyano-2-(((S)-2-oxopyrrolidin-3-yl)methyl)hydrazineyl)-1-oxopent-4-yn-2-yl)-2-oxo-1,2-dihydropyridin-3-yl)carbamate, Compound P50

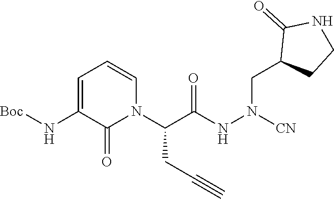 [SMILES: C#CC[C@@H](C(=O)NN(C#N)C[C@@H]1CCNC1=O)n1cccc(NC(=O)OC(C)(C)C)c1=O]

Tert-butyl (2-oxo-1-((S)-1-oxo-1-(2-(((S)-2-oxopyrrolidin-3-yl)methyl)hydrazineyl)pent-4-yn-2-yl)-1,2-dihydropyridin-3-yl)carbamate (16p) is converted to Compound P50 with cyanogen bromide.

### Example P51: N—((S)-1-((S)-1-(2-(2-Chloroacetyl)-2-(((S)-2-oxopyrrolidin-3-yl)methyl)hydrazineyl)-4-methyl-1-oxopentan-2-yl)-2-oxopyrrolidin-3-yl)acetamide, Compound P51

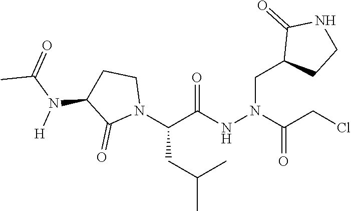 [SMILES: CC(=O)N[C@H]1CCN([C@@H](CC(C)C)C(=O)NN(C[C@@H]2CCNC2=O)C(=O)CCl)C1=O]

Compound P51 is prepared from reacting (S)-2-((S)-3-((tert-butoxycarbonyl)amino)-2-oxopyrrolidin-1-yl)-4-methylpentanoic acid (Freidinger et al. JOC 1982, 47, 104-109) and benzyl (S)-3-((1-((benzyloxy)carbonyl)hydrazineyl)methyl)-2-oxopyrrolidine-1-carboxylate (8p) using the methods described in Example P29, followed by removal of the t-butoxycarbonyl group of with trifluoroacetic acid and reaction of the primary amine salt with acetyl chloride under basic conditions.

### Example P52: N—((S)-1-((S)-1-(2-cyano-2-(((S)-2-oxopyrrolidin-3-yl)methyl)hydrazineyl)-4-methyl-1-oxopentan-2-yl)-2-oxopyrrolidin-3-yl)acetamide, Compound P52

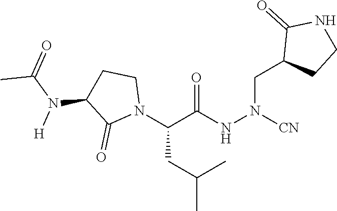 [SMILES: CC(=O)N[C@H]1CCN([C@@H](CC(C)C)C(=O)NN(C#N)C[C@@H]2CCNC2=O)C1=O]

The t-butoxycarbonyl group of (S)-2-((S)-3-((tert-butoxycarbonyl)amino)-2-oxopyrrolidin-1-yl)-4-methylpentanoic acid (Freidinger et al. JOC 1982, 47, 104-109) is removed with trifluoroacetic acid and converted to the corresponding (S)-2-((S)-3-acetamido-2-oxopyrrolidin-1-yl)-4-methylpentanoic acid. The carboxylic acid is converted in 2 steps to N—((S)-1-((S)-4-methyl-1-oxo-1-(2-(((S)-2-oxopyrrolidin-3-yl)methyl)hydrazineyl)pentan-2-yl)-2-oxopyrrolidin-3-yl)acetamide (17p) using an amide forming reaction with benzyl (S)-3-((1-((benzyloxy)carbonyl)hydrazineyl)methyl)-2-oxopyrrolidine-1-carboxylate (8p), followed by catalytic hydrogenation using the methods described in Example P29. N—((S)-1-((S)-4-Methyl-1-oxo-1-(2-(((S)-2-oxopyrrolidin-3-yl)methyl)hydrazineyl)pentan-2-yl)-2-oxopyrrolidin-3-yl)acetamide (17p) is transformed into Compound P52 by reaction with cyanogen bromide.

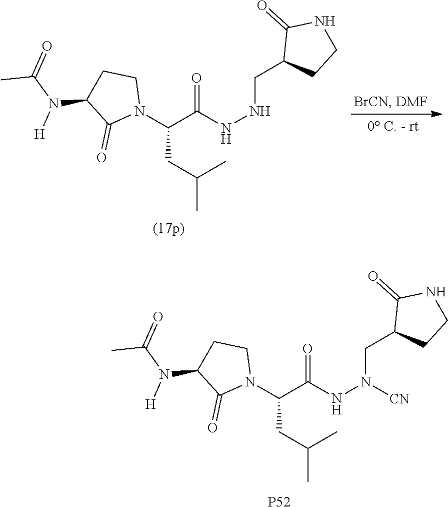

### Example P53: (S)—N′-(2-Chloroacetyl)-4-methyl-2-((S)-3-((1-methyl-1H-pyrazol-5-yl)amino)-2-oxopyrrolidin-1-yl)—N′-(((S)-2-oxopyrrolidin-3-yl)methyl)pentanehydrazide, CompoundP53

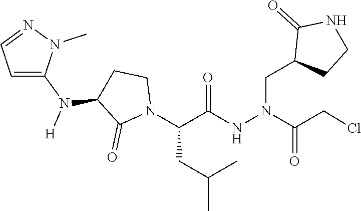 [SMILES: CC(C)C[C@@H](C(=O)NN(C[C@@H]1CCNC1=O)C(=O)CCl)N1CC[C@H](Nc2ccnn2C)C1=O]

Using procedures similar to those described in Example P52, Compound P53 is synthesized from methyl (S)-4-methyl-2-((S)-3-((1-methyl-1H-pyrazol-5-yl)amino)-2-oxopyrrolidin-1-yl)pentanoate, benzyl (S)-3-((1-((benzyloxy)carbonyl)hydrazineyl)methyl)-2-oxopyrrolidine-1-carboxylate (8p), and chloroacetyl chloride.

### Example P54: (S)—N′—Cyano-4-methyl-2-((S)-3-((1-methyl-1H-pyrazol-5-yl)amino)-2-oxopyrrolidin-1-yl)—N′-(((S)-2-oxopyrrolidin-3-yl)methyl)pentanehydrazide, Compound P54

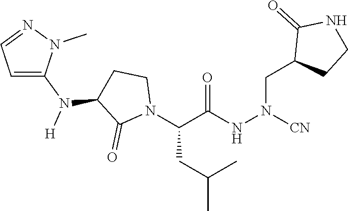 [SMILES: CC(C)C[C@@H](C(=O)NN(C#N)C[C@@H]1CCNC1=O)N1CC[C@H](Nc2ccnn2C)C1=O]

Using procedures similar to those described in Example P52, Compound P54 is synthesized from methyl (S)-4-methyl-2-((S)-3-((1-methyl-1H-pyrazol-5-yl)amino)-2-oxopyrrolidin-1-yl)pentanoate, benzyl (S)-3-((1-((benzyloxy)carbonyl)hydrazineyl)methyl)-2-oxopyrrolidine-1-carboxylate (8p), and cyanogen bromide.

### Example P55: N—((S)-1-((1R,2S,5S)-2-(2-(2-chloroacetyl)-2-(((S)-2-oxopyrrolidin-3-yl)methyl)hydrazine-1-carbonyl)-6,6-dimethyl-3-azabicyclo[3.1.0]hexan-3-yl)-3,3-dimethyl-1-oxobutan-2-yl)-2,2,2-trifluoroacetamide, Compound P55

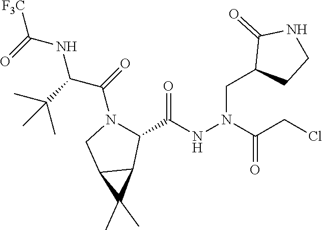 [SMILES: CC(C)(C)[C@H](NC(=O)C(F)(F)F)C(=O)N1C[C@H]2[C@@H]([C@H]1C(=O)NN(C[C@@H]1CCNC1=O)C(=O)CCl)C2(C)C]

Using a similar procedure described in Example P29 to prepare Compound P29, (4-methoxy-1H-indole-2-carbonyl)-L-leucine (9p) is replaced with (1R,2S,5S)-3-((S)-3,3-dimethyl-2-(2,2,2-trifluoroacetamido)butanoyl)-6,6-dimethyl-3-azabicyclo[3.1.0]hexane-2-carboxylic acid (WO2021/250648A1) to synthesize N—((S)-1-((1R,2S,5S)-6,6-dimethyl-2-(2-(((S)-2-oxopyrrolidin-3-yl)methyl)hydrazine-1-carbonyl)-3-azabicyclo[3.1.0]hexan-3-yl)-3,3-dimethyl-1-oxobutan-2-yl)-2,2,2-trifluoroacetamide (18p) which is then further converted to Compound P55 with chloroacetyl chloride using the conditions described to synthesize (13) in Example 1.

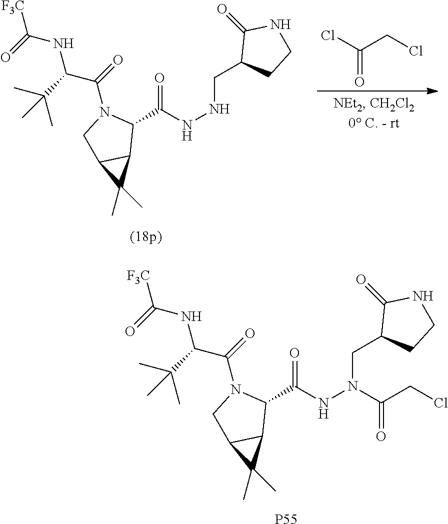

### Example P56: N—((S)-1-((1R,2S,5S)-2-(2-cyano-2-(((S)-2-oxopyrrolidin-3-yl)methyl)hydrazine-1-carbonyl)-6,6-dimethyl-3-azabicyclo[3.1.0]hexan-3-yl)-3,3-dimethyl-1-oxobutan-2-yl)-2,2,2-trifluoroacetamide, Compound P56

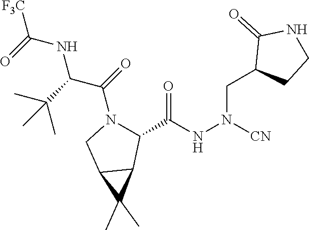 [SMILES: CC(C)(C)[C@H](NC(=O)C(F)(F)F)C(=O)N1C[C@H]2[C@@H]([C@H]1C(=O)NN(C#N)C[C@@H]1CCNC1=O)C2(C)C]

N—((S)-1-((1R,2S,5S)-6,6-Dimethyl-2-(2-(((S)-2-oxopyrrolidin-3-yl)methyl)hydrazine-1-carbonyl)-3-azabicyclo[3.1.0]hexan-3-yl)-3,3-dimethyl-1-oxobutan-2-yl)-2,2,2-trifluoroacetamide (18p) is converted to Compound P56 with cyanogen bromide.

### Example P57: N—((S)-1-((1R,2S,5S)-2-(2-(2-chloroacetyl)-2-(((S)-2-oxopiperidin-3-yl)methyl)hydrazine-1-carbonyl)-6,6-dimethyl-3-azabicyclo[3.1.0]hexan-3-yl)-3,3-dimethyl-1-oxobutan-2-yl)-2,2,2-trifluoroacetamide, Compound P57

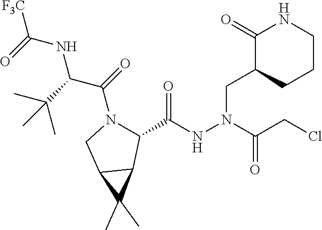 [SMILES: CC(C)(C)[C@H](NC(=O)C(F)(F)F)C(=O)N1C[C@H]2[C@@H]([C@H]1C(=O)NN(C[C@@H]1CCCNC1=O)C(=O)CCl)C2(C)C]

Using a similar procedure described in Example P29 to prepare Compound P29, (4-methoxy-1H-indole-2-carbonyl)-L-leucine (9p) is replaced with (1R,2S,5S)-3-((S)-3,3-dimethyl-2-(2,2,2-trifluoroacetamido)butanoyl)-6,6-dimethyl-3-azabicyclo[3.1.0]hexane-2-carboxylic acid (WO2021/250648A1), and (S)-3-(aminomethyl)pyrrolidin-2-one (9) is replaced with (S)-3-(aminomethyl)piperidin-2-one to synthesize N—((S)-1-((1R,2S,5S)-6,6-dimethyl-2-(2-(((S)-2-oxopiperidin-3-yl)methyl)hydrazine-1-carbonyl)-3-azabicyclo[3.1.0]hexan-3-yl)-3,3-dimethyl-1-oxobutan-2-yl)-2,2,2-trifluoroacetamide (19p), which is then further converted to Compound P57 with chloroacetyl chloride using the conditions described to synthesize (13) in Example 1.

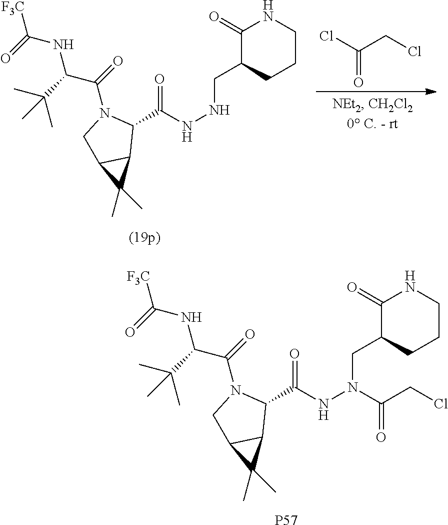

### Example P58: N—((S)-1-((1R,2S,5S)-2-(2-cyano-2-(((S)-2-oxopiperidin-3-yl)methyl)hydrazine-1-carbonyl)-6,6-dimethyl-3-azabicyclo[3.1.0]hexan-3-yl)-3,3-dimethyl-1-oxobutan-2-yl)-2,2,2-trifluoroacetamide, Compound P58

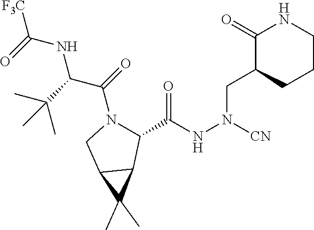 [SMILES: CC(C)(C)[C@H](NC(=O)C(F)(F)F)C(=O)N1C[C@H]2[C@@H]([C@H]1C(=O)NN(C#N)C[C@@H]1CCCNC1=O)C2(C)C]

N—((S)-1-((1R,2S,5S)-6,6-dimethyl-2-(2-(((S)-2-oxopiperidin-3-yl)methyl)hydrazine-1-carbonyl)-3-azabicyclo[3.1.0]hexan-3-yl)-3,3-dimethyl-1-oxobutan-2-yl)-2,2,2-trifluoroacetamide (19p) is converted to Compound P58 with cyanogen bromide. Biological Assays

### Profiling in a Biochemical 3CLpro Protease Assay

Compounds were profiled in a fluorescence intensity based biochemical protease assay using 3CLpro (Polaris Pharmaceuticals, Inc.) and the quenched substrate peptide (SEQ. ID NO: 1) Dabcyl-KTSAVLQSGFRKME-Edans (MedChemExpress, cat# HY-P2295). Reactions were performed in 50 mM Tris-HCl (pH 7.5), 100 mM NaCl, 1 mM DTT and 0.01% Pluronic™ F-127. Briefly, an 11-point 3-fold serial dilution of the corresponding compound in DMSO (final top concentration: 100 μM) was spotted into the assay plate (Corning #4514, 384-well Low Volume Black Round Bottom Polystyrene NBS) and incubated with 40 nM 3CLpro for 30 minutes at room temperature. Subsequently, 35 μM substrate peptide was added and the proteolytic reaction was incubated for 60 minutes at 37° C. Afterwards, the fluorescence intensity was detected on a Safire II Multi-Detection Plate Reader (Tecan) using an excitation wavelength of 360 nm and an emission wavelength of 585 nm. Data was normalized based on wells containing DMSO as high/negative control and wells containing no enzyme as low/positive control. IC₅₀ values were obtained using evaluation software (APlus with a four-parameter non-linear regression fit model).

TABLE 1

| Example No. | IC₅₀ (μM) |
|---|---|
| 1 | A |
| 2 | A |
| 3 | C |
| 4 | C |
| 6 | B |
| 8 | C |
| 10 | C |
| 11 | A |
| 12 | A |
| 13 | A |
| 14 | A |
| 15 | A |
| 16 | C |
| 17 | C |
| 18 | C |
| 20 | A |
| 21 | B |

A = ≤2.00 μM;
B = 2.00 μM < x ≤ 20.0 μM;
C = >20.0 μM

All references cited herein are incorporated by reference to the same extent as if each individual publication, patent application, or patent, was specifically and individually indicated to be incorporated by reference. This statement of incorporation by reference is intended by Applicants, pursuant to 37 C.F.R. § 1.57(b)(1), to relate to each and every individual publication, patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

What is claimed is:

1. A compound having the structure of Formula I: 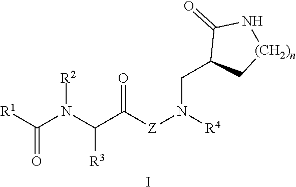
wherein
R¹ is —H, alkyl, —O-alkyl, aryl, alkylene-aryl, —O-aryl, cycloalkyl, alkylene-cycloalkyl, —O— cycloalkyl, heterocyclyl, alkylene-heterocyclyl, —O-heterocyclyl, or —NR⁵R⁶;
R² is —H or alkyl;
or R¹ and R², together with the atoms to which they are bonded, can form a 5- or 6-membered heterocyclic ring;
R³ is —H, alkyl, —CH₂-alkenyl or —CH₂-alkynyl;
or R² and R³, together with the atoms to which they are bonded, can form a 5- or 6-membered heterocyclic ring;
R⁴ is —CN, —C(O)R⁷, —C(S)R⁷, —SO₂R⁸, —C(O)CH₂R⁹, —SO₂CH₂R⁸ or —CH₂R¹⁰;
each of R⁵ and R⁶ are independently —H, alkyl, alkenyl, alkynyl, alkylene-X, —C(O)alkyl, —O— alkyl, or —S(O)ₘalkyl;
or R⁵ and R⁶, when bonded to the same atom can, together with the atom to which they are bonded, form a heterocyclic ring;
R⁷ is —H, alkyl, alkenyl, alkynyl, cycloalkyl, heterocyclyl, alkylene-X, alkenyl-C(O)Oalkyl, alkenyl-S(O)ₘalkyl, —C(O)alkyl, —O-alkyl, —S(O)ₘalkyl, or —S(O)ₘalkylene-aryl;
R⁸ is alkenyl, alkynyl or alkylene-X;
R⁹ is halogen, —CN or —OC(O)alkyl;
R¹⁰ is halogen or —CN;
X is halogen or —OC(O)aryl;
Z is —CH₂—, —NCH₃—, or —NH—;
m is 0, 1, or 2; and
n is 1 or 2;
wherein when Z is —NH— and R³ is alkyl, R⁴ is then —C(O)R⁷, —C(S)R⁷, —SO₂R⁸, —C(O)CH₂R⁹, —SO₂CH₂R⁸ or —CH₂R¹⁰; and,
wherein each alkyl, alkenyl, alkynyl, alkylene, aryl, cycloalkyl, and heterocyclyl is independently optionally substituted with 1, 2 or 3 groups selected from —OH, —CN, —(C₁-C₄) alkylNHC(O)(C₁-C₄)haloalkyl, alkylene-aryl-NHC(O)heteroaryl, —SH, —S(O)NH₂, halogen, —NH₂, —NH(C₁-C₄)alkyl, —N[(C₁-C₄)alkyl]2, —C(O)NH₂, —COOH, —COOMe, acetyl, —(C₁-C₅)alkyl, —O(C₁-C₅)alkyl, (C₂-C₅)alkenyl, (C₂-C₅)alkynyl, thioalkyl, cyanomethylene, —NH-heterocyclyl, —NH—C(O)(C₁-C₄)alkyl, —NH—C(O)— heterocycloalkyl, —NH-heterocycloalkyl, —NH—C(O)-alkylene, —NH—C(O)—O-alkylene, —CH₂-C(O)-alkyl, —C(O)-alkyl, cycloalkyl, —C(O)-cycloalkyl, —CH₂-C(O)-aryl, —CH₂-aryl, —C(O)-aryl, —C(O)-heterocycloalkyl, —CH₂-C(O)-heterocyclyl, —C(O)— heterocyclyl, or heterocyclyl;
or a stereoisomer, a tautomer or a pharmaceutically acceptable salt thereof.

2. The compound of claim 1, wherein R¹ is —O(C₁-C₄)alkyl, —O-alkylene-aryl, —CH[(C₁-C₄)alkyl]NHC(O)(C₁-C₄)haloalkyl, —CH(alkylene-aryl)NHC(O)heteroaryl, 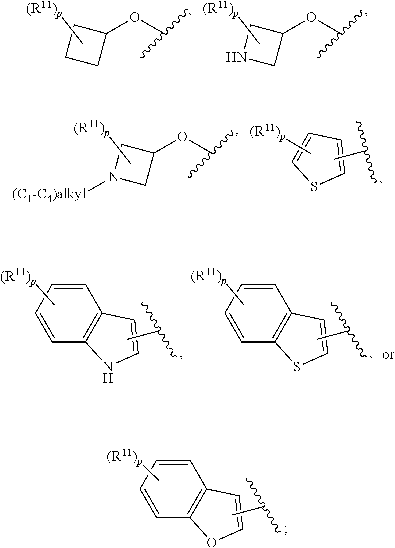
R¹¹ is —H, alkyl, alkenyl, alkynyl, halo, haloalkyl, —O(C₁-C₄)alkyl, —NH₂, —NH(C₁-C₄)alkyl]₂; and, —N[(C₁-C₄)alkyl]2; and,
p is 0, 1 or 2;
or a stereoisomer, a tautomer or a pharmaceutically acceptable salt thereof.

3. The compound of claim 1, wherein R is: 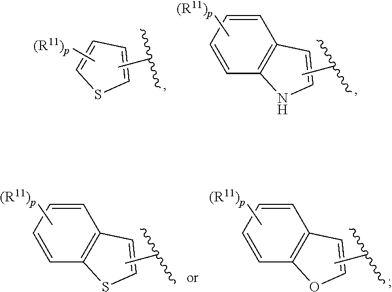
stereoisomer, a tautomer or a pharmaceutically acceptable salt thereof.

4. The compound of claim 1, wherein R¹ is: 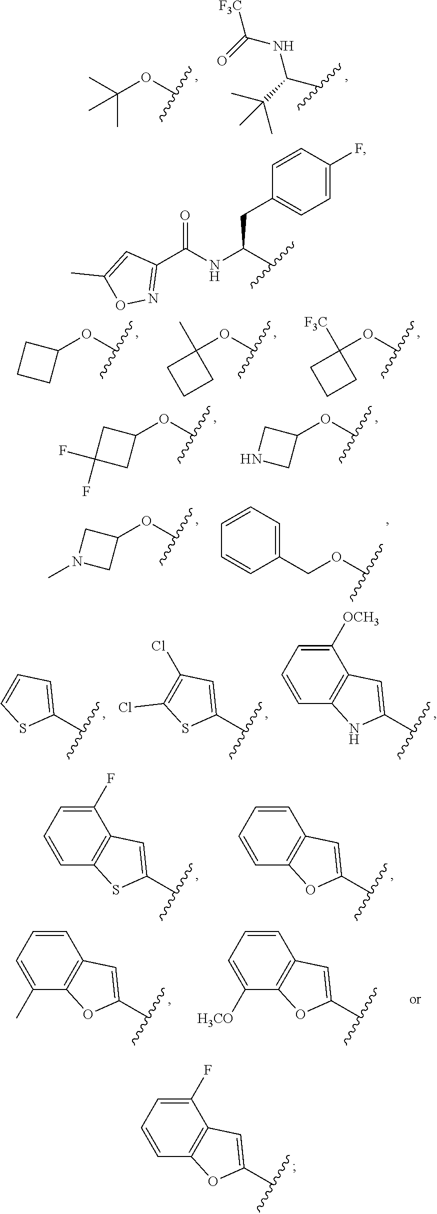
or a stereoisomer, a tautomer or a pharmaceutically acceptable salt thereof.

5. The compound of claim 1, wherein R¹ and R², together with the atoms to which they are bonded, form an optionally substituted 5- or 6-membered heterocyclic ring which is: 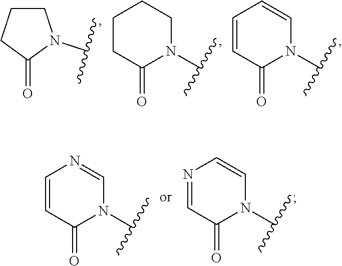
or a stereoisomer, a tautomer or a pharmaceutically acceptable salt thereof.

6. The compound of claim 5, wherein R¹ and R², together with the atoms to which they are bonded, for a 5- or 6-membered heteroaromatic ring or a stereoisomer, a tautomer or a pharmaceutically acceptable salt thereof.

7. The compound of claim 1, wherein R³ is alkyl, or a stereoisomer, a tautomer or a pharmaceutically acceptable salt thereof.

8. The compound of claim 1, wherein R³ is isobutyl, propyne-3-yl, n-propyl, 1-methylpropane, or 1-methylcylohexane, or a stereoisomer, a tautomer or a pharmaceutically acceptable salt thereof.

9. The compound of claim 1, wherein R² and R³, together with the atoms to which they are bonded, form a 5- or 6-membered heterocyclic ring which is: 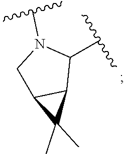 [SMILES: *C1[C@@H]2[C@H](CN1*)C2(C)C]
or a stereoisomer, a tautomer or a pharmaceutically acceptable salt thereof.

10. The compound of claim 1, wherein R⁴ is —C(O)R⁷, or a stereoisomer, a tautomer or a pharmaceutically acceptable salt thereof.

11. The compound of claim 1, wherein R⁴ is —SO₂R⁸, or a stereoisomer, a tautomer or a pharmaceutically acceptable salt thereof.

12. The compound of claim 1, wherein R⁴ is: 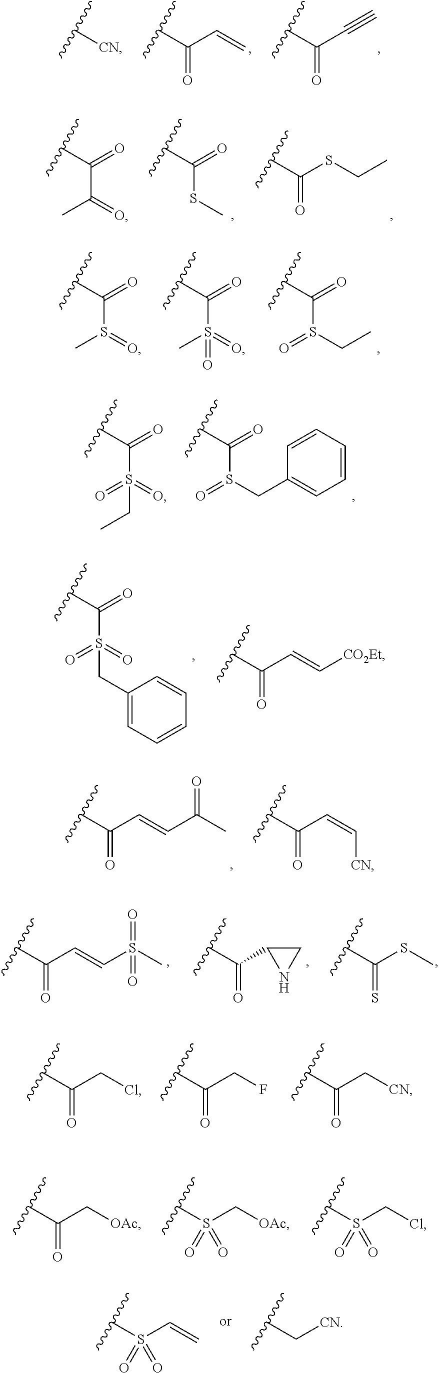
or a stereoisomer, a tautomer or a pharmaceutically acceptable salt thereof.

13. The compound of claim 1, wherein X is —OC(O)aryl, or a stereoisomer, a tautomer or a pharmaceutically acceptable salt thereof.

14. The compound of claim 1, wherein Z is —CH₂—, or a stereoisomer, a tautomer or a pharmaceutically acceptable salt thereof.

15. A compound having a structure which is: 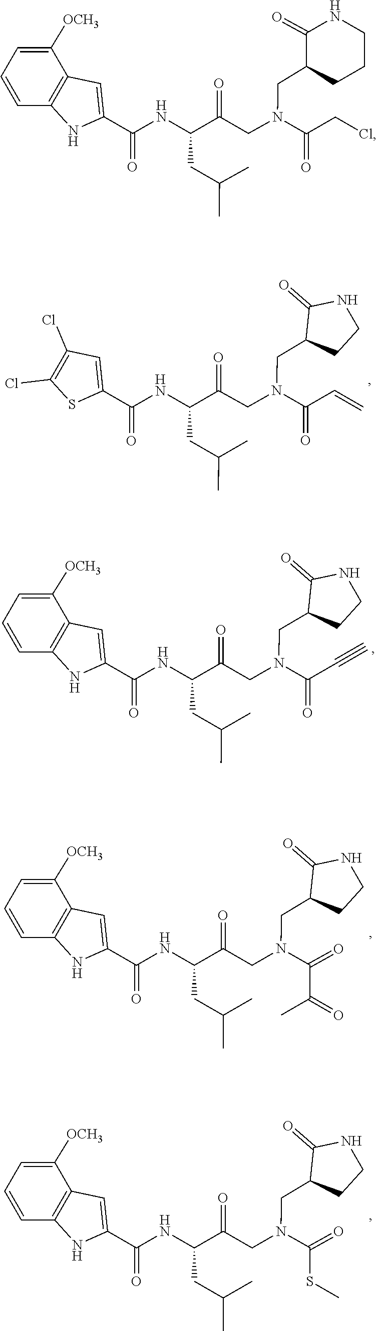 [SMILES: COc1cccc2[nH]c(C(=O)N[C@@H](CC(C)C)C(=O)CN(C[C@@H]3CCCNC3=O)C(=O)CCl)cc12 ; C=CC(=O)N(CC(=O)[C@H](CC(C)C)NC(=O)c1cc(Cl)c(Cl)s1)C[C@@H]1CCNC1=O ; C#CC(=O)N(CC(=O)[C@H](CC(C)C)NC(=O)c1cc2c(OC)cccc2[nH]1)C[C@@H]1CCNC1=O ; COc1cccc2[nH]c(C(=O)N[C@@H](CC(C)C)C(=O)CN(C[C@@H]3CCNC3=O)C(=O)C(C)=O)cc12 ; COc1cccc2[nH]c(C(=O)N[C@@H](CC(C)C)C(=O)CN(C[C@@H]3CCNC3=O)C(=O)SC)cc12] 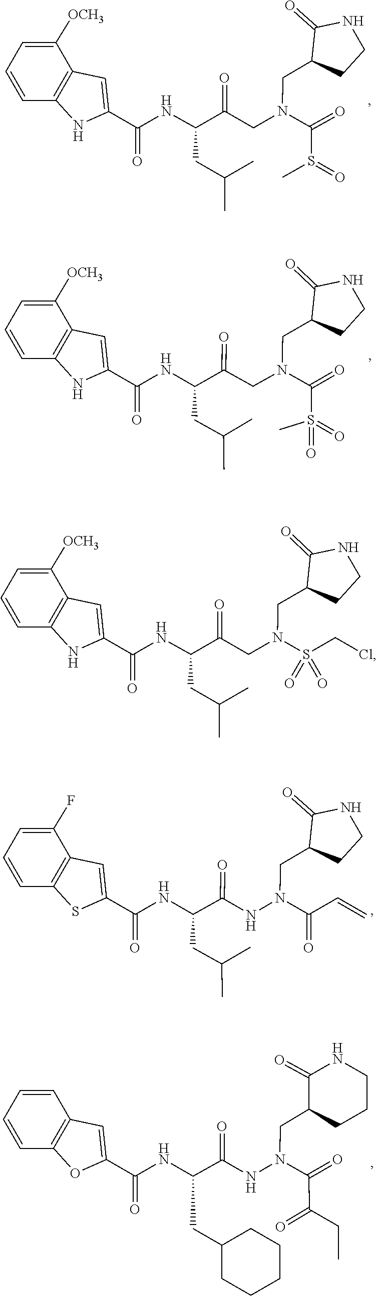 [SMILES: COc1cccc2[nH]c(C(=O)N[C@@H](CC(C)C)C(=O)CN(C[C@@H]3CCNC3=O)C(=O)S(C)=O)cc12 ; COc1cccc2[nH]c(C(=O)N[C@@H](CC(C)C)C(=O)CN(C[C@@H]3CCNC3=O)C(=O)S(C)(=O)=O)cc12 ; COc1cccc2[nH]c(C(=O)N[C@@H](CC(C)C)C(=O)CN(C[C@@H]3CCNC3=O)S(=O)(=O)CCl)cc12 ; C=CC(=O)N(C[C@@H]1CCNC1=O)NC(=O)[C@H](CC(C)C)NC(=O)c1cc2c(F)cccc2s1 ; CCC(=O)C(=O)N(C[C@@H]1CCCNC1=O)NC(=O)[C@H](CC1CCCCC1)NC(=O)c1cc2ccccc2o1] 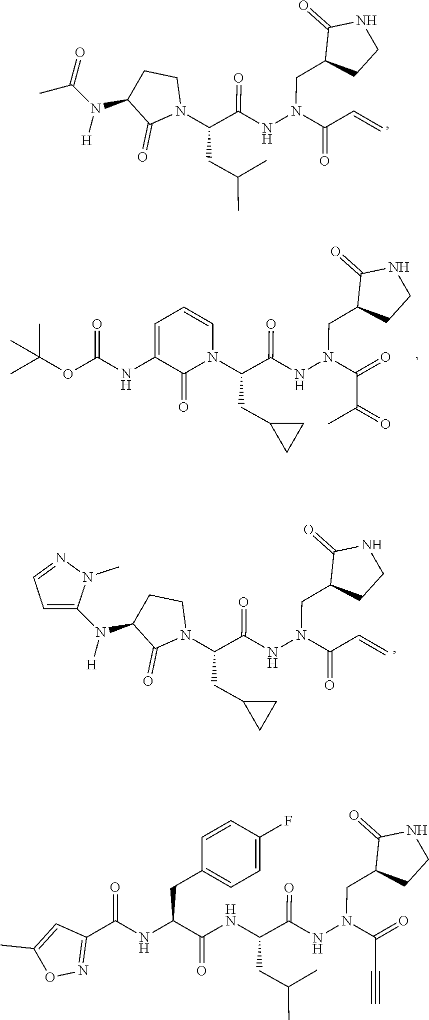 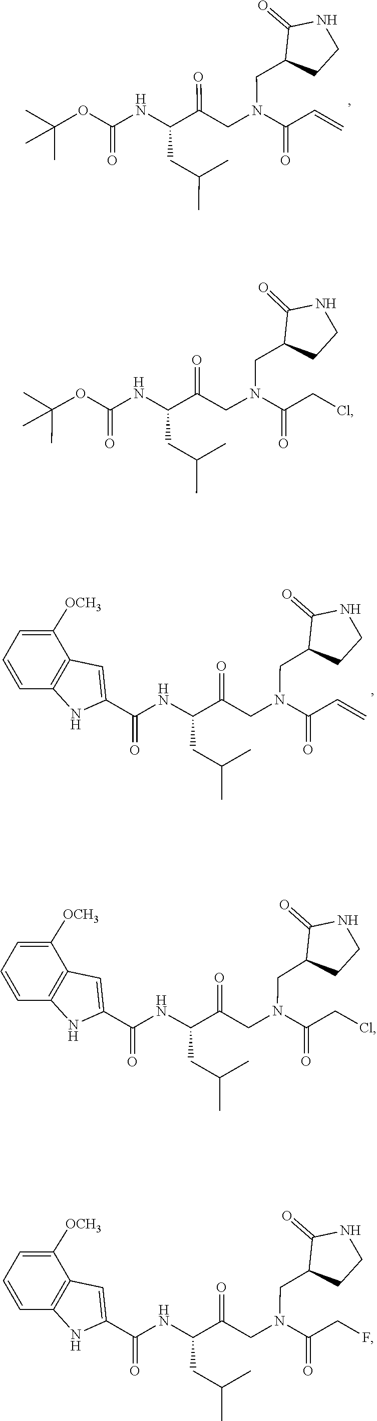 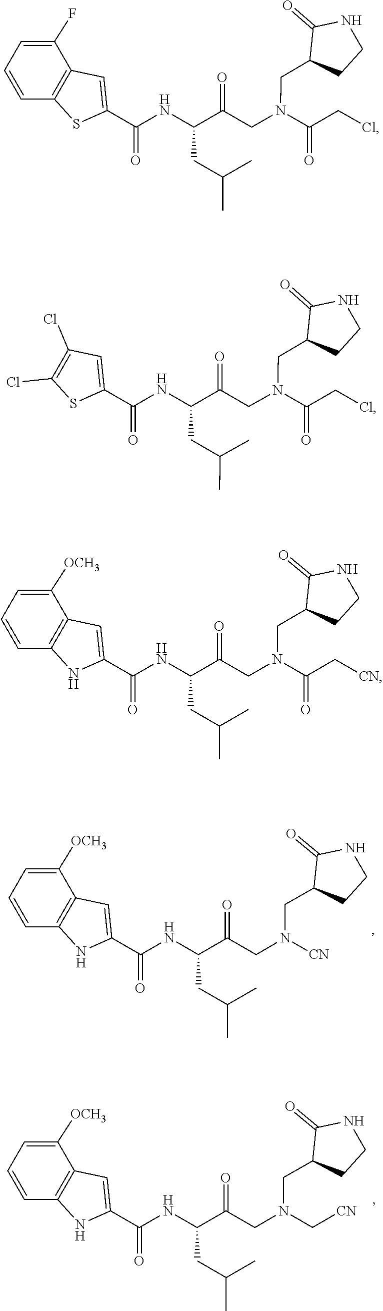 [SMILES: CC(C)C[C@H](NC(=O)c1cc2c(F)cccc2s1)C(=O)CN(C[C@@H]1CCNC1=O)C(=O)CCl ; CC(C)C[C@H](NC(=O)c1cc(Cl)c(Cl)s1)C(=O)CN(C[C@@H]1CCNC1=O)C(=O)CCl ; COc1cccc2[nH]c(C(=O)N[C@@H](CC(C)C)C(=O)CN(C[C@@H]3CCNC3=O)C(=O)CC#N)cc12 ; COc1cccc2[nH]c(C(=O)N[C@@H](CC(C)C)C(=O)CN(C#N)C[C@@H]3CCNC3=O)cc12 ; COc1cccc2[nH]c(C(=O)N[C@@H](CC(C)C)C(=O)CN(CC#N)C[C@@H]3CCNC3=O)cc12] 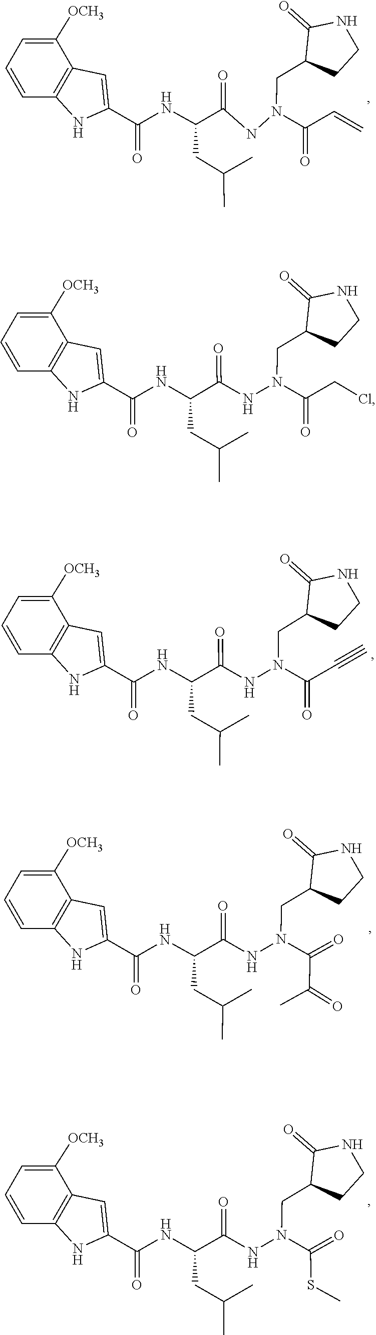 [SMILES: C=CC(=O)N(C[C@@H]1CCNC1=O)NC(=O)[C@H](CC(C)C)NC(=O)c1cc2c(OC)cccc2[nH]1 ; COc1cccc2[nH]c(C(=O)N[C@@H](CC(C)C)C(=O)NN(C[C@@H]3CCNC3=O)C(=O)CCl)cc12 ; C#CC(=O)N(C[C@@H]1CCNC1=O)NC(=O)[C@H](CC(C)C)NC(=O)c1cc2c(OC)cccc2[nH]1 ; COc1cccc2[nH]c(C(=O)N[C@@H](CC(C)C)C(=O)NN(C[C@@H]3CCNC3=O)C(=O)C(C)=O)cc12 ; COc1cccc2[nH]c(C(=O)N[C@@H](CC(C)C)C(=O)NN(C[C@@H]3CCNC3=O)C(=O)SC)cc12] 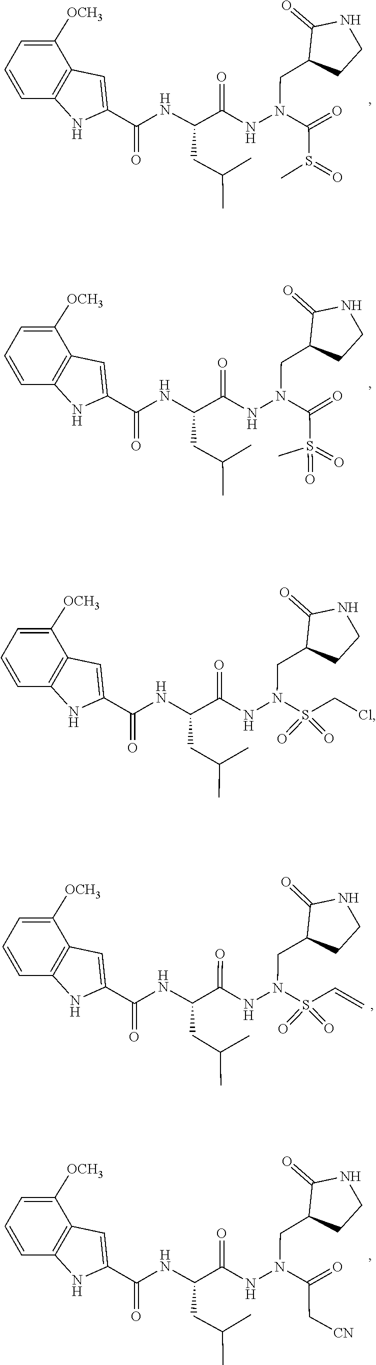 [SMILES: COc1cccc2[nH]c(C(=O)N[C@@H](CC(C)C)C(=O)NN(C[C@@H]3CCNC3=O)C(=O)S(C)=O)cc12 ; COc1cccc2[nH]c(C(=O)N[C@@H](CC(C)C)C(=O)NN(C[C@@H]3CCNC3=O)C(=O)S(C)(=O)=O)cc12 ; COc1cccc2[nH]c(C(=O)N[C@@H](CC(C)C)C(=O)NN(C[C@@H]3CCNC3=O)S(=O)(=O)CCl)cc12 ; C=CS(=O)(=O)N(C[C@@H]1CCNC1=O)NC(=O)[C@H](CC(C)C)NC(=O)c1cc2c(OC)cccc2[nH]1 ; COc1cccc2[nH]c(C(=O)N[C@@H](CC(C)C)C(=O)NN(C[C@@H]3CCNC3=O)C(=O)CC#N)cc12] 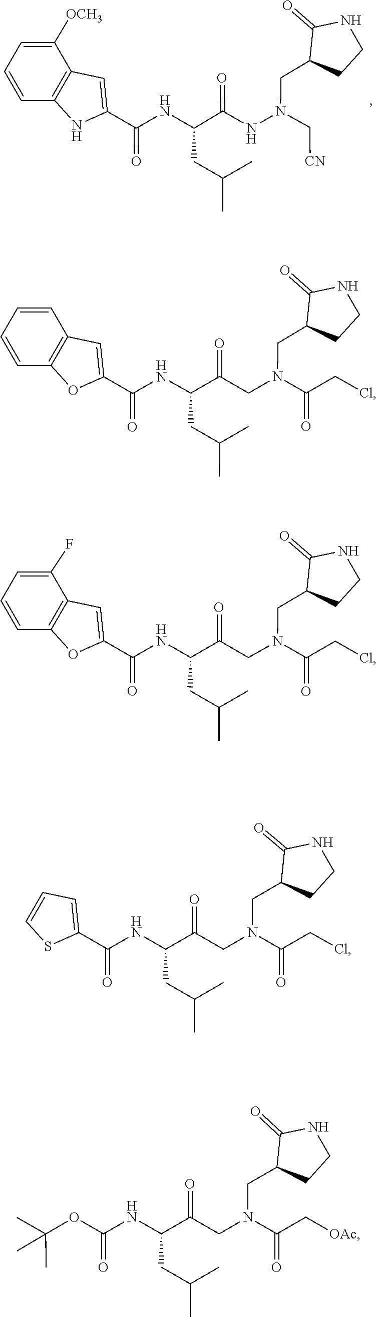 [SMILES: COc1cccc2[nH]c(C(=O)N[C@@H](CC(C)C)C(=O)NN(CC#N)C[C@@H]3CCNC3=O)cc12 ; CC(C)C[C@H](NC(=O)c1cc2ccccc2o1)C(=O)CN(C[C@@H]1CCNC1=O)C(=O)CCl ; CC(C)C[C@H](NC(=O)c1cc2c(F)cccc2o1)C(=O)CN(C[C@@H]1CCNC1=O)C(=O)CCl ; CC(C)C[C@H](NC(=O)c1cccs1)C(=O)CN(C[C@@H]1CCNC1=O)C(=O)CCl ; CC(=O)OCC(=O)N(CC(=O)[C@H](CC(C)C)NC(=O)OC(C)(C)C)C[C@@H]1CCNC1=O] 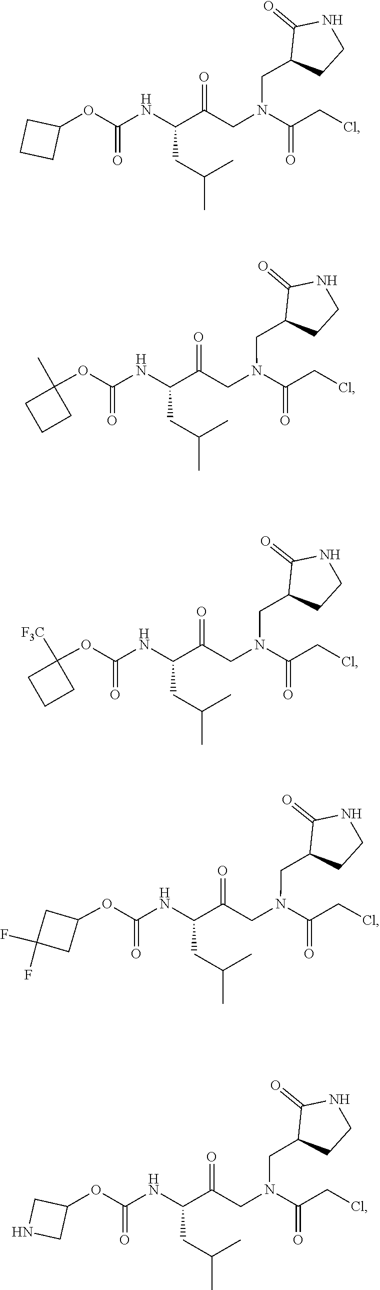 [SMILES: CC(C)C[C@H](NC(=O)OC1CCC1)C(=O)CN(C[C@@H]1CCNC1=O)C(=O)CCl ; CC(C)C[C@H](NC(=O)OC1(C)CCC1)C(=O)CN(C[C@@H]1CCNC1=O)C(=O)CCl ; CC(C)C[C@H](NC(=O)OC1(C(F)(F)F)CCC1)C(=O)CN(C[C@@H]1CCNC1=O)C(=O)CCl ; CC(C)C[C@H](NC(=O)OC1CC(F)(F)C1)C(=O)CN(C[C@@H]1CCNC1=O)C(=O)CCl ; CC(C)C[C@H](NC(=O)OC1CNC1)C(=O)CN(C[C@@H]1CCNC1=O)C(=O)CCl] 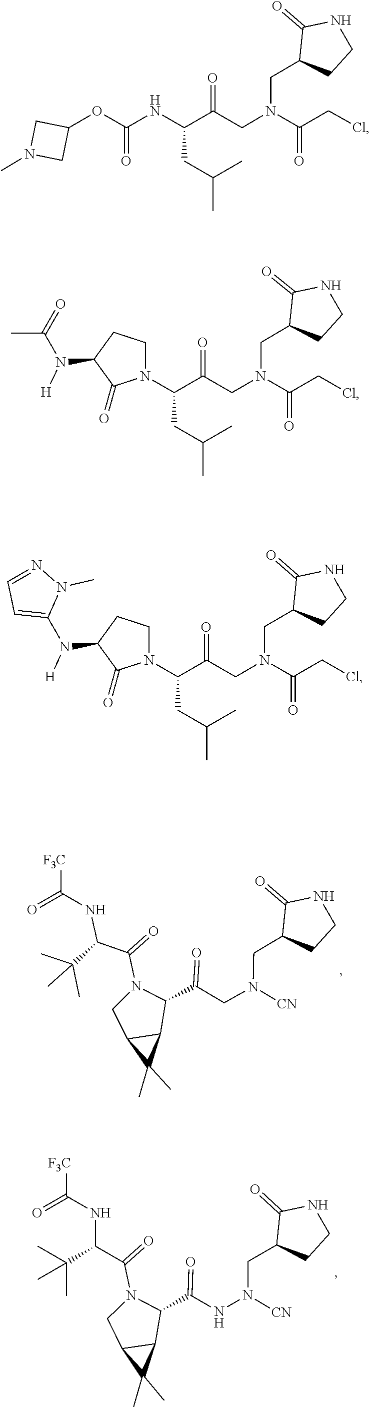 [SMILES: CC(C)C[C@H](NC(=O)OC1CN(C)C1)C(=O)CN(C[C@@H]1CCNC1=O)C(=O)CCl ; CC(=O)N[C@H]1CCN([C@@H](CC(C)C)C(=O)CN(C[C@@H]2CCNC2=O)C(=O)CCl)C1=O ; CC(C)C[C@@H](C(=O)CN(C[C@@H]1CCNC1=O)C(=O)CCl)N1CC[C@H](Nc2ccnn2C)C1=O ; CC(C)(C)[C@H](NC(=O)C(F)(F)F)C(=O)N1C[C@H]2[C@@H]([C@H]1C(=O)CN(C#N)C[C@@H]1CCNC1=O)C2(C)C ; CC(C)(C)[C@H](NC(=O)C(F)(F)F)C(=O)N1C[C@H]2[C@@H]([C@H]1C(=O)NN(C#N)C[C@@H]1CCNC1=O)C2(C)C] 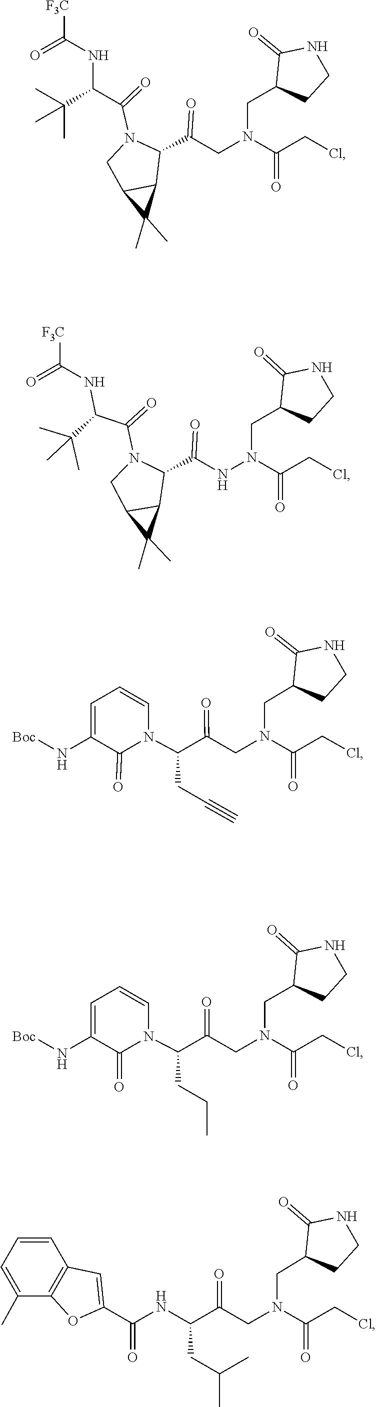 [SMILES: CC(C)(C)[C@H](NC(=O)C(F)(F)F)C(=O)N1C[C@H]2[C@@H]([C@H]1C(=O)CN(C[C@@H]1CCNC1=O)C(=O)CCl)C2(C)C ; CC(C)(C)[C@H](NC(=O)C(F)(F)F)C(=O)N1C[C@H]2[C@@H]([C@H]1C(=O)NN(C[C@@H]1CCNC1=O)C(=O)CCl)C2(C)C ; C#CC[C@@H](C(=O)CN(C[C@@H]1CCNC1=O)C(=O)CCl)n1cccc(NC(=O)OC(C)(C)C)c1=O ; CCC[C@@H](C(=O)CN(C[C@@H]1CCNC1=O)C(=O)CCl)n1cccc(NC(=O)OC(C)(C)C)c1=O ; Cc1cccc2cc(C(=O)N[C@@H](CC(C)C)C(=O)CN(C[C@@H]3CCNC3=O)C(=O)CCl)oc12] 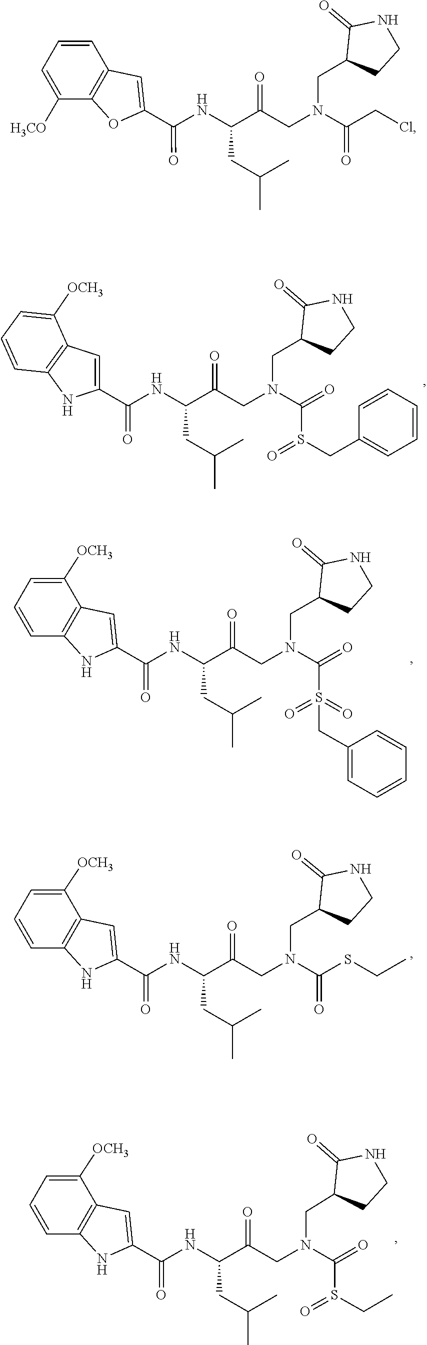 [SMILES: COc1cccc2cc(C(=O)N[C@@H](CC(C)C)C(=O)CN(C[C@@H]3CCNC3=O)C(=O)CCl)oc12 ; COc1cccc2[nH]c(C(=O)N[C@@H](CC(C)C)C(=O)CN(C[C@@H]3CCNC3=O)C(=O)S(=O)Cc3ccccc3)cc12 ; COc1cccc2[nH]c(C(=O)N[C@@H](CC(C)C)C(=O)CN(C[C@@H]3CCNC3=O)C(=O)S(=O)(=O)Cc3ccccc3)cc12 ; CCSC(=O)N(CC(=O)[C@H](CC(C)C)NC(=O)c1cc2c(OC)cccc2[nH]1)C[C@@H]1CCNC1=O ; CCS(=O)C(=O)N(CC(=O)[C@H](CC(C)C)NC(=O)c1cc2c(OC)cccc2[nH]1)C[C@@H]1CCNC1=O] 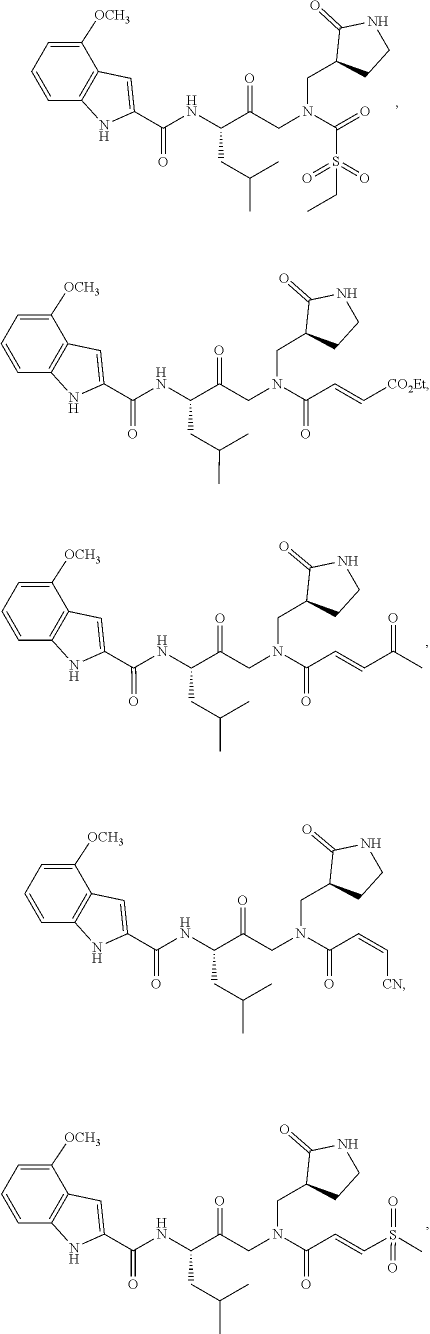 [SMILES: CCS(=O)(=O)C(=O)N(CC(=O)[C@H](CC(C)C)NC(=O)c1cc2c(OC)cccc2[nH]1)C[C@@H]1CCNC1=O ; CCOC(=O)/C=C/C(=O)N(CC(=O)[C@H](CC(C)C)NC(=O)c1cc2c(OC)cccc2[nH]1)C[C@@H]1CCNC1=O ; COc1cccc2[nH]c(C(=O)N[C@@H](CC(C)C)C(=O)CN(C[C@@H]3CCNC3=O)C(=O)/C=C/C(C)=O)cc12 ; COc1cccc2[nH]c(C(=O)N[C@@H](CC(C)C)C(=O)CN(C[C@@H]3CCNC3=O)C(=O)/C=C\C#N)cc12 ; COc1cccc2[nH]c(C(=O)N[C@@H](CC(C)C)C(=O)CN(C[C@@H]3CCNC3=O)C(=O)/C=C/S(C)(=O)=O)cc12] 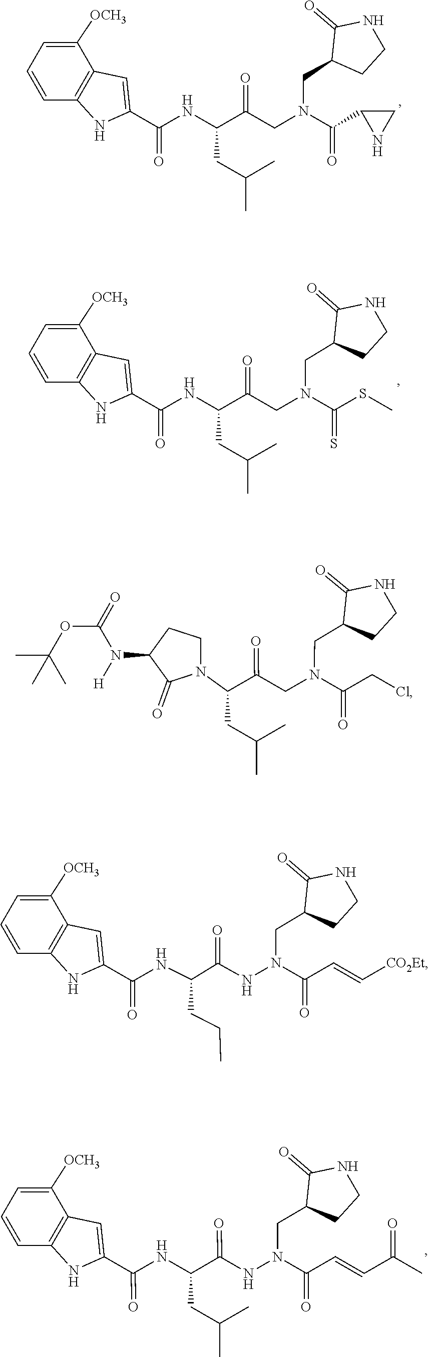 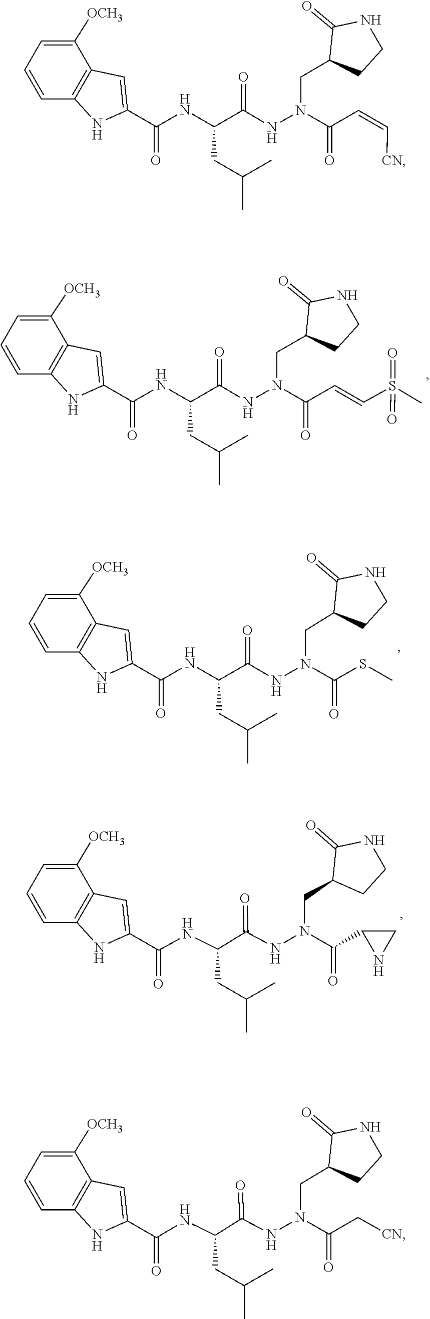 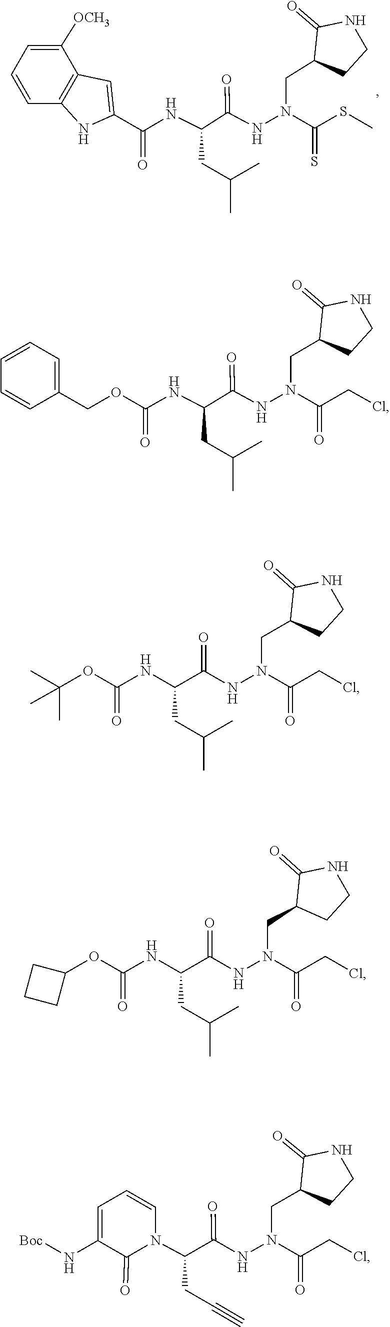 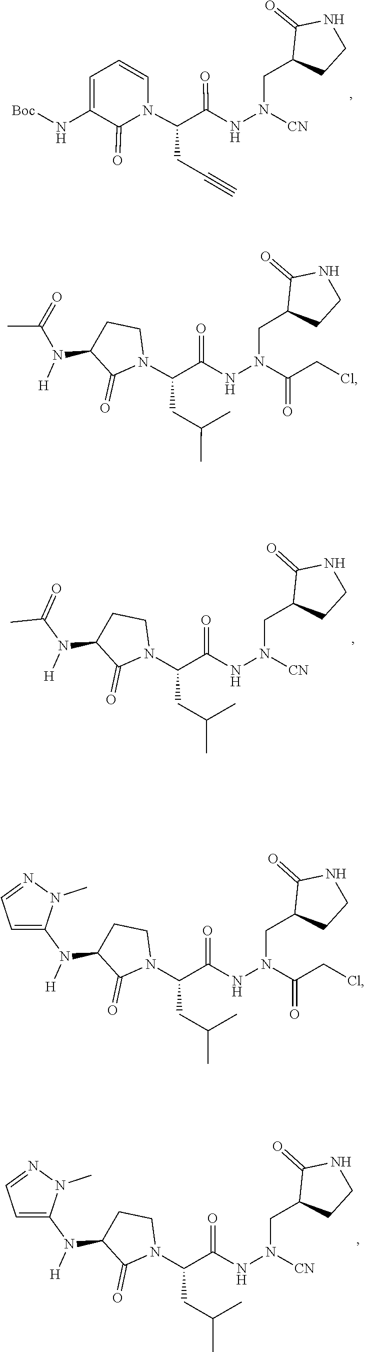 [SMILES: C#CC[C@@H](C(=O)NN(C#N)C[C@@H]1CCNC1=O)n1cccc(NC(=O)OC(C)(C)C)c1=O ; CC(=O)N[C@H]1CCN([C@@H](CC(C)C)C(=O)NN(C[C@@H]2CCNC2=O)C(=O)CCl)C1=O ; CC(=O)N[C@H]1CCN([C@@H](CC(C)C)C(=O)NN(C#N)C[C@@H]2CCNC2=O)C1=O ; CC(C)C[C@@H](C(=O)NN(C[C@@H]1CCNC1=O)C(=O)CCl)N1CC[C@H](Nc2ccnn2C)C1=O ; CC(C)C[C@@H](C(=O)NN(C#N)C[C@@H]1CCNC1=O)N1CC[C@H](Nc2ccnn2C)C1=O] 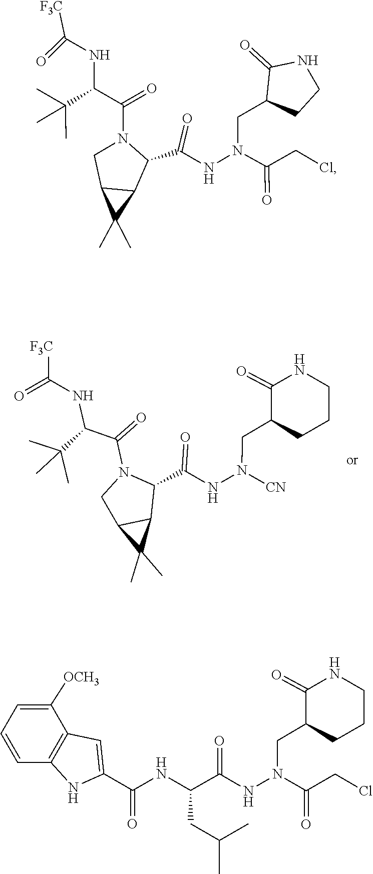 [SMILES: CC(C)(C)[C@H](NC(=O)C(F)(F)F)C(=O)N1C[C@H]2[C@@H]([C@H]1C(=O)NN(C[C@@H]1CCNC1=O)C(=O)CCl)C2(C)C ; CC(C)(C)[C@H](NC(=O)C(F)(F)F)C(=O)N1C[C@H]2[C@@H]([C@H]1C(=O)NN(C#N)C[C@@H]1CCCNC1=O)C2(C)C ; COc1cccc2[nH]c(C(=O)N[C@@H](CC(C)C)C(=O)NN(C[C@@H]3CCCNC3=O)C(=O)CCl)cc12]
or a stereoisomer, a tautomer or a pharmaceutically acceptable salt thereof.

16. A pharmaceutical composition comprising one or more compounds of claim 1, or a stereoisomer, a tautomer or a pharmaceutically acceptable salt thereof, and a pharmaceutically acceptable excipient.

17. A method for treating an individual suffering from a viral infection, the method comprising administering to the individual an effective amount of one or more compounds of claim 1, or a stereoisomer, a tautomer or a pharmaceutically acceptable salt thereof.

18. The method of claim 17, wherein the viral infection is associated with a virus selected form the group consisting of rhinovirus, adenovirus, influenza virus, respiratory syncytial virus, enterovirus D68, enterovirus A71, Coxsackievirus A16, the etiological agents of hand, foot, and mouth disease, (HFMD), Coxsackievirus B3, hepatitis C virus (HCV), West Nile virus, Sindbis virus (SINV), dengue virus, Ebola virus, Marburg virus, Crimean-Congo hemorrhagic fever like orthonairovirus (CCHFV), yellow fever virus, Rift Valley fever virus (RVFV), Omsk hemorrhagic fever virus (OHFV), Kyasanur Forest disease virus (KFDV), Junin virus, Machupo virus, Sabia virus, Guanarito virus, Garissa virus, Ilesha virus, Lassa fever virus, severe acute respiratory syndrome coronavirus (SARS-CoV), Middle East respiratory syndrome coronavirus (MERS-CoV), and severe acute respiratory syndrome coronavirus 2 (SARS-CoV-2).

19. The method of claim 17, wherein the viral infection is associated with SARS-CoV-2.

20. The method of claim 17, wherein the viral infection is associate with a virus in the family Coronaviridae.
